(19) United States
(12) Patent Application Publication
(10) Pub. No.: US 2022/0347268 A1
(43) Pub. Date: Nov. 3, 2022

(54) IL-4R AS A BIOMARKER IN CANCER

(71) Applicant: Medicenna Therapeutics, Inc., Toronto (CA)

(72) Inventors: Fahar Merchant, Vancouver (CA); Rosemina Merchant, Toronto (CA)

(21) Appl. No.: 17/428,697

(22) PCT Filed: Feb. 7, 2020
(86) PCT No.: PCT/CA2020/000013

§ 371 Date: Aug. 5, 2021

Related U.S. Application Data
(60) Provisional application No. 62/802,652, filed on Feb. 7, 2019.

(51) Int. Cl. A61K 38/20; G01N 33/574; G01N 33/68; C07K 14/54

(52) U.S. Cl. CPC A61K 38/2026; G01N 33/57407; G01N 33/6869; C07K 14/5406

(57) ABSTRACT

Methods for using the human interleukin-4 receptor (IL-4) as a biomarker for determining patent populations for treatment, predicting disease treatment efficacy, and predicting disease treatment prognosis in a variety of cancers, in particular glioblastoma and recurrent glioblastoma.

### CROSS REFERENCE TO RELATED APPLICATION

This application is a 371 application of International Application No. PCT/CA20/00013 filed on Aug. 5, 2021, which claims priority to U.S. Provisional Application No. 62/802,652 filed Feb. 7, 2019, which are hereby incorporated by reference in their entirety.

### REFERENCE TO A “SEQUENCE LISTING,” A TABLE, OR A COMPUTER PROGRAM, LISTING APPENDIX SUBMITTED ON A COMPACT DISK

This disclosure incorporates by reference the Sequence Listing text copy submitted herewith via EFS-Web, which was created on Aug. 4, 2021, entitled 117802-5011-US_Sequence_Listing.txt which is 213,662 bytes in size.

### BACKGROUND

First-line treatment for primary GB includes surgical resection of the bulk tumor to the maximal extent possible consistent with neurological preservation, followed by the Stupp protocol, which is established as the standard of care for newly diagnosed GB (Stupp et al., 2005). In the Stupp regimen, patients receive Temozolomide (Temodar®) concurrently with radiotherapy and then again following completion of radiotherapy. Temozolomide is approved for newly diagnosed GB concomitantly with radiotherapy and then as maintenance treatment (New Drug Application No. 021029; approval date: Aug. 11, 1999).

Newly diagnosed GB patients may also be treated with alternative chemotherapies, such as a nitrosourea regimen or insertion of a carmustine wafer (Gliadel®). Gliadel® is a biodegradable polymer wafer saturated with carmustine. Systemic toxicity usually associated with cytotoxic treatment may be reduced by implantation locally within the cranium (Westphal et al., 2006). Gliadel® is indicated for newly-diagnosed, high-grade malignant glioma as an adjunct to surgery and radiation as well as for recurrent GB as an adjunct to surgery (New Drug Application No. 020637; approval date: Feb. 25, 2003). It is implanted into the post-surgical cavity following complete tumor resection. Gliadel provides marginal increased survival of approximately 4-8 weeks (Westphal et al., 2003).

Using current treatment paradigms, most GB patients experience tumor recurrence/progression after standard first line treatment. Treatment options for patients with recurrent GB are very limited and the outcome is generally unsatisfactory. Specifically, chemotherapy regimens for recurrent or progressive GB have been unsuccessful, producing toxicity without benefit (Weller et al., 2013). This is mainly due to the lack of tissue specificity with resultant toxicity to normal tissues and consequently, a narrow therapeutic index. As overall survival remains dismal, novel anti-cancer modalities, with greater tumor specificity, more robust cytotoxic mechanisms and novel delivery techniques are needed for the treatment of recurrent GB.

Treatment options for patients with recurrent or progressive GB are very limited and positive long-term outcomes are rare. Drugs currently approved in the US for treatment of recurrent GB are Gliadel®, as mentioned above for first line treatment, and bevacizumab (Avastin®). In a Phase 3 study, placing a Gliadel implant directly into the tumor cavity after surgical resection of the tumor, 56% of recurrent GB treated subjects survived 6-month and the median survival was 26-weeks (Brem et al., 1995). However, the majority of patients with recurrent GB are not candidates for additional surgery, resulting in a large unmet need for this patient population (Weller et al., 2013).

Avastin® is an anti-angiogenic antibody that targets the vascular endothelial growth factor receptors (VEGF). It is indicated as a single agent for adult patients with recurrent GB (New Drug Application No. 125085; approval date: Feb. 26, 2004) but has not been shown to improve disease-related symptoms or survival. Avastin® was approved on the basis of objective response rate (ORR of 26%) endpoint (Genentech 2016; Cohen et al., 2009; Freidman et al., 2009). In 2013, Avastin® completed its confirmatory trial in newly diagnosed GB patients and did not meet its primary endpoint of overall survival. Based on the results of this trial, Genentech did not receive approval in the European Union (EU) for newly diagnosed GB; however, Avastin® remains indicated in the US and Japan for recurrent GB. Several studies have since compared efficacy with Avastin® or assessed combination approaches.

MDNA55 is a targeted immunotoxin consisting of a bioengineered circularly permuted version of interleukin-4 (cpIL-4), the binding domain, fused to a truncated version of a potent bacterial toxin—Pseudomonas aeruginosa exotoxin (PE) A, the catalytic domain (Kreitman et al., 1994). MDNA55 binds to interleukin-4 receptors (IL-4R) expressed on the surface of cells whereupon the entire complex is endocytosed. Following cleavage and activation by furin-like proteases found in high concentrations in the endosome of cancer cells, the catalytic domain of the truncated PE is released into the cytosol where it induces cell death via ADP-ribosylation of the Elongation Factor-2 and induction of apoptosis through caspase activation (Wedekind et al., 2001). Cells that do not express the IL-4R target do not bind to MDNA55 and are therefore, not subject to PE-mediated cell death. The PE portion was engineered to retain the catalytic domain but not the cell-binding domain.

Glioblastoma is a rapidly progressing and near-universally fatal cancer that is devastating to patients. This aggressive type of brain cancer is associated with substantial morbidity, often in the form of rapid deterioration of cognitive and psychomotor function, and a 1-year survival rate of approximately 25% following failure of front-line treatment (Lamborn et al., 2008). There is no currently effective treatment. MDNA55 represents a potential therapeutic advance. MDNA55 is a rationally designed targeted therapy with the potential to extend the survival of patients with GB. Adverse events associated with the administration and infusion of MDNA55, while serious, are similar to the effects of disease progression itself.

MDNA55 is a novel therapeutic that provides a targeted treatment approach whereby tumor cells are more sensitive to the toxic effects of the drug than normal cells. The target, IL-4R, is an ideal but under-exploited target for the development of cancer therapeutics, as it is frequently and intensely expressed on a wide variety of human carcinomas. Expression levels of IL-4R are low on the surface of healthy and normal cells, but increase several-fold on cancer cells. A majority of cancer biopsy and autopsy samples from adult and pediatric central nervous system (CNS) tumors, including recurrent GB biopsies, have been shown to over-express the IL-4R. There is little or no IL-4R expression in normal adult and pediatric brain tissue (Joshi, et al., 2001; see Table 2 of the reference). This differential expression of the IL-4R provides MDNA55 a wide therapeutic window (see Table 4 of the reference for IC₅₀ data). IL-4 targeted cargo proteins, including for example MDNA55, find use in the treatment of tumors that overexpress IL-4R, including recurrent GB and other CNS tumors that over-express the IL-4R. Cells that do not express the IL-4R target do not bind to MDNA55 and are, therefore, not subject to PE-mediated effects.

The expanding list of agents that have failed Phase 3 trials in GBM highlights the need for identifying biomarkers that are specifically linked to a drug's mechanism of action. In particular, for recurrent GBM (rGBM), the most common and uniformally fatal form of brain cancer. A critical challenge for GBM drug developers is the identification of specific patient subtypes who are most likely to respond to treatment with their drug candidate thereby increasing the likelihood of clinical success. For instance, studies have shown that GBM patients lacking methylation of MGMT promoters do not respond as well to temozolomide (TMZ) therapy and have a worse prognosis (Hegi M E, Diserens A C, Gorlia T, et al. N Engl J Med. MGMT gene silencing and benefit from temozolomide in glioblastoma. 2005 Mar. 10; 352(10)). This was reportedly the first predictive biomarker in brain tumors and potentially allows selection of patients who benefit from treatment with TMZ and radiotherapy. However, at present, MGMT promoter hypermethylation does not guide treatment strategies for patients with GBM. Another biomarker that can influence the prognosis of GBM is the epidermal growth factor receptor variant III (EGFRvIII), a mutated version of EGFR. Approximately 25-33% of GBM patients are thought to harbor this gene variant in their tumors (Johnson H, Del Rosario A M, Bryson B D, et al. Molecular characterization of EGFR and EGFRvIII signaling networks in human glioblastoma tumor xenografts. Mol Cell Proteomics. 2012 December; 11(12):1724-40). EGFRvIII overexpression in the presence of EGFR amplification plays an important role in enhanced tumorigenicity and has been shown to be a strong indicator of poor survival in GBM patients (Ushio Y, Tada K, Shiraishi S, et al., Correlation of molecular genetic analysis of p53, MDM2, p16, PTEN, and EGFR and survival of patients with anaplastic astrocytoma and glioblastoma. Front Biosci. 2003 May 1; 8: e281-8). This variant is being investigated as a promising target for new therapies, the prime example of which is Celldex Therapeutics' cancer vaccine rindopepimut, which is currently being investigated in a Phase 3 trial of newly diagnosed GBM as well as a Phase 2 trial for recurrent GBM.

Similarly, IL-4Rα over-expression in GBM (Husain S R, et al., Cancer Res. 1998; 58:3649-3653; Joshi B H, et al. Cancer Res. 2001; 61:8058-8061; Puri R K, et al., Cancer Res., 56: 5631-5637, 1999; and Pun R K, et al. Int J Cancer. 1994 Aug. 15; 58(4):574-81) as well as its up-regulation in the glioma microenvironment (Kohanbash G., et al. Cancer Res. 2013; 73(21):6413-23). Burt et al found that IL-4Rα is highly expressed in situ by tumor cells in human malignant pleural mesothelioma (MPM), and observed that mesothelioma tumors with high IL-4Rα expression are clinically more aggressive and have worse outcomes after surgical resection (Burt B M, et al., Clin. Cancer Res. 2012 Mar. 15; 18(6):1568-77). The IL-4R target for MDNA55 is an ideal but under-exploited drug target for central nervous system (“CNS”) tumors, including glioblastoma (“GBM”). The majority of cancer biopsy samples from adult and pediatric CNS tumors, including recurrent GBM, over-express the IL-4R while there is little or no IL-4R expression in normal adult and pediatric brain tissue.4 Expression of IL-4R correlates with increased tumorigenicity in mouse models and poor long term survival in clinical studies of patients with GBM2,3. In addition, the IL-4R is known to be expressed by Myeloid Derived Suppressor Cells and Tumor Associated Macrophages, which are known to be key components of the immunosuppressive tumor micro-environment (TME), which hides the tumor from cancer killing immune cells. (Roth F, De La Fuente A C, Vella J L, et al. Aptamer-mediated blockade of IL-4Rα triggers apoptosis of MDSCs and limits tumor progression. Cancer Res. 2012; 72(6):1373-83; and Bankaitis K V and Fingleton B. Targeting IL-4/IL-4R for the treatment of epithelial cancer metastasis. Clin Exp Metastasis. 2015 December; 32(8):847-56.)

To date, the predictive and prognostic value of these observations has yet to be determined in cancers, such as GBM. Ascertaining if IL-4R positive patients respond better to MDNA55 (or other IL-4 targeted cargo protein) and by identifying specific patient subtypes who are most likely to respond, as provided in the present invention, will help address these outstanding issues and may lead to further improved clinical outcomes for patients. Overall, there remains a need in the art for further effective methods for the treatment of these IL-4R expressing tumors, and the use of the level of IL-4R expression as a predictive and/or diagnostic marker in determining and/or predicting treatment regimens as described by present invention meets this need.

### BRIEF SUMMARY

The present invention provides methods for using IL-4R expression levels as a biomarker and/or companion diagnostic in the treatment of cancer.

In some embodiments, the present invention provides a method for determining a cancer patient population for treatment with an IL-4 targeted cargo protein, the method comprising:
a) measuring the level of IL-4 receptor (IL-4R) expression in a biological sample obtained from a cancer or tumor in the cancer patient,
b) quantitating the measurement of the level of IL-4R expression in the biological sample, and
c) treating the cancer patient with an IL-4 targeted cargo protein when the level of IL-4R expression is moderate or high.

In some embodiments, the present invention provides a method for predicting or determining the efficacy of treatment with an IL-4 targeted cargo protein, the method comprising:
a) measuring the level of IL-4 receptor (IL-4R) expression in a biological obtained from a cancer or tumor in the cancer patient,
b) quantitating the measurement of the level of IL-4R expression in the biological sample, and
c) correlating the level of IL-4R with the efficacy of treatment, wherein a moderate or high level of IL-4R expression is indicative of treatment efficacy for treatment with an IL-4 targeted cargo protein.

In some embodiments, the present invention provides a method for altering the regimen of treatment for a patient with cancer, the method comprising:
a) measuring the level of IL-4 receptor (IL-4R) expression in a biological obtained from a cancer or tumor in the cancer patient,
b) quantitating the measurement of the level of IL-4R expression in the biological sample, wherein a moderate or high level of IL-4R expression is indicative of treatment efficacy,
c) correlating the level of IL-4R expression with the efficacy of treatment, wherein a high level of IL-4R expression is indicative of altering the treatment regimen for treatment with an IL-4 targeted cargo protein, and
d) altering the treatment regimen to include an IL-4 targeted cargo protein when a moderate or high level of IL-4R expression is measured.

In some embodiments, the present invention provides a method for predicting or determining cancer disease prognosis and/or progression, the method comprising:
a) measuring the level of IL-4 receptor (IL-4R) expression in a biological sample from a tumor in the cancer patient,
b) quantitating the measurement of the level of IL-4R expression in the biological sample, wherein a moderate or high level of IL-4R expression is indicative of the disease prognosis and/or progression, and
c) correlating the level of IL-4R expression with the disease prognosis and/or progression, wherein a moderate or high level of IL-4R expression is indicative of severe disease prognosis and/or progression.

In some embodiments, a high level of IL-4R expression is measured the method further comprises treating the cancer patient with an IL-4 targeted cargo protein.

In some embodiments, the level of IL-4R expression is a high level of IL-4R expression.

In some embodiments, a high level of IL-4R expression is indicated by a percent score of ≥2+.

In some embodiments, a high level of IL-4R expression is indicated by a percent score of ≥3+.

In some embodiments, a moderate level of IL-4R expression is indicated by a percent score of ≥1+ but <2.

In some embodiments, a moderate level of IL-4R expression is indicated by H-Scores from 76 to 150.

In some embodiments, a high level of IL-4R expression is indicated by H-Scores from 151 to 225.

In some embodiments, high level of IL-4R expression is indicated by H-Scores from 226 to 300.

In some embodiments, the level of IL-4R expression is measured by measuring the level of IL-4Rα expression.

In some embodiments, the level of IL-4R expression is the level of Type 2 IL-4R (Type II IL-R4, comprising IL-4Rα and IL-13Rα1) expression.

In some embodiments, the level of IL-4R expression is measured using immunohistochemical (IHC) staining for IL-4R, including IL-4Rα expression.

In some embodiments, the cancer or tumor is selected from the group consisting of prostate cancer, ovarian cancer, breast cancer, endometrial cancer, multiple myeloma, melanoma, lymphomas, lung cancers including small cell lung cancer, kidney cancer, liver cancer, colon cancer, colorectal cancer, pancreatic cancer, gastric cancer, and brain cancer (including CNS tumors).

In some embodiments, the CNS tumor is selected from the group consisting of glioma, glioblastoma, astrocytoma, medulloblastoma, craniopharyogioma, ependymoma, pinealoma, hemangioblastoma, acoustic neuroma, oligodendroglia, menangioma, meningioma, neuroblastoma, retinoblastoma, medulloblastoma, and adult pituitary adenoma.

In some embodiments, the CNS tumor is a glioblastoma.

In some embodiments, the CNS tumor is a recurrent or refractory glioblastoma.

In some embodiments, the subject has an O6-methylguanine-methyltransferase (MGMT) positive or negative CNS tumor.

In some embodiments, the subject has furin positive CNS tumor.

In some embodiments, the IL-4 targeted cargo protein comprises one or more cargo moieties.

In some embodiments, the IL-4 targeted cargo protein comprises a toxin.

In some embodiments, the toxin comprises a bacterial toxin, animal toxin, or plant toxin.

In some embodiments, the toxin comprises a pore-forming toxin.

In some embodiments, the pore-forming toxin comprises aerolysin or proaerolysin.

In some embodiments, the plant toxin comprises bouganin or ricin.

In some embodiments, the bacterial toxin comprises a toxin selected from the group consisting of Pseudomonas exotoxin, cholera toxin, or diphtheria toxin.

In some embodiments, the IL-4 targeted cargo protein comprises pro-apoptosis member of the BCL-2 family selected from the group consisting of BAX, BAD, BAT, BAK, BIK, BOK, BID BIM, BMF, and BOK.

In some embodiments, the IL-4 targeted cargo protein comprises MDNA55 (SEQ ID NO:65) or a derivative or variant thereof.

In some embodiments, the IL-4 targeted cargo protein is MDNA55.

In some embodiments, the IL-4 targeted cargo protein comprises in IL-4R antibody as the targeting moiety.

In some embodiments, the IL-4R antibody is a humanized antibody.

In some embodiments, the IL-4 targeted cargo protein comprises a fusion protein.

In some embodiments, the IL-4 targeted cargo protein is administered intratumorally.

In some embodiments, the intratumoral administration comprises intracranial administration.

In some embodiments, the IL-4 targeted cargo protein is formulation in an artificial cerebral spinal fluid (CSF) solution and albumin, wherein the formulation is co-administered with a surrogate tracer to a subject in need thereof.

In some embodiments, the surrogate tracer is magnetic resonance imaging (MRI) contrast agent.

In some embodiments, the surrogate tracer is a gadolinium-bound tracer.

In some embodiments, the surrogate tracer is selected from the group consisting of gadolinium-diethylenetriamine pentaacetic acid (Gd-DTPA) and gadolinium-bound albumin (Gd-albumin).

In some embodiments, the albumin is human serum albumin.

In some embodiments, the artificial CSF solution is Elliotts B® solution.

In some embodiments, the IL-4 targeted cargo protein is administered via an intracranial catheter.

In some embodiments, the IL-4 targeted cargo protein is administered by convection-enhanced delivery (CED).

In some embodiments, the IL-4 targeted cargo protein is administered as a single dose via convection-enhanced delivery (CED).

In some embodiments, the IL-4 targeted cargo protein is administered as a single dose.

In some embodiments, the IL-4 targeted cargo protein is administered as a single dose of about 90 μg (1.5 μg/mL in 60 mL), about 240 μg (6 μg/mL in 40 mL), or about 300 μg (3 μg/mL in 100 mL).

In some embodiments, the IL-4 targeted cargo protein is administered at a dosage of 1.5 μg/mL in 60 mL.

In some embodiments, the IL-4 targeted cargo protein is administered at a dosage of 6 μg/mL in 40 mL.

In some embodiments, the IL-4 targeted cargo protein is administered at a dosage of 3 μg/mL in 100 mL.

In some embodiments, the IL-4 targeted cargo protein is administered as a single dose of about 1.5 μg/mL to about 3 μg/mL.

In some embodiments, the IL-4 targeted cargo protein is administered as a single dose over 1 day, 2 days, 3 days, 4 days, 5 days, 6 days, 7 days, or 8 days.

In some embodiments, the IL-4 targeted cargo protein is administered as 1, 2, 3, 4, or 5 infusions.

In some embodiments, the IL-4 targeted cargo protein is administered according to any of the preceding claims, then discontinuing the administration for from about 1 day to about 8 days, optionally discontinuing the administration for 1 day, 2 days, 3 days, 4 days, 5 days, 6 days, 7 days, or 8 days, followed by administration according to any of the preceding claims, and repeating this pattern of administration and discontinuance of administration for as long as necessary for treatment of the CNS tumor.

In some embodiments, the IL-4 targeted cargo protein is administered via one or more intracranial catheters, including 1 to 3 catheters

In some embodiments, the IL-4 targeted cargo protein is administered through the catheter with a flow rate of about 5 μL/min/catheter to about 20 μL/min/catheter or a flow rate of about 15 μL/min/catheter.

In some embodiments, the IL-4 targeted cargo protein is administered through the catheter at a concentration of 1.5 μg/mL and with a flow rate of about 15 μL/min/catheter.

In some embodiments, the IL-4 targeted cargo protein is used in combination with a steroid.

In some embodiments, the IL-4 targeted cargo protein is used in combination with a steroid dosed at equal to or lower than 4 mg/day.

In some embodiments, the present invention provides a kit comprising an IL-4 targeted cargo protein as described in any of the preceding claims, wherein the kit comprises an IL-4R antibody, instructions for using the IL-4R antibody in an immunohistochemistry (IHC)-based assay, and instructions for determining the percent score or the H-Score.

### BRIEF DESCRIPTION OF THE DRAWINGS

FIG. 1. Comparative analysis of the IL-13Rα1- and the IL-13Rα2-selective IL-13 variants Human IL-13 and IL-13Rα1 and IL-13Rα2 selective variants sequences are given for the indicated residue numbers. Kinetic and affinity parameters were determined by surface plasmon resonance.

FIG. 2A-2B. Provides examples of IgG1, IgG2, IgG3, and IgG4 sequences.

FIG. 3A-3C. Exemplary anti-PD-1 antibodies for use with the combinations of the invention.

FIG. 4A-4F. Exemplary anti-PD-L1 antibodies for use with the combinations of the invention.

FIG. 5A-5B. Exemplary oncolytic viruses.

FIG. 6. Data showing Assay Transfer Concordance for IL-4Rα

FIG. 7. Data showing IL-4Rα Scoring in Glioblastoma for CAP/CLIA Sensitivity Testing.

FIG. 8A-8D. 8A) Thresholds of IL-4Rα Positivity by H-Score Values in GBM. 8B) Thresholds of IL-4Rα Positivity by Percent Staining of ≥1+ Intensities in GBM. 8C) Thresholds of IL-4Rα Positivity by Percent Staining of ≥2+ Intensities in GBM. 8D) Thresholds of IL-4Rα Positivity by Percent Staining of ≥3+ Intensities in GBM

FIG. 9A-9B. 9A) IHC Procedure-TechMate Protocol. 9B) Antibody Specifications & Assay Conditions.

FIG. 10. IL-4Rα and Rabbit IgG Staining in GBM. Representative images of various levels of IL-4Rα reactivity in the GBM tissues. This figure also includes a corresponding representative Rabbit IgG negative control. All Rabbit IgG isotype negative controls were nonreactive across the sensitivity panel of GBM tumors tested.

FIG. 11. CAP/CLIA Precision & Reproducibility of IL-4Rα in GBM.

FIG. 12A-12B. IL-4Rα Scoring in Multi-Normal Human Tissues for Specificity Testing

FIG. 13. Schematic representation of the treatment pathway for GBM.

FIG. 14. Image data showing high-flow image guided CED improves distribution.

FIG. 15. Image data showing MDNA55 trajectory planning and distribution analysis (using Brainlab iPlan® Flow software).

FIG. 16. Data showing promising survival at low doses of MDNA55 compared to approved therapies for rGBM (recurrent GBM).

FIG. 17. Image data showing IL-4R Expression in GBM Tissues Obtained at First Diagnosis. Archived tissue obtained at first diagnosis of GBM is analyzed for IL-4Rα expression by immunohistochemistry (IHC). GBM samples are scored for cytoplasmic IL-4Rα reactivity using the H-Score method: H-Scores from 0 to 75=no to low expression; H-Scores from 76 to 150=moderate expression; H-Scores from 151 to 225=high expression; and H-Scores from 226 to 300=very high expression. IL-4R Negative=H-Scores ≤75. IL-4R Positive=H-Scores >75.

FIG. 18. IL-4R Positivity is Associated with More Aggressive Disease (Shorter Time to Relapse from Initial Diagnosis). Time to first relapse from initial diagnosis in subjects treated with MDNA55 (n=24). Kaplan-Meier plot showing time to first relapse from initial diagnosis in subjects with low H-Scores (H-Score ≤75) compared to subjects with moderate to high H-Scores (H-Score >75). Median time to 1^{st} relapse for subjects with H-Scores ≤75 vs. H-Score >75 is 16.7 months vs. 10.3 months, respectively. Log-rank (Mantel-Cox) test p-value=0.2123.

FIG. 19. IL-4R+ Subjects Show Better Survival Outcomes Following Treatment with MDNA55. Survival of subjects in current study according to IL-4R H-Score (n=24). Kaplan-Meier plot showing survival of subjects with low H-Scores (H-Score ≤75) compared to survival of subjects with moderate to high H-Scores (H-Score >75) from the current study. Median OS for subjects with H-Scores ≤75 vs. H-Score >75 is 8.5 months vs. 15.2 months, respectively. Log-rank (Mantel-Cox) test p-value=0.0909. Data cut-off is Jan. 16, 2019.

FIG. 20. Imaging data showing IL-4R+ subjects show tumor responses after MDNA55 treatment.

FIG. 21. MDNA55 Presents a Promising Benefit-Risk Profile Especially in IL-4R+ Recurrent GBM. Top black rectangle: IL-4R+ is associated with more aggressive disease. Bottom black rectangle: MDNA55 improves survival outcomes in IL-4R+ rGBM

FIG. 22. Progression free survival of subjects in current study according to IL-4R H-Score (n=24). Kaplan-Meier plot showing PFS of subjects with low H-Scores (H-Score ≤75) compared to PFS of subjects with moderate to high H-Scores (H-Score >75) from the current study. Median PFS for subjects with H-Scores ≤75 vs. H-Score >75 is 1.9 months vs. 3.7 months, respectively. Log-rank (Mantel-Cox) test p-value=0.1156.

FIG. 23A illustrates the summary of MDNA55 clinical study design. FIG. 23B summarizes patient demographics. KPS is Karnofsky performance score.

FIG. 24A shows percentage of survival of the first 40 subjects enrolled in the clinical study. FIG. 24B shows percentage of survival in subjects with high or low IL-4R expression. 36 out of the 40 subjects were evaluated for IL-4R expression. Medium overall survival (mOS) and 12-month survival rate (OS-12) are shown.

FIG. 25A shows percentage of survival in subjects with or without methylated MGMT gene promoter after MDNA55 treatment. FIG. 25B shows percentage of survival in two groups of subjects after MDNA55 treatment. One group had unmethylated MGMT gene promoter and high level of IL-4R expression and the other group had unmethylated MGMT gene promoter and low level of IL-4R expression.

FIG. 26A shows percentage of survival in subjects having high or low steroid use after the MDNA55 treatment. FIG. 26B shows percentage of survival in two groups of subjects after MDNA55 treatment. One group had low steroid use and high level of IL-4R expression and the other group had low steroid use and low level of IL-4R expression.

FIG. 27 shows brain MRI images of a subject displaying an early onset response after the MDNA55 treatment. The patient has wild-type isocitrate dehydrogenase (IDH), unmethylated MGMT gene promoter, high IL-4R expression and 2 prior relapses.

FIG. 28 shows brain MRI images of a subject displaying a delayed onset response after pseudo-progression after the MDNA55 treatment. The patient has wild-type isocitrate dehydrogenase (IDH), methylated MGMT gene promoter, high IL-4R expression and 1 prior relapse.

FIG. 29 shows brain MRI images of a subject displaying a delayed onset response after pseudo-progression after the MDNA55 treatment. The patient has wild-type isocitrate dehydrogenase (IDH), methylated MGMT gene promoter, high IL-4R expression and 1 prior relapse. Arrows indicate active tumor mass.

FIG. 30 shows a distribution of tumor volume change in subjects treated with MDNA55. The tumor volume change was measured by MRI and the results are evaluated from baseline. Tumor volume is shrinked, stabilized or progressed after the treatment. Tumor control rate is calculated based on the percentage of subjects who had shrinked or stabilized tumor size.

FIG. 31 shows a distribution of tumor volume change in subjects after treatment with MDNA55. The tumor volume change was measured by MRI and the results are evaluated from nadir. Tumor volume is shrinked, stabilized or progressed after the treatment. Tumor control rate is calculated based on the percentage of subjects who had shrinked or stabilized tumor size.

FIGS. 32A and 32B show the percentage of survival in subjects after MDNA55 treatment, wherein the subjects are divided into two groups, with one group having shrinked or stabilized tumor volume after the treatment and the other group having progressed tumor volume after the treatment.

FIG. 33A show medium overall survival (months) of subjects after treatment with cancer drugs including Temozolomide (TMZ), Carmustine (brand name Gliadel®), lomustine (LOM), bevacizumab (brand name Avastin®), or MDNA55. FIG. 33B show 12-month percentage survival rate of subjects after treatment with cancer drugs including Temozolomide (TMZ), Carmustine (brand name Gliadel®), lomustine (LOM), bevacizumab (brand name Avastin®), or MDNA55. n indicates the number of subjects tested.

### DETAILED DESCRIPTION

In order for the present disclosure to be more readily understood, certain terms and phrases are defined below as well as throughout the specification.

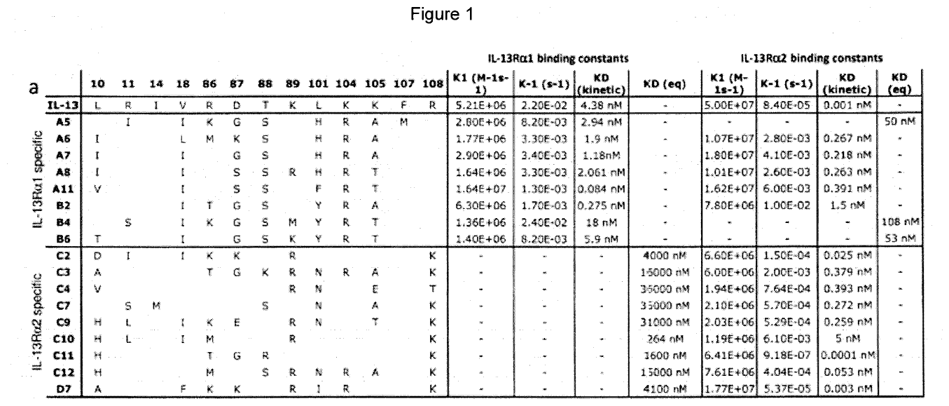

MDNA55 has been co-administered with a tracer (an MRI contrast agent) using convection enhanced delivery (CED) allowing real-time monitoring of drug distribution in and around the tumor. MDNA55 is a targeted immunotoxin consisting of a bioengineered circularly permuted version of interleukin-4 (cpIL-4), the binding domain, fused to a truncated version of a potent bacterial toxin—Pseudomonas aeruginosa exotoxin (PE) A, the catalytic domain (Kreitman et al., 1994). MDNA55 binds to interleukin-4 receptors (IL-4R) expressed on the surface of cells whereupon the entire complex is endocytosed. Following cleavage and activation by furin-like proteases found in high concentrations in the endosome of cancer cells, the catalytic domain of the truncated PE is released into the cytosol where it induces cell death via ADP-ribosylation of the Elongation Factor-2 and induction of apoptosis through caspase activation (Wedekind et al., 2001). Cells that do not express the IL-4R target do not bind to MDNA55 and are therefore, not subject to PE-mediated cell death. The mechanism of action is depicted in FIG. 1. Of note is that the PE portion was engineered to retain the catalytic domain but not the cell-binding domain; the rationale behind this approach was to have a built in safety mechanism whereby in the event PE inadvertently cleaved off from the IL-4, it could not be toxic as the binding domain of the PE was removed and consequently it would be unable to internalize into cells and arrest protein synthesis. However, there remains a need for biomarkers for determining patient populations, treatment efficacy, and treatment outcome in patient populations. The present invention meets that need.

### A. Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russell, Molecular Cloning: A Laboratory Manual 3rd ed., Cold Spring harbor Laboratory Press (Cold Spring Harbor, N.Y. 2001), provide one skilled in the art with a general guide to many terms used in the present disclosure. As appropriate, procedures involving the use of commercially available kits and reagents are generally carried out in accordance with manufacturer defined protocols and/or parameters unless otherwise noted.

As used herein, the abbreviations for the genetically encoded L-enantiomeric amino acids used in the disclosure methods are conventional and are as follows in Table 1.

TABLE 1
Amino acid abbreviations

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

“Hydrophilic Amino Acid” refers to an amino acid exhibiting a hydrophobicity of less than zero according to the normalized consensus hydrophobicity scale of Eisenberg et al., 1984, J. Mol. Biol. 179: 125-142. Genetically encoded hydrophilic amino acids include Thr (T), Ser (5), His (H), Glu (E), Asn (N), Gln (Q), Asp (D), Lys (K) and Arg (R).

Various Terms and abbreviations are provided in Table 2.

TABLE 2
Abbreviations and Terms:

| | |
|---|---|
| PA | Proaerolysin |
| BAD | BCL2-associated agonist of cell death |
| BAχ | BCL2-associated X protein |
| EGF | Epidermal growth factor |
| EpCAM | Epithelial protein cell adhesion molecule |
| GMCSF | Granulocyte-macrophage colony-stimulating factor |
| IL-4 | Interleukin-4 (also IL4) |
| IL-13 | Interleukin-13 (also IL13) |
| PSMA | Prostate specific membrane antigen |

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a IL-4 targeted cargo protein” includes single or plural IL-4 targeted cargo proteins and is considered equivalent to the phrase “comprising at least about one IL-4 targeted cargo protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs.

Accession Numbers: Reference numbers assigned to various nucleic acid and amino acid sequences in the NCBI database (National Center for Biotechnology Information) that is maintained by the National Institute of Health, U.S.A. The accession numbers listed in this specification are herein incorporated by reference as provided in the database as of the date of filing this application.

Administration: Providing or giving a subject an agent, such as a composition that includes an IL-4 targeted cargo protein. Exemplary routes of administration include, but are not limited to, oral, injection (such as subcutaneous, intramuscular, intradermal, intraperitoneal, intratumoral and intravenous), sublingual, rectal or transrectal, transdermal, intranasal, vaginal, cervical, and inhalation routes. In specific examples, intratumoral includes local, regional, focal, or convection enhanced delivery. In other specific examples, administration includes transurethral or transperineal administration. In one example, surrogate magnetic resonance imaging tracers (e.g., gadolinium-bound albumin (Gd-albumin)) can be administered in combination with the IL-4 targeted cargo protein to determine if the IL-4 targeted cargo protein is delivered to a tumor, such as a brain tumor, safely at therapeutic doses while monitoring its distribution in real-time (see for example, Murad et al., Clin. Cancer Res. 12(10):3145-51 2006).

Antibody: Immunoglobulin molecules and immunologically active portions of immunoglobulin molecules, that is, molecules that contain an antigen binding site that specifically binds (immunoreacts with) an epitope, such as an epitope displayed by cancer cells and/or cancer stem cells. Antibodies include monoclonal antibodies, polyclonal antibodies, as well as humanized antibodies. Antibodies also include affibodies. Affibodies mimic monoclonal antibodies in function but are based on Protein A. Affibodies can be engineered as high-affinity ligands for binding to a targeting moiety.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody (scFv) and scFv molecules linked to each other to form a bivalent dimer (diabody) or trivalent trimer (triabody); (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Methods of producing polyclonal and monoclonal antibodies are known to those of ordinary skill in the art, and many antibodies are available. See, e.g., Coligan, Current Protocols in Immunology Wiley/Greene, N.Y., 1991; and Harlow and Lane, Antibodies: A Laboratory Manual Cold Spring Harbor Press, N Y, 1989; Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2nd Edition Freeman and Company, N Y, 1995; McCafferty et al., Antibody Engineering, A Practical Approach, IRL at Oxford Press, Oxford, England, 1996, and Paul Antibody Engineering Protocols Humana Press, Towata, N.J., 1995.

In some examples, an antibody specifically binds to a target protein (e.g., a cell surface receptor such as an IL4 receptor) with a binding constant that is at least 10³ M⁻¹ greater, 10⁴ M⁻¹ greater or 10⁵ M⁻¹ greater than a binding constant for other molecules in a sample. In some examples, a specific binding reagent (such as an antibody (e.g., monoclonal antibody) or fragments thereof) has an equilibrium constant (K_{d}) of 1 nM or less. For example, a specific binding agent may bind to a target protein with a binding affinity of at least about 0.1×10.sup.⁻⁸ M, at least about 0.3×10⁻⁸M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Binds or binding: The association between two or more molecules, wherein the two or more molecules are in close physical proximity to each other, such as the formation of a complex. An exemplary complex is a receptor-ligand pair or an antibody-antigen pair. Generally, the stronger the binding of the molecules in a complex, the slower their rate of dissociation. Specific binding refers to a preferential binding between an agent and a specific target. For example, specific binding refers to when a IL-4 targeted cargo protein that includes a targeting moiety specific for a cancer stem cell antigen binds to the cancer stem cell, but does not significantly bind to other cells that do not display the target in close proximity to the cancer stem cell. Such binding can be a specific non-covalent molecular interaction between the ligand and the receptor. In a particular example, binding is assessed by detecting cancer stem cell growth inhibition using one of the methods described herein after the IL-4 targeted cargo protein has been contacted with the cancer stem cell.

Such interaction is mediated by one or, typically, more noncovalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a protein-protein interaction) can be competitively removed (or displaced) from such association (e.g., protein complex) by excess amounts of either specific binding partner. Such competition assays (or displacement assays) are very well known in the art.

Cancer: Malignant neoplasm that has undergone characteristic anaplasia with loss of differentiation, increased rate of growth, invasion of surrounding tissue, and is capable of metastasis. Residual cancer is cancer that remains in a subject after any form of treatment given to the subject to reduce or eradicate a cancer and recurrent cancer is cancer that recurs after such treatment. Metastatic cancer is a cancer at one or more sites in the body other than the site of origin of the original (primary) cancer from which the metastatic cancer is derived. In the case of a metastatic cancer originating from a solid tumor, one or more (for example, many) additional tumor masses can be present at sites near or distant to the site of the original tumor. The phrase “disseminated metastatic nodules” or “disseminated metastatic tumors” refers to a plurality (typically many) metastatic tumors dispersed to one or more anatomical sites. For example, disseminated metastatic nodules within the peritoneum (that is a disseminated intraperitoneal cancer) can arise from a tumor of an organ residing within or outside the peritoneum, and can be localized to numerous sites within the peritoneum. Such metastatic tumors can themselves be discretely localized to the surface of an organ, or can invade the underlying tissue.

Cargo Moiety: A peptide (e.g., protein fragment or full length protein) or other molecule that can function to significantly reduce or inhibit the growth of a cancer stem cell. In some examples a cargo moiety can trigger cell death (e.g., apoptosis). Exemplary cargo moieties include toxins, such as toxins derived from plants, microorganisms, and animals. In other examples, cargo moieties are proteins that normally contribute to the control of cell life cycles, for example cargo moieties can be any protein that triggers cell death, such as via apoptotic or non-apoptotic pathways. In some examples, the cargo moiety is not a protein, but another molecule that can function to significantly reduce or inhibit the growth of a cancer stem cell, such as thapsigargin. In some examples, a cargo moiety is activated by a tumor-associated protease, such as PSA. Exemplary cargo moieties, and exemplary GenBank accession numbers, are provided in Table 3, below. In addition to native cargo sequences, variant sequences can also be used, such as mutant sequences with greater biological activity than that of the native sequence.

TABLE 3
Exemplary cargo moiety sequences

| Cargo Moiety | Accession Numbers* |
|---|---|
| Aerolysin | ABR14715.1; ABR14714.1 |
| Proaerolysin | AAA21938.1; P09167.2; U.S. Pat. No. 7,282,476 |
| | (proaerolysin sequences therein herein |
| | incorporated by reference) |
| Bouganin | AAL35962 and SEQ ID NO: 9 in U.S. Pat. No. |
| | 6,737,511, as well as variant sequences |
| | provided in U.S. Pat. No. 7,339,031 and WO |
| | 2005/090579 (bouganin sequences therein |
| | herein incorporated by reference) |
| Pseudomonas | 1IKP A; AAB59097.1; AAF90003.1 (also see |
| exotoxin | SEQ ID NO: 1 of U.S. Pat. No. 6,011,002) |
| Bcl-2 pro-apoptotic | BAD: CAG46757; AAH01901.1; CAG46733.1; |
| proteins such as | and sequences provided in U.S. Pat. No. 6,737,511 |
| BAD and BAX | BAX: CAE52909.1; AAO22992.1; EAW52418.1 |
| Cholera toxin | BAA06291.1; ACF35010.1; BAA06288.1; as well |
| | as variant sequences provided in U.S. patent |
| | application Ser. No. 61/058,872 (variant |
| | cholera toxin sequences therein herein |
| | incorporated by reference) |
| Ribonuclease A | BAA05124.1; NP_937877.1; NP_115961.2; |
| | Q5GAN4.1; and sequences provided in PCT |
| | Publication No. W02007/041361 (rapLR1 |
| | sequences therein herein incorporated by |
| | reference) |

*GenBank Numbers are herein incorporated by reference, as well as their corresponding nucleic acid sequences.

Contact or contacting: Refers to the relatively close physical proximity of one object to another object. Generally, contacting involves placing two or more objects in close physical proximity to each other to give the objects and opportunity to interact. For example, contacting a IL-4 targeted cargo protein with a cancer stem cell can be accomplished by placing the IL-4 targeted cargo protein (which can be in a solution) in proximity to the cell, for example by injecting the IL-4 targeted cargo protein into a subject having the cancer. Similarly, a IL-4 targeted cargo protein can be contacted with a cell in vitro, for example by adding the IL-4 targeted cargo protein to culture media in which the cell is growing.

Decrease: To reduce the quality, amount, or strength of something. In one example, a therapy (such as treatment with a IL-4 targeted cargo protein) decreases a cancer stem cell population (such as by decreasing the size of a tumor, the volume of a tumor, the metastasis of a tumor, the number of cancer cells and/or cancer stem cells, or combinations thereof), or one or more symptoms associated with cancer, for example as compared to the response in the absence of the therapy. In a particular example, a therapy decreases the size of a tumor, volume of a tumor, number of cancer cells and/or cancer stem cells, or the metastasis of a cancer, or combinations thereof, subsequent to the therapy, such as a decrease of at least about 10%, at least about 20%, at least about 50%, or even at least about 90%. Such decreases can be measured using the methods disclosed herein.

Diagnose: The process of identifying a medical condition or disease, for example from the results of one or more diagnostic procedures. In particular examples, includes determining the prognosis of a subject (e.g., likelihood of survival over a period of time, such as likelihood of survival in 6-months, 1-year, or 5-years). In a specific example, cancer is diagnosed by detecting the presence of a cancer stem cell in a sample using one or more of the targets on the cancer stem cell surface. For example, diagnoses can include determining the particular stage of cancer or the presence of a site of metastasis.

Linker: A molecule used to connect one or more agents to one or more other agents. For example, a linker can be used to connect one or more cargo moieties to one or more targeting moieties. Particular non-limiting examples of linkers include dendrimers, such as synthetic polymers, peptides, proteins and carbohydrates. Linkers additionally can contain one or more protease cleavage sites or be sensitive to cleavage via oxidation and/or reduction.

Pharmaceutically acceptable carriers: The term “pharmaceutically acceptable carriers” refers to pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic or diagnostic agents, such as one or more of the IL-4 targeted cargo protein molecules provided herein.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Pharmaceutical agent or drug: A chemical compound or composition capable of inducing a desired therapeutic effect when administered to a subject, alone or in combination with another therapeutic agent(s) or pharmaceutically acceptable carriers. In a particular example, a pharmaceutical agent (such as one that includes a IL-4 targeted cargo protein) treats a cancer, for example by reducing the size of the tumor (such as the volume or reducing the number of cancer cells and/or cancer stem cells), reducing metastasis of the cancer, or combinations thereof.

Recombinant: A recombinant molecule (such as a recombinant nucleic acid molecule or protein) has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. A recombinant protein is one that results from expressing a recombinant nucleic acid encoding the protein. IL-4 targeted cargo proteins of the present disclosure are generally recombinant.

Sample: Biological specimens such as samples containing biomolecules, such as nucleic acid molecules, proteins, or both. Exemplary samples are those containing cells or cell lysates from a subject, such as those present in peripheral blood (or a fraction thereof such as serum), urine, saliva, tissue biopsy, cheek swabs, surgical specimen, fine needle aspirates, cervical samples, and autopsy material. In a specific example, a sample is obtained from a tumor (for example a section of tissue from a biopsy), which can include tumor cells that are both non-cancer cells and/or cancer stem cells and cancer cells and/or cancer stem cells. In some embodiments, the tumor sample is from a central nervous system (CNS) tumor.

Sequence identity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN can be used to compare nucleic acid sequences, while BLASTP can be used to compare amino acid sequences. To compare two nucleic acid sequences, the options can be set as follows: -i is set to a file containing the first nucleic acid sequence to be compared (such as C:\seq1.txt); --j is set to a file containing the second nucleic acid sequence to be compared (such as C:\seq2.txt); --p is set to blastn; --o is set to any desired file name (such as C:\output.txt); --q is set to --1; --r is set to 2; and all other options are left at their default setting. For example, the following command can be used to generate an output file containing a comparison between two sequences: C:\B12seq --i c:\seq1.txt --j c:\seq2.txt --p blastn --o c:\output.txt --q --1 --r 2.

To compare two amino acid sequences, the options of B12seq can be set as follows: -i is set to a file containing the first amino acid sequence to be compared (such as C:\seq1.txt); --j is set to a file containing the second amino acid sequence to be compared (such as C:\seq2.txt); --p is set to blastp; --o is set to any desired file name (such as C:\output.txt); and all other options are left at their default setting. For example, the following command can be used to generate an output file containing a comparison between two amino acid sequences: C:\B12seq --i c:\seq1.txt --j c:\seq2.txt --p blastp --o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166/1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a cargo protein or targeting moiety provided herein.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a cargo moiety or targeting moiety provided herein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

Subject: Living multi-cellular vertebrate organisms, a category that includes human and non-human mammals (such as laboratory or veterinary subjects).

IL-4 targeted cargo protein: Any protein that binds specifically to a cancer stem cell and reduces or inhibits cancer stem cell growth, or kills cancer cells and/or cancer stem cells. In some examples, IL-4 targeted cargo proteins can target both cancer cells and/or cancer stem cells and tumor (e.g., cancer) cells that are not cancer cells and/or cancer stem cells. IL-4 targeted cargo proteins include a targeting moiety and a cargo moiety, the targeting moiety specifically binds with the cancer stem cell and the cargo moiety significantly reduces or inhibits the growth of the cancer stem cell or kills cancer stem cells. In some examples the cargo moiety causes the death of the cancer stem cell that it is associated with. Because in some examples the cargo moiety is not a protein, such as a chemotherapeutic agent, and in some examples the targeting moiety is not a protein, the IL-4 targeted cargo protein in some examples is not actually a protein.

Targeting moiety: Any compound that binds to a molecule (herein referred to as a target) displayed by a cancer stem cell, for example a targeting moiety can be an antibody that binds to a target (e.g., receptor), a ligand (e.g., a cytokine or growth factor) that binds to a receptor, a permuted ligand that binds to a receptor, or a peptide sequence sensitive to cleavage by a tumor-associated protease. In some examples, a targeting moiety is activated by a tumor-associated protease, such as PSA. Typically, targeting moieties selectively bind to one type of cell displaying a target more effectively than they bind to other types of cells that do not display the target. Targeting moieties can be chosen to selectively bind to subsets of tumor cells, such as cancer cells and/or cancer stem cells. Targeting moieties include specific binding agents such as antibodies, natural ligands of the target on the stem cell, such as IL-4, derivatives of such natural ligands, and immunoglobulin A. In some examples, the targeting moiety is not biologically active (e.g., cannot activate a receptor), but retains the ability to bind to the target and thus direct the IL-4 targeted cargo protein to the appropriate cells.

Table 2 provides information relating to the sequences of exemplary natural ligands as well as other antigens that can be used as targeting moieties. In some examples, circular permuted ligands, such as circular permuted IL-4, can be used to bind cancer cells and/or cancer stem cells. As additional research is performed, new cancer stem cell specific targets will be identified. These additional markers can be used as targets for binding to targeting moieties and IL-4 targeted cargo proteins can be made to inhibit the growth of (or kill) cancer cells and/or cancer stem cells displaying such ligands. One of ordinary skill in the art will appreciate that once a marker is known, standard methods of making antibodies to the identified marker can be used to make targeting moieties specific for the cancer stem cell marker, thus, allowing for the development of a specific IL-4 targeted cargo protein.

TABLE 4
Exemplary targeting moiety sequences

| Receptor or Antigen | |
|---|---|
| to be Targeted | Accession Numbers* |
| IL-4 | AAH70123; CAA57444.1; AAH67515.1 (also see |
| | SEQ ID NO: 2 and various circularly permuted |
| | ligands described in U.S. Pat. No. 6,011,002) |
| MDNA55 | SEQ ID NO: 65 |
| IL-13 | AAH96141.2; AAH96138.1; AAH96139.1 |

*GenBank Numbers are herein incorporated by reference, as well as their corresponding nucleic acid sequences.

Targets on cancer cells and/or cancer cells and/or cancer stem cells include small molecules displayed on the surface of cancer cells and/or cancer stem cells. Antibodies directed to such targets can be used as targeting moieties as well as the natural ligands of the targets and derivatives thereof.

Therapeutically effective amount: An amount of an agent that alone, or together with a pharmaceutically acceptable carrier or one or more additional therapeutic agents, induces the desired response. A therapeutic agent, such as a IL-4 targeted cargo protein, is administered in therapeutically effective amounts that stimulate the desired response, for example reduction of symptoms of cancer in subjects known to have a cancer that includes cancer cells and/or cancer stem cells.

Effective amounts of a therapeutic agent can be determined in many different ways, such as assaying for improvement of a physiological condition of a subject having cancer. Effective amounts also can be determined through various in vitro, in vivo or in situ assays.

Therapeutic agents can be administered in a single dose, or in several doses, for example weekly, monthly, or bi-monthly, during a course of treatment. However, the effective amount of can be dependent on the source applied, the subject being treated, the severity and type of the condition being treated, and the manner of administration.

In one example, it is an amount sufficient to partially or completely alleviate symptoms of cancer in a subject. Treatment can involve only slowing the progression of the cancer temporarily, but can also include halting or reversing the progression of the cancer permanently. For example, a pharmaceutical preparation can decrease one or more symptoms of the cancer (such as the size of a tumor or the number of tumors or number of cancer cells and/or cancer stem cells), for example decrease a symptom by at least about 20%, at least about 50%, at least about 70%, at least about 90%, at least about 98%, or even at least about 100%, as compared to an amount in the absence of the therapeutic preparation.

Treating a disease: A therapeutic intervention that ameliorates a sign or symptom of a disease or pathological condition, such a sign or symptom of cancer. Treatment can also induce remission or cure of a condition, such as cancer and in particular a central nervous system (CNS) cancer or tumor. In particular examples, treatment includes preventing a disease, for example by inhibiting the full development of a disease, such as preventing development of tumor metastasis. Prevention of a disease does not require a total absence of a dysplasia or cancer. For example, a decrease of at least about 50% can be sufficient.

Tumor: Is a neoplasm or an abnormal mass of tissue that is not inflammatory, which arises from cells of preexistent tissue. A tumor can be either benign (noncancerous) or malignant (cancerous). Examples of hematological tumors include, but are not limited to: central nervous system (CNS) cancers or tumors. Examples of solid tumors, such as sarcomas and carcinomas, include, but are not limited to brain tumors, and CNS tumors (such as a glioma, glioblastoma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma). Tumors include recurrent and/or refractory CNS tumors.

Refractory: A disease or condition which does not respond to attempted forms of treatment, for example a tumor that does not respond to the standard treatment methods.

Under conditions sufficient for: A phrase that is used to describe any environment that permits the desired activity. In one example, includes incubating a IL-4 targeted cargo protein with tumor stem cell under conditions that allow the IL-4 targeted cargo protein to specifically bind to a cancer stem cell in the sample. In another example, includes contacting one or more IL-4 targeted cargo proteins with one or more cancer cells and/or cancer stem cells in a subject sufficient to allow the desired activity. In particular examples, the desired activity is decreasing growth or multiplication of such cancer cells and/or cancer stem cells or killing cancer cells and/or cancer stem cells.

Unit dose: A physically discrete unit containing a predetermined quantity of an active material (such a IL-4 targeted cargo protein) calculated to individually or collectively produce a desired effect such as a therapeutic effect. A single unit dose or a plurality of unit doses can be used to provide the desired effect, such as a therapeutic effect.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

As used herein, the term “anti-PD-1 antibody” refers to any antibody that binds to PD-1, including inhibitory antibodies. An “anti-PD-1 inhibitor” refers to an inhibitor that binds to and inhibits PD-1. Such anti-PD-1 antibodies and/or inhibitors include but are not limited to nivolumab, BMS-936558, MDX-1106, ONO-4538, AMP224, CT-011, and MK-3475, among others.

As used herein, the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, reproductive systems, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus. Cancers generally can include prostate cancer, ovarian cancer, breast cancer, endometrial cancer, multiple myeloma, melanoma, lymphomas, lung cancers including small cell lung cancer, kidney cancer, colorectal cancer, pancreatic cancer, gastric cancer, and brain cancer.

The term “carcinoma” is art-recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

As used herein, the term “hematopoietic neoplastic disorders” refers to diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Stemberg disease.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease. A therapeutically effective amount can be an amount that reduces tumor number, tumor size, and/or increases survival.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an IL-4 mutein to a subject.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, the therapeutic effect is to reduce tumor number. In some embodiments, the therapeutic effect is to reduce tumor size. In some embodiments, the therapeutic effect is to increase survival.

In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. IL-4 muteins in combination with anti-PD-1 antibodies, and pharmaceutical compositions thereof can be packaged in a single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue or vasculature of a subject (for example, liver tissue or veins). Other effective dosages can be readily established by one of ordinary skill in the art through routine trials establishing dose response curves.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome. In some embodiments, the effective amount is an amount sufficient to reduce tumor number. In some embodiments, the effective amount is an amount sufficient to reduce tumor size. In some embodiments, the effective amount is an amount sufficient to increase survival.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

### II. IL-4 and IL-13 Fusions

Described herein are IL-4 and/or IL-13 fusion proteins that target cancer cells and/or cancer stem cells and inhibit growth of and/or kill cancer cells and/or cancer stem cells, including for example MDNA55. These molecules, herein after collectively referred to as IL-4 targeted cargo proteins, include a targeting moiety that binds to a target (e.g., in some embodiments IL-4R) displayed by the cancer stem cell as well as a cargo moiety that provides the cell growth inhibiting (or cell killing) activity. The targeting moiety can be bound to the cargo moiety directly or through one or more of a variety of linkers that are further described herein. Cancer cells and/or cancer stem cells generally have the ability to self-renew and thus generate progeny with similar properties as themselves. In some examples, the disclosed IL-4 targeted cargo proteins can target both cancer cells and/or cancer stem cells and tumor (e.g., cancer) cells that are not cancer cells and/or cancer stem cells. Therefore, in some examples IL-4 targeted cargo proteins can kill or inhibit the growth of cancer cells and/or cancer stem cells and tumor (e.g., cancer) cells that are not cancer cells and/or cancer stem cells. In other examples, such as with a targeting moiety directed to CD 133, the IL-4 targeted cargo proteins kill or inhibit the growth of cancer cells and/or cancer stem cells in the tumor, but not tumor cells that are not cancer cells and/or cancer stem cells.

Targeting moieties include proteins and other agents that function to specifically bind to a target on a cancer stem cell (but in some examples the target may also be present on other cancer cells). Targeting moieties include specific binding agents, such as antibodies, affibodies, or receptor ligands. In some examples, the targeting moiety is derived from the natural ligand to the target (e.g., cell surface receptor) displayed by the cancer stem cell. The targeting moiety that is derived from a natural ligand can include the complete amino acid sequence of the ligand (e.g. the same sequence that the ligand would have if it was isolated from nature), or the amino acid sequence of the targeting moiety can share at least about 95%, at least about 90%, at least about 80%, at least about 70%, at least about 60%, at least about 50%, or at least about 40% sequence identity with the natural ligand (e.g., at least about this amount of sequence identity to the GenBank Accession Nos. listed in Table 2), as long as the variant retains or has enhanced biological activity of the native ligand. In some examples, such variants have an increased binding affinity for their target relative to the native ligand. A targeting moiety that is derived from a natural ligand can also be a fragment of the native sequence that is capable of binding to the target displayed by the cancer stem cell. In some examples, the ligand is a circularly permuted version of a natural ligand (e.g., see U.S. Pat. No. 6,011,002). Circularly permuted molecules include those in which the termini of a linear molecule (e.g., ligand) have been joined together, either directly or via a linker, to produce a circular molecule, and then the circular molecule is opened at another location to produce a new linear molecule with termini different from the termini in the original molecule. In some examples, the targeting moiety has one or more amino acid mutations (relative to the native sequence), which alters binding to the target, such as mutations that increase binding of a ligand to its target.

Cargo moieties can reduce, inhibit the growth of, and/or kill cancer cells and/or cancer stem cells, and in some examples also inhibit the growth of, and/or kill bulk cancer cells (e.g., non stem cancer cells). These molecules can be native proteins, or proteins that have been engineered, as well as other molecules that inhibit the growth of, and/or kill cancer cells and/or cancer stem cells, and in some examples also inhibit the growth of, and/or kill bulk cancer cells (e.g., non stem cancer cells). One example of such a molecule is a chemotherapeutic agent, such as thapsigargin. Cargo moieties can be linked to targeting moieties (a linked cargo moiety and targeting moiety is referred to herein as a IL-4 targeted cargo protein) that bind to cancer cells and/or cancer stem cells. Thus, the cargo moiety linked to the targeting moiety will bind to the cancer stem cell and inhibit the growth of (or kill) the cancer stem cell. In some examples, the cargo moiety can cause cancer stem cell death and in some examples the cancer stem cell death is caused by apoptosis. In some examples cargo moieties are toxins (including plant or microorganism derived toxins), active fragments of toxins, or derivatives of toxins that share at least about 95%, at least about 90%, at least about 80%, at least about 70%, at least about 60%, at least about 50%, or at least about 40% sequence identity with the natural toxin and retains or has enhanced biological activity of the native toxin, for example with the cargo moieties provided in Table 1. In other examples the cargo moieties are derived from proteins that modulate cell life cycles or are part of natural immune responses in animals. For example, some cargo moieties are derived from proteins that are known to induce apoptosis. In some examples cargo moieties are derived from pro-apoptotic proteins, active fragments of such proteins, or derivatives of such proteins that share at least about 95%, at least about 90%, at least about 80%, at least about 70%, at least about 60%, at least about 50%, or at least about 40% sequence identity with the natural moiety (see Table 1 for sequence accession numbers), as long as the variant retains or has enhanced biological activity of the native moiety. In additional examples a cargo moiety can be inactive when administered as part of a IL-4 targeted cargo protein, and then upon contacting another molecule in the subject become active. A more detailed description of cargo moieties is provided herein.

The description also includes methods of treating subjects having (or had) cancer with the IL-4 targeted cargo protein. For example, the method can include administering one or more disclosed IL-4 targeted cargo proteins to the subject, thereby treating cancer cells and/or cancer stem cells in the subject (e.g., reducing the number or volume of stem cells). For example, the IL-4 targeted cargo proteins can be used to treat subjects with recurrent cancer or cancer that is refractory. In such examples the subject is treated with a traditional anti-cancer therapy, for example radiation, surgery, or chemotherapy and then tested to determine the effectiveness of the treatment. If the traditional therapy did not alter the cancer in a desired way, the subject can then be treated with a IL-4 targeted cargo protein.

In some examples treatment regimes that include IL-4 targeted cargo proteins and additional anticancer therapeutics can be administered to a subject. The IL-4 targeted cargo protein and the additional anticancer therapeutic will vary depending upon the type of cancer stem cell being targeted.

In specific examples, a subject is administered one or more of the following specific IL-4 targeted cargo proteins to treat cancer cells and/or cancer stem cells: circularly permuted IL-4-Pseudomonas exotoxin (see U.S. Pat. No. 6,011,002), IL-4-BAD, as well as MDNA55.

A. IL-4 and/or IL-13 Mutein Fusion Proteins

The IL-4 and/or IL-13 muteins can be prepared as fusion or chimeric polypeptides that include a subject IL-4 and/or IL-13 mutein and a heterologous polypeptide (i.e., a polypeptide that is not IL-4 and/or IL-13 or a mutant thereof) (see, e.g., U.S. Pat. No. 6,451,308). Exemplary heterologous polypeptides can increase the circulating half-life of the chimeric polypeptide in vivo, and may, therefore, further enhance the properties of the mutant IL-4 and/or IL-13 polypeptides. In various embodiments, the polypeptide that increases the circulating half-life may be a serum albumin, such as human serum albumin, PEG, PEG-derivatives, or the Fc region of the IgG subclass of antibodies that lacks the IgG heavy chain variable region. Exemplary Fc regions can include a mutation that inhibits complement fixation and Fc receptor binding, or it may be lytic, i.e., able to bind complement or to lyse cells via another mechanism, such as antibody-dependent complement lysis (ADCC; U.S. Ser. No. 08/355,502 filed Dec. 12, 1994).

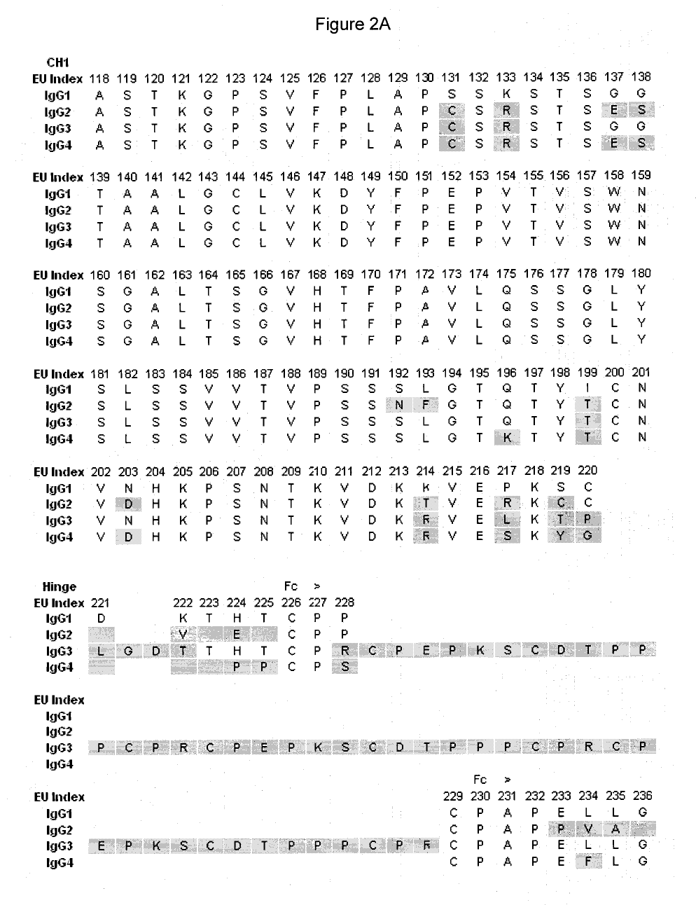

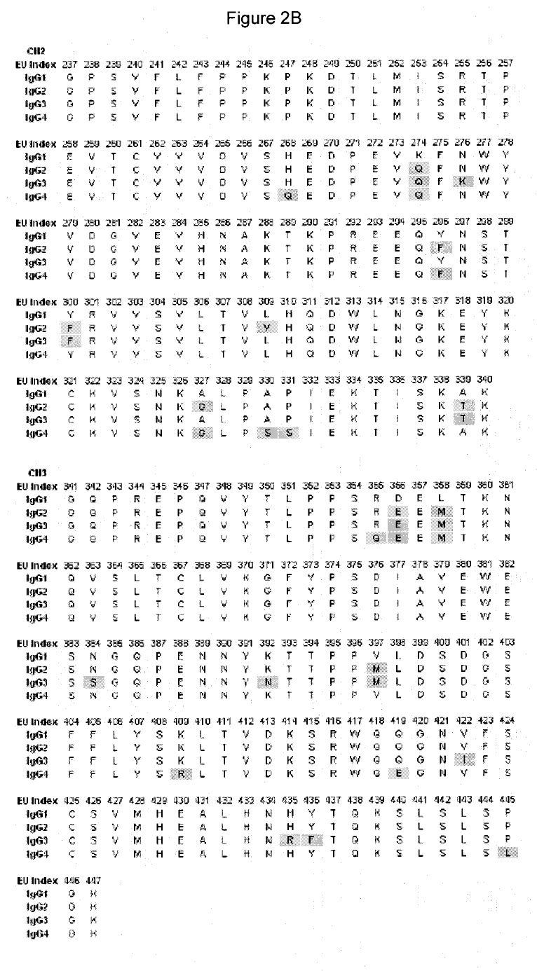

The “Fc region” can be a naturally occurring or synthetic polypeptide that is homologous to the IgG C-terminal domain produced by digestion of IgG with papain. IgG Fc has a molecular weight of approximately 50 kDa. The mutant IL-4 and/or IL-13 polypeptides can include the entire Fc region, or a smaller portion that retains the ability to extend the circulating half-life of a chimeric polypeptide of which it is a part. In addition, full-length or fragmented Fc regions can be variants of the wild-type molecule. In some embodiments, the IL-4 and/or IL-13 mutein fusion protein (e.g., an IL-4 and/or IL-13 mutein as described herein) includes an IgG1, IgG2, IgG3, or IgG4 Fc region (see, for example, sequences in FIG. 2A-2B). In some embodiments, the Fc region comprises the substitution N297A.

In some embodiments, the IL-4 and/or IL-13 mutein is linked directly or indirectly to the heterologous fusion polypeptide.

In some embodiments, the IL-4 and/or IL-13 mutein is linked directly to the Fc region. In some embodiments, the IL-4 and/or IL-13 mutein is linked to the Fc region via a linker peptide, such as GGGGS. In some embodiments, the linker is (GGGGS)n, wherein n is an integer between 1 and 10. In some embodiments, the linker is GGGGS. In some embodiments, the linker is GGGGSGGGGS (SEQ ID NO:70). In some embodiments, the linker is GGGGSGGGGSGGGGS (SEQ ID NO:71). In some embodiments, the linker is GGGGSGGGGSGGGGSGGGGS (SEQ ID NO:72). In some embodiments, the linker is GGGGSGGGGSGGGGSGGGGS (SEQ ID NO:73).

The Fc region can be “lytic” or “non-lytic,” but is typically non-lytic. A non-lytic Fc region typically lacks a high affinity Fc receptor binding site and a C′1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In other embodiments, the chimeric polypeptide can include a subject IL-4 and/or IL-13 mutein and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

In other embodiments, the chimeric polypeptide includes the mutant IL-4 and/or IL-13 polypeptide and a heterologous polypeptide that functions to enhance expression or direct cellular localization of the mutant IL-4 and/or IL-13 polypeptide, such as the Aga2p agglutinin subunit (see, e.g., Boder and Wittrup, Nature Biotechnol. 15:553-7, 1997).

In other embodiments, a chimeric polypeptide including a mutant IL-4 and/or IL-13 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the chimeric polypeptide comprises a fusion to an antibody or an antigen-binding portion thereof that disrupts the interaction between the PD-1 receptor and its ligand, PD-L1, and or is an antibody to a component of the PD-1/PD-L1 signaling pathway. Antibodies known in the art which bind to PD-1 and disrupt the interaction between the PD-1 and its ligand, PD-L1, and stimulate an anti-tumor immune response, are suitable for use in the chimeric polypeptides disclosed herein. In some embodiments, the antibody or antigen-binding portion thereof binds specifically to PD-1. For example, antibodies that target PD-1 and which can find used in the present invention include, e.g., but are not limited to nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (cemiplimab, Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies include anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in the chimeric polypeptides disclosed herein. For example, antibodies that target PD-L1 and are in clinical trials, include BMS-936559 (Bristol-Myers Squibb) and MPDL3280A (Genetech). Other suitable antibodies that target PD-L1 are disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, is suitable for use in the chimeric polypeptides disclosed herein. In some embodiments, the chimeric polypeptide comprises a fusion to an anti-PD-1 antibody. In some embodiments, the chimeric polypeptide comprises a fusion to an anti-PD-L1 antibody.

In some embodiments, the chimeric polypeptide comprises a fusion to an antibody or an antigen-binding portion thereof that targets CTLA-4 and disrupts its interaction with CD80 and CD86. Exemplary antibodies that target CTLA-4 include ipilimumab (MDX-010, MDX-101, Bristol-Myers Squibb), which is FDA approved, and tremelimumab (ticilimumab, CP-675, 206, Pfizer), currently undergoing human trials. Other suitable antibodies that target CTLA-4 are disclosed in WO 2012/120125, U.S. Pat. Nos. 6,984,720, 6,682,7368, and U.S. Patent Applications 2002/0039581, 2002/0086014, and 2005/0201994, herein incorporated by reference. It will be understood by one of ordinary skill that any antibody which binds to CTLA-4, disrupts its interaction with CD80 and CD86, and stimulates an anti-tumor immune response, is suitable for use in the chimeric polypeptides disclosed herein. In some embodiments, the chimeric polypeptide comprises a fusion to an anti-CTLA-4 antibody.

In some embodiments, the chimeric polypeptide comprises a fusion to an antibody or an antigen-binding portion thereof that targets LAG-3 and disrupts its interaction with MHC class II molecules. An exemplary antibody that targets LAG-3 is IMP321 (Immutep), currently undergoing human trials. Other suitable antibodies that target LAG-3 are disclosed in U.S. Patent Application 2011/0150892, herein incorporated by reference. It will be understood by one of ordinary skill that any antibody which binds to LAG-3, disrupts its interaction with MHC class II molecules, and stimulates an anti-tumor immune response, is suitable for use in the chimeric polypeptides disclosed herein. In some embodiments, the chimeric polypeptide comprises a fusion to an anti-LAG-3 antibody.

In some embodiments, the chimeric polypeptide comprises a fusion to an antibody or an antigen-binding portion thereof that targets B7-H3 or B7-H4. The B7 family does not have any defined receptors but these ligands are upregulated on tumor cells or tumor-infiltrating cells. An exemplary antibody that targets B7-H3 is MGA271 (Macrogenics) is currently undergoing human trials. Other suitable antibodies that target B7 family members are disclosed in U.S. Patent Application 2013/0149236, herein incorporated by reference. It will be understood by one of ordinary skill that any antibody which binds to B7-H3 or H4, and stimulates an anti-tumor immune response, is suitable for use in the chimeric polypeptides disclosed herein. In some embodiments, the chimeric polypeptide comprises a fusion to an anti-B7-H3 or B7-H4 antibody.

In some embodiments, the chimeric polypeptide comprises a fusion to an antibody or an antigen-binding portion thereof that targets TIM-3 and disrupts its interaction with galectin 9. Suitable antibodies that target TIM-3 are disclosed in U.S. Patent Application 2013/0022623, herein incorporated by reference. It will be understood by one of ordinary skill that any antibody which binds to TIM-3, disrupts its interaction with galectin 9, and stimulates an anti-tumor immune response, is suitable for use in the chimeric polypeptides disclosed herein. In some embodiments, the chimeric polypeptide comprises a fusion to an anti-TIM-3 antibody.

In some embodiments, the chimeric polypeptide comprises a fusion to an antibody or an antigen-binding portion thereof that targets 4-1BB/CD137 and disrupts its interaction with CD137L. It will be understood by one of ordinary skill that any antibody which binds to 4-1BB/CD137, disrupts its interaction with CD137L or another ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-4-1BB/CD137 antibody.

In some embodiments, the chimeric polypeptide comprises a fusion to an antibody or an antigen-binding portion thereof that targets GITR and disrupts its interaction with its ligand. It will be understood by one of ordinary skill that any antibody which binds to GITR, disrupts its interaction with GITRL or another ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-GITR antibody.

In some embodiments, the chimeric polypeptide comprises a fusion to an antibody or an antigen-binding portion thereof that targets OX40 and disrupts its interaction with its ligand. It will be understood by one of ordinary skill that any antibody which binds to OX40, disrupts its interaction with OX40L or another ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-OX40 antibody.

In some embodiments, the chimeric polypeptide comprises a fusion to an antibody or an antigen-binding portion thereof that targets CD40 and disrupts its interaction with its ligand. It will be understood by one of ordinary skill that any antibody which binds to CD40, disrupts its interaction with its ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-CD40 antibody

In some embodiments, the chimeric polypeptide comprises a fusion to an antibody or an antigen-binding portion thereof that targets ICOS and disrupts its interaction with its ligand. It will be understood by one of ordinary skill that any antibody which binds to ICOS, disrupts its interaction with its ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-ICOS antibody.

In some embodiments, the chimeric polypeptide comprises a fusion to an antibody or an antigen-binding portion thereof that targets CD28 and disrupts its interaction with its ligand. It will be understood by one of ordinary skill that any antibody which binds to CD28, disrupts its interaction with its ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-CD28 antibody.

In some embodiments, the chimeric polypeptide comprises a fusion to an antibody or an antigen-binding portion thereof that targets IFNα and disrupts its interaction with its ligand. It will be understood by one of ordinary skill that any antibody which binds to IFNα, disrupts its interaction with its ligand, and stimulates an anti-tumor immune response or an immune stimulatory response that results in anti-tumor activity overall, is suitable for use in the chimeric polypeptides disclosed herein. In some embodiments, the chimeric polypeptide comprises a fusion to an anti-IFNα antibody.

In some embodiments, the chimeric polypeptide comprises a fusion to a tumor antigen or polypeptide targeting a tumor antigen. Generally, tumor antigens allow for distinguishing the tumor cells from their normal cellular counterparts and can include, for example, tumor-specific antigens (TSA) as well as tumor-associated antigens (TAA). In some embodiments, a tumor antigen is a protooncogene and/or a tumor suppressor, as well as overexpressed or aberrantly expressed cellular proteins, tumor antigens produced by oncogenic viruses, oncofetal antigens, altered cell surface glycolipids and glycoproteins, and/or cell type-specific differentiation antigens. Such tumor antigens can include melanoma antigens, cancer-testis antigens, epithelial tumor antigens, cell cycle regulatory proteins, prostate specific antigens (including prostate carcinoma antigens, such as for example those disclosed in U.S. Pat. No. 5,538,866) lymphoma (U.S. Pat. Nos. 4,816,249; 5,068,177; and 5,227,159). Tumor antigens can include for example, but are not limited to, HMW mucins bound by 2G3 and 369F10, c-erbB-2 related tumor antigen (an approximately 42 kD or 55 kD glycoprotein), the approximately 40, 60, 100 and 200 kD antigens bound by 113F1, 9-O-acetyl GD3, p97, alphafetoprotein (AFP) (for example, for germ cell tumors and/or hepatocellular carcinoma), carcinoembryonic antigen (CEA) (for example, for bowel cancers occasional lung or breast cancer), CA-125 (for example, for ovarian cancer), MUC-1 (for example, for breast cancer), epithelial tumor antigen (ETA) (for example, for breast cancer), tyrosinase (for example, for malignant melanoma), melanoma-associated antigen (MAGE) (for example, for malignant melanoma), cancer/testis antigen 1 (CTAG1B), melanoma-associated antigen 1 (MAGEA1), abnormal Ras products, abnormal p53 products, overexpression of cyclins (including, for example, cyclin B1), mutation in fibronectin, posttranslational alteration in the MUC1 glycoprotein, secreted tumor antigens (including, for example, gangliosides).

B. IL-4 Targeted Cargo Proteins

IL-4 targeted cargo proteins are proteins that include a targeting moiety linked to a cargo moiety. IL-4 targeted cargo proteins function to specifically bind to cancer cells and/or cancer stem cells and reduce or inhibit cancer stem cell growth, as well as targeting the immunosuppressive cells in the tumor microenvironment (TME). In some embodiments, IL-4 targeted cargo proteins comprise an IL-4R targeting moiety. In some embodiments, IL-4 targeted cargo proteins comprise an IL-4R targeting moiety comprising IL-4 or a variant thereof as described herein. In some embodiments, IL-4 targeted cargo proteins comprise an IL-4R targeting moiety comprising IL-13 or a variant thereof as described herein. In some embodiments, the IL-4 targeted cargo protein comprises MDNA55 (SEQ ID NO:65) or a variant thereof. In some embodiments, the IL-4 targeted cargo protein is MDNA55 (SEQ ID NO:65).

The IL-4R targeting moiety can comprise an IL-4 sequence or variant thereof. Exemplary polypeptide sequences are provided in SEQ ID NO:51-SEQ ID NO:55, SEQ ID NO:58-SEQ ID NO:62, and SEQ ID NO:64-SEQ ID NO:69. In some embodiments, the polypeptide sequence is as provided in any one of SEQ ID NO through 51-SEQ ID NO:55, SEQ ID NO:58 through SEQ ID NO:62, and/or SEQ ID NO:64 through SEQ ID NO: 66. In some embodiments, the polypeptide sequence is SEQ ID NO:51. In some embodiments, the polypeptide sequence is SEQ ID NO:52. In some embodiments, the polypeptide sequence is SEQ ID NO:53. In some embodiments, the polypeptide sequence is SEQ ID NO:54. In some embodiments, the polypeptide sequence is SEQ ID NO:55. In some embodiments, the polypeptide sequence is SEQ ID NO:58. In some embodiments, the polypeptide sequence is SEQ ID NO:59. In some embodiments, the polypeptide sequence is SEQ ID NO:60. In some embodiments, the polypeptide sequence is SEQ ID NO:61. In some embodiments, the polypeptide sequence is SEQ ID NO:62. In some embodiments, the polypeptide sequence is SEQ ID NO:64. In some embodiments, the polypeptide sequence is SEQ ID NO:65. In some embodiments, the polypeptide sequence is SEQ ID NO:66. In some embodiments, the polypeptide sequence is SEQ ID NO:67. In some embodiments, the polypeptide sequence is SEQ ID NO:68. In some embodiments, the polypeptide sequence is SEQ ID NO:69. In some embodiments, the polypeptide sequence is 98% identical to any one of SEQ ID NO through 51-SEQ ID NO:55, SEQ ID NO:58 through SEQ ID NO:62, and/or SEQ ID NO:64 through SEQ ID NO:66. In some embodiments, the polypeptide sequence is 99% identical to any one of SEQ ID NO through 51-SEQ ID NO:55, SEQ ID NO:58 through SEQ ID NO:62, and/or SEQ ID NO:64 through SEQ ID NO:66. In some embodiments, any one of SEQ ID NO through 51-SEQ ID NO:55, SEQ ID NO:58 through SEQ ID NO:62, and/or SEQ ID NO:64 through SEQ ID NO:66 are part of the IL-4R targeting moiety. In some embodiments, SEQ ID NO:51 is part of the IL-4R targeting moiety. In some embodiments, SEQ ID NO:52 is part of the IL-4R targeting moiety. In some embodiments, SEQ ID NO:53 is part of the IL-4R targeting moiety. In some embodiments, SEQ ID NO:54 is part of the IL-4R targeting moiety. In some embodiments, SEQ ID NO:55 is part of the IL-4R targeting moiety. In some embodiments, SEQ ID NO:58 is part of the IL-4R targeting moiety. In some embodiments, SEQ ID NO:59 is part of the IL-4R targeting moiety. In some embodiments, SEQ ID NO:60 is part of the IL-4R targeting moiety. In some embodiments, SEQ ID NO:61 is part of the IL-4R targeting moiety. In some embodiments, SEQ ID NO:62 is part of the IL-4R targeting moiety. In some embodiments, SEQ ID NO:64 is part of the IL-4R targeting moiety. In some embodiments, SEQ ID NO:65 is part of the IL-4R targeting moiety. In some embodiments, SEQ ID NO:66 is part of the IL-4R targeting moiety. In some embodiments, SEQ ID NO:67 is part of the IL-4R targeting moiety. In some embodiments, SEQ ID NO:68 is part of the IL-4R targeting moiety. In some embodiments, SEQ ID NO:69 is part of the IL-4R targeting moiety.

The IL-13 superkine component of the construct may be at least about 50 amino acids in length, at least about 75, at least about 100, at least about 110, at least about 115 amino acids in length, up to the full-length of the wild-type protein at the transmembrane domain, i.e. about 116 amino acids in length. For example, the superkine may be fused to the hinge, transmembrane or signaling domains of a CAR. Exemplary polypeptide sequences are provided in SEQ ID NO:2-SEQ ID NO:48, SEQ ID NO:56, SEQ ID NO:57, and SEQ ID NO:63. In some embodiments, the polypeptide sequence is as provided in any one of SEQ ID NO:2 through SEQ ID NO:38. In some embodiments, the polypeptide sequence is SEQ ID NO:2. In some embodiments, the polypeptide sequence is SEQ ID NO:2. In some embodiments, the polypeptide sequence is SEQ ID NO:3. In some embodiments, the polypeptide sequence is SEQ ID NO:4. In some embodiments, the polypeptide sequence is SEQ ID NO:5. In some embodiments, the polypeptide sequence is SEQ ID NO:6. In some embodiments, the polypeptide sequence is SEQ ID NO:7. In some embodiments, the polypeptide sequence is SEQ ID NO:8. In some embodiments, the polypeptide sequence is SEQ ID NO:9. In some embodiments, the polypeptide sequence is SEQ ID NO:10. In some embodiments, the polypeptide sequence is SEQ ID NO: 11. In some embodiments, the polypeptide sequence is SEQ ID NO:12. In some embodiments, the polypeptide sequence is SEQ ID NO:13. In some embodiments, the polypeptide sequence is SEQ ID NO:14. In some embodiments, the polypeptide sequence is SEQ ID NO:15. In some embodiments, the polypeptide sequence is SEQ ID NO:16. In some embodiments, the polypeptide sequence is SEQ ID NO:17. In some embodiments, the polypeptide sequence is SEQ ID NO: 18. In some embodiments, the polypeptide sequence is SEQ ID NO:19. In some embodiments, the polypeptide sequence is SEQ ID NO:20. In some embodiments, the polypeptide sequence is SEQ ID NO:21. In some embodiments, the polypeptide sequence is SEQ ID NO:22. In some embodiments, the polypeptide sequence is SEQ ID NO:23. In some embodiments, the polypeptide sequence is SEQ ID NO:24. In some embodiments, the polypeptide sequence is SEQ ID NO:25. In some embodiments, the polypeptide sequence is SEQ ID NO:26. In some embodiments, the polypeptide sequence is SEQ ID NO:27. In some embodiments, the polypeptide sequence is SEQ ID NO:28. In some embodiments, the polypeptide sequence is SEQ ID NO:29. In some embodiments, the polypeptide sequence is SEQ ID NO:30. In some embodiments, the polypeptide sequence is SEQ ID NO:31. In some embodiments, the polypeptide sequence is SEQ ID NO:32. In some embodiments, the polypeptide sequence is SEQ ID NO:33. In some embodiments, the polypeptide sequence is SEQ ID NO:34. In some embodiments, the polypeptide sequence is SEQ ID NO:35. In some embodiments, the polypeptide sequence is SEQ ID NO:36. In some embodiments, the polypeptide sequence is SEQ ID NO:37. In some embodiments, the polypeptide sequence is SEQ ID NO:38. In some embodiments, the polypeptide sequence is 90% identical to any one of SEQ ID NO:2 through SEQ ID NO:38. In some embodiments, the polypeptide sequence is 95% identical to any one of SEQ ID NO:2 through SEQ ID NO:38. In some embodiments, the polypeptide sequence is 98% identical to any one of SEQ ID NO:2 through SEQ ID NO:38. In some embodiments, the polypeptide sequence is 99% identical to any one of SEQ ID NO:2 through SEQ ID NO:38. In some embodiments, any one of SEQ ID NO:2 through SEQ ID NO:38 is part of the IL-4R targeting moiety. In some embodiments, SEQ ID NO:2 is part of the IL-4R targeting moiety. In some embodiments, SEQ ID NO:3 is part of the IL-4R targeting moiety. In some embodiments, SEQ ID NO:4 is part of the IL-4R targeting moiety. In some embodiments, SEQ ID NO:5 is part of the IL-4R targeting moiety. In some embodiments, SEQ ID NO:6 is part of the IL-4R targeting moiety. In some embodiments, SEQ ID NO:7 is part of the IL-4R targeting moiety. In some embodiments, SEQ ID NO:8 is part of the IL-4R targeting moiety. In some embodiments, SEQ ID NO:9 is part of the IL-4R targeting moiety. In some embodiments, SEQ ID NO:10 is part of the IL-4R targeting moiety. In some embodiments, SEQ ID NO:11 is part of the IL-4R targeting moiety. In some embodiments, SEQ ID NO:12 is part of the IL-4R targeting moiety. In some embodiments, SEQ ID NO:13 is part of the IL-4R targeting moiety. In some embodiments, SEQ ID NO:14 is part of the IL-4R targeting moiety. In some embodiments, SEQ ID NO:15 is part of the IL-4R targeting moiety. In some embodiments, SEQ ID NO:16 is part of the IL-4R targeting moiety. In some embodiments, SEQ ID NO:17 is part of the IL-4R targeting moiety. In some embodiments, SEQ ID NO:18 is part of the IL-4R targeting moiety. In some embodiments, SEQ ID NO:19 is part of the IL-4R targeting moiety. In some embodiments, SEQ ID NO:20 is part of the IL-4R targeting moiety. In some embodiments, SEQ ID NO:21 is part of the IL-4R targeting moiety. In some embodiments, SEQ ID NO:22 is part of the IL-4R targeting moiety. In some embodiments, SEQ ID NO:23 is part of the IL-4R targeting moiety. In some embodiments, SEQ ID NO:24 is part of the IL-4R targeting moiety. In some embodiments, SEQ ID NO:25 is part of the IL-4R targeting moiety. In some embodiments, SEQ ID NO:26 is part of the IL-4R targeting moiety. In some embodiments, SEQ ID NO:27 is part of the IL-4R targeting moiety. In some embodiments, SEQ ID NO:28 is part of the IL-4R targeting moiety. In some embodiments, SEQ ID NO:29 is part of the IL-4R targeting moiety. In some embodiments, SEQ ID NO:30 is part of the IL-4R targeting moiety. In some embodiments, SEQ ID NO:31 is part of the IL-4R targeting moiety. In some embodiments, SEQ ID NO:32 is part of the IL-4R targeting moiety. In some embodiments, SEQ ID NO:33 is part of the IL-4R targeting moiety. In some embodiments, SEQ ID NO:34 is part of the IL-4R targeting moiety. In some embodiments, SEQ ID NO:35 is part of the IL-4R targeting moiety. In some embodiments, SEQ ID NO:36 is part of the IL-4R targeting moiety. In some embodiments, SEQ ID NO:37 is part of the IL-4R targeting moiety. In some embodiments, SEQ ID NO:38 is part of the IL-4R targeting moiety. In some embodiments, SEQ ID NO:40 is part of the IL-4R targeting moiety. In some embodiments, SEQ ID NO:41 is part of the IL-4R targeting moiety. In some embodiments, SEQ ID NO:43 is part of the IL-4R targeting moiety. In some embodiments, SEQ ID NO:44 is part of the IL-4R targeting moiety. In some embodiments, SEQ ID NO:45 is part of the IL-4R targeting moiety. In some embodiments, SEQ ID NO:46 is part of the IL-4R targeting moiety. In some embodiments, SEQ ID NO:47 is part of the IL-4R targeting moiety. In some embodiments, SEQ ID NO:48 is part of the IL-4R targeting moiety.

Table of IL-13 sequences is provided below.

TABLE 5
List of IL-13 Amino Acid Sequences

| SEQ ID NO: | |
|---|---|
| (Information) | Amino acid sequence |
| SEQ ID NO: 1 | PGPVPPSTALRELIEELVNITQNQKAPLCNGSMVW |
| (IL-13 wildtype) | SINLTAGMYCAALESLINVSGCSAIEKTQRMLSGF |
| | CPHKVSAGQFSSLHVRDTKIEVAQFVKDLLLHLKK |
| | LFREGQFN |
| SEQ ID NO: 2 | PGPVPPSTAVRALIEELINITQNQKAPLCNGSMVW |
| | SINRTAGMYCAALESLINVSGCSAIEKTQDMLSGF |
| | CPHKVSAGQFSSLHVRSSKIEVAQFVKDLLFHLRT |
| | LFREGQFN |
| SEQ ID NO: 3 | PGPVPPSTAIRELIEELINITQNQKAPLCNGSMVW |
| | SINLTAGMYCAALESLINVSGCSAIEKTQRMLSGF |
| | CPHKVSAGQFSSLHVRGSKIEVAQFVKDLLHHLRA |
| | LFREGQFN |
| SEQ ID NO: 4 | PGPVPPSTAVRELIEELINITQNQKAPLCNGSMVW |
| | SINRTAGMYCAALESLINVSGCSAIEKTQRMLSGF |
| | CPHKVSAGQFSSLHVRSSKIEVAQFVKDLLFHLRT |
| | LFREGQFN |
| SEQ ID NO: 5 | PGPVPPSTALIELIEELINITQNQKAPLCNGSMVW |
| | SINLTAGIYCAALESLINVSGCSAIEKTQRMLSGF |
| | CPHKVSAGQFSSLHVKGSKIEVAQFVKDLLHHLRA |
| | LMREGQFN |
| SEQ ID NO: 6 | PGPVPPSTAIRELIEELLNITQNQKAPLCNGSMVW |
| | SINLTAGMYCAALESLINVSGCSAIEKTQRMLSGF |
| | CPHKVSAGQFSSLHVMKSKIEVAQFVKDLLHHLRA |
| | LFREGQFN |
| SEQ ID NO: 7 | PGPVPPSTAIRELIEELINITQNQKAPLCNGSMVW |
| | SINLTAGMYCAALESLINVSGCSAIEKTQRMLSGF |
| | CPHKVSAGQFSSLHVRSSRIEVAQFVKDLLHHLRT |
| | LFREGQFN |
| SEQ ID NO: 8 | PGPVPPSTALRELIEELINITQNEKAPLCNGSMVW |
| | SINLTAGIYCAALESLINVSGCSAIEKTQRMLSGF |
| | CPHKVSAGQFSSLHVTGSKIEVAQFVKDLLYHLRA |
| | LFREGQFN |
| SEQ ID NO: 9 | PGPVPPSTALSELIEELINITQNQKAPLCNGSMVW |
| | SINPTAGMYCAALESLINVSGCSAIEKTQRMLSGF |
| | CPHKVAAGQFSSLHDKGSMIEVAQFVKDLLYHLRT |
| | LFREGQFN |
| SEQ ID NO: 10 | PGPVPPSTATRELIEELINITQNQKAPLCNGSMVW |
| | SINLTADMYCAALESLINVSGCSAIEKTQRMLSGF |
| | CPHKVSVGQFSSLHVRGSKIEVAQFVKDLLYHLRT |
| | LFREGQFN |
| SEQ ID NO: 11 | PGPVPPSTADIELIAELINITQNQKAPLCNGSMVW |
| | SINLTADMYCAALESLINVSGCSAIEKTQRMLSGF |
| | CPHKVSAGQFSSLHVKKTRIEVAQFVKDLLLHLKK |
| | LFKEGQFN |
| SEQ ID NO: 12 | PGPVPPSTAARELIEELVNITQNQKAPLCNGSMVW |
| | SINLTAGMYCAALESLINVSGCSAIEKTQRMLSGF |
| | CPHKVSAGQLSSLHVTGKRIEVAQFVKDLLNHLRA |
| | LFKEGQFN |
| SEQ ID NO: 13 | PGPVPPSTAVRELIEELVNITQNQKAPLCNGSMVW |
| | SINLTAGMYCAALESLINVSGCSAIEKTQRMLSGF |
| | CPHKVSAGQFSSLHVRDTRIEVAQFVKDLLNHLKE |
| | LFTEGQFN |
| SEQ ID NO: 14 | PGPVPPSTALSELMEELVNITQNQKAPLCNGSMVW |
| | SINLTAGMYCAALESLINVSGCSAIEKTQRMLSGF |
| | CPHKVSAGQFSSLHVRDSKIEVAQFVKDLLNHLKA |
| | LFKEGQFN |
| SEQ ID NO: 15 | GPVPPSTAFRELIEELVNITQNQKAPLCNGSMVWS |
| | INLTAGMYCAALESLINVSGCSAIEKTQRMLSGFC |
| | PHKVSPGQFSSLHVTNSRIEVAQFVKDLLNHLKAL |
| | FKEGQYN |
| SEQ ID NO: 16 | GPVPPSTAHLELIEELINITQNQKAPLCNGSMVWS |
| | INLTAGMYCAALESLINVSGCSAIEKTQRMLSGFC |
| | PHKVSAGQFSSLHVKETRIEVAQFVKDLLNHLKTL |
| | FKEGQFN |
| SEQ ID NO: 17 | PGPVPPSTAHLELIEELINITQNQKAPLCNGSMVW |
| | SINPTAGMYCAALESLINVSGCSAIEKTQRMLSGF |
| | CPHKVSAGQFSSLHVMDTRIEVAQFVKDLLLHLKK |
| | LFKEGQFN |
| SEQ ID NO: 18 | PGPVPPSTAHRELIEELVNITQNQKAPLCNGSMVW |
| | SINLTAGMYCAALESLINVSGCSAIEKTQRMLSGF |
| | CPHKVSAGQFSSLHVTGRKIEVAQFVKDLLLHLKK |
| | LFKEGQFN |
| SEQ ID NO: 19 | PGPVPPSTAHRELIEELVNITQNQKAPLCNGSMVW |
| | RINRTAGMYCAALESLINVSGCSAIEKTQRMLSGF |
| | CPHKVSAGQFSSLHVMDSRIEVAQFVKDLLNHLRA |
| | LFKEGQFN |
| SEQ ID NO: 20 | PGPVPPSTAARELIEELFNITQNQKAPLCNGSMVW |
| | SINLTAGMYCAALESLINVSGCSAIEKTKRMLSGF |
| | CPHKVSAGQFPSLHVKKTRIEVAQFVKDLLIHLRK |
| | LFKEGQFN |
| SEQ ID NO: 21 | PGPVPPSTALIELIEELINITQNQKAPLCNGSMVWS |
| (Exemplary sequence | INLTAGMYCAALESLINVSGCSAIEKTQRMLSGFCP |
| comprising R11I, V18I, R86K, | HKVSAGQFSSLHVKGSKIEVAQFVKDLLHHLRALMR |
| D87G, T88S, L101H, K104R, | EGQFN |
| K105A, F107M, referred to | |
| herein as A5) | |
| SEQ ID NO: 22 | PGPVPPSTAIRELIEELLNITQNQKAPLCNGSMVWS |
| (Exemplary sequence | INLTAGMYCAALESLINVSGCSAIEKTQRMLSGFCP |
| comprising L10I, V18L, R86M, | HKVSAGQFSSLHVMKSKIEVAQFVKDLLHHLRALFR |
| D87K, T88S, L101H, K104R, | EGQFN |
| K105A, referred to herein as A6) | |
| SEQ ID NO: 23 | PGPVPPSTAIRELIEELINITQNQKAPLCNGSMVWS |
| (Exemplary sequence | INLTAGMYCAALESLINVSGCSAIEKTQRMLSGFCP |
| comprising L10I, V18I, D87G, | HKVSAGQFSSLHVRGSKIEVAQFVKDLLHHLRALFR |
| T88S, L101H, K104R, K105A, | EGQFN |
| referred to herein as A7) | |
| SEQ ID NO: 24 | PGPVPPSTAIRELIEELINITQNQKAPLCNGSMVWS |
| (Exemplary sequence | INLTAGMYCAALESLINVSGCSAIEKTQRMLSGFCP |
| comprising L10I, V18I, D87S, | HKVSAGQFSSLHVRSSRIEVAQFVKDLLHHLRTLFR |
| T88S, K89R, L101H, K104R, | EGQFN |
| K105T; referred to herein as A8) | |
| SEQ ID NO: 25 | PGPVPPSTAVRELIEELINITQNQKAPLCNGSMVWS |
| (Exemplary sequence | INLTAGMYCAALESLINVSGCSAIEKTQRMLSGFCP |
| comprising L10V, V18I, D87S, | HKVSAGQFSSLHVRSSKIEVAQFVKDLLFHLRTLFR |
| T88S, L101F, K104R, K105T, | EGQFN |
| referred to herein as A11 variant | |
| 1) | |
| SEQ ID NO: 25 | PGPVPPSTAVRELIEELINITQNQKAPLCNGSMVWS |
| (Exemplary sequence | INRTAGMYCAALESLINVSGCSAIEKTQRMLSGFCP |
| comprising L10V, V18I, D87S, | HKVSAGQFSSLHVRSSKIEVAQFVKDLLFHLRTLFR |
| T88S, L101F, K104R, K105T, | EGQFN |
| referred to herein as A11 variant | |
| 2) | |
| SEQ ID NO: 26 | PGPVPPSTALRELIEELINITQNQKAPLCNGSMVW |
| (Exemplary sequence | SINLTAGMYCAALESLINVSGCSAIEKTQRMLSGF |
| comprising V18I, R86T, D87G, | CPHKVSAGQFSSLHVTGSKIEVAQFVKDLLYHLRA |
| T88S, L101Y, K104R, K105A, | LFREGQFN |
| referred to herein as B2) | |
| SEQ ID NO: 27 | PGPVPPSTALSELIEELINITQNQKAPLCNGSMVW |
| (Exemplary sequence | SINLTAGMYCAALESLINVSGCSAIEKTQRMLSGF |
| comprising R11S, V18I, R86K, | CPHKVSAGQFSSLHVKGSMIEVAQFVKDLLYHLRT |
| D87G, T88S, K89M, L101Y, | LFREGQFN |
| K104R, K105T, referred to | |
| herein as B4) | |
| SEQ ID NO: 28 | PGPVPPSTATRELIEELINITQNQKAPLCNGSMVW |
| (Exemplary sequence | SINLTAGMYCAALESLINVSGCSAIEKTQRMLSGF |
| comprising L10T, V18I, D87G, | CPHKVSAGQFSSLHVRGSKIEVAQFVKDLLYHLRT |
| T88S, K89K, L10Y1, K104R, | LFREGQFN |
| K105T, referred to herein as B6) | |
| SEQ ID NO: 29 | PGPVPPSTADIELIEELINITQNQKAPLCNGSMVW |
| (Exemplary sequence | SINLTAGMYCAALESLINVSGCSAIEKTQRMLSGF |
| comprising L10D, R11I, V18I, | CPHKVSAGQFSSLHVKKTRIEVAQFVKDLLLHLKK |
| R86K, D87K, K89R, R108K, | LFKEGQFN |
| referred to herein as C2) | |
| SEQ ID NO: 30 | PGPVPPSTAARELIEELVNITQNQKAPLCNGSMVW |
| (Exemplary sequence | SINLTAGMYCAALESLINVSGCSAIEKTQRMLSGF |
| comprising L10A, R86T, D87G, | CPHKVSAGQFSSLHVTGKRIEVAQFVKDLLNHLRA |
| T88K, K89R, L101N, K104R, | LFKEGQFN |
| K105A, R108K, referred to | |
| herein as C3) | |
| SEQ ID NO: 31 | PGPVPPSTAVRELIEELVNITQNQKAPLCNGSMVW |
| (Exemplary sequence | SINLTAGMYCAALESLINVSGCSAIEKTQRMLSGF |
| comprising L10V, K89R, L101N, | CPHKVSAGQFSSLHVRDTRIEVAQFVKDLLNHLKE |
| K105E, R108T, referred to | LFTEGQFN |
| herein as C4) | |
| SEQ ID NO: 32 | PGPVPPSTALSELMEELVNITQNQKAPLCNGSMVW |
| (Exemplary sequence | SINLTAGMYCAALESLINVSGCSAIEKTQRMLSGF |
| comprising R11S, I14M, T88S, | CPHKVSAGQFSSLHVRDSKIEVAQFVKDLLNHLKA |
| L101N, K105A, R108K, referred | LFKEGQFN |
| to herein as C7) | |
| SEQ ID NO: 33 | PGPVPPSTAHLELIEELINITQNQKAPLCNGSMVW |
| (Exemplary sequence | SINLTAGMYCAALESLINVSGCSAIEKTQRMLSGF |
| comprising L10H, R11L, V18I, | CPHKVSAGQFSSLHVKETRIEVAQFVKDLLNHLKT |
| R86K, D87E, K89R, L101N, | LFKEGQFN |
| K105T, R108K, refered to herein | |
| as C9) | |
| SEQ ID NO: 34 | PGPVPPSTAHLELIEELINITQNQKAPLCNGSMVW |
| (Exemplary sequence | SINLTAGMYCAALESLINVSGCSAIEKTQRMLSGF |
| comprising L10H, R11L, V18I, | CPHKVSAGQFSSLHVMDTRIEVAQFVKDLLLHLKK |
| R86M, K89R, R108K, referred to | LFKEGQFN |
| herein as C10) | |
| SEQ ID NO: 35 | PGPVPPSTAHRELIEELVNITQNQKAPLCNGSMVW |
| (Exemplary sequence | SINLTAGMYCAALESLINVSGCSAIEKTQRMLSGF |
| comprising L10H, R86T, D87G, | CPHKVSAGQFSSLHVTGRKIEVAQFVKDLLLHLKK |
| T88R, R108K, referred to herein | LFKEGQFN |
| as C11) | |
| SEQ ID NO: 36 | PGPVPPSTAHRELIEELVNITQNQKAPLCNGSMVW |
| (Exemplary sequence | SINLTAGMYCAALESLINVSGCSAIEKTQRMLSGF |
| comprising L10H, R86M, T88S, | CPHKVSAGQFSSLHVMDSRIEVAQFVKDLLNHLRA |
| K89R, L101N, K104R, K105A, | LFKEGQFN |
| R108K, referred to herein as | |
| C12) | |
| SEQ ID NO: 37 | PGPVPPSTAARELIEELFNITQNQKAPLCNGSMVW |
| (Exemplary sequence | SINLTAGMYCAALESLINVSGCSAIEKTQRMLSGF |
| comprising L10A, V18F, R86F, | CPHKVSAGQFSSLHVKKTRIEVAQFVKDLLIHLRK |
| D87F, K89R, L101I, K104R, | LFKEGQFN |
| R108K, referred to herein as D7) | |
| SEQ ID NO: | PGPVPPSTAVRALIEELINITQNQKAPLCNGSMVW |
| (Exemplary sequence | SINLTAGMYCAALESLINVSGCSAIEKTQDMLSGF |
| comprising L10V, E12A, V18I, | CPHKVSAGQFSSLHVRSSKIEVAQFVKDLLFHLRT |
| R65D, D87S, T88S, L101F, | LFREGQFN |
| K104R, K105T, referred to | |
| herein as IL-13dn) | |
| SEQ ID NO: 39 | MHPLLNPLLLALGLMALLLTTVIALTCLGGFASPG |
| **signal peptide** | PVPPSTAHRELIEELVNITQNQKAPLCNGSMVWSI |
| | NLTAGMYCAALESLINVSGCSAIEKTQRMLSGFCP |
| | HKVSAGQFSSLHVTGRKIEVAQFVKDLLLHLKKLF |
| | KEGQFN |
| SEQ ID NO: 40 | PGPVPPSTAVRALIEELINITQNQKAPLCNGSMVW |
| (Exemplary sequence | SINRTAGMYCAALESLINVSGCSAIEKTQDMLSGF |
| comprising L10V, E12A, V18I, | CPHKVSAGQFSSLHVRSSKIEVAQFVKDLLFHLRT |
| R65D, D87S, T88S, L101F, | LFREGQFN |
| K104R, K105T, referred to | |
| herein as IL-13DN variant 1) | |
| SEQ ID NO: 41 | PGPVPPSTAVRALIEELINITQNQKAPLCNGSMVW |
| (Exemplary sequence | SINLTAGMYCAALESLINVSGCSAIEKTQDMLSGF |
| comprising L10V, E12A, V18I, | CPHKVSAGQFSSLHVRSSKIEVAQFVKDLLFHLRT |
| R65D, D87S, T88S, L101F, | LFREGQFN |
| K104R, K105T, referred to | |
| herein as IL-13DN variant 2) | |
| SEQ ID NO: 42 | MPGPVPPSTALRELIEELVNITQNQKAPLCNGSMV |
| wild-type IL-13 including an | WSINLTAGMYCAALESLINVSGCSAIEKTQRMLSG |
| additional methionine at the N- | FCPHKVSAGQFSSLHVRDTKIEVAQFVKDLLLHLK |
| terminus | KLFREGQFN |
| SEQ ID NO: 43 | MYCAALESLINVSGCSAIEKTQRMLSGFCPHKVSA |
| circularly permuted IL-13 | GQFSSLHVRDTKIEVAQFVKDLLLHLKKLFREGQF |
| | NGGSGPGPVPPSTALRELIEELVNITQNQKAPLCN |
| | GSMVWSINLTAG |
| SEQ ID NO: 44 | MYCAALESLINVSGCSAIEKTQRMLSGFCPHKVSA |
| Circularly permuted IL-13 | GQFSSLHVRDTKIEVAQFVKDLLLHLKKLFREGQF |
| | NGGSGMPGPVPPSTALRELIEELVNITQNQKAPLC |
| | NGSMVWSINLTAG |
| SEQ ID NO: 5 | MYCAALESLINVSGCSAIEKTQRMLSGFCPHKVSA |
| circularly permuted IL-13 ″A11″ | GQFSSLHVRSSKIEVAQFVKDLLFHLRTLFREGQF |
| variant | NGGSGPGPVPPSTAVRELIEELINITQNQKAPLCN |
| | GSMVWSINRTAG |
| SEQ ID NO: 46 | MYCAALESLINVSGCSAIEKTQRMLSGFCPHKVSA |
| circularly permuted IL-13 | GQFSSLHVRSSKIEVAQFVKDLLFHLRTLFREGQF |
| | NGGSGMPGPVPPSTAVRELIEELINITQNQKAPLC |
| | NGSMVWSINRTAG |
| SEQ ID NO: 47 | MYCAALESLINVSGCSAIEKTQDMLSGFCPHKVSA |
| circularly permuted IL-13 ″DN″ | GQFSSLHVRSSKIEVAQFVKDLLFHLRTLFREGQF |
| variant | NGGSGPGPVPPSTAVRALIEELINITQNQKAPLCN |
| | GSMVWSINLTAG |
| SEQ ID NO: 48 | MYCAALESLINVSGCSAIEKTQDMLSGFCPHKVSA |
| circular permuted IL-13 | GQFSSLHVRSSKIEVAQFVKDLLFHLRTLFREGQF |
| | NGGSGMPGPVPPSTAVRALIEELINITQNQKAPLC |
| | NGSMVWSINLTAG |

Table of IL-4 sequences is provided below.

TABLE 6
List of IL-4 Amino Acid Sequences

| SEQ ID NO: | |
|---|---|
| (Information) | Amino acid sequence |
| SEQ ID NO: 49 | MGLTSQLLPPLFELLACAGNEVHGHKCDITLQEII |
| (IL-4 wildtype with signal | KTLNSLTEQKTLCTELTVTDIFAASKNTTEKETFC |
| peptide) | RAATVLRQFYSHHEKDTRCLGATAQQFHRHKQLIR |
| | FLKRLDRNLWGLAGLNSCPVKEANQSTLENFLERL |
| | KTIMREKYSKCSS |
| SEQ ID NO: 50 | MHKCDITLQEIIKTLNSLTEQKTLCTELTVTDIFA |
| IL-4 including an additional | ASKDTTEKETFCRAATVLRQFYSHHEKDTRCLGAT |
| methionine at the N-terminus″ | AQQFHRHKQLIRFLKRLDRNLWGLAGLNSCPVKEA |
| starting | NQSTLENFLERLKTIMREKYSKCSS |
| SEQ ID NO: 51 | KCDITLQEIIKTLNSLTEQKTLCTELTVTDIFAAS |
| KFR | KNTTEKETFCRAATVLRQFYSHHEKDTRCLGATAQ |
| | QFHRHKQLIRFLKRLDRNLWGLAGLNSCPVKEANQ |
| | STLENFLERLKTIMKEKFRKCSS |
| SEQ ID NO: 52 | MDTTEKETFCRAATVLRQFYSHHEKDTRCLGATAQ |
| RGA | QFHRHKQLIRFLKRLDRNLWGLAGLNSCPVKEANQ |
| | STLENFLERLRVIMQSKWFKCGAGGNGGHKCDITL |
| | QEIIKTLNSLTEQKTLCTELTVTDIFAAS |
| SEQ ID NO: 53 | MDTTEKETFCRAATVLRQFYSHHEKDTRCLGATAQ |
| cirularly permuted wild-type IL-4 | QFHRHKQLIRFLKRLDRNLWGLAGLNSCPVKEANQ |
| | STLENFLERLKTIMREKYSKCSSGGNGGHKCDITL |
| | QEIIKTLNSLTEQKTLCTELTVTDIFAAS |
| SEQ ID NO: 54 | MDTTEKETFCRAATVLRQFYSHHEKDTRCLGATAQ |
| circularly permuted ″KFR″ IL-4 | QFHRHKQLIRFLKRLDRNLWGLAGLNSCPVKEANQ |
| variant | STLENFLERLKTIMKEKFRKCSSGGNGGHKCDITL |
| | QEIIKTLNSLTEQKTLCTELTVTDIFAASRQFYSH |
| | HEKDTRCLGATAQQFHRHKQLIRFLKRLDRNLWGL |
| | AGLNSCPVKEANQSTLENFLERLRVIMQSKWFKCG |
| | AGGNGGHKCDITLQEIIKTLNSLTEQKTLCTELTV |
| | TDIFAAS |
| SEQ ID NO: 55 | MDTTEKETFCRAATVLRQFYSHHEKDTRCLGATAQ |
| circularly permuted ″KF″ IL-4 | QFHRHKQLIRFLKRLDRNLWGLAGLNSCPVKEANQ |
| variant | STLENFLERLKTIMKEKFKCSSGGNGGHKCDITLQ |
| | EIIKTLNSLTEQKTLCTELTVTDIFAAS |

Table of cytokine fusions containing either IL-4 or IL-13 sequences is provided below.

TABLE 7
List of Amino Acid Sequences

| SEQ ID NO: | |
|---|---|
| (Information) | Amino acid sequence |
| SEQ ID NO: 56 | PGPVPPSTAHRELIEELVNITQNQKAPLCNGSMVWS |
| IL13-BAD (targeting IL13Ra2; | INLTAGMYCAALESLINVSGCSAIEKTQRMLSGFCP |
| referred to as C11; GGGGS | HKVSAGQFSSLHVTGRKIEVAQFVKDLLLHLKKLFK |
| linker) | EGQFNGGGGSMFQIPEFEPSEQEDSSSAERGLGPS |
| | PAGDGPSGSGKHHRQAPGLLWDASHQQEQPTSSSHH |
| | GGAGAVEIRSRHSAYPAGTEDDEGMGEEPSPFRGRS |
| | RAAPPNLWAAQRYGRELRRMSDEFVDSFKKGLPRPK |
| | SAGTATQMRQSSSWTRVFQSWWDRNLGRGSSAPSQ |
| SEQ ID NO: 57 | PGPVPPSTAVRELIEELINITQNQKAPLCNGSMVWS |
| A11-BAD (A11 is an IL13Ra1 | INRTAGMYCAALESLINVSGCSAIEKTQRMLSGFCP |
| agonist; GGGGS linker) | HKVSAGQFSSLHVRSSKIEVAQFVKDLLFHLRTLFR |
| | EGQFNGGGGSMFQIPEFEPSEQEDSSSAERGLGPS |
| | PAGDGPSGSGKHHRQAPGLLWDASHQQEQPTSSSHH |
| | GGAGAVEIRSRHSAYPAGTEDDEGMGEEPSPFRGRS |
| | RAAPPNLWAAQRYGRELRRMSDEFVDSFKKGLPRPK |
| | SAGTATQMRQSSSWTRVFQSWWDRNLGRGSSAPSQ |
| SEQ ID NO: 58 | KCDITLQEIIKTLNSLTEQKTLCTELTVTDIFA |
| KFR-BAD (KFR targets Type 2 | ASKNTTEKETFCRAATVLRQFYSHHEKDTRCLGATA |
| IL-4R; GGGGS linker) | QQFHRHKQLIRFLKRLDRNLWGLAGLNSCPVKEANQ |
| | STLENFLERLKTIMKEKFRKCSSGGGGSMFQIPEFE |
| | PSEQEDSSSAERGLGPSPAGDGPSGSGKHHRQAPGL |
| | LWDASHQQEQPTSSSHHGGAGAVEIRSRHSAYPAGT |
| | EDDEGMGEEPSPFRGRSRAAPPNLWAAQRYGRELRR |
| | MSDEFVDSFKKGLPRPKSAGTATQMRQSSSWTRVFQ |
| | SWWDRNLGRGSSAPSQ |
| SEQ ID NO: 59 | MDTTEKETFCRAATVLRQFYSHHEKDTRCLGATAQQ |
| pKFR4-Bad-H6 | FHRHKQLIRFLKRLDRNLWGLAGLNSCPVKEANQST |
| | IKTLNSLTEQKTLCTELTVTDIFAASGSFQIPEFEP |
| | SEQEDSSSAERGLGPSPAGDGPSGSGKHHRQAPGLL |
| | WDASHQQEQPTSSSHHGGAGAVEIRSRHSAYPAGTE |
| | DDEGMGEEPSPFRGRSRAAPPNLWAAQRYGRELRRM |
| | SDEFVDSFKKGLPRPKSAGTATQMRQSSSWTRVFQS |
| | WWDRNLGRGSSAPSQHHHHHH |
| SEQ ID NO: 60 | MDTTEKETFCRAATVLRQFYSHHEKDTRCLGATAQQ |
| cpKFR4-Bad fusion; GS linker | FHRHKQLIRFLKRLDRNLWGLAGLNSCPVKEANQST |
| | LENFLERLKTIMKEKFRKCSSGGNGGHKCDITLQEI |
| | IKTLNSLTEQKTLCTELTVTDIFAASGSFQIPEFEP |
| | SEQEDSSSAERGLGPSPAGDGPSGSGKHHRQAPGLL |
| | WDASHQQEQPTSSSHHGGAGAVEIRSRHSAYPAGTE |
| | DDEGMGEEPSPFRGRSRAAPPNLWAAQRYGRELRRM |
| | SDEFVDSFKKGLPRPKSAGTATQMRQSSSWTRVFQS |
| | WWDRNLGRGSSAPSQ |
| SEQ ID NO: 61 | MDTTEKETFCRAATVLRQFYSHHEKDTRCLGAT |
| cpIL4-BAD; GS linker | AQQFHRHKQLIRFLKRLDRNLWGLAGLNSCPVKEAN |
| | QSTLENFLERLKTIMREKYSKCSSGGNGGHKCDITL |
| | QEIIKTLNSLTEQKTLCTELTVTDIFAASGSFQIPE |
| | FEPSEQEDSSSAERGLGPSPAGDGPSGSGKHHRQAP |
| | GLLWDASHQQEQPTSSSHHGGAGAVEIRSRHSAYPA |
| | GTEDDEGMGEEPSPFRGRSRAAPPNLWAAQRYGREL |
| | RRMSDEFVDSFKKGLPRPKSAGTATQMRQSSSWTRV |
| | FQSWWDRNLGRGSSAPSQ |
| SEQ ID NO: 62 | MDTTEKETFCRAATVLRQFYSHHEKDTRCLGATAQQ |
| cpIL-4-BAD H6; GS linker | FHRHKQLIRFLKRLDRNLWGLAGLNSCPVKEANQST |
| | LENFLERLKTIMREKYSKCSSGGNGGHKCDITLQEI |
| | IKTLNSLTEQKTLCTELTVTDIFAASGSFQIPEFEP |
| | SEQEDSSSAERGLGPSPAGDGPSGSGKHHRQAPGLL |
| | WDASHQQEQPTSSSHHGGAGAVEIRSRHSAYPAGTE |
| | DDEGMGEEPSPFRGRSRAAPPNLWAAQRYGRELRRM |
| | SDEFVDSFKKGLPRPKSAGTATQMRQSSSWTRVFQS |
| | WWDRNLGRGSSAPSQHHHHHH |
| SEQ ID NO: 63 | PGPVPPSTAVRALIEELINITQNQKAPLCNGSMVWS |
| IL13-BAD (targets IL13Ra1 and | INRTAGMYCAALESLINVSGCSAIEKTQDMLSGFCP |
| is referred to as IL13DN) | HKVSAGQFSSLHVRSSKIEVAQFVKDLLFHLRTLFR |
| | EGQFNGGGGSGGGGSGGGGSFQIPEFEPSEQEDSSS |
| | AERGLGPSPAGDGPSGSGKHHRQAPGLLWDASHQQE |
| | QPTSSSHHGGAGAVEIRSRHSAYPAGTEDDEGMGEE |
| | PSPFRGRSRAAPPNLWAAQRYGRELRRMSDEFVDSF |
| | KKGLPRPKSAGTATQMRQSSSWTRVFQSWWDRNLGR |
| | GSSAPSQ |
| SEQ ID NO: 64 | KCDITLQEIIKTLNSLTEQKTLCTELTVTDIFAASK |
| IL-4-BclxL; GGGGS linker | NTTEKETFCRAATVLRQFYSHHEKDTRCLGATAQQF |
| | HRHKQLIRFLKRLDRNLWGLAGLNSCPVKEANQSTL |
| | ENFLERLKTIMREKYSKCSSGGGGSMSQSNRELVV |
| | DFLSYKLSQKGYSWSQFSDVEENRTEAPEGTESEME |
| | TPSAINGNPSWHLADSPAVNGATGHSSSLDAREVIP |
| | MAAVKQALREAGDEFELRYRRAFSDLTSQLHITPGT |
| | AYQSFEQVVNELFRDGVNWGRIVAFFSFGGALCVES |
| | VDKEMQVLVSRIAAWMATYLNDHLEPWIQENGGWDT |
| | FVELYGNNAAAESRKGQERFNRWFLTGMTVAGVVLL |
| | GSLFSRK |
| SEQ ID NO: 65 | MDTTEKETFCRAATVLRQFYSHHEKDTRCLGATAQQ |
| MDNA55 | FHRHKQLIRFLKLRDRNLWGLAGLNSCPVKEANQST |
| | LENFLERLKTIMREKYSKCSSGGNGGHKCDITLQEI |
| | IKTLNSLTEQKTLCTELTVTDIFAASKASGGPEGGS |
| | LAALTAHQACHLPLETFTRHRQPRGWEQLEQCGYPV |
| | QRLVALYLAARLSWNQVDQVIRNALASPGSGGDLGE |
| | AIREQPEQARLALTLAAAESERFVRQGTGNDEAGAA |
| | NGPADSGDALLERNYPTGAEFLGDGGDVSFSTRGTQ |
| | NWTVERLLQAHRQLEERGYVFVGYHGTFLEAAQSIV |
| | FGGVRARSQDLDAIWRGFYIAGDPALAYGYAQDQEP |
| | DARGRIRNGALLRVYVPRSSLPGFYRTSLTLAAPEA |
| | AGEVERLIGHPLPLRLDAITGPEEEGGRLETILGWP |
| | LAERTVVIPSAIPTDPRNVGGDLDPSSIPDKEQAIS |
| | ALPDYASQPGKPPKDEL |
| SEQ ID NO: 66 | MDTTEKETFCRAATVLRQFYSHHEKDTRCLGATAQQ |
| cpS4-Bad-H6 | FHRHKQLIRFLKRLDRNLWGLAGLNSCPVKEANQST |
| | LENFLERLRVIMQSKWFKCGAGGNGGHKCDITLQEI |
| | IKTLNSLTEQKTLCTELTVTDIFAASGSFQIPEFEP |
| | SEQEDSSSAERGLGPSPAGDGPSGSGKHHRQAPGLL |
| | WDASHQQEQPTSSSHHGGAGAVEIRSRHSAYPAGTE |
| | DDEGMGEEPSPFRGRSRAAPPNLWAAQRYGRELRRM |
| | SDEFVDSFKKGLPRPKSAGTATQMRQSSSWTRVFQS |
| | WWDRNLGRGSSAPSQHHHHHH |
| SEQ ID NO: 67 | MDTTEKETFCRAATVLRQFYSHHEKDTRCLGATAQQ |
| cpS4-Bad | FHRHKQLIRFLKRLDRNLWGLAGLNSCPVKEANQST |
| | LENFLERLRVIMQSKWFKCGAGGNGGHKCDITLQEI |
| | IKTLNSLTEQKTLCTELTVTDIFAASGSFQIPEFEP |
| | SEQEDSSSAERGLGPSPAGDGPSGSGKHHRQAPGLL |
| | WDASHQQEQPTSSSHHGGAGAVEIRSRHSAYPAGTE |
| | DDEGMGEEPSPFRGRSRAAPPNLWAAQRYGRELRRM |
| | SDEFVDSFKKGLPRPKSAGTATQMRQSSSWTRVFQS |
| | WWDRNLGRGSSAPSQ |
| SEQ ID NO: 68 | MHKCDITLQEIIKTLNSLTEQKTLCTELTVTDIFAA |
| IL-4-Bad-H6 | SKDTTEKETFCRAATVLRQFYSHHEKDTRCLGATAQ |
| | QFHRHKQLIRFLKRLDRNLWGLAGLNSCPVKEANQS |
| | TLENFLERLKTIMREKYSKCSSGSFQIPEFEPSEQE |
| | DSSSAERGLGPSPAGDGPSGSGKHHRQAPGLLWDAS |
| | HQQEQPTSSSHHGGAGAVEIRSRHSAYPAGTEDDEG |
| | MGEEPSPFRGRSRAAPPNLWAAQRYGRELRRMSDEF |
| | VDSFKKGLPRPKSAGTATQMRQSSSWTRVFQSWWDR |
| | NLGRGSSAPSQHHHHHH |
| SEQ ID NO: 69 | MHKCDITLQEIIKTLNSLTEQKTLCTELTVTDIFAA |
| IL-4-Bad-H6 | SKDTTEKETFCRAATVLRQFYSHHEKDTRCLGATAQ |
| | QFHRHKQLIRFLKRLDRNLWGLAGLNSCPVKEANQS |
| | TLENFLERLKTIMREKYSKCSSGSFQIPEFEPSEQE |
| | DSSSAERGLGPSPAGDGPSGSGKHHRQAPGLLWDAS |
| | HQQEQPTSSSHHGGAGAVEIRSRHSAYPAGTEDDEG |
| | MGEEPSPFRGRSRAAPPNLWAAQRYGRELRRMSDEF |
| | VDSFKKGLPRPKSAGTATQMRQSSSWTRVFQSWWDR |
| | NLGRGSSAPSQ |

TABLE 4
List of Selected Fusuion Partners

| SEQ ID NO: | |
|---|---|
| (Information) | Amino acid sequence |
| SEQ ID NO: 38 | MFQIP EFEPSEQEDS SSAERGLGPS PAGDGPSGSG |
| BAD amino acid sequence | KHHRQAPGLL WDASHQQEQP |
| | TSSSHHGGAG AVEIRSRHSA YPAGTEDDEG |
| | MGEEPSPFRG RSRAAPPNLW AAQRYGRELR |
| | RMSDEFVDSF KKGLPRPKSA GTATQMRQSS |
| | SWTRVFQSWW DRNLGRGSSA PSQ |
| SEQ ID NO: 39 | MAHAGRTGYD NREIVMKYIH YKLSQRGYEW |
| Bcl-2 amino acid sequence | DAGDVGAAPP GAAPAPGIFS SQPGHTPHPA |
| | ASRDPVARTS PLQTPAAPGA AAGPALSPVP |
| | PVVHLTLRQA GDDFSRRYRR DFAEMSSQLH |
| | LTPFTARGRF ATVVEELFRD GVNWGRIVAF |
| | FEFGGVMCVE SVNREMSPLV DNIALWMTEY |
| | LNRHLHTWIQ DNGGWDAFVE |
| | LYGPSMRPLF DFSWLSLKTL LSLALVGACI |
| | TLGAYLGHK |
| SEQ ID NO: | MFQIPEFEPS EQEDSSSAER GLGPSPAGDG |
| >HsBAD_Q92934-1(UniProtKB) | PSGSGKHHRQ APGLLWDASH |
| | QQEQPTSSSH HGGAGAVEIR SRHSSYPAGT |
| | EDDEGMGEEP SPFRGRSRSA |
| | PPNLWAAQRY GRELRRMSDE FVDSFKKGLP |
| | RPKSAGTATQ MRQSSSWTRV |
| | FQSWWDRNLG RGSSAPSQ |
| SEQ ID NO: | MDGSGEQPRG GGPTSSEQIM KTGALLLQGF |
| >HsBAX_Q07812-1(UniProtKB) | IQDRAGRMGG EAPELALDPV |
| | PQDASTKKLS ECLKRIGDEL DSNMELQRMI |
| | AAVDTDSPRE VFFRVAADMF |
| | SDGNFNWGRV VALFYFASKL VLKALCTKVP |
| | ELIRTIMGWT LDFLRERLLG |
| | WIQDQGGWDG LLSYFGTPTW QTVTIFVAGV |
| | LTASLTIWKK MG |
| SEQ ID NO: | MASGQGPGPP RQECGEPALP SASEEQVAQD |
| >HsBAK1_Q16611-1(UniProtKB) | TEEVFRSYVF YRHQQEQEAE |
| | GVAAPADPEM VTLPLQPSST MGQVGRQLAI |
| | IGDDINRRYD SEFQTMLQHL |
| | QPTAENAYEY FTKIATSLFE SGINWGRVVA |
| | LLGFGYRLAL HVYQHGLTGF |
| | LGQVTRFVVD FMLHHCIARW IAQRGGWVAA |
| | LNLGNGPILN VLVVLGVVLL |
| | GQFVVRRFFK S |
| SEQ ID NO: | MSEVRPLSRD ILMETLLYEQ LLEPPTMEVL |
| >HsBIK_Q13323-1(UniProtKB) | GMTDSEEDLD PMEDFDSLEC |
| | MEGSDALALR LACIGDEMDV SLRAPRLAQL |
| | SEVAMHSLGL AFIYDQTEDI |
| | RDVLRSFMDG FTTLKENIMR FWRSPNPGSW |
| | VSCEQVLLAL LLLLALLLPL |
| | LSGGLHLLLK |
| SEQ ID NO: | MDCEVNNGSS LRDECITNLL VFGFLQSCSD |
| >HsBID_P55957-1(UniProtKB) | NSFRRELDAL GHELPVLAPQ |
| | WEGYDELQTD GNRSSHSRLG RIEADSESQE |
| | DIIRNIARHL AQVGDSMDRS |
| | IPPGLVNGLA LQLRNTSRSE EDRNRDLATA |
| | LEQLLQAYPR DMEKEKTMLV |
| | LALLLAKKVA SHTPSLLRDV FHTTVNFINQ |
| | NLRTYVRSLA RNGMD |

C. Cargo Moieties

Cargo moieties reduce or inhibit cancer stem cell growth, or kill cancer cells and/or cancer stem cells. In some examples cargo moieties are not proteins, but other molecules that reduce or inhibit cancer stem cell growth, or kill cancer cells and/or cancer stem cells, such as chemotherapeutic agents. In some examples, cargo moieties also reduce or inhibit bulk cancer cell growth, or kill cancer cells. Any protein or other agent that functions to reduce or inhibit cancer stem cell growth, or kill such cells, can be used as a cargo moiety. For example, toxins and proteins that function to control cell life cycles can be used as cargo moieties. Toxins that can be used as cargo moieties include toxins made by microorganisms, plants or animals, as well as toxins made by human cells. Similarly, any natural cell growth controlling protein can be used as a cargo moiety. For example, proteins that trigger cell death during the normal life cycle of an organism can be used as cargo moieties. In some examples, an oncolytic virus (e.g., see Allen et al., Mol. Ther. 16:1556-64, 2008) or liposomes carrying cytotoxic agents (e.g., see Madhankumar et al., Mol. Cancer. Ther. 5:3162-9, 2006) is used as the cargo protein.

In one example, the cargo moiety is a toxin. Exemplary toxins that can be used include pore-forming toxins, and toxins that upon internalization inhibit cell growth. In other examples, cargo moieties are proteins that are apoptotic triggering proteins, and cell growth inhibiting proteins. In some examples, the toxin is a modified bacterial toxin such that the resulting toxin is less immunogenic than the native toxin. Such modified toxins, such as a modified Pseudomonas exotoxin A, can reduce the patient's immunogenic response, thereby allowing repeated administration.

Pore forming toxins are toxins that form pores in the cell membrane thereby killing the cell via cell lyses. Exemplary pore forming toxins include but are not limited to human toxins such as perform or bacterial toxins such as aerolysin as well as modified pore-forming protein toxins that are derived from naturally occurring pore-forming protein toxins (nPPTs) such as aerolysin or aerolysin-related polypeptides. Suitable aerolysin-related nPPTs have the following features: a pore-forming activity that is activated by removal of an inhibitory domain via protease cleavage, and the ability to bind to receptors that are present on cell membranes through one or more binding domains. In some examples the linker can be engineered to be sensitive to a protease or be chemically liable. Additional examples of pore forming toxins that can be used as cargo moieties include, but are not limited to, proaerolysin from Aeromonas hydrophila, Aeromonas trota and Aeromonas salmonicida, alpha toxin from Clostridium septicum, anthrax protective antigen, Vibrio cholerae VCC toxin, epsilon toxin from Clostridium perfringens, and Bacillus thuringiensis delta toxins. A detailed description of the engineering of proaerolysin can be found in U.S. Pat. No. 7,282,476, which is herein incorporated by reference.

Additional toxins that can be used as cargo moieties include toxins that act within a cell. For example, anthrax, diphtheria, cholera, and botulinum toxins include a portion that acts in the cytoplasm, as well as a portion that acts to bind to the cell surface. These toxins, or portions thereof, can be linked to a targeting moiety and used to inhibit cancer stem cell growth. Select members of the ribonuclease A (RNase A) superfamily are potent cytotoxins. These cytotoxic ribonucleases enter the cytosol, where they degrade cellular RNA and cause cell death.

In some examples ribosome inactivating proteins can be used as toxins. In these examples the cargo moiety is a polypeptide having ribosome-inactivating activity including, without limitation, gelonin, bouganin, saporin, ricin, ricin A chain, bryodin, restrictocin, and variants thereof. Diphtheria toxin and Pseudomonas exotoxin A inhibit protein synthesis via ADP-ribosylation of elongation factor 2. When the cargo moiety is a ribosome-inactivating protein or inhibits protein synthesis via ADP-ribosylation of elongation factor 2, the IL-4 targeted cargo protein can be internalized upon binding to the cancer stem cell. Cargo moieties that induce apoptosis can also be used to target cancer cells and/or cancer stem cells. Examples of cargo moieties that induce apoptosis include caspases, granzymes and BCL-2 pro-apoptotic related proteins such as BAX (e.g., Accession no: CAE52910), BAD (e.g., Accession no: CAG46757), BAT (e.g., Accession no: AA107425), BAK (e.g., Accession no: AAA74466), BIK (e.g., Accession no: CAG30276), BOK (e.g., Accession no: AAH06203), BID (e.g., Accession no: CAG28531), BIM (e.g., Accession no: NP_619527) and BMF (e.g., Accession no: AAH69328). These cargo moieties can be used alone of in combination to reduce or inhibit cancer stem cell growth.

Aerolysin is a channel-forming toxin produced as an inactive protoxin called proaerolysin (PA). Exemplary aerolysin and PA sequences that can be used in a IL-4 targeted cargo protein are provided in Table 1. The PA protein contains many discrete functionalities that include a binding domain, a toxin domain, and a C-terminal inhibitory peptide domain that contains a protease activation site. The binding domain recognizes and binds to glycophosphatidylinositol (GPI) membrane anchors, such as are found in Thy-1 on T lymphocytes, the PIGA gene product found in erythrocyte membranes and Prostate Stem Cell Antigen (PSCA). The activation or proteolysis site within proaerolysin is a six amino acid sequence that is recognized as a proteolytic substrate by the furin family of proteases. PA is activated upon hydrolysis of a C-terminal inhibitory segment by furin. Activated aerolysin binds to GPI-anchored proteins in the cell membrane and forms a heptamer that inserts into the membrane producing well-defined channels of about.17 angstroms. Channel formation leads to rapid cell death. Wild-type aerolysin is toxic to mammalian cells, including erythrocytes, for example at 1 nanomolar or less.

In some examples, a target cargo protein is an PA molecule with the native furin site replaced with a different cleavage site, such as prostate-specific protease cleavage site (e.g., a PSA-specific cleavage site, which permits activation of the variant PA in the presence of a prostate-specific protease such as PSA, PMSA, or HK2). In one example, a prostate-specific protease cleavage site is inserted into the native furin cleavage site of PA, such that PA is activated in the presence of a prostate-specific protease, but not furin. In another example, a variant PA molecule further includes a functionally deleted binding domain (e.g., about amino acids 1-83 of a native PA protein sequence). Functional deletions can be made using any method known in the art, such as deletions, insertions, mutations, or substitutions. In some examples, IL-4 targeted cargo proteins include variant PA molecules in which the native binding domain is functionally deleted and replaced with a prostate-tissue or other tissue-specific binding domain. In other examples, variant PA molecules include a furin cleavage site and a functionally deleted binding domain which is replaced with a prostate-tissue specific binding domain. Such variant PA molecules are targeted to prostate cells via the prostate-tissue specific binding domain, and activated in the presence of furin.

Bouganin is a ribosome-binding protein originally isolated from Bougainvillea speotabilis (see U.S. Pat. No. 6,680,296). Exemplary modified bouganins are described in WO 2005/090579 and U.S. Pat. No. 7,339,031. Bouganin damages ribosomes and leads to a cessation of protein synthesis and cell death. Exemplary bouganin proteins that can be used in the IL-4 targeted cargo proteins of the present disclosure include those in GenBank Accession No. AAL35962, as well as those native and modified bouganin sequences provided in U.S. Pat. Nos. 6,680,296; 7,339,031 and PCT publication WO 2005/090579 (bouganin sequences herein incorporated by reference), as well as sequences having at least 60% sequence identity, at least 75%, at least 80%, at least 85%, at least 90%, at least 95%, at least 98% or even at least 99% sequence identity to such sequences. BAD, BCL2-associated agonist of cell death, is a regulator of programmed cell death (apoptosis). BAD positively regulates cell apoptosis by forming heterodimers with BCL-xL and BCL-2, and reversing their death repressor activity. Proapoptotic activity of BAD is regulated through its phosphorylation. Exemplary BAD proteins that can be used in the IL-4 targeted cargo proteins of the present disclosure include those in GenBank Accession Nos. CAG46757; AAH01901.1; and CAG46733.1, as well as those sequences provided in U.S. Pat. No. 6,737,511 (sequences herein incorporated by reference), as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native BAD protein.

BAX, BCL2-associated X protein, is a regulator of programmed cell death (apoptosis). This protein forms a heterodimer with BCL2, and functions as an apoptotic activator. BAX interacts with, and increases the opening of, the mitochondrial voltage-dependent anion channel (VDAC), which leads to the loss in membrane potential and the release of cytochrome c. Exemplary BAX proteins that can be used in the IL-4 targeted cargo proteins of the present disclosure include those provided by GenBank Accession Nos. CAE52909.1; AA022992.1; EAW52418.1, U.S. Pat. No. 6,645,490 (Bax in the IL2-Bax construct is a Bax-alpha variant that can be used in the present disclosure), as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native BAX protein.

In some examples, the BAX protein of a IL-4 targeted cargo protein may be modified such that the C-terminal anchor domain has been deleted and replaced with a CaaX sequence. CaaX is a peptide with the sequence Cysteine-a-a-X where “X” is any amino acid and “a” is an aliphatic amino acid. Because membrane association of BAX is needed for optimal apoptosis activity, addition of membrane binding domains such as CaaX can enhance their pro-apoptotic activities. Proteins with CaaX sequence are farnesylated. Farnesylated proteins are targeted to membranes (e.g., see Wright and Philip, J. Lipid Res., 2006, 47(5): 883-91). Potential BAX variants containing a CaaX sequence may or may not contain the C-terminal anchor domain.

Pseudomonas exotoxin (PE) is a toxin secreted by Pseudomonas. Native PE is cytotoxic for mammalian cells due to its ability to enter cells by receptor-mediated endocytosis and then, after a series of intracellular processing steps, translocate to the cell cytosol and ADP-ribosylate elongation factor 2. This results in the inhibition of protein synthesis and cell death. PE has three functional domains: an amino-terminal receptor-binding domain, a middle translocation domain, and a carboxyl-terminal ADP-ribosylation domain. Modified PE molecules can include elimination of domain Ia, as well as deletions in domains II and III. Exemplary PE proteins that can be used in the IL-4 targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native PE protein.

Thapsigargin is an inhibitor of sarco/endoplasmic reticulum Ca2+ ATPases. Thapsigargin is classified as a sesquiterpene lactone, and raises cytosolic calcium concentration by blocking the ability of the cell to pump calcium into the sarcoplasmic and endoplasmic reticulum which causes these stores to become depleted. Store-depletion can secondarily activate plasma membrane calcium channels, allowing an influx of calcium into the cytosol.

Ribonuclease A (RNAseA) is an endonuclease that cleaves single-stranded RNA. RNAse A toxins can be obtained from mammals and reptiles. Exemplary RNAse A proteins that can be used in the IL-4 targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native RNAseA toxin.

The cargo moiety used can include native sequences (such as the GenBank Accession Nos. and sequences present in the patents referenced in Table 1 and listed above), as well as variants thereof, such as a variant having at least 98%, at least 95%, at least 90%, at least 80%, at least 70%, or at least 60% sequence identity with the native cargo moiety, as long as the variant retains or has enhanced biological activity of the native cargo moiety (e.g., at least about this amount of sequence identity to the GenBank Accession Nos. listed in Table 1 and listed above). In some examples, variant sequences retain substantially the same amount (or even more) of the native biological function of the cargo moiety, such as the ability to kill or inhibit the growth of a cancer stem cell. A cargo moiety can also be a fragment of the native sequence that retains a substantial amount of the native biological function of the protein.

The cargo moieties are engineered to target cancer cells and/or cancer stem cells by linking them to targeting moieties. Targeting moieties include agents that can bind to cancer stem cell surface targets.

D. Oncolytic Viruses Targeting Moieties

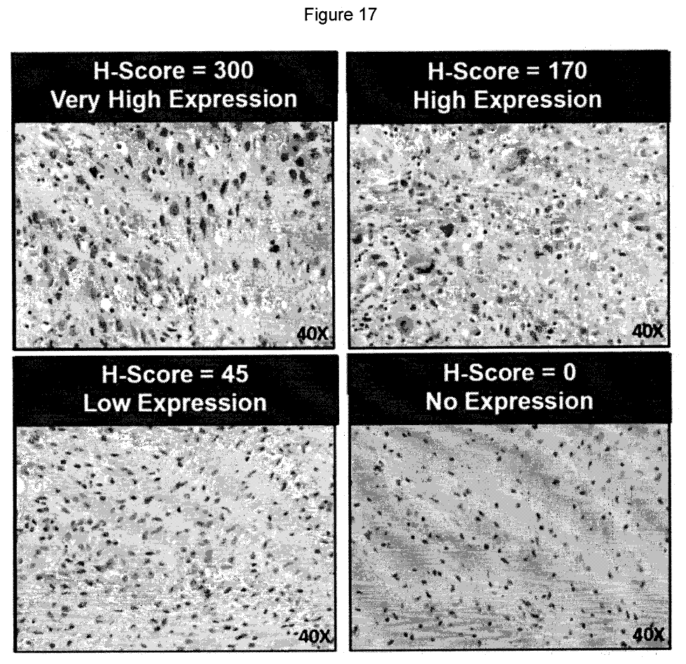

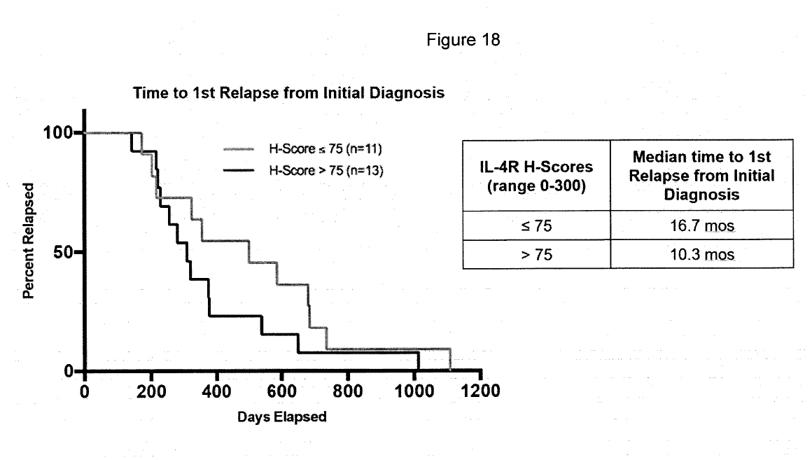

In some examples, cargo moieties of the present invention can be employed to target an oncolytic virus (e.g., see Allen et al., Mol. Ther. 16:1556-64, 2008). Numerouns virus can be employed as the oncolytic virus, including adenoviruses as well as self-replicating alphavirus such for example those provided in FIG. 17, as well as oncolyctic vaccinia viruses (see, for eample WO2013038066, incorporated herein by reference in its entirety).

Other oncolytic viruses can include Seneca Valley Virus, Newcastle disease Virus (also referred to as Newcastle virus), Maraba virus, VSV, Herpes virus (including HSV-1), Measles virus, poliovirus, reovirus, coxsackie virus, a lentivirus, a morbillivirus, an influenza virus, Sinbis virus, myxoma virus, and/or retrovirus (see, for example, Twumasi-Boateng, et al., “Oncolytic viruses as engineering platforms for combination immunotherapy”, Nature Reviews Cancer, 2018), and Kaufman et al., Cancer Immunotherapy, 14:642-662 (2015), all of which are incorporated by reference herein their entireties). In some embodiments, the oncolytic virus includes but is not limited to an adenovirus, a self-replicating alphavirus, a vaccinia virus, a Seneca Valley Virus, a Newcastle disease Virus, a Maraba virus, vesicular stomatitis virus (VSV), a Herpes virus (including HSV-1 and HSV-2), a measles virus, a poliovirus, a reovirus, a coxsackie virus, a lentivirus, a morbillivirus, an influenza virus, Sinbis virus, myxoma virus, and/or a retrovirus. Other oncolytic viruses include can include, for example, oncoVex/T-VEC, which involves the intratumoral injection of replication-conditional herpes simplex virus which preferentially infects cancer cells. The virus, which is also engineered to express GM-CSF, is able to replicate inside a cancer cell causing its lysis, releasing new viruses and an array of tumor antigens, and secreting GM-CSF in the process. Such oncolytic virus vaccines enhance DCs function in the tumor microenvironment to stimulate anti-tumor immune responses. These oncolytic viruses can be used to target or deliver the IL-4 and/or IL-13 muteins described herein to the tumor. In some embodiments, the IL-4 and/or IL-13 mutein is any IL-4 and/or IL-13 mutein or variant disclosed herein. In some embodiments, the IL-4 and/or IL-13 mutein sequence is 90% identical to any one of SEQ ID NO:2-SEQ ID NO:48 and/or SEQ ID NO:51-SEQ ID NO:69. In some embodiments, the IL-4 and/or IL-13 mutein incudes any one of SEQ ID NO:2-SEQ ID NO:48 and/or SEQ ID NO:51-SEQ ID NO:69. In some embodiments, the oncolytic virus comprises a transgene capable of expressing an IL-4 and/or IL-13 mutein as described herein. In some embodiments, the oncolytic virus comprises a transgene capable of expressing an IL-4 and/or IL-13 mutein comprising any one of SEQ ID NO:2-SEQ ID NO:48 and/or SEQ ID NO:51-SEQ ID NO:69. In some embodiments, the oncolytic virus comprises a nucleic acid encoding an IL-4 and/or IL-13 mutein comprising any one of SEQ ID NO:2-SEQ ID NO:48 and/or SEQ ID NO:51-SEQ ID NO:69. In some embodiments, the oncolytic virus comprises a transgene that is expressed as a therapeutic payload. In some embodiments, the therapeutic payload is an IL-4 and/or IL-13 as described herein. In some embodiments, the therapeutic payload is IL-4 and/or IL-13 mutein comprises any one of SEQ ID NO:2-SEQ ID NO:48 and/or SEQ ID NO:51-SEQ ID NO:69.

In some embodiments, the IL-4R targeting moiety can comprise an IL-4 sequence or variant thereof that targets immunosuppressive cells of the TME (tumor microenvironment) such as tumor associated macrophages and MDSCs (myeloid-derived suppressor cells) in order for oncyltic viruses to provide an improved therapeutic benefit. In some embodiments, the IL-4R targeting moiety comprises any IL-13 and/or IL-4 sequence as described herein. In some embodiments, the IL-4R targeting moiety comprises any one of SEQ ID NO:2-SEQ ID NO:48 and/or SEQ ID NO:51-SEQ ID NO:69. In some embodiments, IL-4R targeting moiety comprises an IL-13 variant/IL-13 superkine including those targeting Type 2 IL-4R and/or targeting IL13ra2 which can direct the oncyltic viruses to tumor antigens. In some embodiments, the IL-4R targeting moiety comprises an IL-13 variant/IL-13 superkine including those provided in SEQ ID NO:2-SEQ ID NO:48, SEQ ID NO:56, SEQ ID NO:57, and/or SEQ ID NO:63, can also direct the oncyltic viruses to tumor antigens. In some embodiments, the oncolytic virus is targeted by one cytokine and expresses another. In some embodiments, the oncolytic virus comprises a transgene that is expressed as a therapeutic payload. In some embodiments, the therapeutic payload that is expressed is an IL-4 sequence as described herein. In some embodiments, the therapeutic payload that is expressed is an IL-13 sequence as described herein.

In some embodiments, the oncolytic virus is an oncolytic vaccinia virus. In some embodiments, the oncolytic vaccinia virus vector is characterized in that the virus particle is of the type intracellular mature virus (IMV), intracellular enveloped virus (IEV), cell-associated enveloped virus (CEV), or extracellular enveloped virus (EEV). In some embodiments, the oncolytic vaccinia virus particle is of the type EEV or IMV. In some embodiments, the oncolytic vaccinia virus particle is of the type EEV.

Generally, construction of oncolytic vaccinia virus recombinants and cells and pharmaceutical compositions comprising said vectors which preferentially replicate in tumor cells and express at least one transgene to facilitate antitumor efficacy and apoptosis induction and to modulate host immune responses in a subject. According to the present invention, oncolytic adenoviruses and oncolytic vaccinia viruses can be combined with IL-4 and/or IL-13 targeting moieties as described herein in order to target the oncolytic vaccinia virus or the oncolytic adenovirus. Oncolysis releases tumor antigens and provides costimulatory danger signals. However, arming the virus can improve efficacy further. For example, CD40 ligand (CD40L, CD154) is known to induce apoptosis of tumor cells and it also triggers several immune mechanisms. One of these is a T-helper type 1 (Th1) response that leads to activation of cytotoxic T-cells and reduction of immune suppression. The present invention provides for oncolytic viruses that are targeted (for example, “armed”) with the IL-4 and/or IL-13 targeting moieties of the present invention.

In some embodiments, the oncolytic virus is a modified vaccinia virus vector, a virus particle, a host cell, a pharmaceutical composition and a kit comprising vaccinia virus genome wherein the thymidine kinase gene is inactivated by either a substitution in the thymidine kinase (TK) gene and/or an open reading frame ablating deletion of at least one nucleotide providing a partially deleted thymidine kinase gene, the vaccinia growth factor gene is deleted, and the modified vaccinia virus vector comprises at least one nucleic acid sequence encoding a non-viral protein (e.g., an IL-4 and/or IL-13 targeting moiety as described herein). In another aspect is provided the modified vaccinia virus vector, the virus particle, the pharmaceutical composition or the kit can be used for cancer therapy, for eliciting immune response in a subject, for use in a method of inhibiting malignant cell proliferation in a mammal, for use in a therapy or prophylaxis of cancer, for detecting the presence of the modified vaccinia virus in a subject, and as an in situ cancer vaccine, optionally in combination with adenovirus. In some embodiments, the invention provides method of producing a modified vaccinia virus comprising vaccinia virus genome wherein the thymidine kinase gene is inactivated by a substitution in the thymidine kinase (TK) gene and/or an open reading frame ablating deletion of at least one nucleotide providing a partially deleted thymidine kinase gene, the vaccinia growth factor gene is deleted, and the modified vaccinia virus vector comprises at least one nucleic acid sequence encoding a non-viral protein (e.g., an IL-4 and/or IL-13 targeting moiety as described herein), comprising the steps of providing producer cells capable of sustaining production of vaccinia virus particles and carrying the modified vaccinia vector; culturing the producer cells in conditions suitable for virus replication and production; and harvesting the virus particles.

Generally, the present invention also provides methods of administer an oncolytic virus “armed” or targeted with an IL-4 and/or IL-13 moiety as described herein. The routes of administration vary, naturally, with the location and nature of the tumor, and include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration. Compositions are formulated relative to the particular administration route.

1. Oncolytic Vaccinia Virus

Vaccinia virus is a member of the Orthopoxvirus genus of the Poxviridae. It has large double-stranded DNA genome (˜200 kb, ˜200 genes) and a complex morphogenic pathway produces distinct forms of infectious virions from each infected cell. Viral particles contain lipid membranes(s) around a core. Virus core contains viral structural proteins, tightly compacted viral DNA genome, and transcriptional enzymes. Dimensions of vaccinia virus are ˜360×270×250 nm, and weight of ˜5-10 fg. Genes are tightly packed with little non-coding DNA and open-reading frames (ORFs) lack introns. Three classes of genes (early, intermediate, late) exists. Early genes (˜100 genes; immediate and delayed) code for proteins mainly related to immune modulation and virus DNA replication. Intermediate genes code for regulatory proteins which are required for the expression of late genes (e.g. transcription factors) and late genes code for proteins required to make virus particles and enzymes that are packaged within new virions to initiate the next round of infection. Vaccinia virus replicates in the cell cytoplasm.

Different strains of vaccinia viruses have been identified (as an example: Copenhagen, modified virus Ankara (MVA), Lister, Tian Tan, Wyeth (=New York City Board of Health), Western Reserve (WR)). The genome of WR vaccinia has been sequenced (Accession number AY243312). In some embodiments, the oncolytic vaccinia virus is a Copenhagen, modified virus Ankara (MVA), Lister, Tian Tan, Wyeth, or Western Reserve (WR) vaccinia virus.

Different forms of viral particles have different roles in the virus life cycle Several forms of viral particles exist: intracellular mature virus (IMV), intracellular enveloped virus (IEV), cell-associated enveloped virus (CEV), extracellular enveloped virus (EEV). EEV particles have an extra membrane derived from the trans-Golgi network. This outer membrane has two important roles: a) it protects the internal IMV from immune aggression and, b) it mediates the binding of the virus onto the cell surface.

CEVs and EEVs help virus to evade host antibody and complement by being wrapped in a host-derived membrane. IMV and EEV particles have several differences in their biological properties and they play different roles in the virus life cycle. EEV and IMV bind to different (unknown) receptors (1) and they enter cells by different mechanisms. EEV particles enter the cell via endocytosis and the process is pH sensitive. After internalization, the outer membrane of EEV is ruptured within an acidified endosome and the exposed IMV is fused with the endosomal membrane and the virus core is released into the cytoplasm. IMV, on the other hand, enters the cell by fusion of cell membrane and virus membrane and this process is pH-independent. In addition to this, CEV induces the formation of actin tails from the cell surface that drive virions towards uninfected neighboring cells.

Furthermore, EEV is resistant to neutralization by antibodies (NAb) and complement toxicity, while IMV is not. Therefore, EEV mediates long range dissemination in vitro and in vivo. Comet-inhibition test has become one way of measuring EEV-specific antibodies since even if free EEV cannot be neutralized by EEV NAb, the release of EEV from infected cells is blocked by EEV NAb and comet shaped plaques cannot be seen. EEV has higher specific infectivity in comparison to IMV particles (lower particle/pfu ratio) which makes EEV an interesting candidate for therapeutic use. However, the outer membrane of EEV is an extremely fragile structure and EEV particles need to be handled with caution which makes it difficult to obtain EEV particles in quantities required for therapeutic applications. EEV outer membrane is ruptured in low pH (pH ˜6). Once EEV outer membrane is ruptured, the virus particles inside the envelope retain full infectivity as an IMV.

Some host-cell derived proteins co-localize with EEV preparations, but not with IMV, and the amount of cell-derived proteins is dependent on the host cell line and the virus strain. For instance, WR EEV contains more cell-derived proteins in comparison to VV IHD-J strain. Host cell derived proteins can modify biological effects of EEV particles. As an example, incorporation of the host membrane protein CD55 in the surface of EEV makes it resistance to complement toxicity. In the present invention it is shown that human A549 cell derived proteins in the surface of EEV particles may target virus towards human cancer cells. Similar phenomenon has been demonstrated in the study with human immunodeficiency virus type 1, where host-derived ICAM-1 glycoproteins increased viral infectivity. IEV membrane contains at least 9 proteins, two of those not existing in CEV/EEV. F12L and A36R proteins are involved in IEV transport to the cell surface where they are left behind and are not part of CEV/EEV (9, 11). 7 proteins are common in (IEV)/CEV/EEV: F13L, A33R, A34R, A56R, B5R, E2, (K2L). For Western Reserve strain of vaccinia virus, a maximum of 1% of virus particles are normally EEV and released into the culture supernatant before oncolysis of the producer cell. 50-fold more EEV particles are released from International Health Department (IHD)-J strain of vaccinia. IHD has not been studied for use in cancer therapy of humans however. The IHD-W phenotype was attributed largely to a point mutation within the A34R EEV lectin-like protein. Also, deletion of A34R increases the number of EEVs released. EEV particles can be first detected on cell surface 6 hours post-infection (as CEV) and 5 hours later in the supernatant (IHD-J strain). Infection with a low multiplicity of infection (MOI) results in higher rate of EEV in comparison to high viral dose. The balance between CEV and EEV is influenced by the host cell and strain of virus.

Vaccinia has been used for eradication of smallpox and later, as an expression vector for foreign genes and as a live recombinant vaccine for infectious diseases and cancer. Vaccinia virus is the most widely used pox virus in humans and therefore safety data for human use is extensive. During worldwide smallpox vaccination programs, hundreds of thousands humans have been vaccinated safety with modified vaccinia virus strains and only very rare severe adverse events have been reported. Those are generalized vaccinia (systemic spread of vaccinia in the body), erythema multiforme (toxic/allergic reaction), eczema vaccinatum (widespread infection of the skin), progressive vaccinia (tissue destruction), and postvaccinia! encephalitis.

All together 44 melanoma patients have been treated in early clinical trials with wild type vaccinia virus in 1960s-1990s and the overall objective response rate of injected tumors was 50%. Also some beneficial immunological responses were seen (36). Wild type vaccinia virus has been used also for treatment of bladder cancer, lung and kidney cancer, and myeloma and only mild adverse events were seen. JX-594, an oncolytic Wyeth strain vaccinia virus coding for GM-CSF, has been successfully evaluated in three phase I studies and preliminary results from randomized phase II trial has been presented in the scientific meeting.

Vaccinia virus is appealing for cancer gene therapy due to several characteristics. It has natural tropism towards cancer cells and the selectivity can be significantly enhanced by deleting some of the viral genes. The present invention relates to the use of double deleted vaccinia virus (vvdd) in which two viral genes, viral thymidine kinase (TK) and vaccinia growth factor (VGF), are at least partially deleted. TK and VGF genes are needed for virus to replicate in normal but not in cancer cells. The partial TK deletion may be engineered in the TK region conferring activity.

TK deleted vaccinia viruses are dependent on cellular nucleotide pool present in dividing cells for DNA synthesis and replication. IN some embodiments, the TK deletion limits virus replication significantly in resting cells allowing efficient virus replication to occur only in actively dividing cells (e.g., cancer cells). VGF is secreted from infected cells and has a paracrine priming effect on surrounding cells by acting as a mitogen. Replication of VGF deleted vaccinia viruses is highly attenuated in resting (non-cancer) cells. The effects of TK and VGF deletions have been shown to be synergistic.

2. Oncolytic Adenovirus

Generally, adenovirus is a 36 kb, linear, double-stranded DNA virus (Grunhaus and Horwitz, 1992). The term “adenovirus” or “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV 9_hu14, avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc.

Adenoviral infection of host cells results in adenoviral DNA being maintained episornally, which reduces the potential genotoxicity associated with integrating vectors. Also, adenoviruses are structurally stable, and no genome rearrangement has been detected after extensive amplification. Adenovirus can infect virtually all epithelial cells regardless of their cell cycle stage. (See, for example, US20060147420, incorporated by reference herein in its entirety.) Moreover, the E1a and E4 regions of adenovirus are essential for an efficient and productive infection of human cells. The E1a gene is the first viral gene to be transcribed in a productive infection, and its transcription is not dependent on the action of any other viral gene products. However, the transcription of the remaining early viral genes requires E1a gene expression. The E1a promoter, in addition to regulating the expression of the E1a gene, also integrates signals for packaging of the viral genome as well as sites required for the initiation of viral DNA replication. See, Schmid, S. I., and Hearing, P. in Current Topics in Microbiology and Immunology, vol. 199: pages 67-80 (1995).

In some embodiments, the oncolytic virus is an oncolytic adenovirus. It has been established that naturally occurring viruses can be engineered to produce an oncolytic effect in tumor cells (Wildner, 2001; Jacotat, 1967; Kim, 2001; Geoerger et al., 2002; Yan et al., 2003; Vile et al., 2002, each of which is incorporated herein by reference). In the case of adenoviruses, specific deletions within their adenoviral genome can attenuate their ability to replicate within normal quiescent cells, while they retain the ability to replicate in tumor cells. One such conditionally replicating adenovirus, Δ24, has been described by Fueyo et al. (2000), see also U.S. Patent Application No. 20030138405, each of which are incorporated herein by reference. The A24 adenovirus is derived from adenovirus type 5 (Ad-5) and contains a 24-base-pair deletion within the CR2 portion of the E1A gene. See, for example WO2001036650A2 (incorporated by reference herein in it's entirety.

Oncolytic adenoviruses include conditionally replicating adenoviruses (CRADs), such as Delta 24, which have several properties that make them candidates for use as biotherapeutic agents. One such property is the ability to replicate in a permissive cell or tissue, which amplifies the original input dose of the oncolytic virus and helps the agent spread to adjacent tumor cells providing a direct antitumor effect.

In some embodiments, the oncolytic component of Delta 24 with a transgene expression approach to produce an armed Delta 24. Armed Delta 24 adenoviruses may be used for producing or enhancing bystander effects within a tumor and/or producing or enhancing detection/imaging of an oncolytic adenovirus in a patient, or tumor associated tissue and/or cell. In some embodiments, the combination of oncolytic adenovirus with various transgene strategies (e.g., targeting with an IL-4 and/or IL-13 moiety) will improve the therapeutic potential including for example, potential against a variety of refractory tumors, as w ell as provide for improved imaging capabilities. In certain embodiments, an oncolytic adenovirus may be administered with a replication defective adenovirus, another oncolytic virus, a replication competent adenovirus, and/or a wildtype adenovirus. Each of which may be adminstered concurrently, before or after the other adenoviruses.

In some embodiments, an E1a adenoviral vectors involves the replacement of the basic adenovirus E1a promoter, including the CAAT box, TATA box and start site for transcription initiation, with a basic promoter that exhibits tumor specificity, and preferably is E2F responsive, and more preferably is the human E2F-1 promoter. Thus, this virus will be repressed in cells that lack molecules, or such molecules are non functional, that activate transcription from the E2F responsive promoter. Normal non dividing, or quiescent cells, fall in this class, as the transcription factor, E2F, is bound to pRb, or retinoblastoma protein, thus making E2F unavailable to bind to and activate the E2F responsive promoter. In contrast, cells that contain free E2F should support E2F based transcription. An example of such cells are neoplastic cells that lack pRb function, allowing for a productive viral infection to occur. In some embodiments, an E1a adenoviral vector is targeted use an IL-4 and/or IL-13 moiety as described herein.

Retention of the enhancer sequences, packaging signals, and DNA replication start sites which lie in the E1a promoter will ensure that the adenovirus infection proceeds to wild type levels in the neoplastic cells that lack pRb function. In essence, the modified E1a promoter confers tumor specific transcriptional activation resulting in substantial tumor specific killing, yet provides for enhanced safety in normal cells.

In some embodiments, an E1a adenoviral vector is prepared by substituting the endogenous E1a promoter with the E2F responsive promoter, the elements upstream of nucleotide 375 in the adenoviral 5 genome are kept intact. The nucleotide numbering is as described by See, Schmid, S. I., and Hearing, P. Current Topics in Microbiology and Immunology, vol. 199: pages 67-80 (1995). This includes all of the seven A repeat motifs identified for packaging of the viral genome (See FIG. 2 of Schmid and Hearing, above.) Sequences from nucleotide 375 to nucleotide 536 are deleted by a BsaAI to BsrBI restriction start site, while still retaining 23 base pairs upstream of the translational initiation codon for the E1A protein. An E2F responsive promoter, preferably human E2F-1 is substituted for the deleted endogenous E1a promoter sequences using known materials and methods. The E2F-1 promoter may be isolated as described in Example 1.

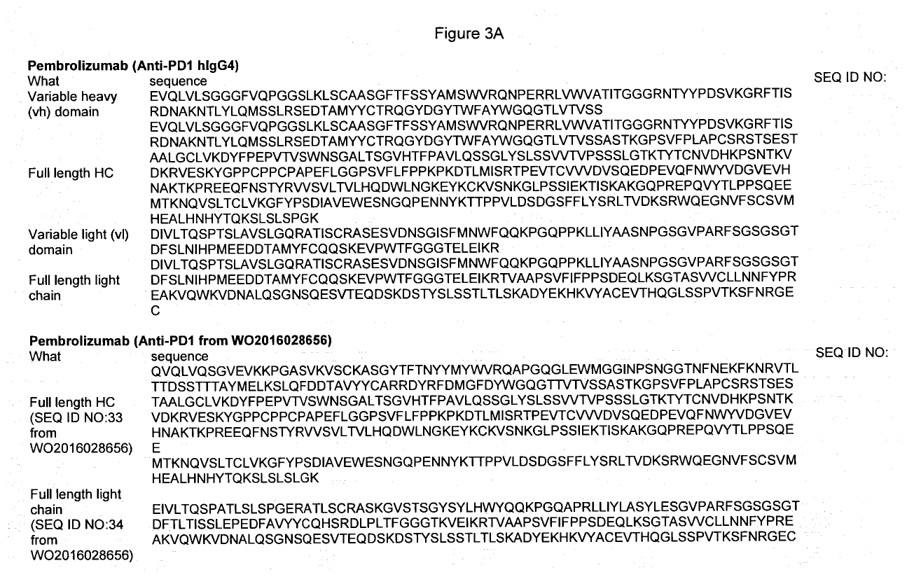

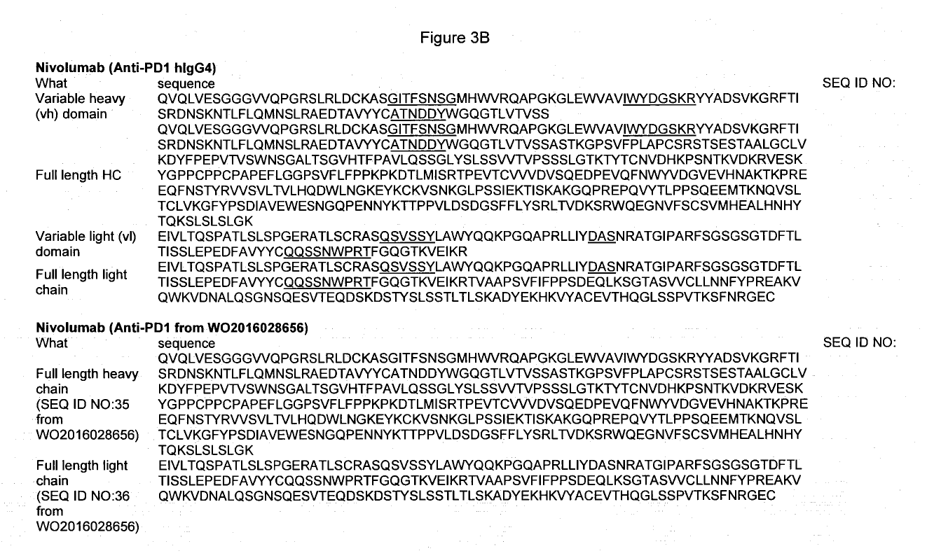

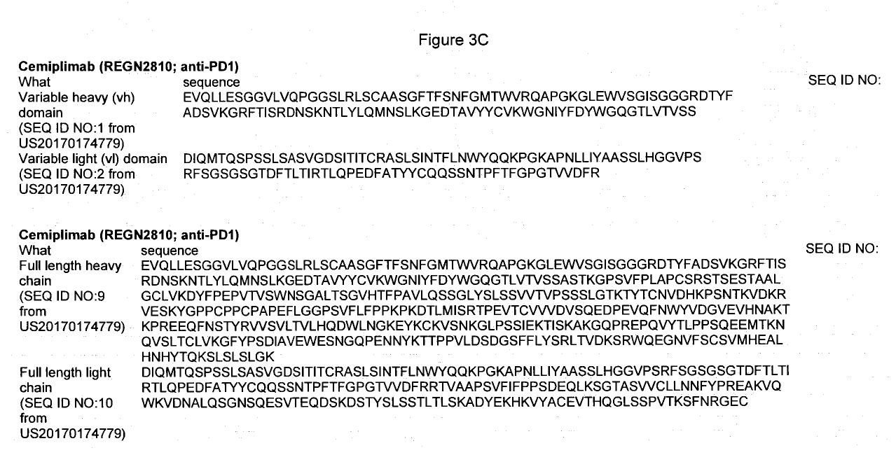

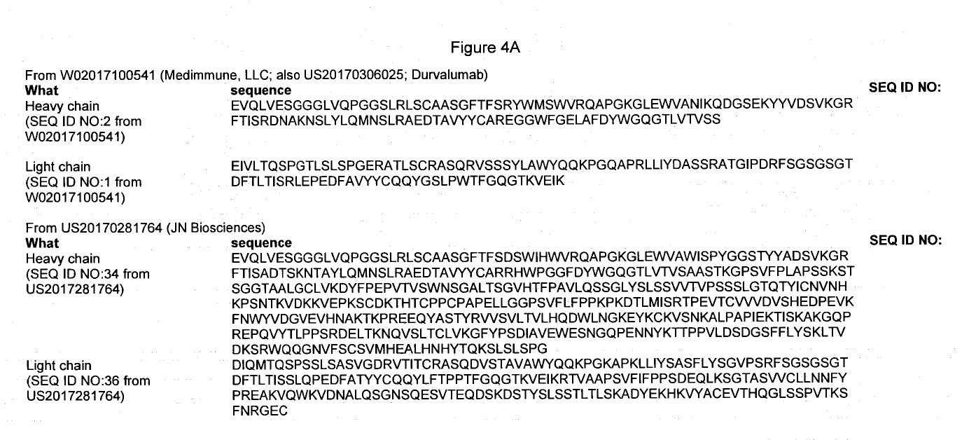

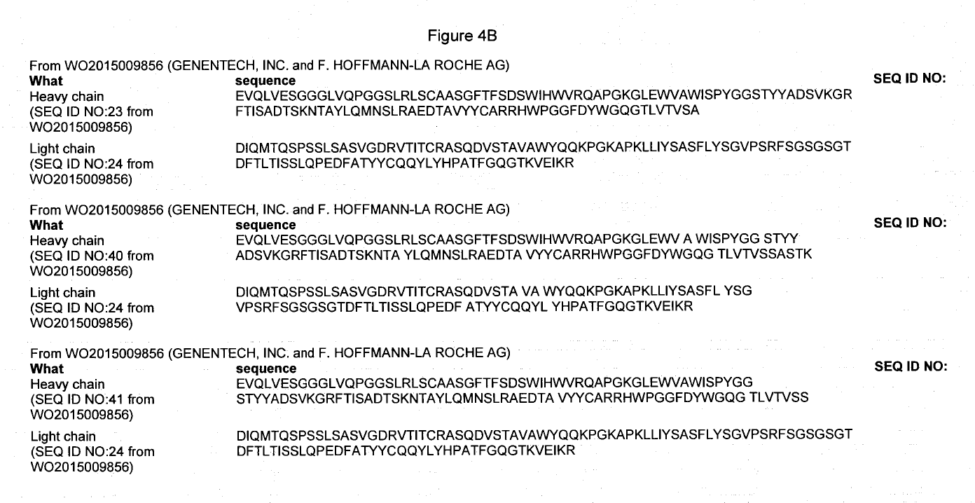

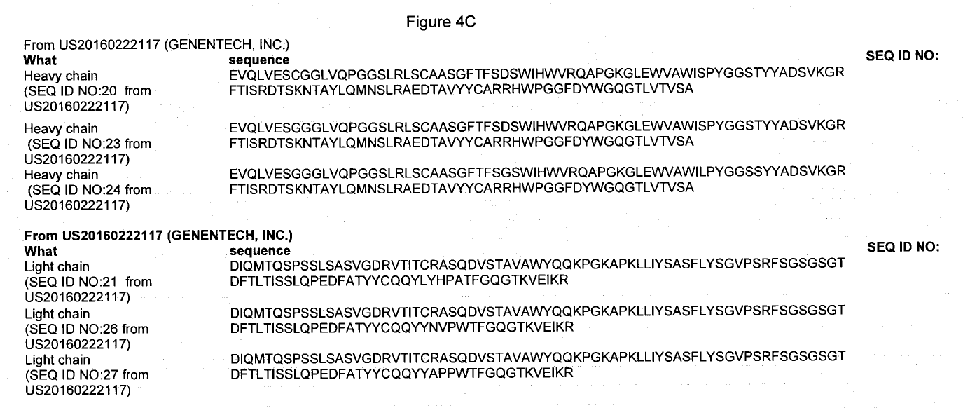

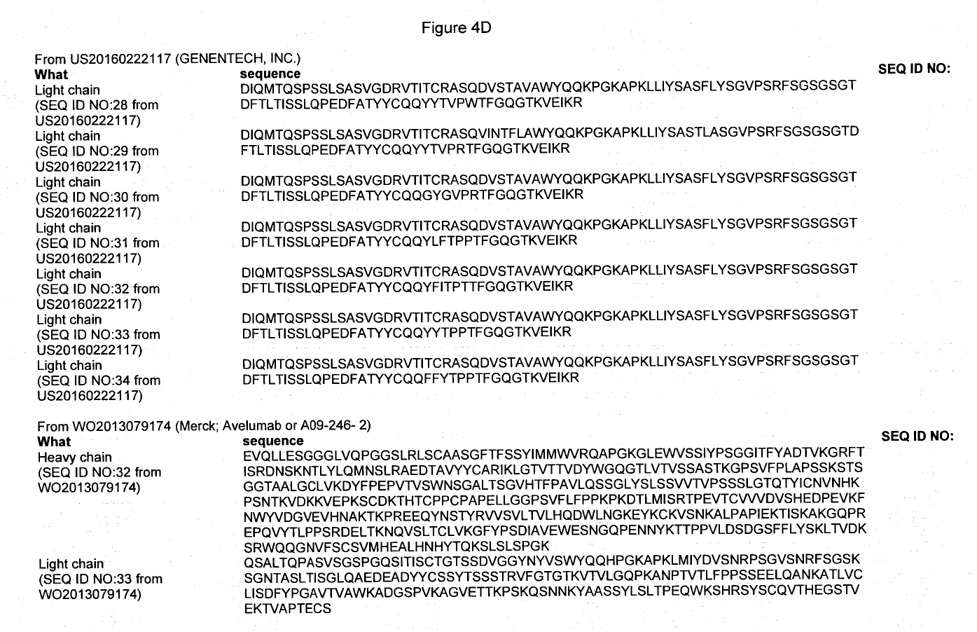

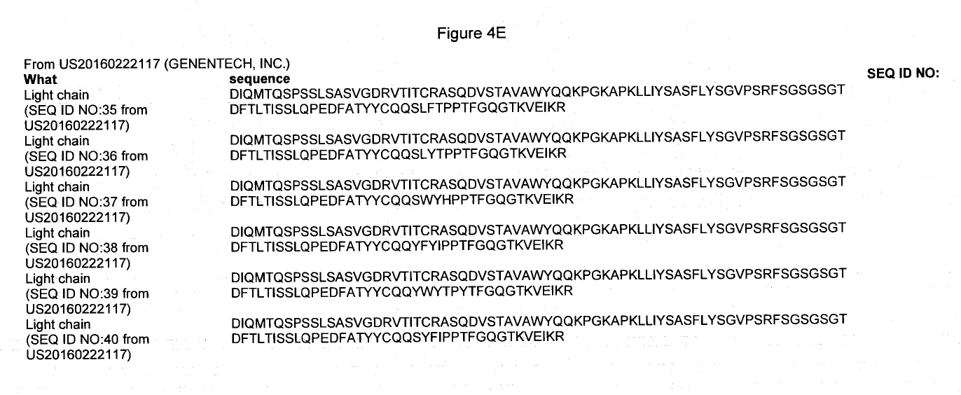

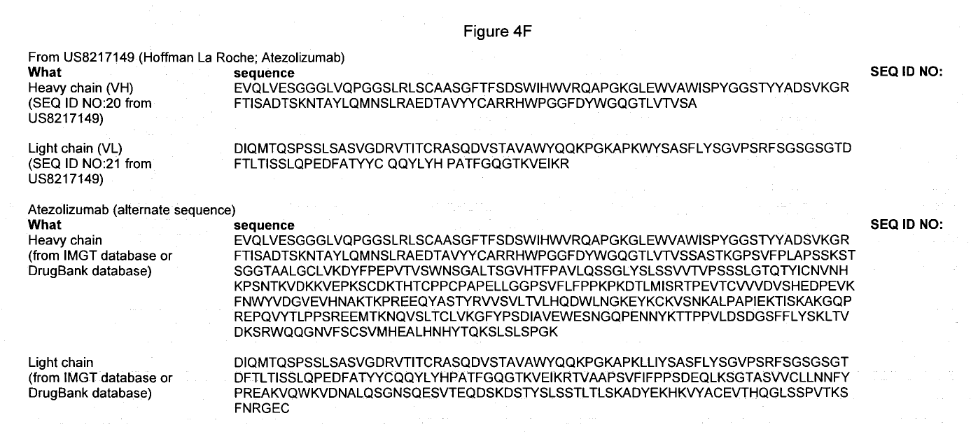

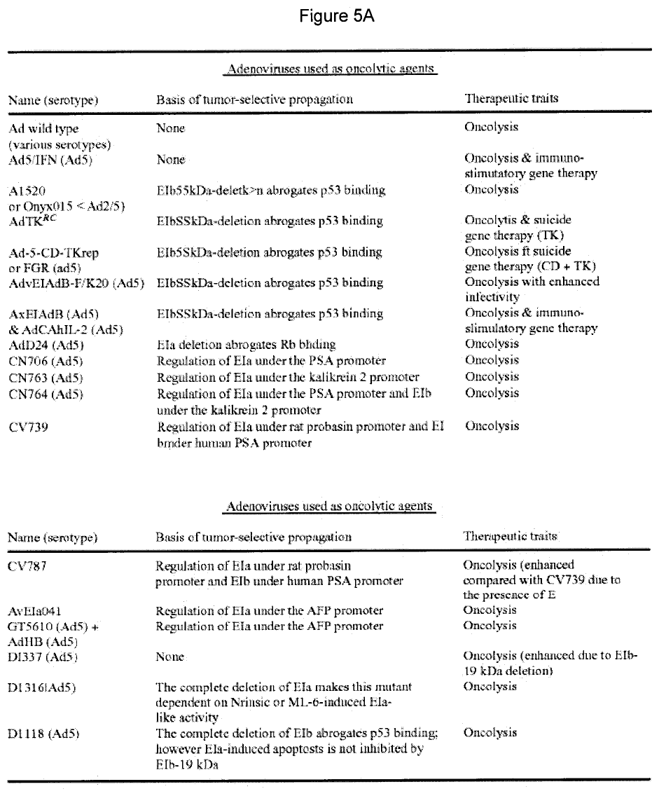

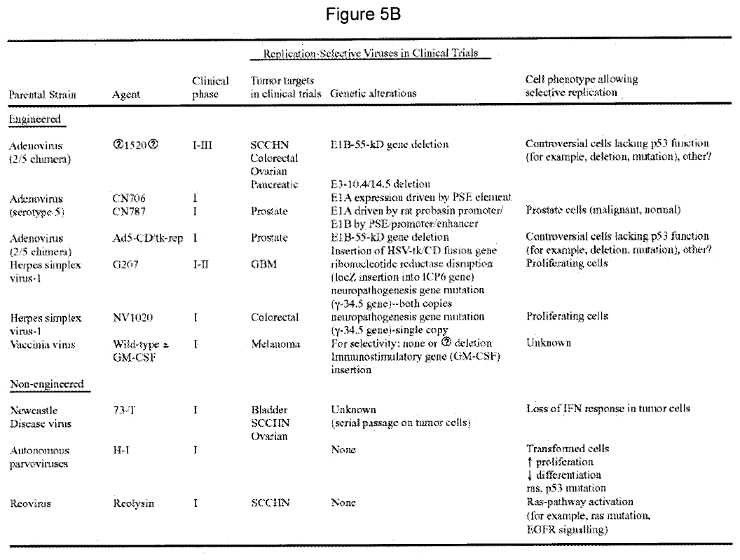

The E4 region has been implicated in many of the events that occur late in adenoviral infection, and is required for efficient viral DNA replication, late mRNA accumulation and protein synthesis, splicing, and the shutoff of host cell protein synthesis. Adenoviruses that are deficient for most of the E4 transcription unit are severely replication defective and, in general, must be propagated in E4 complementing cell lines to achieve high titers. The E4 promoter is positioned near the right end of the viral genome and governs the transcription of multiple open reading frames (ORF). A number of regulatory elements have been characterized in this promoter that are critical for mediating maximal transcriptional activity. In addition to these sequences, the E4 promoter region contains regulatory sequences that are required for viral DNA replication. A depiction of the E4 promoter and the position of these regulatory sequences can be seen in FIGS. 2 and 3 of U.S. Pat. No. 7,001,596, incorporated by reference herein in it entirety.

In some embodiments, the adenoviral vector that has the E4 basic promoter substituted with one that has been demonstrated to show tumor specificity, preferably an E2F responsive promoter, and more preferably the human E2F-1 promoter. The reasons for preferring an E2F responsive promoter to drive E4 expression are the same as were discussed above in the context of an E1a adenoviral vector having the E1a promoter substituted with an E2F responsive promoter. The tumor suppressor function of pRb correlates with its ability to repress E2F-responsive promoters such as the E2F-1 promoter (Adams, P. D., and W. G. Kaelin, Jr. 1995, Cancer Biol. 6:99-108; Sellers, W. R., and W. G. Kaelin. 1996, published erratum appears in Biochim Biophys Acta 1996 Dec. 9; 1288(3):E-1, Biochim Biophys Acta. 1288:M1-5. Sellers, W. R., J. W. Rodgers, and W. G. Kaelin, Jr. 1995, Proc Natl Acad Sci USA. 92:11544-8.) The human E2F-1 promoter has been extensively characterized and shown to be responsive to the pRb signaling pathway, including pRb/p107, E2F-1/-2/-3, and G1 cyclin/cdk complexes, and ETA (Johnson, D. G., K. Ohtani, and J. R. Nevins. 1994, Genes Dev. 8:1514-25; Neuman, E., E. K. Flemington, W. R. Sellers, and W. G. Kaelin, Jr. 1995. Mol Cell Biol. 15:4660; Neuman, E., W. R. Sellers, J. A. McNeil, J. B. Lawrence, and W. G. Kaelin, Jr. 1996, Gene. 173:163-9.) Most, if not all, of this regulation has been attributed to the presence of multiple E2F sites present within the E2F-1 promoter. Hence, a virus carrying this (these) modification(s) would be expected to be attenuated in normal cells that contain an intact (wild type) pRb pathway, yet exhibit a normal infection/replication profile in cells that are deficient for pRb's repressive function. In order to maintain the normal infection/replication profile of this mutant virus we have retained the inverted terminal repeat (ITR) at the distal end of the E4 promoter as this contains all of the regulatory elements that are required for viral DNA replication (Hatfield, L. and P. Hearing. 1993, J. Virol. 67:3931-9; Rawlins, D. R., P. J. Rosenfeld, R. J. Wides, M. D. Challberg, and T. J. Kelly, Jr. 1984, Cell. 37:309-19; Rosenfeld, P. J., E. A. O'Neill, R. J. Wides, and T. J. Kelly. 1987, Mol Cell Biol. 7:875-86; Wides, R. J., M. D. Challberg, D. R. Rawlins, and T. J. Kelly. 1987, Mol Cell Biol. 7:864-74). This facilitates attaining wild type levels of virus in pRb pathway deficient tumor cells infected with this virus.

In some embodiments, the E4 promoter is positioned near the right end of the viral genome and it governs the transcription of multiple open reading frames (ORFs) (Freyer, G. A., Y. Katoh, and R. J. Roberts. 1984, Nucleic Acids Res. 12:3503-19; Tigges, M. A., and H. J. Raskas. 1984. Splice junctions in adenovirus 2 early region 4 mRNAs: multiple splice sites produce 18 to 24 RNAs. J. Virol. 50:106-17; Virtanen, A. P. Gilardi, A. Naslund, J. M. LeMoullec, U. Pettersson, and M. Perricaudet. 1984, J. Virol. 51:822-31.) A number of regulatory elements have been characterized in this promoter that mediate transcriptional activity (Berk, A. J. 1986, Annu Rev Genet. 20:45-79; Gilardi, P., and M. Perricaudet. 1986, Nucleic Acids Res. 14:9035-49; Gilardi, P., and M. Perricaudet. 1984, Nucleic Acids Res. 12:7877-88; Hanaka, S., T. Nishigaki, P. A. Sharp, and H. Handa. 1987, Mol Cell Biol. 7:2578-87; Jones, C., and K. A. Lee. 1991, Mol Cell Biol. 11:4297-305; Lee, K. A., and M. R. Green. 1987, Embo J. 6:1345-53.) In addition to these sequences, the E4 promoter region contains elements that are involved in viral DNA replication (Hatfield, L., and P. Hearing. 1993, J Virol. 67:3931-9; Rawlins, D. R., P. J. Rosenfeld, R. J. Wides, M. D. Challberg, and T. J. Kelly, Jr. 1984, Cell. 37:309-19; Rosenfeld, P. J., E. A. O'Neill, R. J. Wides, and T. J. Kelly. 1987, Mol Cell Biol. 7:875-86; Wides, R. J., M. D. Challberg, D. R. Rawlins, and T. J. Kelly. 1987, Mol Cell Biol. 7:864-74.) A depiction of the E4 promoter and the position of these regulatory sequences can be seen in FIGS. 1 and 2. See, also, Jones, C., and K. A. Lee. Mol Cell Biol. 11:4297-305 (1991). With these considerations in mind, an E4 promoter shuttle was designed by creating two novel restriction endonuclease sites: a XhoI site at nucleotide 35,576 and a SpeI site at nucleotide 35,815 (see FIG. 3). Digestion with both XhoI and SpeI removes nucleotides from 35,581 to 35,817. This effectively eliminates bases −208 to +29 relative to the E4 transcriptional start site, including all of the sequences that have been shown to have maximal influence on E4 transcription. In particular, this encompasses the two inverted repeats of E4F binding sites that have been demonstrated to have the most significant effect on promoter activation. However, all three Sp1 binding sites, two of the five ATF binding sites, and both of the NF1 and NFIII/Oct-1 binding sites that are critical for viral DNA replication are retained.

In some embodiments, the E2F responsive promoter is the human E2F-1 promoter. Key regulatory elements in the E2F-1 promoter that mediate the response to the pRb pathway have been mapped both in vitro and in vivo (Johnson, D. G., K. Ohtani, and J. R. Nevins. 1994, Genes Dev. 8:1514-25; Neuman, E., E. K. Flemington, W. R. Sellers, and W. G. Kaelin, Jr. 1995, Mol Cell Biol. 15:4660; Parr, M. J., Y. Manome, T. Tanaka, P. Wen, D. W. Kufe, W. G. Kaelin, Jr., and H. A. Fine. 1997, Nat Med. 3:1145-9.) Thus, we isolated the human E2F-1 promoter fragment from base pairs −218 to +51, relative to the transcriptional start site, by PCR with primers that incorporated a SpeI and XhoI site into them. This creates the same sites present within the E4 promoter shuttle and allows for direct substitution of the E4 promoter with the E2F-1 promoter.

E. Chimerica Antigen Receptors (Cars)

Targeted immunotherapy has emerged as promising field of research in the treatment of malignancies and has received a great deal of interest in recent years. Indeed, cures have been reported of lymphoma patients with engineered or genetically modified T cells targeting CD19 malignant cells. This has increased the focus towards antigens present on cancer cells as targets for gene- and immunotherapy. These CARS can be used to target or deliver the IL-4 muteins described herein to the tumor, or even allow for systemic IL-4 mutein expression. In some embodiments, the IL-4 mutein is any IL-4 mutein or variant disclosed herein. In some embodiments, the IL-4 mutein sequence is 90% identical to any one of the IL-4 sequences provided herein.

Genetic manipulation of autologous or allogeneic T cells or NK cells to specifically target a particular tumor antigen provides a strategy to bypass the failure of cytotoxic immune response induction by most tumor cells. In some embodiments, these genetically manipulated T-cells or NK cells can be used to target the IL-4 muteins described herein to the tumor, for example, so that the IL-4 mutein is expressed at the tumor location. These technologies are based on the genetic modification of human immune cells, where the cells may be extracted from a patient or donor by leukapheresis. Specific cells, usually T-cells, are purified and engineered to express a receptor targeting a cancer antigen of interest. Engineering may utilize transduction by retroviral, lentiviral, transposon, mRNA electroporation, and the like. The immune cells may be expanded to the desired dose, and introduced into a patient. The engineered cells can specifically kill cancer cells through cell-mediated toxicity (cytotoxic T-cells) and/or eliciting an immune response to the cancer cell by immune recognition of tumor, cytokine release and immune cell recruitment.

For example, the application of chimeric antigen receptors (CAR) for immunogene therapy of malignant tumors is a promising strategy in which an antibody or ligand binding domain is fused with the zeta signaling chain of the T cell receptor. The resulting CAR immune cells are redirected by the neospecificity to attack tumors expressing the surface antigen or receptors recognized by the gene-modified T cell receptors and provide cellular therapy that attacks the tumor through normal host immune response in a highly regulated fashion. These cells are free to circulate throughout the brain and systemic circulation, making the need for colocalization and bioavailability less of a problem.

A number of generations of CAR immune cells have been developed. CARs are created by the fusion of a tumour-specific scFv antibody or other extracellular ligand binding domain to either the TCR-associated CD3ζ signalling domain or another intracellular signalling domains from co-stimulatory protein receptors. This structure allows CARs to have the tumor specificity of the B cell antigen receptor, and to activate T cells through the T cell antigen receptor independently of MHC binding. The first-generation CAR contained one intracellular signalling domain, typically with the CD3ζ signalling domain to allow for TCR signalling. Second-generation CARs have two intracellular signalling domains: a co-stimulatory domain comprising either a CD28 or a 4-1BB signalling domain, coupled with a CD3ζ signalling domain. This arrangement enables T-cell activation and proliferation upon antigen recognition by the scFv region of the CAR. The third-generation CARs have two co-stimulatory domains and a CD3ζ signalling domain. The first co-stimulatory domain is either a CD28 or a 4-1BB domain, with the second co-stimulatory domain consisting of either a CD28, a 4-1BB or a OX40 domain. Fourth-generation “armoured CAR T cells” combine a second-generation CAR with the addition of various genes, including cytokine and co-stimulatory ligands, to enhance the tumoricidal effect of the CAR T cells. See, for example, Batlevi et al. (2016) Nature Reviews Clinical Oncology 13:25-40. See also, U.S. Pat. No. 7,741,465 and International Patent Publication No. WO2014127261; all of which are incorporated by reference herein in their entireties.

Alternative approaches to T cell targeting include T cell antigen couplers, as described in International application WO2015/117229, entitled “Trifunctional T cell antigen Coupler and Methods and Uses thereof”, herein specifically incorporated by reference. The T cell antigen coupler system comprises three linked domains: a target-specific polypeptide ligand; a ligand that binds a protein associated with the TCR complex, for example an scFv binding to CD3 (TCR, T-cell receptor) to stimulate T cell activation; and a T cell receptor signaling domain, for example a CD4 transmembrane and intracellular domain to amplify T cell activation. By stimulating T cell activation through the TCR, TACs were engineered to work with the T cell's essential molecular machinery.

Antibody coupled T cell receptors are another approach to T cell targeting. ACTRs are a hybrid approach to CARs and the established monoclonal antibody oncology therapeutics. ACTRs are composed of a typical CAR construct that can bind the heavy chain of an antibody through a high-affinity variant of the Fc receptor CD16. ACTR-T cells can target tumours by binding a ligand targeted to a specific cancer antigen. T cell activation is performed by the CAR module.

Bispecific T cell exchangers (BiTEs) are bispecific antibodies that can bind the TCR of T cells and target tumour cells through two modules: a cancer targeting ligand; and a CD3-binding scFv domain that bridges T cells to the tumor.

Targeted therapies have been developed against IL13Rα2, including bacterial toxins conjugated to IL13, nanoparticles, oncolytic virus, as well as immunotherapies using monoclonal antibodies, IL13Rα2-pulsed dendritic cells, and IL13Rα2-targeted chimeric antigen receptors (see Kahlon et al. (2004) Cancer Research. 64(24):9160-9166; Kong et al. (2012) Clinical Cancer Research. 18(21):5949-5960; Thaci et al. (2014) Neuro-Oncology; and clinical trials NCT02208362, NCT00730613 and NCT01082926). In some emnodiemtns, these targeted therapies can be used to deliver the IL-4 muteins to the tumor.

Biologicals that provide for selective alteration of IL-13 activity are of interest for a number of therapeutic purposes, including the treatment of certain cancers with by engineering of T cell specificities. The present invention addresses this issue.

Methods and compositions are provided for enhancing anti-tumor immune effector cells, e.g. T cells, NK cells, etc. with targeted compositions, including without limitation chimeric antigen receptors (CARs); T cell antigen couplers (TACs); antibody coupled T cell receptors (ACTRs); and bispecific T cell exchangers (BiTEs), where an IL-13 or IL-4 superkine provides the target-specific ligand. In further embodiments, the immune effector cell expresses an IL-4 mutein.

Immune cell targeting or expression constructs comprising IL-4 mutein sequences are provided and can include any IL-4 sequence as described herein. Superkines are useful for targeting immune cells to cells, e.g. tumor cells, expressing the at least one receptor. In some embodiments, the IL-4 mutein is any IL-4 mutein or variant disclosed herein. In some embodiments, the IL-4 mutein sequence is 90% identical to any one of the IL-4 sequences provided herein.

The IL-4 or mutein component of the construct may be at least about 50 amino acids in length, at least about 75, at least about 100, at least about 110, at least about 115 amino acids in length, up to the full-length of the wild-type protein at the transmembrane domain, i.e. about 116 amino acids in length. For example, the superkine or mutein may be fused to the hinge, transmembrane or signaling domains of a CAR. Exemplary polypeptide sequences are provided

Included as superkines or muteins are amino acid and nucleic acid coding sequences that are 90%, 95%, 98% or 99% identical to these sequences, longer sequences that comprise those sequences but also include additional nucleotides at the 3′ or 5′ end, for example any number of additional nucleotides or codons, such as 3, 6, 9, 12 or more nucleotides, or up to about 12, 20, 50 or 100 additional nucleotides, and any sequence that encodes the same amino acid sequence as these nucleic acids due to the degeneracy of the genetic code. In particular, sequences that are codon optimized (CO) for expression by the desired host are contemplated as part of the invention. In some embodiments, the amino acid sequence is 90% identical. In some embodiments, the amino acid sequence is 95% identical. In some embodiments, the amino acid sequence is 98% identical. In some embodiments, the amino acid sequence is 99% identical. In some embodiments, the polypeptide is linked to an IL-4 mutein immune cell targeting or expression construct. In some embodiments, an IL-4 mutein immune cell targeting or expression construct comprises one or more signaling domains derived from CD3-ζ, CD28, DAP10, OX-40, ICOS and CD137. In some embodiments, an IL-4 mutein immune cell targeting or expression construct or expression comprises one or more signaling domains derived from CD3-ζ. In some embodiments, an IL-4 mutein immune cell targeting or expression construct comprises one or more signaling domains derived from CD28. In some embodiments, an IL-4 mutein immune cell targeting or expression construct comprises one or more signaling domains derived from DAP10. In some embodiments, an IL-4 mutein immune cell targeting or expression construct comprises one or more signaling domains derived from OX-40. In some embodiments, an IL-4 mutein immune cell targeting or expression construct comprises one or more signaling domains derived from CD137. In some embodiments, an IL-4 mutein immune cell targeting or expression construct comprises an IL-4 variant/IL-4 mutein including those provided herein. In some embodiments, an IL-4 mutein immune cell targeting or expression construct comprises an IL-4 variant/IL-4 mutein including those provided herein.

1. NK Cells

In some embodiments the immune cells are natural killer (NK) cells. NK cells recognize infected or transformed cells through multiple cell surface receptors including NKG2D, CD16, and natural cytotoxicity receptors (NCRs) such as NKp44, NKp46, and NKp30. These receptors activate signaling adapter proteins such as DAP10, DAP12, and CD3ζ, which contain immuno-tyrosine activation motifs (ITAMs) that initiate the release of cytolytic granules containing perforin and granzymes, as well as mediate production and release of cytokines and chemokines such as IFN-γ and TNF-α. Importantly, NK cell-mediated cytotoxicity does not rely on the presentation of self HLA. Therefore, NK cells hold significant clinical interest as a cell-based therapy for cancer because of their ability to be used in an allogeneic setting and potentially provide an off-the-shelf cellular product.

Natural killer cells provide an alternative to the use of T cells for adoptive immunotherapy since they do not require HLA matching, so can be used as allogeneic effector cells. Clinical trials of adoptively transferred allogeneic NK cells demonstrate these cells can survive in patients for several weeks to months. Additionally, expression of CARs in NK cells allow these cells to more effectively kill solid tumors that are often resistant to NK cell-mediated activity compared to hematologic malignancies (especially acute myelogenous leukemia) that are typically more NK cell-sensitive. CARs useful in NK cell targeting include, for example, first generation CAR constructs that contain CD3 as the sole signaling domain. Second and third generation CARs are also useful in NK cells. In some embodiments the ectodomain of NKG2D, an NK cell activation receptor, is linked directly to CD3ζ.

NK cells for modification include cell lines, or peripheral blood NK cells, which can be isolated from donors through simple blood draws or by apheresis if larger numbers of cells are needed. Activated PB-NK cells express a wider range of activating receptors, such as CD16, NKp44, and NKp46 as well as KIRs, which play an important role in NK cell licensing. In addition, PB-NK cells can be given without irradiating the cells so have the ability to expand in vivo. Another source of NK cells suitable for CAR expression are NK cells derived from human pluripotent stem cells—both induced pluripotent stem cells (iPSCs) or human embryonic stem cells (hESCs). These NK cells display a similar phenotype to PB-NK cells, and hESC/iPSC-NK cells can be grown on a clinical scale.

2. Chimerica Antigen Receptors (CARs)

In addition to the superkine sequence, CARs contain the signaling domain for CD3ζ and the signaling domains of one or more costimulatory receptors that further promote the recycling, survival and/or expansion of immune cells expressing the CARs. The signaling domains of the costimulatory receptors are the intracellular portions of each receptor protein that generate the activating signal in the cell. Examples are amino acids 180-220 of the native CD28 molecule and amino acids 214-255 of the native 4-1BB molecule.

Examples of suitable hinge and transmembrane regions to link the superkine to the signaling region may include without limitation the constant (Fc) regions of immunoglobins, human CD8a, and artificial linkers that serve to move the targeting moiety away from the cell surface for improved access to and binding on target cells. Examples of suitable transmembrane domains include the transmembrane domains of the leukocyte CD markers, preferably that of CD4 or CD28. Examples of intracellular receptor signaling domains include the T cell antigen receptor complex, preferably the zeta chain of CD3, however any transmembrane region sufficient to anchor the CAR in the membrane can be used. Persons of skill are aware of numerous transmembrane regions and the structural elements (such as lipophilic amino acid regions) that produce transmembrane domains in numerous membrane proteins and therefore can substitute any convenient sequence. T cell costimulatory signaling receptors suitable for improving the function and activity of CAR-expressing cells include, but are not limited to, CD28, CD137, and OX-40.

Signaling via CD28 is required for IL2 production and proliferation, but does not play a primary role in sustaining T cell function and activity. CD137 (a tumor necrosis factor-receptor family member expressed following CD28 activation) and OX-40 are involved in driving long-term survival of T cells, and accumulation of T cells. The ligands for these receptors typically are expressed on professional antigen presenting cells such as dendritic cells and activated macrophages, but not on tumor cells. Expressing a CAR that incorporates CD28 and/or 4-1BB signaling domains in CD4+ T cells enhances the activity and anti-tumor potency of those cells compared to those expressing a CAR that contains only the CD3ζ signaling domain, which constructs may be referred to as second or third generation CARs.

Included as CAR constructs of interest are tandem CARs, e.g. see Hegde et al. (2016) J. Clin. Invest 126(8):3036-3052, herein specifically incorporated by reference. In such constructs a binding moiety for a tumor specific antigen is combined in tandem with an IL-13 superkine. The binding moiety may be, for example, an scFv specific for a tumor cell antigen, including without limitation HER-2, EGFR, CD20, etc. as known in the art.

In various embodiments, the antigen binding domain binds to an antigen on a target cell, e.g., a cancer cell. The antigen binding domain can bind an antigen, such as but not limited to a tumor target antigen. In some case, the antigen binding domain binds one or more antigens. Exemplary antigen binding domains can bind to an antigen including, but not limited to, D19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3; TNF receptor family member B cell maturation (BCMA); Tn antigen ((Tn Ag) or (GalNAca Ser/Thr)); prostate-specific membrane antigen (PSMA); Receptor tyrosine kinase-like orphan receptor 1 (RORI); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Rα2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp 100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTi); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART 1); Rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antigen binding domain comprises a monoclonal antibody, a polyclonal antibody, a synthetic antibody, a human antibody, a humanized antibody, a non-human antibody, a nanobody, a single-chain variable fragment (scFv), F(ab′)2, Fab′, Fab, Fv, and the like. The antigen binding domain can be linked to the transmembrane domain of the CAR. In some embodiments, a nucleic acid encoding the antigen binding domain is operably linked to a nucleic acid encoding a transmembrane domain of the CAR.

In some embodiments, the transmembrane domain can be derived from a membrane-bound or transmembrane protein. In certain embodiments, the transmembrane domain comprises one or more, e.g., 1, 2, 3, 4, 5, 6, 7, 8 or more amino acid modifications (e.g., substitutions, insertions, and deletions) compared to the wild-type amino acid sequence of the transmembrane domain of the membrane-bound or transmembrane protein. Non-limiting examples of a transmembrane domain of a CAR include at least the transmembrane region(s) of the alpha, beta or zeta chain of the T-cell receptor, CD28, CD3 epsilon (CD3), CD45, CD4, CD5, CD8, CD9, CD16, CD22, CD33, CD37, CD64, CD80, CD86, CD134, CD137, CD154, or an erythropoietin receptor. In some embodiments, the transmembrane domain includes a human immunoglobulin (Ig) hinge region, e.g., an IgG4Fc hinge. In other embodiments, the transmembrane domain is a recombinant or synthetic domain comprising hydrophobic amino acid residues (e.g., leucine and valine). In some cases, the transmembrane domain includes a phenylalanine, tryptophan and valine at one or both ends of the domain.

The transmembrane domain links the antigen binding domain to the intracellular signaling domain of the CAR. In some embodiments, the nucleic acid encoding the antigen binding domain is operably linked to the nucleic acid encoding the transmembrane domain that is operably linked to the nucleic acid encoding the intracellular signaling domain.

In some embodiments, the intracellular signaling domain of a CAR comprises a signal activation or signal transduction domain. As such, an intracellular signaling domain includes any portion of an intracellular signaling domain of a protein sufficient to transduce or transmit a signal, e.g., an activation signal or to mediate a cellular response within a cell. Non-limiting examples include TCR, CD2, CD3 zeta, CD3 gamma, CD3 delta, CD3 epsilon, CD7, CD27, CD86, common FcR gamma, FcR beta, CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8 beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, any derivative, variant, or fragment thereof. In certain embodiments, the intracellular signaling domain comprises an intracellular domain of a co-stimulatory molecule such as from CD3, CD27, CD28, CD127, ICOS, 4-1BB (CD137), PD-1, T cell receptor (TCR), any derivative thereof, or any variant thereof. In some embodiments, the intracellular signaling domain of the CAR is selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

1. BiTES

Bi-specific T-cell engagers (BiTEs) are fusion proteins comprising an IL-13 superkine fused to an antibody variable region that specifically binds to CD3. In some embodiments the antibody variable region in a single-chain variable fragments (scFvs). The superkine may be fused to the variable region through a linker. An Fc region is optionally provided.

2. TACs

A TAC construct comprises an IL-4 mutein fused to a ligand that binds a protein associated with the TCR complex; fused to a T cell receptor signaling domain polypeptide. The domains may be separated by linkers. The protein associated with the TCR complex may be CD3. The ligand that binds a protein associated with the TCR complex may be a single chain antibody. The ligand that binds a protein associated with the TCR complex may be UCHT1, or a variant thereof. The T cell receptor signaling domain polypeptide may comprise a cytosolic domain and a transmembrane domain. The cytosolic domain may be a CD4 cytosolic domain and the transmembrane domain is a CD4 transmembrane domain.

3. ACTRs

ACTRs are a hybrid approach to CARs and the established monoclonal antibody oncology therapeutics. ACTRs are composed of a typical CAR construct that can bind the heavy chain of an antibody through a high-affinity variant of the Fc receptor CD16. A superkine is fused to a moiety recognized by the CAR, which may include, without limitation, an Fc region of an antibody with high affinity for CD16.

An immune cell targeting or expression construct coding sequence can be produced by any means known in the art, including recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region may be inserted into an expression vector and used to transform a suitable expression host cell line, e.g. a population of allogeneic or autologous T lymphocytes, allogeneic or autologous NK cells, including primary cultures, cell lines, iPSC derived cells, etc. The methods can be used on cells in vitro (e.g., in a cell-free system), in culture, e.g. in vitro or ex vivo. For example, IL-4 mutein CAR-expressing cells can be cultured and expanded in vitro in culture medium.

An non-IL-4 mutein immune cell targeting or expression construct can also be used specifically direct immune cells to target specific tumor cells. Anti-tumor effector cells, e.g. CD4⁺ or CD8⁺ effector T cells, are generated to be re-directed to recognize such tumor cells by introducing into the T cells an superkine immune cell targeting or expression construct comprising one or more signaling domains derived from CD3-ζ, CD28, DAP10, OX-40, ICOS and CD137. In some embodiments, the cells can further comprise a transgene capable of expressing an IL-4 mutein as described herein. An IL-4 mutein immune cell targeting or expression construct can specifically direct immune cells to target IL-4R expressing cell, including tumor cells. Anti-tumor effector cells, e.g. CD4⁺ or CD8⁺ effector T cells, are generated to be re-directed to recognize such tumor cells by introducing into the T cells an IL-4 mutein immune cell targeting or expression construct comprising one or more signaling domains derived from CD3-ζ, CD28, DAP10, OX-40, ICOS and CD137.

The IL-4 mutein immune cell targeting or expression construct is infected or transfected into human immune cells, e.g. using a non-viral plasmid vector and electroporation methods; a viral vector and infection methods, etc. as known in the art. A CAR comprising co-stimulatory signaling domains may enhance the duration and/or retention of anti-tumor activity in a manner that can significantly improve the clinical efficacy of adoptive therapy protocols. CD4⁺ and CD8⁺ T cell effector functions, and NK cell functions can be triggered via these receptors, therefore these cell types are contemplated for use with the invention. CD8⁺ T cells expressing the IL13 superkine CARs of this invention may be used to lyse target cells, among the other functions of these cells. Expression of the appropriate costimulatory CAR in either or both CD4⁺ and CD8⁺ T cells is used to provide the most effective population of cells for adoptive immunotherapy, consisting therefore of either or both professional helper and killer T cells that exhibit enhanced and/or long term viability and anti-tumor activity. In some embodiments, an IL-4 mutein immune cell targeting or expression construct comprises an IL-4 variant/IL-4 mutein including those provided in FIG. 1. In some embodiments, an IL-4 mutein immune cell targeting or expression construct comprises an IL-4 variant/IL-4 mutein including any of those provided herein.

Polypeptides of the present invention can be further modified, e.g., joined to a wide variety of other oligopeptides or proteins for a variety of purposes. For example, post-translationally modified, for example by prenylation, acetylation, amidation, carboxylation, glycosylation, pegylation, etc. Such modifications can also include modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes.

Methods which are well known to those skilled in the art can be used to construct T cell targeting construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art.

According to the present invention, immune cell targeting or expression construct vectors and immune cell targeting or expression construct modified cells can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, pharmaceutical compositions of the present invention include one or more therapeutic entities of the present invention in combination with another therapeutic agent, e.g., another anti-tumor agent.

Therapeutic entities of the present invention are often administered as pharmaceutical compositions comprising an active therapeutic agent and another pharmaceutically acceptable excipient. Such formulations can include one or more non-toxic pharmaceutically acceptable carriers, diluents, excipients and/or adjuvants. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

The maximum tolerated dose (MTD) of CAR immune cells may be determined during clinical trial development, for example at up to about 10⁴ T cells/kg of body weight, up to about 10⁵ cells/kg of body weight, up to about 10⁶ cells/kg of body weight, up to about 5×10⁶ cells/kg of body weight, up to about 10⁷ cells/kg of body weight, up to about 5×10⁷ cells/kg of body weight, or more, as empirically determined. In some embodiments, the maximum tolerated dose (MTD) of CAR immune cells is up to about 10⁴ T cells/kg of body weight. In some embodiments, the maximum tolerated dose (MTD) of CAR immune cells is up to about 10⁵ T cells/kg of body weight. In some embodiments, the maximum tolerated dose (MTD) of CAR immune cells is up to about 10⁶ T cells/kg of body weight. In some embodiments, the maximum tolerated dose (MTD) of CAR immune cells is up to about 10⁷ T cells/kg of body weight. In some embodiments, the maximum tolerated dose (MTD) of CAR immune cells is up to about 5×10⁶ T cells/kg of body weight. In some embodiments, the maximum tolerated dose (MTD) of CAR immune cells is up to about 5×10⁷ T cells/kg of body weight.

Toxicity of the cells described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

After a dose escalation phase, patients in the expansion cohort are treated with immune cells at the MTD. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient.

In prophylactic applications, e.g. to maintain remission in a patient, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Examples of additional therapeutic agents that can be coadministered and/or coformulated with an immune cell targeting or expression construct include: anti-proliferative, or cytoreductive therapy, which is used therapeutically to eliminate tumor cells and other undesirable cells in a host, and includes the use of therapies such as delivery of ionizing radiation, and administration of chemotherapeutic agents. Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g. the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Nucleotide analogs include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g. cisplatin (cis-DDP), carboplatin, oxaliplatin, etc.; ureas, e.g. hydroxyurea; and hydrazines, e.g. N-methylhydrazine.

For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells. Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

Treatment may also be combined with immunoregulatory modulating agents, including an agent that agonizes an immune costimulatory molecule, e.g. CD40, OX40, etc.; and/or (iii) an agent that antagonizes an immune inhibitory molecule, e.g. CTLA-4, PD-1, PD-L1, etc. The active agents are administered within a period of time to produce an additive or synergistic effect on depletion of cancer cells in the host. Methods of administration include, without limitation, systemic administration, intra-tumoral administration, etc.

In some embodiments, an individual cancer is selected for treatment with a combination therapy because the cancer is a cancer type that is responsive to a checkpoint inhibitor, e.g. a PD-1 antagonist, a PD-L1 antagonist, a CTLA4 antagonist, a TIM-3 antagonist, a BTLA antagonist, a VISTA antagonist, a LAG3 antagonist; etc. In some embodiments, such an immunoregulatory agent is a CTLA-4, PD1 or PDL1 antagonist, e.g. avelumab, nivolumab, pembrolizumab, ipilimumab, and the like. In some such embodiments the cancer is, without limitation, melanoma or small cell lung cancer. In some such embodiments, the cancer is a type that has a high neoantigen, or mutagenesis, burden (see Vogelstein et al. (2013) Science 339(6127):1546-1558, herein specifically incorporated by reference).

In some embodiments, an individual cancer is selected for treatment with a combination therapy of the present invention because the cancer is a cancer type that is responsive to an immune response agonist, e.g. a CD28 agonist, an OX40 agonist; a GITR agonist, a CD137 agonist, a CD27 agonist, an HVEM agonist, etc. In some embodiments, such an immunoregulatory agent is an OX40, CD137, or GITR agonist e.g. tremelimumab, and the like. In some such embodiments the cancer is, without limitation, melanoma or small cell lung cancer. In some such embodiments, the cancer is a type that has a high neoantigen, or mutagenesis, burden.

In some embodiments, the combination therapy includes an antibody known in the art which binds to PD-1 and disrupt the interaction between the PD-1 and its ligand, PD-L1, and stimulate an anti-tumor immune response. In some embodiments, the antibody or antigen-binding portion thereof binds specifically to PD-1. For example, antibodies that target PD-1 and which can find used in the present invention include, e.g., but are not limited to nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies include anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in the combination treatment methods disclosed herein. For example, antibodies that target PD-L1 and are in clinical trials, include BMS-936559 (Bristol-Myers Squibb) and MPDL3280A (Genetech). Other suitable antibodies that target PD-L1 are disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, is suitable for use in the combination treatment methods.

In some embodiments, the combination therapy includes an antibody known in the art which binds CTLA-4 and disrupts its interaction with CD80 and CD86. Exemplary antibodies that target CTLA-4 include ipilimumab (MDX-010, MDX-101, Bristol-Myers Squibb), which is FDA approved, and tremelimumab (ticilimumab, CP-675, 206, Pfizer), currently undergoing human trials. Other suitable antibodies that target CTLA-4 are disclosed in WO 2012/120125, U.S. Pat. Nos. 6,984,720, 6,682,7368, and U.S. Patent Applications 2002/0039581, 2002/0086014, and 2005/0201994, herein incorporated by reference. It will be understood by one of ordinary skill that any antibody which binds to CTLA-4, disrupts its interaction with CD80 and CD86, and stimulates an anti-tumor immune response, is suitable for use in the combination treatment methods. In some embodiments, the combination therapy includes an antibody known in the art which binds LAG-3 and disrupts its interaction with MHC class II molecules. An exemplary antibody that targets LAG-3 is IMP321 (Immutep), currently undergoing human trials. Other suitable antibodies that target LAG-3 are disclosed in U.S. Patent Application 2011/0150892, herein incorporated by reference. It will be understood by one of ordinary skill that any antibody which binds to LAG-3, disrupts its interaction with MHC class II molecules, and stimulates an anti-tumor immune response, is suitable for use in the combination treatment methods.

In some embodiments, the combination therapy includes an antibody known in the art which binds TIM-3 and disrupts its interaction with galectin 9. Suitable antibodies that target TIM-3 are disclosed in U.S. Patent Application 2013/0022623, herein incorporated by reference. It will be understood by one of ordinary skill that any antibody which binds to TIM-3, disrupts its interaction with galectin 9, and stimulates an anti-tumor immune response, is suitable for use in the combination treatment methods.

In some embodiments, the combination therapy includes an antibody known in the art which binds 4-1BB/CD137 and disrupts its interaction with CD137L. It will be understood by one of ordinary skill that any antibody which binds to 4-1BB/CD137, disrupts its interaction with CD137L or another ligand, and stimulates an anti-tumor immune response or an immune stimulatory response that results in anti-tumor activity overall, is suitable for use in the combination treatment methods.

In some embodiments, the combination therapy includes an antibody known in the art which binds GITR and disrupts its interaction with its ligand. It will be understood by one of ordinary skill that any antibody which binds to GITR, disrupts its interaction with GITRL or another ligand, and stimulates an anti-tumor immune response or an immune stimulatory response that results in anti-tumor activity overall, is suitable for use in the combination treatment methods.

In some embodiments, the combination therapy includes an antibody known in the art which binds OX40 and disrupts its interaction with its ligand. It will be understood by one of ordinary skill that any antibody which binds to OX40, disrupts its interaction with OX40L or another ligand, and stimulates an anti-tumor immune response or an immune stimulatory response that results in anti-tumor activity overall, is suitable for use in the combination treatment methods.

In some embodiments, the combination therapy includes an antibody known in the art which binds CD40 and disrupts its interaction with its ligand. It will be understood by one of ordinary skill that any antibody which binds to CD40, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds ICOS and disrupts its interaction with its ligand. It will be understood by one of ordinary skill that any antibody which binds to ICOS, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds CD28 and disrupts its interaction with its ligand. It will be understood by one of ordinary skill that any antibody which binds to CD28, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds IFNα and disrupts its interaction with its ligand. It will be understood by one of ordinary skill that any antibody which binds to IFNα, disrupts its interaction with its ligand, and stimulates an anti-tumor immune response or an immune stimulatory response that results in anti-tumor activity overall, is suitable for use in the combination treatment methods.

An “anti-cancer therapeutic” is a compound, composition, or treatment (e.g., surgery) that prevents or delays the growth and/or metastasis of cancer cells. Such anti-cancer therapeutics include, but are not limited to, surgery (e.g., removal of all or part of a tumor), chemotherapeutic drug treatment, radiation, gene therapy, hormonal manipulation, immunotherapy (e.g., therapeutic antibodies and cancer vaccines) and antisense or RNAi oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C, Avastin, Herceptin®, flurouracil, and temozolamide and the like. The compounds are also suitable for use with standard combination therapies employing two or more chemotherapeutic agents. It is to be understood that anti-cancer therapeutics includes novel compounds or treatments developed in the future.

The pharmaceutical compositions and/or formulations described above include one or more therapeutic entities in an amount effective to achieve the intended purpose. Thus the term “therapeutically effective dose” refers to the amount of the therapeutic entities that ameliorates the symptoms of cancer. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans, using standard methods known in those of ordinary skill in the art.

Also within the scope of the invention are kits comprising the compositions of the invention and instructions for use. The kit may further contain a least one additional reagent, e.g. a chemotherapeutic drug, anti-tumor antibody, etc. Kits typically include a label indicating the intended use of the contents of the kit. The term label includes any writing, or recorded material supplied on or with the kit, or which otherwise accompanies the kit.

The invention now being fully described, it will be apparent to one of ordinary skill in the art that various changes and modifications can be made without departing from the spirit or scope of the invention. In some embodiments, the kit comprises an IL-4 mutein immune cell targeting or expression construct comprising an IL-4 variant/IL-4 mutein as described herein. In some embodiments, the kit comprises an IL-4 superkine immune cell targeting or expression construct comprising an IL-4 variant/IL-4 mutein including those provided herein. In some embodiments, an IL-4 mutein immune cell targeting or expression construct comprises an IL-4 variant/IL-4 mutein including those provided herein.

F. Cancer Stem Cell Targeting Moieties

Targeting moieties are the portion of the IL-4 targeted cargo proteins that target the IL-4 targeted cargo protein to cancer cells, and including cancer cells and/or cancer stem cells and bulk cancer cells. Targeting moieties function to specifically bind to a cancer stem cell. However, it is appreciated that the targeting moiety need not retain its native biological activity (e.g., the ability to activate a receptor or ability to prevent a ligand from binding to its receptor) as long as it permits the IL-4 targeted cargo protein to bind with high specificity to cancer cells and/or cancer stem cells (and in some examples also cancer cells). In certain examples, the targeting moiety is a natural ligand of a target displayed by the cancer stem cell or a derivative of a natural ligand. In other examples the targeting moiety is an antibody, such as a humanized antibody or antibody fragment, which specifically binds to a target displayed on the surface of the cancer stem cell (e.g., targets a receptor). Targeting moieties can be linked to cargo moieties using any method known in the art, for example via chemical or recombinant technology.

A non-limiting list of compounds that could be used to target cancer cells and/or cancer stem cells includes antibodies, natural ligands, engineered ligands and combinations thereof that bind to one or more cancer cells and/or cancer stem cells. Exemplary ligands include cytokines and growth factors. Exemplary targets on cancer cells and/or cancer stem cells include, for example, IL-4R.

Of particular interest are targeting moieties that are molecules that are natural ligands or derivatives of the natural ligands to the target on the cancer cells and/or cancer stem cells. For example, if the cancer stem cell expresses IL-4 receptors (IL-4R), IL-4 ligand can be used as the targeting moiety. The IL-4 can be chemically or recombinantly linked to one or more of the cargo moieties described herein. Examples of derivatives of natural ligands include the circularized cytokine ligands described in U.S. Pat. No. 6,011,002 to Pastan et al., which is herein incorporated by reference. In addition to IL-4 ligands, IL-13 can also be used as a ligand targeting moiety since the IL-4 and IL-13 receptors share some sequence and biological functions. IL-4 targeted cargo proteins include those comprising IL-4 and IL-13 ligands and variants thereof.

In some examples, antibodies (including fragments, humanized antibodies and the like as described above) that target IL-4R. Antibodies are commercially available from various companies such as Millipore, Bedford, Mass. or custom made antibodies can be ordered from companies such as Cambridge Research Biochemicals, Billingham, Cleveland. Methods routine in the art can be used to generate such antibodies if desired. Such antibodies will specifically bind to cancer cells and/or cancer stem cells (and may also bind to bulk cancer cells) and function to place the cargo moiety in contact with a cancer stem cell.

IL-4 is a pleiotropic cytokine produced by activated T cells, and is the ligand for the IL-4 receptor. The IL-4 receptor also binds to IL-13. Thus, IL-13 can also be used as a targeting moiety to target the IL-4 receptor. IL-4, IL-3, IL-5, IL-13, and CSF2 form a cytokine gene cluster on human chromosome 5q, with this gene particularly close to IL-13. Exemplary IL-4 and IL-13 proteins that can be used in the IL-4 targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind the IL-4 receptor.

The targeting moiety used can include native sequences (such as the GenBank Accession Nos. and sequences present in the patents referenced in Table 2 and listed above), as well as variants thereof, such as a variant having at least 98%, at least 95%, at least 90%, at least 80%, at least 70%, or at least 60% sequence identity with the native targeting moiety protein (e.g., at least about this amount of sequence identity to the GenBank Accession Nos. listed in Table 2 and listed above). In some examples, variant sequences retain substantially the same amount (or even more) of the native biological function of the targeting moiety protein, such as the ability to activate an intracellular signal cascade. However, variant targeting moiety molecules may in some examples retain little or no native biological activity, but retain the ability to bind the appropriate target (e.g., bind to the appropriate cell surface receptor or protein) with high specificity.

In some embodiments, the cancer stem cells are from a cancer selected from the group consisting of glioblastoma, head and neck cancer, lung cancer, breast cancer, pancreatic cancer, cervical cancer, prostate cancer, and soft tissue sarcoma.

G. Linkers

Linking of a cargo moiety to a targeting moiety may be direct meaning that one portion of the cargo moiety is directly attached to a portion of the targeting moiety. For example, one end of the amino acid sequence of a cargo protein can be directly attached to an end of the amino acid sequence of the targeting moiety. For example, the C-terminus of the cargo protein can be linked to the N-terminus of the targeting moiety, or the C-terminus of the targeting moiety can be linked to the N-terminus of the cargo protein. Methods of generating such fusion proteins are routine in the art, for example using recombinant molecular biology methods.

In another example, the cargo moiety is linked to the targeting moiety indirectly through a linker. The linker can serve, for example, simply as a convenient way to link the two entities, as a means to spatially separate the two entities, to provide an additional functionality to the IL-4 targeted cargo protein, or a combination thereof.

In general, the linker joining the targeting moiety and the cargo moiety can be designed to (1) allow the two molecules to fold and act independently of each other, (2) not have a propensity for developing an ordered secondary structure which could interfere with the functional domains of the two moieties, (3) have minimal hydrophobic or charged characteristic which could interact with the functional protein domains and/or (4) provide steric separation of the two regions. For example, in some instances it may be desirable to spatially separate the targeting moiety and the cargo moiety to prevent the targeting moiety from interfering with the inhibitory activity of the targeted cargo moiety and/or the cargo moiety interfering with the targeting activity of the targeting moiety. The linker can also be used to provide, for example, lability to the connection between the targeting moiety and the cargo moiety, an enzyme cleavage site (for example a cleavage site for a protease), a stability sequence, a molecular tag, a detectable label, or various combinations thereof.

The linker can be bifunctional or polyfunctional, e.g. contains at least about a first reactive functionality at, or proximal to, a first end of the linker that is capable of bonding to, or being modified to bond to, the targeting moiety and a second reactive functionality at, or proximal to, the opposite end of the linker that is capable of bonding to, or being modified to bond to, the cargo moiety being modified. The two or more reactive functionalities can be the same (i.e. the linker is homobifunctional) or they can be different (i.e. the linker is heterobifunctional). A variety of bifunctional or polyfunctional cross-linking agents are known in the art that are suitable for use as linkers (for example, those commercially available from Pierce Chemical Co., Rockford, Ill.), such as avidin and biotin. Alternatively, these reagents can be used to add the linker to the targeting moiety and/or cargo moiety.

The length and composition of the linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, the linker should not significantly interfere with the ability of the targeting moiety to target the IL-4 targeted cargo protein to a cancer stem cell, or with the activity of the IL-4 targeted cargo protein relating to activation, pore-forming ability, or toxin activity.

Linkers suitable for use may be branched, unbranched, saturated, or unsaturated hydrocarbon chains, as well as peptides as noted above. Furthermore, if the linker is a peptide, the linker can be attached to the targeting moiety and/or the cargo moiety using recombinant DNA technology. Such methods are well-known in the art and details of this technology can be found, for example, in Sambrook et al., supra.

In one example, the linker is a branched or unbranched, saturated or unsaturated, hydrocarbon chain having from 1 to 100 carbon atoms, wherein one or more of the carbon atoms is optionally replaced by —O— or —NR— (wherein R is H, or C1 to C6 alkyl), and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (.dbd.O), carboxy, aryl, aryloxy, heteroaryl, and heteroaryloxy.

Examples of suitable linkers include, but are not limited to, peptides having a chain length of 1 to 500 amino acid residues (such as 1 to 100, 1 to 50, 6 to 30, such as less than 30 amino acids). Typically surface amino acids in flexible protein regions include Gly, Asn and Ser. Other neutral amino acids, such as Thr and Ala, can also be used in the linker sequence. Additional amino acids can be included in the linker to provide unique restriction sites in the linker sequence to facilitate construction of the fusions. Other exemplary linkers include those derived from groups such as ethanolamine, ethylene glycol, polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains.

In one example, the linker is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (.dbd.O), carboxy, aryl and aryloxy.

In a specific example, the linker is a peptide having a chain length of 1 to 50 amino acid residues, such as 1 to 40, 1 to 20, or 5 to 10 amino acid residues.

Peptide linkers that are susceptible to cleavage by enzymes of the complement system, urokinase, tissue plasminogen activator, trypsin, plasmin, or another enzyme having proteolytic activity may be used in one example. According to another example, the IL-4 targeted cargo protein includes a targeting moiety attached via a linker susceptible to cleavage by enzymes having a proteolytic activity such as a urokinase, a tissue plasminogen activator, plasmin, thrombin or trypsin. In addition, targeting moieties may be attached to the cargo moiety via disulfide bonds (for example, the disulfide bonds on a cysteine molecule). Since many tumors naturally release high levels of glutathione (a reducing agent) this can reduce the disulfide bonds with subsequent release of the cargo moiety at the site of delivery.

In one example, the IL-4 targeted cargo protein includes a targeting moiety linked by a cleavable linker region. In another example, the cleavable linker region is a protease-cleavable linker, although other linkers, cleavable for example by small molecules, may be used. Examples of protease cleavage sites are those cleaved by factor Xa, thrombin and collagenase. In one example, the protease cleavage site is one that is cleaved by a protease that is associated with a disease. In another example, the protease cleavage site is one that is cleaved by a protease that is up-regulated or associated with cancers in general. Examples of such proteases are uPA, the matrix metalloproteinase (MMP) family, the caspases, elastase, prostate specific antigen (PSA, a serine protease), and the plasminogen activator family, as well as fibroblast activation protein. In still another example, the cleavage site is cleaved by a protease secreted by cancer-associated cells. Examples of these proteases include matrixmetalloproteases, elastase, plasmin, thrombin, and uPA. In another example, the protease cleavage site is one that is up-regulated or associated with a specific cancer. The precise sequences are available in the art and the skilled person will have no difficulty in selecting a suitable cleavage site. By way of example, the protease cleavage region targeted by Factor Xa is I E G R. The protease cleavage region targeted by enterokinase is D D D D K. The protease cleavage region targeted by thrombin is L V P R G. In one example, the cleavable linker region is one which is targeted by endocellular proteases.

As known in the art, the attachment of a linker to cargo moiety (or of a linker element to a cleavable element, or a cleavable element to another cargo moiety) need not be a particular mode of attachment or reaction.

H. Exemplary Cargo Moiety/Targeting Moiety Combinations

1. MDNA55

MDNA55 has been developed for the treatment of recurrent/progressive glioblastoma (GB). Using current treatment paradigms, most GB patients experience tumor recurrence/progression after standard first line treatment. Treatment options for patients with recurrent GB are very limited and the outcome is generally unsatisfactory. Specifically, chemotherapy regimens for recurrent or progressive GB have been unsuccessful, producing toxicity without benefit (Weller et al., 2013). This is mainly due to the lack of tissue specificity with resultant toxicity to normal tissues and consequently, a narrow therapeutic index. As overall survival remains dismal, novel anti-cancer modalities, with greater tumor specificity, more robust cytotoxic mechanisms and novel delivery techniques are needed for the treatment of recurrent GB.

MDNA55 is a novel therapeutic that provides a targeted treatment approach whereby tumor cells are more sensitive to the toxic effects of the drug than normal cells. The target, IL-4R, is an ideal but under-exploited target for the development of cancer therapeutics, as it is frequently and intensely expressed on a wide variety of human carcinomas. Expression levels of IL-4R are low on the surface of healthy and normal cells, but increase several-fold on cancer cells. A majority of cancer biopsy and autopsy samples from adult and pediatric central nervous system (CNS) tumors, including recurrent GB biopsies, have been shown to over-express the IL-4R. There is little or no IL-4R expression in normal adult and pediatric brain tissue (Joshi, et al., 2001; Table 2 of the reference). This differential expression of the IL-4R provides MDNA55 a wide therapeutic window (see Table 4 of the reference for IC50 data). This feature alone makes MDNA55 an ideal candidate for the treatment of recurrent GB and other CNS tumors that over-express the IL-4R. Cells that do not express the IL-4R target do not bind to MDNA55 and are, therefore, not subject to PE-mediated effects.

2. Other Combinations

Any combination of cargo moiety and IL-4 based targeting moiety can be employed according to the present invention. In this section exemplary combinations of targeting moieties and cargo moieties are provided. In all examples that targeting moiety can be an antibody that specifically binds to a target, such as a fully humanized antibody.

IL-4 (including IL-4 circularly permuted ligands and other IL-4 receptor binding proteins such as IL-13) is another targeting moiety that can be linked to BCL-2 family proteins, such as BAX, BAD, BAT, BAK, BIK, BOK, BID BIM, BMF and BOK, or a toxin such as aerolysin, proaerolysin, Pseudomonas exotoxin, or combinations thereof. Any form or derivative of IL-4 can be used as the targeting moiety. For example, IL-4 or fragments of IL-4 that bind to the IL-4 receptor can be used. Additionally, multiple cargo moieties can be linked to IL-4 or multiple IL-4 proteins can be linked to cargo moieties.

Any form or derivative of IL-4 can be used as the targeting moiety. For example, IL-4 or fragments of IL-4 that bind to the IL-4 receptor can be used. Additionally, multiple cargo moieties can be linked to IL-4 or multiple IL-4 proteins can be linked to cargo moieties.

A circularly permuted ligand, for example a circularly permuted ligand derived from IL-4 can be employed as the targeting moiety. Pseudomonas exotoxin can be employed as the cargo moiety. Any form or derivative of circularly permuted IL-4 ligand can be used as the targeting moiety. Additionally, multiple cargo moieties can be linked to a circularly permuted ligand or multiple circularly permuted ligand proteins can be linked to cargo moieties.

I. Recombinant Expression of IL-4 Muteins, Expression Vectors and Host Cells

In various embodiments, polypeptides used in the practice of the instant invention are synthetic, or are produced by expression of a recombinant nucleic acid molecule. In the event the polypeptide is a chimera (e.g., a fusion protein containing at least a mutant IL-4 polypeptide and a heterologous polypeptide), it can be encoded by a hybrid nucleic acid molecule containing one sequence that encodes all or part of the IL-4 mutein, and a second sequence that encodes all or part of the heterologous polypeptide. For example, subject IL-4 muteins described herein may be fused to a hexa-histidine tag to facilitate purification of bacterially expressed protein, or to a hemagglutinin tag to facilitate purification of protein expressed in eukaryotic cells.

Methods for constructing a DNA sequence encoding the IL-4 muteins and expressing those sequences in a suitably transformed host include, but are not limited to, using a PCR-assisted mutagenesis technique. Mutations that consist of deletions or additions of amino acid residues to an IL-4 polypeptide can also be made with standard recombinant techniques. In the event of a deletion or addition, the nucleic acid molecule encoding IL-4 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

The complete amino acid sequence can be used to construct a back-translated gene. A DNA oligomer containing a nucleotide sequence coding for IL-4 mutein can be synthesized. For example, several small oligonucleotides coding for portions of the desired polypeptide can be synthesized and then ligated. The individual oligonucleotides typically contain 5′ or 3′ overhangs for complementary assembly.

In addition to generating mutant polypeptides via expression of nucleic acid molecules that have been altered by recombinant molecular biological techniques, subject IL-4 muteins can be chemically synthesized. Chemically synthesized polypeptides are routinely generated by those of skill in the art.

Once assembled (by synthesis, site-directed mutagenesis or another method), the DNA sequences encoding an IL-4 mutein will be inserted into an expression vector and operatively linked to an expression control sequence appropriate for expression of the IL-4 mutein in the desired transformed host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

The DNA sequence encoding the IL-4 mutein, whether prepared by site directed mutagenesis, chemical synthesis or other methods, can also include DNA sequences that encode a signal sequence. Such signal sequence, if present, should be one recognized by the cell chosen for expression of the IL-4 mutein. It can be prokaryotic, eukaryotic or a combination of the two. It can also be the signal sequence of native IL-4. The inclusion of a signal sequence depends on whether it is desired to secrete the IL-4 mutein from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild-type IL-4 signal sequence be used.

J. Nucleic Acid Molecules Encoding Mutant IL-4

In some embodiments the subject IL-4 mutein, either alone or as a part of a chimeric polypeptide, such as those described above, can be obtained by expression of a nucleic acid molecule. Just as IL-4 muteins can be described in terms of their identity with wild-type IL-4 polypeptides, the nucleic acid molecules encoding them will necessarily have a certain identity with those that encode wild-type IL-4. For example, the nucleic acid molecule encoding a subject IL-4 mutein can be at least 50%, at least 65%, preferably at least 75%, more preferably at least 85%, and most preferably at least 95% (e.g., 99%) identical to the nucleic acid encoding wild-type IL-4.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-4) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a mutant IL-4) is incorporated into a vector (e.g., a plasmid or viral vector) or into the genome of a heterologous cell (or the genome of a homologous cell, at a position other than the natural chromosomal location).

As described above, the subject IL-4 mutein may exist as a part of a chimeric polypeptide. In addition to, or in place of, the heterologous polypeptides described above, a subject nucleic acid molecule can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-hosphotransferase (HPH), thymidine kinase (TK), lacz (encoding β-galactosidase), and xanthine guanine phosphoribosyltransferase (XGPRT). One of skill in the art will be aware of additional useful reagents, for example, of additional sequences that can serve the function of a marker or reporter.

The subject nucleic acid molecules can be obtained by introducing a mutation into IL-4-encoding DNA obtained from any biological cell, such as the cell of a mammal. Thus, the subject nucleic acids (and the polypeptides they encode) can be those of a mouse, rat, guinea pig, cow, sheep, horse, pig, rabbit, monkey, baboon, dog, or cat. In one embodiment, the nucleic acid molecules will be those of a human.

### III. Making IL-4 Targeted Cargo Proteins

IL-4 targeted cargo proteins can be prepared by many routine methods as known in the art. IL-4 targeted cargo proteins, as well as modifications thereto, can be made, for example, by engineering the nucleic acid encoding the IL-4 targeted cargo protein using recombinant DNA technology or by peptide synthesis. Modifications to the IL-4 targeted cargo protein may be made, for example, by modifying the IL-4 targeted cargo protein polypeptide itself, using chemical modifications and/or limited proteolysis. Combinations of these methods may also be used to prepare the IL-4 targeted cargo proteins.

Methods of cloning and expressing proteins are well-known in the art, detailed descriptions of techniques and systems for the expression of recombinant proteins can be found, for example, in Current Protocols in Protein Science (Coligan, J. E., et al., Wiley & Sons, New York). Those skilled in the art will understand that a wide variety of expression systems can be used to provide the recombinant protein. Accordingly, the IL-4 targeted cargo proteins can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; or insect cells). The IL-4 targeted cargo proteins can be purified from the host cells by standard techniques known in the art.

Sequences for various exemplary cargo moieties and targeting moieties are provided in the Tables 1 and 2. Variants and homologs of these sequences can be cloned, if an alternative sequence is desired, using standard techniques [see, for example, Ausubel et al., Current Protocols in Molecular Biology, Wiley & Sons, NY (1997 and updates); Sambrook et al., supra]. For example, the nucleic acid sequence can be obtained directly from a suitable organism, such as Aeromonas hydrophila, by extracting mRNA and then synthesizing cDNA from the mRNA template (for example by RT-PCR) or by PCR-amplifying the gene from genomic DNA. Alternatively, the nucleic acid sequence encoding either the targeting moiety or the cargo moiety can be obtained from an appropriate cDNA library by standard procedures. The isolated cDNA is then inserted into a suitable vector, such as a cloning vector or an expression vector.

Mutations (if desired) can be introduced at specific, pre-selected locations by in vitro site-directed mutagenesis techniques well-known in the art. Mutations can be introduced by deletion, insertion, substitution, inversion, or a combination thereof, of one or more of the appropriate nucleotides making up the coding sequence.

The expression vector can further include regulatory elements, such as transcriptional elements, required for efficient transcription of the IL-4 targeted cargo protein-encoding sequences. Examples of regulatory elements that can be incorporated into the vector include, but are not limited to, promoters, enhancers, terminators, and polyadenylation signals. Vectors that include a regulatory element operatively linked to a nucleic acid sequence encoding a genetically engineered IL-4 targeted cargo protein can be used to produce the IL-4 targeted cargo protein.

The expression vector may additionally contain heterologous nucleic acid sequences that facilitate the purification of the expressed IL-4 targeted cargo protein, such as affinity tags such (e.g., metal-affinity tags, histidine tags, avidin/streptavidin encoding sequences, glutathione-S-transferase (GST) encoding sequences, and biotin encoding sequences). In one example, such tags are attached to the N- or C-terminus of a IL-4 targeted cargo protein, or can be located within the IL-4 targeted cargo protein. The tags can be removed from the expressed IL-4 targeted cargo protein prior to use according to methods known in the art. Alternatively, the tags can be retained on the IL-4 targeted cargo protein, providing that they do not interfere with the ability of the IL-4 targeted cargo protein to target and kill (or decrease growth of) cancer cells and/or cancer stem cells.

As an alternative to a directed approach to introducing mutations into naturally occurring pore-forming proteins, a cloned gene expressing a pore-forming protein can be subjected to random mutagenesis by techniques known in the art. Subsequent expression and screening of the mutant forms of the protein thus generated would allow the identification and isolation of targeted cargo moieties.

The IL-4 targeted cargo proteins can also be prepared as fragments or fusion proteins. A fusion protein is one which includes a IL-4 targeted cargo protein linked to other amino acid sequences that do not inhibit the ability of the IL-4 targeted cargo protein to selectively target and inhibit cancer stem cell growth or kill cancer cells and/or cancer stem cells. In an alternative example, the other amino acid sequences are short sequences of, for example, up to about 5, about 6, about 7, about 8, about 9, about 10, about 20, about 30, about 50 or about 100 amino acid residues in length. These short sequences can be linker sequences as described above.

Methods for making fusion proteins are well known to those skilled in the art. For example U.S. Pat. No. 6,057,133 discloses methods for making fusion molecules composed of human interleukin-3 (hIL-3) variant or mutant proteins functionally joined to a second colony stimulating factor, cytokine, lymphokine, interleukin, hematopoietic growth factor or IL-3 variant. U.S. Pat. No. 6,072,041 to Davis et al. discloses the generation of fusion proteins comprising a single chain Fv molecule directed against a transcytotic receptor covalently linked to a therapeutic protein.

The IL-4 targeted cargo protein can include one or more linkers, as well as other moieties, as desired. These can include a binding region, such as avidin or an epitope, or a tag such as a polyhistidine tag, which can be useful for purification and processing of the fusion protein. In addition, detectable markers can be attached to the fusion protein, so that the traffic of the fusion protein through a body or cell can be monitored conveniently. Such markers include radionuclides, enzymes, fluorophores, chromophores, and the like.

One of ordinary skill in the art will appreciate that the DNA can be altered in numerous ways without affecting the biological activity of the encoded protein. For example, PCR can be used to produce variations in the DNA sequence which encodes a IL-4 targeted cargo protein. Such variations in the DNA sequence encoding a IL-4 targeted cargo protein can be used to optimize for codon preference in a host cell used to express the protein, or may contain other sequence changes that facilitate expression.

A covalent linkage of a targeting moiety directly to a cargo moiety or via a linker may take various forms as is known in the art. For example, the covalent linkage may be in the form of a disulfide bond. The DNA encoding one of the components can be engineered to contain a unique cysteine codon. The second component can be derivatized with a sulfhydryl group reactive with the cysteine of the first component. Alternatively, a sulfhydryl group, either by itself or as part of a cysteine residue, can be introduced using solid phase polypeptide techniques. For example, the introduction of sulfhydryl groups into peptides is described by Hiskey (Peptides 3:137, 1981).

Proteins also can be chemically modified by standard techniques to add a sulfhydryl group. For example, Traut's reagent (2-iminothiolane-HCl) (Pierce Chemicals, Rockford, Ill.) can be used to introduce a sulfhydryl group on primary amines, such as lysine residues or N-terminal amines. A protein or peptide modified with Traut's reagent can then react with a protein or peptide which has been modified with reagents such as N-succinimidyl 3-(2-pyridyldithio) propionate (SPDP) or succinimidyl 4-(N-maleimidomethyl)cyclohexane-1-carboxylate (SMCC) (Pierce Chemicals, Rockford, Ill.).

The components can also be joined using the polymer, monomethoxy-polyethylene glycol (mPEG), as described in Maiti et al., Int. J. Cancer Suppl., 3:17-22, 1988.

The targeting moiety and the cargo moiety can also be conjugated through the use of standard conjugation chemistries as is known in the art, such as carbodiimide-mediated coupling (for example, DCC, EDC or activated EDC), and the use of 2-iminothiolane to convert epsilon amino groups to thiols for crosslinking and m-maleimidobenzoyl-n-hydroxysuccinimidyl ester (MBS) as a crosslinking agent.

I. Testing IL-4 Targeted Cargo Proteins

IL-4 targeted cargo proteins can be tested using standard techniques known in the art. Exemplary methods of testing candidate IL-4 targeted cargo proteins are provided below and in the examples included herein. One of ordinary skill in the art will understand that other methods of testing the IL-4 targeted cargo proteins are known in the art and are also suitable for testing candidate IL-4 targeted cargo proteins. For example, methods known in the art for testing for anti-tumor activity can be used. The IL-4 targeted cargo proteins can initially be screened against a panel of cancer cell lines or cancer stem cell lines. A cell proliferation assay, such as the WST-1 kit sold by Roche, can be used. Potency can be evaluated using different drug concentrations in the presence or absence of agents that inhibit cancer cells or sensitize cancer cells and/or cancer stem cells. Selected drug candidates from the initial cancer stem cell screen can be further characterized through additional in vitro assays and in relevant xenograft models to examine anti-tumor activity.

A. In Vitro

IL-4 targeted cargo proteins can be tested for their ability to kill cancer stem cells or significantly reduce or inhibit the growth of cancer cells and/or cancer stem cells using known methods. For example, the ability of the IL-4 targeted cargo proteins to kill or inhibit growth of cells can be assayed in vitro using suitable cells, typically a cell line expressing the target or a stem cancer cell. In general, cells of the selected test cell line are grown to an appropriate density and the candidate IL-4 targeted cargo protein is added. The IL-4 targeted cargo protein can be added to the culture at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL. In some examples, serial dilutions are tested. After an appropriate incubation time (for example, about 48 to 72 hours), cell survival or growth is assessed. Methods of determining cell survival are well known in the art and include, but are not limited to, the resazurin reduction test (see Fields & Lancaster Am. Biotechnol. Lab., 11:48-50, 1993; O'Brien et al., Eur. J. Biochem., 267:5421-5426, 2000 and U.S. Pat. No. 5,501,959), the sulforhodamine assay (Rubinstein et al., J. Natl. Cancer Inst., 82:113-118, 1999) or the neutral red dye test (Kitano et al., Euro. J. Clin. Investg., 21:53-58, 1991; West et al., J. Investigative Derm., 99:95-100, 1992) or trypan blue assay. Numerous commercially available kits may also be used, for example the CellTiter 96® AQueous One Solution Cell Proliferation Assay (Promega). Cytotoxicity is determined by comparison of cell survival in the treated culture with cell survival in one or more control cultures, for example, untreated cultures and/or cultures pre-treated with a control compound (typically a known therapeutic), or other appropriate control. IL-4 targeted cargo proteins considered to be effective in killing or reducing the growth of cancer cells and/or cancer stem cells are capable of decreasing cell survival or growth, for example, by at least about 10%, at least about 20%, at least about 30%, at least about 40%, or at least about 50%.

In some examples the IL-4 targeted cargo protein can be not significantly toxic to non-cancer cells and/or cancer stem cells. For example, the IL-4 targeted cargo protein when incubated at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL in cell culture with cells not displaying the target (e.g., does not express IL-4R) will kill less than about 50%, less than about 40%, less than about 30%, less than about 20%, or less than about 10% of the non-cancer cells and/or cancer stem cells. In some examples, the IL-4 targeted cargo protein when incubated at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL in cell culture with cells not displaying the target (e.g., does not express IL-4R) will have at least a 10-fold greater LD₅₀ toward the non-cancer cells and/or cancer stem cells, such as an at least 20-fold greater, at least 50-fold greater, or at least 100-fold greater LD.sub.50 toward the non-cancer cells and/or cancer stem cells.

In some examples IL-4 targeted cargo proteins include a toxin that contains one or more modifications to an activation sequence. These activatable IL-4 targeted cargo proteins can be tested for their ability to be cleaved by the appropriate activating agent according to methods known in the art. For example, if the one or more modifications result in the addition of one or more protease cleavage sites, the IL-4 targeted cargo protein can be incubated with varying concentrations of the appropriate protease(s). The incubation products can be electrophoresed on SDS-PAGE gels and cleavage of the IL-4 targeted cargo protein can be assessed by examining the size of the polypeptide on the gel.

In order to determine if the activatable IL-4 targeted cargo proteins that have been incubated with protease retain pore-forming activity, and thus the ability to kill cells, after incubation with the protease, the reaction products can be tested in a hemolysis assay as is known in the art. An example of a suitable assay is described in Howard and Buckley, J. Bacteriol., 163:336-40, 1985, which is herein incorporated by reference.

IL-4 targeted cargo proteins that confer selectivity for a specific type of cancer may be tested for their ability to target that specific cancer cell type. For example, a IL-4 targeted cargo protein comprising an IL-4 that targets cancer cells and/or cancer stem cells displaying IL-4R can be assessed for its ability to selectively target cancer cells and/or cancer stem cells by comparing the ability of the IL-4 targeted cargo protein to kill cancer cells and/or cancer stem cells to its ability to kill a normal cell, or a different type of cancer cell (e.g., one that does not express IL-4R). Alternatively, flow cytometric methods, as are known in the art, may be used to determine if a IL-4 targeted cargo protein comprising an IL-4 targeting moiety is able to selectively target a specific type of cancer stem cell. Binding of a labeled antibody to the bound IL-4 targeted cargo protein will indicate binding of the IL-4 targeted cargo protein to the target.

A variety of cancer cell-lines suitable for testing the candidate IL-4 targeted cargo proteins are known in the art and many are commercially available (for example, from the American Type Culture Collection, Manassas, Va.). In one example, in vitro testing of the candidate compounds is conducted in a human cancer cell-line. In another example, cancer cells and/or cancer stem cells are isolated and cultured as described in US Patent Application No. 2007/0292389 to Stassi et al. The cultured stem cells are used to test the activity of the IL-4 targeted cargo protein. Initial testing of the targeting moiety can be performed by linking the targeting moiety to a detectable label such as a fluorescent label and contacting a sample known to contain the appropriate cancer cells and/or cancer stem cells with the targeting moiety and observing the associated fluorescent label bound to the cancer stem cell.

Additional in vitro testing of IL-4 targeted cargo proteins can be accomplished using cell lines that have been engineered to express the desired target. An antibody specific for the target can be used to ensure that the target is being expressed. Upon binding to the cell expressing the target, the IL-4 targeted cargo protein may cause cell lysis which can be detected using methods known in the art.

B. In Vivo

The ability of the IL-4 targeted cargo proteins to kill tumor cells in vivo can be determined in an appropriate animal model using standard techniques known in the art (see, for example, Enna, et al., Current Protocols in Pharmacology, J. Wiley & Sons, Inc., New York, N.Y.).

Current animal models for screening anti-tumor compounds include xenograft models, in which a human tumor has been implanted into an animal. Using these techniques cancer cells and/or cancer stem cells can be transplanted and the presence, size and morphology of the resulting tumor can be assessed. Examples of xenograft models of human cancer include, but are not limited to, human solid tumor xenografts, implanted by sub-cutaneous injection or implantation and used in tumor growth assays; human solid tumor isografts, implanted by fat pad injection and used in tumor growth assays; human solid tumor orthotopic xenografts, implanted directly into the relevant tissue and used in tumor growth assays; experimental models of lymphoma and leukemia in mice, used in survival assays, and experimental models of lung metastasis in mice. In addition to the implanted human tumor cells, the xenograft models can further comprise transplanted human peripheral blood leukocytes, which allow for evaluation of the anti-cancer immune response.

Alternatively, murine cancer models can be used for screening anti-tumor compounds. Examples of appropriate murine cancer models are known in the art and include, but are not limited to, implantation models in which murine cancer cells are implanted by intravenous, subcutaneous, fat pad or orthotopic injection; murine metastasis models; transgenic mouse models; and knockout mouse models.

For example, the IL-4 targeted cargo proteins can be tested in vivo on solid tumors using mice that are subcutaneously grafted bilaterally with 30 to 60 mg of a tumor fragment, or implanted with an appropriate number of cancer cells and/or cancer stem cells (e.g., at least 10.sup.3, at least 10.sup.4, or at least at least 10.sup.6 cancer cells and/or cancer stem cells, such as from about 10 to about 10.sup.5, from about 50 to about 10.sup.4, or from about 75 to about 10.sup.3), on day 0. The animals bearing tumors are randomized before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The IL-4 targeted cargo proteins can be administered to the animals, for example, by i.p. injection, intravenous injection, direct injection into the tumor (or into the organ having the tumor), or bolus infusion. The amount of IL-4 targeted cargo protein that is injected can be determined using the in vitro testing results described above. For example, at least about 1 ng/kg body weight, at least 1 μg/kg body weight, or at least 1 mg/kg body weight, such as from about 0.01 μg/kg body weight to about 1 mg/kg body weight, from about 0.10 μg/kg body weight to about 1.0 g/kg body weight, from about 1 mg/kg body weight to about 4 mg/kg body weight. The different animal groups are weighed about 3 or 4 times a week until the maximum weight loss is attained, after which the groups are weighed at least about once a week until the end of the trial.

The tumors are measured after a pre-determined time period, or they can be monitored continuously by measuring about 2 or 3 times a week until the tumor reaches a pre-determined size and/or weight, or until the animal dies if this occurs before the tumor reaches the pre-determined size/weight. The animals are then sacrificed and the tissue histology, size and/or proliferation of the tumor assessed. Orthotopic xenograft models are an alternative to subcutaneous models and may more accurately reflect the cancer development process. In this model, tumor cells are implanted at the site of the organ of origin and develop internally. Daily evaluation of the size of the tumors is thus more difficult than in a subcutaneous model. A recently developed technique using green fluorescent protein (GFP) expressing tumors in non-invasive whole-body imaging can help to address this issue (Yang et al., Proc. Nat. Aca. Sci., 1206-1211, 2000). This technique utilizes human or murine tumors that stably express very high levels of green fluorescent protein (GFP). The GFP expressing tumors can be visualized by means of externally placed video detectors, allowing for monitoring of details of tumor growth, angiogenesis and metastatic spread. Angiogenesis can be measured over time by monitoring the blood vessel density within the tumor(s). The use of this model thus allows for simultaneous monitoring of several features associated with tumor progression and has high preclinical and clinical relevance.

For the study of the effect of the compositions on leukemias, the animals are grafted with a particular number of cells, and the anti-tumor activity is determined by the increase in the survival time of the treated mice relative to the controls.

To study the effect of a particular IL-4 targeted cargo protein on tumor metastasis, tumor cells are typically treated with the composition ex vivo and then injected into a suitable test animal. The spread of the tumor cells from the site of injection is then monitored over a suitable period of time.

IL-4 targeted cargo proteins that are sufficiently effective at inhibiting cancer stem cell growth (as evidenced by in vitro cell survival assays, metastasis inhibition assays, and/or xenograph model systems) can be chosen for use in humans. IL-4 targeted cargo proteins can also be chosen for trial and eventual therapeutic use in humans based upon their relative toxicity at the potential therapeutic dosage range indicated by the assays. Therapeutic dosages and toxicity are further described below.

II. Formulations/Compositions

Pharmaceutical compositions can include one or more IL-4 targeted cargo proteins and one or more non-toxic pharmaceutically acceptable carriers, diluents, excipients and/or adjuvants. If desired, other active ingredients may be included in the compositions. As indicated above, such compositions are suitable for use in the treatment of cancer. The term “pharmaceutically acceptable carrier” refers to a carrier medium which does not interfere with the effectiveness of the biological activity of the active ingredients and which is not toxic to the host or patient. Representative examples are provided below.

The pharmaceutical compositions may comprise, for example, from about 1% to about 95% of a IL-4 targeted cargo protein. Compositions formulated for administration in a single dose form may comprise, for example, about 20% to about 90% of the IL-4 targeted cargo proteins, whereas compositions that are not in a single dose form may comprise, for example, from about 5% to about 20% of the IL-4 targeted cargo proteins. Concentration of the IL-4 targeted cargo protein in the final formulation can be at least 1 ng/mL, such as at least 1 μg/mL or at least 1 mg/mL. For example, the concentration in the final formulation can be between about 0.01 μg/mL and about 1,000 μg/mL. In one example, the concentration in the final formulation is between about 0.01 mg/mL and about 100 mg/mL.

The composition can be a liquid solution, suspension, emulsion, sustained release formulation, or powder. The composition can be formulated as a suppository, with traditional binders and carriers such as triglycerides.

The IL-4 targeted cargo proteins can be delivered along with a pharmaceutically acceptable vehicle. In one example, the vehicle may enhance the stability and/or delivery properties. Thus, the disclosure also provides for formulation of the IL-4 targeted cargo protein with a suitable vehicle, such as an artificial membrane vesicle (including a liposome, noisome, nanosome and the like), microparticle or microcapsule, or as a colloidal formulation that comprises a pharmaceutically acceptable polymer. The use of such vehicles/polymers may be beneficial in achieving sustained release of the IL-4 targeted cargo proteins. Alternatively, or in addition, the IL-4 targeted cargo protein formulations can include additives to stabilize the protein in vivo, such as human serum albumin, or other stabilizers for protein therapeutics known in the art. IL-4 targeted cargo protein formulations can also include one or more viscosity enhancing agents which act to prevent backflow of the formulation when it is administered, for example by injection or via catheter. Such viscosity enhancing agents include, but are not limited to, biocompatible glycols and sucrose.

Pharmaceutical compositions formulated as aqueous suspensions contain the active compound(s) in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-.beta.-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, or one or more coloring agents.

Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above.

Pharmaceutical compositions can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

The pharmaceutical compositions containing one or more IL-4 targeted cargo proteins can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

In one example, the IL-4 targeted cargo protein is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. patent Pub. No. 20050106148 and references cited therein. In one example the polymer is a pH-sensitive polymers designed to enhance the release of drugs from the acidic endosomal compartment to the cytoplasm (see for example, Henry et al., Biomacromolecules 7(8):2407-14, 2006).

IL-4 targeted cargo proteins can also be administered in therapeutically effective amounts together with one or more anti-cancer therapeutics. The compound(s) can be administered before, during or after treatment with the anti-cancer therapeutic.

An “anti-cancer therapeutic” is a compound, composition, or treatment (e.g., surgery) that prevents or delays the growth and/or metastasis of cancer cells. Such anti-cancer therapeutics include, but are not limited to, surgery (e.g., removal of all or part of a tumor), chemotherapeutic drug treatment, radiation, gene therapy, hormonal manipulation, immunotherapy (e.g., therapeutic antibodies and cancer vaccines) and antisense or RNAi oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C, Avastin, Herceptin®, flurouracil, and temozolamide and the like. The compounds are also suitable for use with standard combination therapies employing two or more chemotherapeutic agents. It is to be understood that anti-cancer therapeutics includes novel compounds or treatments developed in the future.

The pharmaceutical compositions described above include one or more IL-4 targeted cargo proteins in an amount effective to achieve the intended purpose. Thus the term “therapeutically effective dose” refers to the amount of the IL-4 targeted cargo protein that ameliorates the symptoms of cancer. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans, using standard methods known in those of ordinary skill in the art.

Therapeutic efficacy and toxicity can also be determined by standard pharmaceutical procedures such as, for example, by determination of the median effective dose, or ED.sub.50 (i.e. the dose therapeutically effective in 50% of the population) and the median lethal dose, or LD.sub.50 (i.e. the dose lethal to 50% of the population). The dose ratio between therapeutic and toxic effects is known as the “therapeutic index,” which can be expressed as the ratio, LD.sub.50/ED.sub.50. The data obtained from cell culture assays and animal studies can be used to formulate a range of dosage for human or animal use. The dosage contained in such compositions is usually within a range of concentrations that include the ED.sub.50 and demonstrate little or no toxicity. The dosage varies within this range depending upon the dosage form employed, sensitivity of the subject, and the route of administration and the like. Exemplary dosage ranges that can be used include at least 1 ng/g tumor, at least 1 μg/g tumor, or at least 1 mg/g tumor, such as dosage ranges from about 0.01 μg/g tumor to about 50 μg/g tumor, from about 0.02 p/g tumor to about 40 μg/g tumor, from about 0.02 μg/g tumor to about 35 μg/g tumor, 0.03 μg/g tumor to about 25 μg/g tumor, from about 0.04 μg/g tumor to about 20 μg/g tumor, from about 0.04 μg/g tumor to about 10 μg/g tumor, and from about 0.5 μg/g tumor to about 2 μg/g tumor.

One of ordinary skill in the art will appreciate that the dosage will depend, among other things, upon the type of IL-4 targeted cargo protein being used and the type of cancer stem cell being treated.

In some embodiments, the IL-4 targeted cargo protein is MDNA55 of SEQ ID NO:65:

MDTTEKETFCRAATVLRQFYSHHEKDTRCLGATAQQFHRHKQLIRF
LKLRDRNLWGLAGLNSCPVKEANQSTLENFLERLKTIMREKYSKCS
SGGNGGHKCDITLQEIIKTLNSLTEQKTLCTELTVTDIFAASKASG
GPEGGSLAALTAHQACHLPLETFTRHRQPRGWEQLEQCGYPVQRLV
ALYLAARLSWNQVDQVIRNALASPGSGGDLGEAIREQPEQARLALT
LAAAESERFVRQGTGNDEAGAANGPADSGDALLERNYPTGAEFLGD
GGDVSFSTRGTQNWTVERLLQAHRQLEERGYVFVGYHGTFLEAAQS
IVFGGVRARSQDLDAIWRGFYIAGDPALAYGYAQDQEPDARGRIRN
GALLRVYVPRSSLPGFYRTSLTLAAPEAAGEVERLIGHPLPLRLDA
ITGPEEEGGRLETILGWPLAERTVVIPSAIPTDPRNVGGDLDPSSI
PDKEQAISALPDYASQPGKPPKDEL

MDNA55 has also been described in US Patent Publication NO. 2016/0271231, incorporated by reference herein in its entirety for all purposes.

In some embodiments, the IL-4 targeted cargo protein is diluted in artificial CSF. In some embodiments, the MDNA55 is diluted in an artificial cerebral spinal fluid (artificial CSF). In some embodiments, the artificial CSF comprises calcium chloride, dextrose, magnesium sulfate, potassium chloride, sodium bicarbonate, sodium chloride, sodium phosphate, dibasic, and is diluted in water. In some embodiments, the artificial CSF is Elliotts B® solution. In some embodiments, the artificial CSF is employed to produce an infusate having a final composition of MDNA55 at 3 μg/mL. In some embodiments, the artificial CSF is employed to produce an infusate having a final composition of MDNA55 at 3 μg/mL. In some embodiments, the artificial CSF is employed to produce an infusate having a final composition of MDNA55 at 3 μg/mL, 0.02% human serum albumin and gadolinium-diethylenetriamine pentaacetic acid (Gd-DTPA, Magnevist®) at 7 mM.

In some embodiments, the formulation and routes of administration described herein allow for about 80%, about 85%, about 90%, about 95%, or about 100% of the tumor and the 1 cm margin around it (at risk for tumor spread) to be successfully covered. In some embodiments, the formulation and routes of administration described herein allow for about 80% to about 100% of the tumor and the 1 cm margin around it (at risk for tumor spread) to be successfully covered. In some embodiments, the formulation and routes of administration described herein allow for about 85% to about 100% of the tumor and the 1 cm margin around it (at risk for tumor spread) to be successfully covered. In some embodiments, the formulation and routes of administration described herein allow for about 90% to about 100% of the tumor and the 1 cm margin around it (at risk for tumor spread) to be successfully covered. In some embodiments, the formulation and routes of administration described herein allow for about 95% to about 100% of the tumor and the 1 cm margin around it (at risk for tumor spread) to be successfully covered. In some embodiments, the formulation and routes of administration described herein allow for about 100% of the tumor and the 1 cm margin around it (at risk for tumor spread) to be successfully covered.

TABLE 7
Reagents used in the Preparation of Infusate

| | | | Manufacturer/ |
|---|---|---|---|
| Reagent | Type | Grade | Distributor |
| MDNA55 | Drug Product | CGMP, sterile | Medicenna |
| | | | Therapeutics Inc. |
| Elliotts B ® | Excipient | USP, sterile | Lukare Medical, |
| Solution | | | LLC |
| HSA 5% | Excipient | USP, sterile | Octapharma |
| (aqueous) | | | |
| Solution | | | |
| Gd-DTPA, | Excipient | USP, sterile | Bayer Healthcare |
| Magnevist ® | | | Pharmaceuticals Inc. |
| 469.1 | | | |

Abbreviations:
CGMP, Current Good Manufacturing Practice;
NDC, National Drug Code;
USP, United States Pharmacopeia

A. MDNA55 Formulation Embodiment

Composition of MDNA55: Drug product is supplied as a sterile frozen solution of MDNA55 at a concentration of 500 μg/mL contained in 0.5 mL Phosphate Buffered Saline (10 mM sodium phosphate, 500 mM sodium chloride, pH 7.4±0.1), filled in a sterile, single-use, 2 mL Type 1 USP dehydrogenated clear glass vial sealed with 13 mm Teflon-faced stopper and labeled as shown below:

MDNA55 Vial: PRX-321 contains 0.5 mL of MDNA55 (500 μg/m) and should be stored at ≤−70° C. The vial is labeled with “Sterile Single Dose Vials for Intratumoral Administration via Stereotactically Placed Catheters”.

Storage: Drug product is stored at −70° C.+/−10° C. in its secondary packaging until required for preparation of infusate. Hospital pharmacy temperature monitoring records must be provided for all periods in which drug product vial(s) are stored for review by the study monitor.

Handling: Infusate will be prepared, using aseptic technique using a pre-sanitized biological safety (vertical flow) cabinet. After the preparation of the infusate, the used drug product vial should be discarded according to the hospital pharmacy's standard operating procedure. Excipients

Upon receipt of shipment, the shipping container will be opened by the hospital Pharmacist who must inspect condition of the contents and ensure that the excipient kits are undamaged. The pharmacist must follow the instructions that will be included in the shipment for downloading the temp tale monitor data as well as complete/return the proof of receipt documentation that arrives with the shipment whereby condition of receipt will be documented. The hospital pharmacist must record inventory of the shipment using the Excipient Kit Inventory Form (Appendix 3). In the event that there is an issue identified during receipt of a excipient kit shipment, the hospital pharmacy should notify the contacts specified in Section 3.0 of this manual immediately.

In some embodiments, the IL-4 targeted cargo protein is provided as a kit. In some embodiments, the MDNA55 is provided as a kit. In some embodiments, the kit contains 4 components:
Human Serum Albumin (HSA)
Elliotts B Solution
Magnevist (Gd-DTPA)
Empty IV Bag

The container has a tamper seal at the opening end to secure closure. One Excipient Kit is to be used for one infusate preparation.

Excipient Kit components:
1×250 mL bottle HSA 5% (aqueous) Solution
1× unit Elliotts B Solution (10×10 mL ampules)
1×5 mL vial of Gd-DTPA
1× empty (150 mL size) IV Bag

The excipient kit components are to be used in MDNA55 infusate preparation as described in the present example. The kit provides materials for single (1×) MDNA55 infusate preparation.

Storage: Excipient kit is stored at controlled room temperature until required for preparation of infusate.

Handling: Excipient kit should be handled with care and stored right side up (label of kit in at the top).

### Human Serum Albumin

In some embodiments, Human Serum Albumin (HSA) is added to the infusate, at a final concentration of 0.02%, to prevent adsorption of MDNA55 to the inner surfaces of the syringes, tubes and catheter used in the infusion assembly.

Supply: 1×250 mL bottle (Octapharma HSA 5% (aqueous) Solution, NCT #68982-0623-02)

Storage: at controlled room temperature as recommended by the manufacturer.

Handling: HSA should be handled using aseptic techniques in a pre-sanitized biological safety cabinet. Once opened and or used, the remaining HSA should be discarded according to the hospital pharmacy's standard operating procedure.

### Buffered Intrathecal Electrolyte/Dextrose Injection (Elliotts B® Solution)

MDNA55 drug product is diluted in Elliotts B® Solution.

TABLE 8
Composition/Information on Ingredients:

| Specific Chemical | | | Quantity | |
|---|---|---|---|---|
| Identity | CAS # | Chemical Formula | per mL | |
| Calcium Chloride | 10035-04-8 | CaC1₂ | 0.2 | mg |
| Dextrose | 50-99-7 | C₆H₁₂O₆ | 0.8 | mg |
| Magnesium Sulfate | 10034-99-8 | MgSO₄ 7 H₂O | 0.3 | mg |
| Potassium Chloride | 7447-40-7 | KCl | 0.3 | mg |
| Sodium Bicarbonate | 144-55-8 | NaHCO₃ | 1.9 | mg |
| Sodium Chloride | 7647-14-5 | NaCl | 7.3 | mg |
| Sodium Phosphate, | 7782-85-6 | Na₂HPO₄ 7H₂O | 0.2 | mg |
| Dibasic | | | | |
| Water for Injection | 7732-18-5 | H₂O | 1 | mL |

Further information on the Elliott's B Solution. Elliotts B® Solution is a sterile, nonpyrogenic, isotonic solution containing no bacteriostatic preservatives. Elliotts B Solution is a diluent for intrathecal administration of methotrexate sodium and cytarabine. Each 10 mL of Elliotts B Solution contains:

TABLE 9
Composition per 10 mL

| Specific Chemical Identity | Quantity per 10 mL | |
|---|---|---|
| Sodium Chloride, USP | 73 | mg |
| Sodium Bicarbonate, USP | 19 | mg |
| Dextrose, USP | 8 | mg |
| Magnesium Sulfate•7H2O, USP | 3 | mg |
| Potassium Chloride, USP | 3 | mg |
| Calcium Chloride•2H2O, USP | 2 | mg |
| Sodium Phosphate, dibasic•7H2O, USP | 2 | mg |

| | |
|---|---|
| Water for Injection, USP qs 10 mL | To 10 mL |

TABLE 10
Concentration of Electrolytes:

| | | | | | |
|---|---|---|---|---|---|
| Sodium | 149 | mEq/liter | Bicarbonate | 22.6 | mEq/liter |
| Potassium | 4.0 | mEq/liter | Chloride | 132 | mEq/liter |
| Calcium | 2.7 | mEq/liter | Sulfate | 2.4 | mEq/liter |
| Magnesium | 2.4 | mEq/liter | Phosphate | 1.5 | mEq/liter |

TABLE 11
formulae and molecular weights of the ingredients:

| | MOLECULAR | MOLECULAR |
|---|---|---|
| INGREDIENT | FORMULA | WEIGHT |
| Sodium Chloride | NaCl | 58.44 |
| Sodium Bicarbonate | NaHCO3 | 84.01 |
| Dextrose | C6H12O6 | 180.16 |
| Magnesium Sulfate•7H2O | Mg2SO4•7H2O | 246.48 |
| Potassium Chloride | KCl | 74.55 |
| Calcium Chloride•2H2O | CaCl2•2H2O | 147.01 |
| Sodium Phosphate, dibasic•7H2O | Na2HPO4• [symbol] | 268.07 |

[symbol]

indicates data missing or illegible when filed

The pH of Elliotts B Solution is 6.0-7.5, and the osmolarity is 288 mOsmol per liter (calculated).

Elliotts B Solution provides a buffered salt solution for use as a diluent for the intrathecal administration of methotrexate sodium and cytarabine. It has been demonstrated that Elliotts B Solution is comparable to cerebrospinal fluid in pH, electrolyte composition, glucose content, and osmolarity:

TABLE 12
Comparison of Electrolyte Composition, pH and Nonelectrolytic
Constituents of Elliotts B Solution and CSF:

| | Na+ | K+ | Co++ | Mg++ | HCO3− | Cl− | | Phosphorus | Glucose |
|---|---|---|---|---|---|---|---|---|---|
| Solution | mEq/L | mEq/L | mEq/L | mEq/L | mEq7L | mEq/L | pH | mg/dL | mg/dL |
| Cerebrospinal | 117-137 | 2.3-4.6 | 2.2 | 2.2 | 22.9 | 113-127 | 7.31 | 1.2-2.1 | 45-80 |
| Fluid | | | | | | | | | |
| Elliotts B | 149 | 4.0 | 2.7 | 2.4 | 22.6 | 132 | 6.0-7.5 | 2.3 | 80 |
| Solution | | | | | | | | | |

The approximate buffer capacity of Elliotts B Solution is 1.1×10⁻² equivalents when the challenge solution is 0.01 N HCl and 7.8×10-3 equivalents when the challenge solution is 0.01 N NaOH. Compatibility studies with methotrexate sodium and cytarabine indicate these drugs are physically compatible with Elliotts B Solution.

Elliott's B solution is a diluent used in the preparation of infusate; it is comparable to cerebrospinal fluid in pH, electrolyte composition, glucose content, osmolarity and buffering capacity.

### Gadolinium-Diethylenetriamine Pentaacetic Acid (Gd-DTPA) Magnevist®

In some embodiments, Gd-DTPA (diluted to ˜1:70) is added to the infusate as a contrast agent as co-infusion of this surrogate tracer during infusion allows real-time monitoring of MDNA55 infusate distribution.

Supply: 1×5 mL single use vial of Gd-DTPA (Bayer HealthCare Pharmaceuticals Inc. Magnevist®; 469.1 mg/mL, NDC #50419-188-05).

Storage: stored according to manufacturer's instructions.

Handling: Gd-DTPA (Magnevist®) should be handled using aseptic techniques in a pre-sanitized biological safety cabinet. Once opened or used, the remaining should be discarded in accordance with regulations dealing with the disposal of such materials and according to the hospital pharmacy's standard operating procedure.

B. Pharmaceutical Compositions and Methods of Administration

In some embodiments, subject IL-4 muteins and nucleic acids can be incorporated into compositions, including pharmaceutical compositions. Such compositions typically include the polypeptide or nucleic acid molecule and a pharmaceutically acceptable carrier. Such compositions can also comprise anti-PD-1 antibodies. In some embodiments, the composition comprises an IL-4 mutein that is a fusion protein and/or is associated with a CAR-T construct and/or expressed by or associated with an oncolytic virus.

The anti-PD-1 antibodies and IL-4 muteins can be administered as a co-composition, simultaneously as two separate compositions, and/or sequentially as two separate compositions. In some embodiments, the anti-PD-1 antibody or inhibitor and IL-4 mutein are administered together as a single co-composition (i.e., co-formulated). In some embodiments, the anti-PD-1 antibody or inhibitor and IL-4 mutein are administered simultaneously as two separate compositions (i.e., separate formulations). In some embodiments, the anti-PD-1 antibody or inhibitor and IL-4 mutein are administered sequentially as separate compositions (i.e., separate formulations). In some embodiments, when the anti-PD-1 antibody or inhibitor and IL-4 mutein are administered sequentially as separate compositions, the anti-PD-1 antibody or inhibitor is administered before the IL-4 mutein. In some embodiments, when the anti-PD-1 antibody or inhibitor and IL-4 mutein are administered sequentially as separate compositions, the IL-4 mutein is administered before the anti-PD-1 antibody or inhibitor. In some embodiments, the anti-PD-1 antibodies include but are not limited to nivolumab, BMS-936558, MDX-1106, ONO-4538, AMP224, CT-011, and MK-3475.

The other immunotherapy agents as described and IL-4 muteins can be administered as a co-composition, simultaneously as two separate compositions, and/or sequentially as two separate compositions. In some embodiments, the other immunotherapy agents and IL-4 mutein are administered together as a single co-composition (i.e., co-formulated). In some embodiments, the other immunotherapy agents and IL-4 mutein are administered simultaneously as two separate compositions (i.e., separate formulations). In some embodiments, the other immunotherapy agents and IL-4 mutein are administered sequentially as separate compositions (i.e., separate formulations). In some embodiments, when the other immunotherapy agents and IL-4 mutein are administered sequentially as separate compositions, the anti-PD-1 antibody or inhibitor is administered before the IL-4 mutein. In some embodiments, when other immunotherapy agents and IL-4 mutein are administered sequentially as separate compositions, the IL-4 mutein is administered before other immunotherapy agents.

A pharmaceutical composition is formulated to be compatible with its intended route of administration. The anti-PD-1 antibodies and/or mutant IL-4 polypeptides of the invention may be given orally, but it is more likely that they will be administered through a parenteral route, including for example intravenous administration. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, anti-PD-1 antibodies and/or IL-4 muteins, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the anti-PD-1 antibodies and/or IL-4 muteins or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, compounds (anti-PD-1 antibodies and/or mutant IL-4 polypeptides or nucleic acids) can also be prepared in the form of suppositories (e.g., with conventional suppository bases such as cocoa butter and other glycerides) or retention enemas for rectal delivery.

In some embodiments, compounds (subject IL-4 muteins or nucleic acids) can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In one embodiment, the anti-PD-1 antibodies and/or IL-4 muteins or nucleic acids are prepared with carriers that will protect the anti-PD-1 antibodies and/or mutant IL-4 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such anti-PD-1 antibodies, IL-4 muteins, or nucleic acids compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a subject IL-4 mutein (i.e., an effective dosage) and/or the anti-PD-1 antibody or inhibitor depends on the polypeptide or antibody selected. In some embodiments, single dose amounts of the IL-4 mutein can be in the range of approximately 0.001 mg/kg to 0.1 mg/kg of patient body weight can be administered. In some embodiments, single dose amounts of the anti-PD-1 antibody or inhibitor can be in the range of approximately 1 mg/kg to 20 mg/kg, or about 5 mg/kg to about 15 mg/kg, or about 10 mg/kg of patient body weight can be administered. In some embodiments, doses of the anti-PD-1 antibody or inhibitor and/or the IL-4 mutein of about 0.005 mg/kg, 0.01 mg/kg, 0.025 mg/kg, 0.05 mg/kg, 0.1 mg/kg, 0.25 mg/kg, 0.5 mg/kg, 1.0 mg/kg, 5.0 mg/kg, 10.0 mg/kg may be administered. In some embodiments, 600,000 IU/kg is administered (IU can be determined by a lymphocyte proliferation bioassay and is expressed in International Units (IU) as established by the World Health Organization 1st International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject IL-4 muteins can include a single treatment or, can include a series of treatments. In one embodiment, the compositions are administered every 8 hours for five days, followed by a rest period of 2 to 14 days, e.g., 9 days, followed by an additional five days of administration every 8 hours. In some embodiments, administration is 3 doses administered every 4 days.

The pharmaceutical compositions can be included in a container, pack, or dispenser together with instructions for administration.

The following examples are provided to describe certain embodiments of the invention provided herein and are not to be construed to as limiting.

C. Administration and Dosing

The IL-4 targeted cargo proteins can be used to treat, stabilize or prevent CNS cancer, including for example the IL-4 targeted cargo protein MDNA55. IL-4 targeted cargo proteins can also be used in the treatment of indolent cancers, recurrent cancers including locally recurrent, distantly recurrent and/or refractory cancers (i.e. cancers that have not responded to other anti-cancer treatments), metastatic cancers, locally advanced cancers and aggressive cancers. In these contexts, the IL-4 targeted cargo proteins may exert either a cytotoxic or cytostatic effect resulting in, for example, a reduction in the number or growth of cancer cells and/or cancer stem cells, a reduction in the size of a tumor, the slowing or prevention of an increase in the size of a tumor, an increase in the disease-free survival time between the disappearance or removal of a tumor and its reappearance, prevention of an initial or subsequent occurrence of a tumor (e.g. metastasis), an increase in the time to progression, reduction of one or more adverse symptoms associated with a tumor, or an increase in the overall survival time of a subject having cancer.

Typically, in the treatment of cancer, IL-4 targeted cargo proteins are administered systemically to patients, for example, by bolus injection or continuous infusion into a patient's bloodstream. Alternatively, the IL-4 targeted cargo proteins may be administered locally, at the site of a tumor (intratumorally). When a IL-4 targeted cargo protein is administered intratumorally, the administration can be via any route, e.g., locally, regionally, focally, systemic, convection enhanced delivery or combinations thereof.

When used in conjunction with one or more known chemotherapeutic agents, the compounds can be administered prior to, or after, administration of the chemotherapeutic agents, or they can be administered concomitantly. The one or more chemotherapeutics may be administered systemically, for example, by bolus injection or continuous infusion, or they may be administered orally.

For administration to an animal, the pharmaceutical compositions can be formulated for administration by a variety of routes. For example, the compositions can be formulated for topical, rectal or parenteral administration or for administration by inhalation or spray. The term parenteral as used herein includes subcutaneous injections, intravenous, intramuscular, intrathecal, intrasternal injection or infusion techniques. Direct injection or infusion into a tumor is also contemplated. Convection enhanced delivery can also be used to administer the IL-4 targeted cargo protein.

In one example, the IL-4 targeted cargo protein can be injected into a subject having cancer, using an administration approach similar to the multiple injection approach of brachytherapy. For example, multiple aliquots of the purified IL-4 targeted cargo protein in the form of a pharmaceutical composition or formulation and in the appropriate dosage units, may be injected using a needle. Alternative methods of administration of the IL-4 targeted cargo proteins will be evident to one of ordinary skill in the art. Such methods include, for example, the use of catheters, or implantable pumps to provide continuous infusion of the IL-4 targeted cargo protein to the subject in need of therapy.

As is known in the art, software planning programs can be used in combination with brachytherapy treatment and ultrasound, for example, for placement of catheters for infusing IL-4 targeted cargo proteins to treat, for example, brain tumors or other localized tumors. For example, the positioning and placement of the needle can generally be achieved under ultrasound guidance. The total volume, and therefore the number of injections and deposits administered to a patient, can be adjusted, for example, according to the volume or area of the organ to be treated. An example of a suitable software planning program is the brachytherapy treatment planning program Variseed 7.1 (Varian Medical Systems, Palo Alto, Calif.). Such approaches have been successfully implemented in the treatment of prostate cancer among others.

If necessary to reduce a systemic immune response to the IL-4 targeted cargo proteins, immunosuppressive therapies can be administered in combination with the IL-4 targeted cargo proteins. Examples of immunosuppressive therapies include, but are not limited to, systemic or topical corticosteroids (Suga et al., Ann. Thorac. Surg., 73:1092-7, 2002), cyclosporin A (Fang et al., Hum. Gene Ther., 6:1039-44, 1995), cyclophosphamide (Smith et al., Gene Ther., 3:496-502, 1996), deoxyspergualin (Kaplan et al., Hum. Gene Ther., 8:1095-1104, 1997) and antibodies to T and/or B cells [e.g. anti-CD40 ligand, anti CD4 antibodies, anti-CD20 antibody (Rituximab)](Manning et al., Hum. Gene Ther., 9:477-85, 1998). Such agents can be administered before, during, or subsequent to administration of the IL-4 targeted cargo proteins. Such agents can be administered from about 10 mg/week to about 1000 mg/week, from about 40 mg/week to about 700 mg/week, or from about 200 mg/week to about 500 mg/week for 2, 3, 4, 5, 6, or 7 weeks. Courses of treatment can be repeated as necessary if the subject remains responsive (e.g., the symptoms of cancer are static or decreasing).

The IL-4 targeted cargo protein can also be administered in combination with a sensitizing agent, such as a radio-sensitizers (see for example Diehn et al., J. Natl. Cancer Inst. 98:1755-7, 2006). Generally, a sensitizing agent is any agent that increases the activity of a IL-4 targeted cargo protein. For example, a sensitizing agent will increase the ability of a IL-4 targeted cargo protein to inhibit cancer stem cell growth or kill cancer cells and/or cancer stem cells. Exemplary sensitizing agents include antibodies to IL-10, bone morphogenic proteins and HDAC inhibitors (see for example Sakariassen et al., Neoplasia 9(11):882-92, 2007). These sensitizing agents can be administered before or during treatment with the IL-4 targeted cargo protein. Exemplary dosages of such sensitizing agents include at least 1 μg/mL, such as at least 10 μg/mL, at least 100 μg/mL, for example 5-100 μg/mL or 10-90 μg/mL. The sensitizing agents can be administered daily, three times a week, twice a week, once a week or once every two weeks. Sensitizing agent can also be administered after treatment with the IL-4 targeted cargo protein is finished.

The IL-4 targeted cargo proteins may be used as part of a neo-adjuvant therapy (to primary therapy), as part of an adjuvant therapy regimen, where the intention is to cure the cancer in a subject. The IL-4 targeted cargo proteins can also be administered at various stages in tumor development and progression, including in the treatment of advanced and/or aggressive neoplasias (e.g., overt disease in a subject that is not amenable to cure by local modalities of treatment, such as surgery or radiotherapy), metastatic disease, locally advanced disease and/or refractory tumors (e.g., a cancer or tumor that has not responded to treatment).

“Primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is begun soon after primary therapy, for example 2, 3, 4, 5, or 6 weeks after the last primary therapy treatment to delay recurrence, prolong survival or cure a subject. As noted above, it is contemplated that the IL-4 targeted cargo proteins can be used alone or in combination with one or more other chemotherapeutic agents as part of an adjuvant therapy. Combinations of the IL-4 targeted cargo proteins and standard chemotherapeutics may act to improve the efficacy of the chemotherapeutic and, therefore, can be used to improve standard cancer therapies. This application can be particularly important in the treatment of drug-resistant cancers which are not responsive to standard treatment. The dosage to be administered is not subject to defined limits, but it will usually be an effective amount. The compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The unit dosage forms may be administered once or multiple unit dosages may be administered, for example, throughout an organ, or solid tumor. Examples of ranges for the IL-4 targeted cargo protein(s) in each dosage unit are from about 0.0005 to about 100 mg, or more usually, from about 1.0 to about 1000 mg. Daily dosages of the IL-4 targeted cargo proteins typically are at least 1 ng/kg of body weight, at least 1 μg/kg of body weight, at least 1 mg/kg of body weight, for example fall within the range of about 0.01 to about 100 mg/kg of body weight, in single or divided dose. However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended to limit the scope in any way. In some instances, dosage levels below the lower limit of the aforesaid range may be more than adequate, while in other cases still larger doses may be employed without causing harmful side effects, for example, by first dividing the larger dose into several smaller doses for administration throughout the day.

The IL-4 targeted cargo proteins can be used to treat and/or manage cancer, the methods include administering to a subject in need thereof a prophylactically or therapeutically effective regimen, the regimen comprising administering one or more therapies to the subject, wherein the regimen results in the stabilization or reduction in the cancer stem cell population and does not result in a reduction or only results in a small reduction of the circulating endothelial cell population and/or the circulating endothelial progenitor population. In one example, the regimen achieves a 5%-40%, a 10%-60%, or a 20 to 99% reduction in the cancer stem cell population and/or less than a 25%, less than a 15%, or less than a 10% reduction in the circulating endothelial cell population. In another example, the regimen achieves a 5%-40%, a 10%-60%, or a 20 to 99% reduction in the cancer stem cell population and/or less than a 25%, less than a 15%, or less than a 10% reduction in the circulating endothelial progenitor population. In another example, the regimen achieves a 5%-40%, a 10%-60%, or a 20 to 99% reduction in the cancer stem cell population and/or less than a 25%, less than a 15%, or less than a 10% reduction in the circulating endothelial cell population and the circulating endothelial progenitor population. In a specific example, the stabilization or reduction in the cancer stem cell population is achieved after two weeks, a month, two months, three months, four months, six month, nine months, 1 year, 2 years, 3 years, 4 years or more of administration of one or more of the therapies. In a particular example, the stabilization or reduction in the cancer stem cell population can be determined using any method known in the art. In certain examples, in accordance with the regimen, the circulating cancer stem cell population, the circulating endothelial cell population and/or the circulating endothelial progenitor population is monitored periodically (e.g., after 2, 5, 10, 20, 30 or more doses of one or more of the therapies or after 2 weeks, 1 month, 2 months, 6 months, 1 year, or more of receiving one or more therapies).

In some embodiments, a single infusion of the IL-4 targeted cargo protein, such as for example MDNA55, is administered at a concentration of 1.5 μg/mL (and up to 3 μg/mL) (see, for example, Examples 1 and 2). In some embodiments, infusion volume and parameters can be personalized for each subject/patient to achieve target coverage to the maximum extent possible. In some embodiments, infused volume will range from approximately 7 mL (smallest tumor) to 60 mL (largest tumor). In some embodiments, the duration of infusion will be approximately 6 to 32 hours depending on tumor volume, flow rate and number of catheters. In some embodiments, the maximum delivered dose will be 90 μg. In some embodiments, the dosage is administered intra-cranially. In some embodiments, the IL-4 targeted cargo protein is administered as a single dose of about 90 μg (1.5 μg/mL in 60 mL), about 240 μg (6 μg/mL in 40 mL), or about 300 μg (3 μg/mL in 100 mL). In some embodiments, the IL-4 targeted cargo protein is administered as a single dose of about 1.5 μg/mL to about 3 μg/mL.

In some embodiments, the dosing is 180 μg, or 3 μg/mL×60 mL, of MDNA55 per subject. In some embodiments, the dosing is from about 1.5 μg/mL to about 3.0 μg/mL. In some embodiments, the dosing is about 1.5 μg/m, 2 μg/mL, 2.5 about 3.0 μg/mL or about 3.5 μg/mL In some embodiments, the dosage is for any IL-4 targeted cargo protein described herein. mL. In some embodiments, the dosage is for MDNA55.

In some embodiments, the dosing flow rate is about 5 μL/min/catheter to about 20 μL/min/catheter. In some embodiments, the dosing flow rate is about 10 μL/min/catheter to about 15 μL/min/catheter. In some embodiments, the dosing flow rate is about 15 μL/min/catheter. In some embodiments, 1-4 catheters are employed. In some embodiments, 1-3 catheters are employed. In some embodiments, 1-3 catheters are employed and the flow-rates of up to 15 μL/min/catheter. In some embodiments, 1.5 μg/mL is administered via 1-3 catheters and the flow-rates of up to 15 μL/min/catheter. In some embodiments, 1.5 μg/mL is administered via 1-3 catheters and the flow-rates of up to 15 μL/min/catheter with a total dosage of 90 μg of MDNA55.

### III. Therapeutic Uses

The IL-4 targeted cargo proteins described herein can be used for a variety of therapeutic purposes. Prior to administration for therapeutic purposes the IL-4 targeted cargo protein may need to be modified or adapted for the particular purpose, for example the concentration of IL-4 targeted cargo protein needed for whole body administration may differ from that used for local administration. Similarly, the toxicity of the therapeutic may change depending upon the mode of administration and overall composition being used (e.g., buffer, diluent, additional chemotherapeutic, etc.).

A. Toxicity

Therapeutic proteins may elicit some level of antibody response when administered to a subject, which in some cases may lead to undesirable side effects. Therefore, if necessary, the antigenicity of the IL-4 targeted cargo proteins can be assessed as known in the art and described below. In addition, methods to reduce potential antigenicity are described.

In vivo toxic effects of the IL-4 targeted cargo proteins can be evaluated by measuring their effect on animal body weight during treatment and by performing hematological profiles and liver enzyme analysis after the animal has been sacrificed. The general toxicity of the IL-4 targeted cargo proteins can be tested according to methods known in the art. For example, the overall systemic toxicity of the IL-4 targeted cargo proteins can be tested by determining the dose that kills 100% of mice (i.e. LD100) following a single intravenous injection. Doses that were at least about 2, 5, or 10-fold less than the LD100 or LD50 can be selected for administration into other mammals, such as a human.

The kinetics and magnitude of the antibody response to the IL-4 targeted cargo proteins described herein can be determined, for example, in immunocompetent mice and can be used to facilitate the development of a dosing regimen that can be used in an immunocompetent human. Immunocompetent mice such as the strain C57-BL6 are administered intravenous doses of IL-4 targeted cargo protein. The mice are sacrificed at varying intervals (e.g. following single dose, following multiple doses) and serum obtained. An ELISA-based assay can be used to detect the presence of anti-IL-4 targeted cargo protein antibodies.

To decrease antigenicity of IL-4 targeted cargo proteins the native binding domain of the toxin used as the cargo moiety can be functionally deleted and replaced, for example with a targeting moiety to make the IL-4 targeted cargo protein. The antigenicity of such IL-4 targeted cargo proteins can be determined following exposure to varying schedules of the IL-4 targeted cargo protein which lack portions of the native binding domain using the methods described above. IL-4 targeted cargo proteins that utilize fully humanized antibodies can also be used to minimize antigenicity.

Another method that can be used to allow continued treatment with IL-4 targeted cargo proteins is to use sequentially administered alternative IL-4 targeted cargo proteins derived from other cargo proteins with non-overlapping antigenicity. For example, a IL-4 targeted cargo protein derived from proaerolysin can be used alternately with a IL-4 targeted cargo protein derived from Clostridium septicum alpha toxin or Bacillus thuringiensis delta-toxin. All of these IL-4 targeted cargo proteins would target cancer cells and/or cancer stem cells, but would not be recognized or neutralized by the same antibodies.

Serum samples from these mice can be assessed for the presence of anti-IL-4 targeted cargo protein antibodies as known in the art. As another example, epitope mapping can also be used to determine antigenicity of proteins as described in Stickler, et al., J. Immunotherapy, 23:654-660, 2000. Briefly, immune cells known as dendritic cells and CD4+ T cells are isolated from the blood of community donors who have not been exposed to the protein of interest. Small synthetic peptides spanning the length of the protein are then added to the cells in culture. Proliferation in response to the presence of a particular peptide suggests that a T cell epitope is encompassed in the sequence. This peptide sequence can subsequently be deleted or modified in the IL-4 targeted cargo protein thereby reducing its antigenicity.

B. Treatment of Glioblastoma

In some embodiments, the IL-4 targeted cargo protein is employed for the treatment of a brain tumor. In some embodiments, the brain tumors is glioblastoma (GB). Glioblastoma (GB) is an aggressive brain tumor characterized by rapid proliferation of undifferentiated cells, extensive infiltration, and a high propensity to recur (Hamstra et al., 2005). It is a rapidly progressing and universally fatal cancer. For adults treated with concurrent Temozolomide (Termodar®) and radiotherapy, median survival is 14.6 months, two-year survival is approximately 30%, and five-year survival approximately 10%. Clinical impact is defined by rapid neurologic deterioration which affects the ability to perform everyday functions, such as eating, walking, and talking. There can also be distortion of personality and identity, such as mood, memory, emotion, and intelligence. GB does not typically metastasize outside of the CNS and death usually results due to increased intracranial pressure and herniation caused by uncontrolled growth of tumor within the bone-encased brain cavity. Annual worldwide incidence of primary GB in well-resourced countries is approximately 27,500 (Decision Recourses, 2013).

In some embodiments, the IL-4 targeted cargo protein is employed for the treatment of a brain tumor over-expressing IL-4R, for example, mixed adult glioma, mixed pediatric glioma, diffuse intrinsic pontine gliomas (DIPG), medulloblastoma, adult pituitary adenoma, meningioma.

C. Biomarkers and Patient Populations

The IL-4 targeted cargo proteins of the invention, including for example MDNA55 finds use for the treatment of GBM including recurrent GBM, brain metastasis, newly diagnosed GBM, and diffuse intrinsic pontine glioma in particular patient populations.

In some embodiments, the cancer biopsy and autopsy samples are from adult and pediatric CNS tumors (e.g., brain tumors). In some embodiments, the patient has glioblastoma (also called glioblastoma multiform—GBM). In some embodiments, the patient has recurrent GBM. In some embodiments, the patient has brain metastasis from GBM. In some embodiments, the patient has newly diagnosed GBM. In some embodiments, the patient has diffuse intrinsic pontine glioma. In some embodiments, the patient tumor samples have been shown to over-express the IL-4R as compared to little or no IL-4R expression in normal adult and pediatric brain tissue (Puri et al., 1994a; Kawakami et al., 2002a; Joshi, et al., 2001; Konanbash et al., 2013). While not being bound by theory, cells that do not express the IL-4R target do not bind to MDNA55 and are, therefore, not subject to PE-mediated effects (Kawakami et al., 2002).

In some embodiments, the IL-4 targeted cargo proteins, including for example MDNA55, induce tumor growth killing that is not growth-rate dependent (Li and Hall, 2010). In some embodiments, quiescent cancer cells and/or cancer stem cells and slower growing non-malignant cells of the tumor microenvironment (TME) may be as sensitive to MDNA55 as rapidly dividing tumor cells.

In some embodiments, the cancer cells are O⁶-methylguanine-methyltransferase (MGMT) positive. In some embodiments, the cancer cells are O⁶-methylguanine-methyltransferase (MGMT) negative. In some embodiments, the cancer cells have methylated MGMT gene promoter. In some embodiments, the cancer cells have unmethylated MGMT gene promoter. In some embodiments, O⁶-methylguanine-methyltransferase (MGMT) positive cancer cells (harboring unmethylated MGMT promoters and therefore resistant to Temozolomide) are sensitive to MDNA55. Exemplary sensitive CNS cancer cell lines include T98G (glioblastoma) and have been shown to over-express MGMT. Such cell lines are resistant to alkylating agents such as Temozolomide (Huang et al., 2012; Kuo et al., 2007; Kokkinakis et al., 2003), but can be sensitive to MDNA55. In some embodiments, cancer cells harboring methylated MGMT gene promoter are sensitive to MDNA55.

In some embodiments, IL-4R-expressing cell lines show picomolar sensitivity to MDNA55. See, for example, Puri et al., 1996b; Kreitman et al., 1995; Shimamura et al., 2007. In some embodiments, IL-4R-expressing tumors exhibit picomolar sensitivity to the IL-4 targeted cargo proteins of the present invention. In some embodiments, IL-4R-expressing tumors exhibit picomolar sensitivity to MDNA55. In some embodiments, MGMT expressing tumors exhibit sensitivity to the IL-4 targeted cargo proteins of the present invention. In some embodiments, IL-4R-expressing gliobalstomas exhibit sensitivity to MDNA55. In some embodiments, MGMT-expressing tumors exhibit sensitivity to MDNA55. In some embodiments, MGMT-expressing gliobalstomas exhibit sensitivity to MDNA55.

Furin like protease cleavage of MDNA55 and result in activation of the PE toxin (Chironi et al., 1997; Shapira and Benhar, 2010) and glioblastomas often express furin (Mercapide, et al., 2002; Wick et al., 2004). The higher expression levels of furin in glioma cells as opposed to normal cells provides additional tumor specificity and also a contributes to factor to the exceptional picomolar sensitivity of cancer cells to MDNA55. In some embodiments, the tumor expresses furin. In some embodiments, the tumor expressing furin is more sensitive to the IL-4 targeted cargo proteins, such as MDNA55, than normal non-tumor cells.

IL-4R is over-expressed not only by CNS tumors but also by non-malignant cells (MDSCs and TAMs) of the immunosuppressive TME. In some embodiments, the IL-4R IL-4 targeted cargo proteins, including MDNA55, find use in the treatment adult and pediatric patients with aggressive forms of primary and metastatic brain cancer.

GBM has a robust immunosuppressive TME and may comprise up to 40% of the tumor mass (Kennedy et al., 2013). Recently, it has been shown that malignant gliomas have a T-helper cell type-2 (Th2) bias and are heavily infiltrated by myeloid derived suppressor cells (MDSCs) and tumor associated macrophages (TAMs) and that the IL4/IL-4R bias mediates their immunosuppressive functions (Harshyne, et al., 2016). Furthermore, IL-4R is up-regulated on glioma-infiltrating myeloid cells but not in the periphery or in normal brain (Kohanbash et al., 2013). In some embodiments, purging Th2 cells, MDSCs, and TAMs using the IL-4 targeted cargo proteins of the present invention, including MDNA55, may alleviate the immune block associated with cancer. In some embodiments, the alleviation of immune block promotes anti-tumor immunity and aid in long-term disease control and/or disease treatment.

D. IL-4R as a Biomarker or Companion Diagnostic

In some embodiments, the level of IL-4R (also referred to as “IL4R”) expression can be employed as a biomarker or companion diagnostic for use in the determining treatment regimens as well as predicting or determining treatment efficacy. In some embodiments, the level of Type 2 IL-4R (Type II IL-R4, comprising IL-4Rα and IL-13Rα1) expression can be employed as a biomarker or companion diagnostic for use in the determining treatment regimens as well as predicting or determining treatment efficacy. In some embodiments, IL-4Rα is reactive in the cytoplasm of tumor cells. However, IL-4Rα also be observed in serum and occasionally in the cytoplasm of normal cells and normal tissue components.

In some embodiments, the level of IL-4R expression is determined by measuring IL-4Rα expression. In some embodiments, the level of IL-4R expression, including the level of IL-4Rα expression, is scored by a board-certified pathologist. In some embodiments, the level of expression of Type 2 IL-4R (Type II IL-R4, comprising IL-4Rα and IL-13Rα1) is determined by measuring IL-4Rα expression. In some embodiments, the level of expression of Type 2 IL-4R (Type II IL-R4, comprising IL-4Rα and IL-13Rα1) is scored by a board-certified pathologist.

There are two main components to scoring malignant tumor cells, which include Percent Scores and an H-Scores (derived from percentages that are recorded at differential intensities) as described below. In some embodiments, any IL-4Rα staining observed in cells that are clearly non-neoplastic can be excluded. In some embodiments, malignant cells are considered to express IL-4Rα if cytoplasmic tumor cell staining is recognized.

### Percent Score Method

Percent Scores are calculated by summing the percentages of intensities in tumor cells at either ≥1+, ≥2+ or ≥3+. Thus, scores range from 0 to 100.
Percent Score ≥1+=(% at 1+)+(% at 2+)+(% at 3+)
Percent Score ≥2+=(% at 2+)+(% at 3+)
Percent Score ≥3+=(% at 3+)

In some embodiments, a high level of IL-4R expression is indicated by a percent score of ≥2+. In some embodiments, a high level of IL-4R expression is indicated by a percent score of ≥3+.

In some embodiments, a moderate level of IL-4R expression is indicated by a percent score of ≥1+ but <2.

In some embodiments, no detectable level of IL-4R expression is indicated by a percent score of 0. In some embodiments, a low level of IL-4R expression is indicated by a percent score of ≥1+.

### H-Score Method

The H-Score is calculated by summing the percentage of tumor cells with intensity of expression (brown staining) multiplied by their corresponding intensity a four-point semi-quantitative scale (0, 1+, 2+, 3+). Thus, scores range from 0 to 300.

H-Score=[(% at <1)×0]+[(% at 1+)×1]+[(% at 2+)×2]+[(% at 3+)×3]

For both the Percent Score and H-Score methods, the four-point semi-quantitative intensity scale is described as follows: 0—null, negative or non-specific staining, 1+—low or weak staining, 2+—medium or moderate staining, and 3+—high or strong staining. The percentage at each intensity is estimated directly and typically reported as one of the following, though other increments can also be used: 0, 1, 2, 3, 4, 5, 10, 15, 20, 25, 30, 35, 40, 45, 50, 55, 60, 65, 70, 75, 80, 85, 90, 95, 96, 97, 98, 99, or 100%.

In some embodiments, no level of IL-4R expression to a low level of IL-4R expression is indicated by H-Scores from 0 to 75 (e.g., no to low expression).

In some embodiments, a moderate level of IL-4R expression is indicated by H-Scores from 76 to 150 (e.g., moderate expression).

In some embodiments, a high level of IL-4R expression is indicated by H-Scores from 151 to 225 (e.g., high expression).

In some embodiments, a high level of IL-4R expression is indicated by H-Scores from 226 to 300 (e.g., very high expression).

In some embodiments, a moderate or high level of IL-4R expression is indicated by H-Scores >75. In some embodiments, a moderate or high level of IL-4R expression is indicated by H-Scores from 76 to 300. In some embodiments, a moderate or high level of IL-4R expression is indicated by H-Scores from 80 to 300. In some embodiments, a moderate or high level of IL-4R expression is indicated by H-Scores from 90 to 300. In some embodiments, a moderate or high level of IL-4R expression is indicated by H-Scores from 95 to 300. In some embodiments, a moderate or high level of IL-4R expression is indicated by H-Scores from 100 to 300. In some embodiments, a moderate or high level of IL-4R expression is indicated by H-Scores from 105 to 300. In some embodiments, a moderate or high level of IL-4R expression is indicated by H-Scores from 110 to 300. In some embodiments, a moderate or high level of IL-4R expression is indicated by H-Scores from 115 to 300. In some embodiments, a moderate or high level of IL-4R expression is indicated by H-Scores from 120 to 300. In some embodiments, a moderate or high level of IL-4R expression is indicated by H-Scores from 125 to 300. In some embodiments, a moderate or high level of IL-4R expression is indicated by H-Scores from 130 to 300. In some embodiments, a moderate or high level of IL-4R expression is indicated by H-Scores from 135 to 300. In some embodiments, a moderate or high level of IL-4R expression is indicated by H-Scores from 140 to 300. In some embodiments, a moderate or high level of IL-4R expression is indicated by H-Scores from 145 to 300. In some embodiments, a moderate or high level of IL-4R expression is indicated by H-Scores from 150 to 300. In some embodiments, a moderate or high level of IL-4R expression is indicated by H-Scores from 155 to 300. In some embodiments, a moderate or high level of IL-4R expression is indicated by H-Scores from 160 to 300. In some embodiments, a moderate or high level of IL-4R expression is indicated by H-Scores from 165 to 300. In some embodiments, a moderate or high level of IL-4R expression is indicated by H-Scores from 170 to 300. In some embodiments, a moderate or high level of IL-4R expression is indicated by H-Scores from 175 to 300. In some embodiments, a moderate or high level of IL-4R expression is indicated by H-Scores from 180 to 300. In some embodiments, a moderate or high level of IL-4R expression is indicated by H-Scores from 185 to 300. In some embodiments, a moderate or high level of IL-4R expression is indicated by H-Scores from 190 to 300. In some embodiments, a moderate or high level of IL-4R expression is indicated by H-Scores from 195 to 300. In some embodiments, a moderate or high level of IL-4R expression is indicated by H-Scores from 200 to 300. In some embodiments, a moderate or high level of IL-4R expression is indicated by H-Scores from 205 to 300. In some embodiments, a moderate or high level of IL-4R expression is indicated by H-Scores from 210 to 300. In some embodiments, a moderate or high level of IL-4R expression is indicated by H-Scores from 215 to 300. In some embodiments, a moderate or high level of IL-4R expression is indicated by H-Scores from 220 to 300. In some embodiments, a moderate or high level of IL-4R expression is indicated by H-Scores from 225 to 300. In some embodiments, a moderate or high level of IL-4R expression is indicated by H-Scores from 230 to 300. In some embodiments, a moderate or high level of IL-4R expression is indicated by H-Scores from 235 to 300. In some embodiments, a moderate or high level of IL-4R expression is indicated by H-Scores from 240 to 300. In some embodiments, a moderate or high level of IL-4R expression is indicated by H-Scores from 245 to 300. In some embodiments, a moderate or high level of IL-4R expression is indicated by H-Scores from 250 to 300. In some embodiments, a moderate or high level of IL-4R expression is indicated by H-Scores from 255 to 300. In some embodiments, a moderate or high level of IL-4R expression is indicated by H-Scores from 265 to 300. In some embodiments, a moderate or high level of IL-4R expression is indicated by H-Scores from 270 to 300. In some embodiments, a moderate or high level of IL-4R expression is indicated by H-Scores from 275 to 300. In some embodiments, a moderate or high level of IL-4R expression is indicated by H-Scores from 280 to 300. In some embodiments, a moderate or high level of IL-4R expression is indicated by H-Scores from 285 to 300. In some embodiments, a moderate or high level of IL-4R expression is indicated by H-Scores from 290 to 300. In some embodiments, a moderate or high level of IL-4R expression is indicated by H-Scores from 295 to 300.

Occasionally, cancer samples, including GBM samples included background IL-4Rα staining throughout benign tissue. When present, such interstitial staining was captured with an average intensity score of 1+, 2+, or 3+ to record the level of background cytoplasmic staining present around tumor cells. When absent, this value was recorded as NA (not applicable). In some embodiments, high background reactivity could contribute to higher IL-4Rα expression in tumor cells. As such, in some embodiments, the interstitial staining score should be taken into consideration when evaluating reactivity scores for malignant tumor cells.

E. Kits

IL-4R expression can be detected using either IHC or RT-PCR analyses. In some embodiment, and RT-PCR based method and associated kit can be employed. In some embodiments, an IL-4R antibody based method for detection and associated kit can be employed. Antibodies to IL-4R that find use in such kits can include commercially available as well as other known or developed IL-4R antibodies. In some embodiments, an IL-4R antibody can be employed in an immunohistochemistry (IHC)-based assay for detecting IL-4R expression. In some embodiments, the IL-4R is a monoclonal antibody to the IL-4Rα chain, Joshi et al., (Joshi B H, et al., In situ expression of interleukin-4 (IL-4) receptors in human brain tumors and cytotoxicity of a recombinant IL-4 cytotoxin in primary glioblastoma cell cultures. Cancer Res. 2001; 61:8058-8061) evaluated expression in surgical/biopsy samples of brain tumor tissues by IHC. 83% (Ichinose, M., et al., Cancer Res. 2002, and Johnson H, et al., Mol. Cell Proteomics. 2012 December; 11(12):1724-40) of GBM tumors were moderately to intensely positive for IL-4Rα (Joshi B H, et al. In situ expression of interleukin-4 (IL-4) receptors in human brain tumors and cytotoxicity of a recombinant IL-4 cytotoxin in primary glioblastoma cell cultures. Cancer Res. 2001; 61:8058-8061), whereas 11 of 11 normal brain samples showed no detectable staining for IL-4R, demonstrating tumor specificity.

In some embodiments, the level of IL-4R can be employed as a companion diagnostic and/or predictive marker to select IL-4R positive patients for therapeutic treatment with an IL-4 targeted cargo protein of the present invention. In some embodiments, the level of Type 2 IL-4R (Type II IL-R4, comprising IL-4Rα and IL-13Rα1) can be employed as a companion diagnostic and/or predictive marker to select IL-4R positive patients for therapeutic treatment with an IL-4 targeted cargo protein of the present invention.

In some embodiments, the present invention provides a kit for detecting IL-4R expression. In some embodiments, the present invention provides a kit for detecting Type 2 IL-4R (Type II IL-R4, comprising IL-4Rα and IL-13Rα1) expression. In some embodiments, the kit provides the components for RT-PCR based detection of IL-4R mRNA expression levels. In some embodiments, the kit provides the components for an immunohistochemistry (IHC)-based assay for detecting or measuring IL-4R expression. In some embodiments the kit comprises an IL-4R antibody and instructions for using the IL-4R antibody in an immunohistochemistry (IHC)-based assay. In some embodiments, the kit further comprises instructions for determining the percent score. In some embodiments, the kit further comprises instructions for determining the H-Score. In some embodiments the kit comprises an IL-4R antibody, instructions for using the IL-4R antibody in an immunohistochemistry (IHC)-based assay, and instructions for determining the percent score. In some embodiments the kit comprises an IL-4R antibody, instructions for using the IL-4R antibody in an immunohistochemistry (IHC)-based assay, and instructions for determining the H-Score. In some embodiments the kit comprises an IL-4Rα antibody, instructions for using the IL-4Rα antibody in an immunohistochemistry (IHC)-based assay, and instructions for determining the percent score. In some embodiments the kit comprises an IL-4Rα antibody, instructions for using the IL-4Rα antibody in an immunohistochemistry (IHC)-based assay, and instructions for determining the H-Score.

F. Convection Enhanced Delivery (CED)

The present invention contemplates the use of CED for delivery of therapeutics directly into the tumor. CED has been described in Patel et al., Neurosurgery 56: 1243-52, 2005, (incorporated by reference herein in its entirety). This enables high local drug concentrations to be achieved while limiting systemic toxicity. The procedure has been used in the treatment of recurrent GBM and other CNS disorders from early clinical development through to Phase 3 clinical trials with a good safety profile. In some embodiments, MDNA55 is delivered by convection-enhanced delivery (CED) intratumorally. In some embodiments, CED is performed by direct infusion through intracranial catheters (1 or more, depending on the size of the tumor) under constant pressure. In some embodiments, this is over a period of 1 to 7 days. The total dose of MDNA55 is about 90-100 μg. In some embodiments, the dosage can be adjusted within the range of range 5 μg to 1 mg. In some embodiments, MRI imaging prior to, during and following infusion is used to monitor drug distribution and tumor response. In some embodiments, subjects/patients are monitored by clinical evaluation and MRI on an ongoing basis after treatment.

In some embodiments, CED will be employed to administer the IL-4 targeted cargo proteins to the CNS tumor. In some embodiments, CED will be employed to administer MDNA55 for the treatment of CNS tumors. In some embodiments, CED will be employed to administer MDNA55 for the treatment of GBM. In some embodiments, CED will be employed to administer MDNA55 for the treatment of progressive and/or recurrent GBM.

In some embodiments, the CED process will employ the use of planning high precision planning software (e.g. iPlan® Flow Infusion Version 3.0.6, Brainlab AG) for determining catheter placement. In some embodiments, the CED process will employ catheters specifically designed for brain usage. In some embodiments, the CED process will not employ large diameter ventricular catheters, which can be prone to drug leakage from the intended delivery site (see, for example 3).

In some embodiments, the CED process will include co-infusion of a surrogate tracer, for example, a magnetic resonance imaging (MRI) contrast agent, will allow real-time monitoring of MDNA55 distribution ensuring adequate coverage of the tumor and the infiltrative edges.

In some embodiments, the surrogate tracer molecule can include but is no limited to any magnetic resonance imaging tracer. In some embodiments, the surrogate tracer is a gadolinium bound tracer. In some embodiments, the surrogate tracer is selected from the group consisting of gadolinium-diethylenetriamine pentaacetic acid [Magnevist®] [Gd-DTPA]; commercially available from Bayer Healthcare Pharmaceuticals, Inc.) and gadolinium-bound albumin (Gd-albumin). In some embodiments, the surrogate tracer used during CED will enable effective real-time monitoring of drug distribution. In some embodiments, the real-time monitoring allows for ensuring adequate coverage of the tumor and the peritumoral infiltrating margin with the IL-4 targeted cargo protein, including for example, MDNA55. In some embodiments, the surrogate tracer can be administered in combination with the targeted cargo protein to determine if the targeted cargo protein is delivered to a tumor, such as a brain tumor, safely at therapeutic doses while monitoring its distribution in real-time.

For further information regarding on CED and surrogate tracers, see for example, Chittiboina et al., 2014; Jahangiri et al., 2016; and Murad et al., Clin. Cancer Res. 12(10):3145-51, 2006), all of which are incorporated herein by reference in their entireties.

G. Monitoring Treatment

Any in vitro or in vivo (ex vivo) assays known to one of ordinary skill in the art that can detect and/or quantify cancer cells and/or cancer stem cells can be used to monitor cancer cells and/or cancer stem cells in order to evaluate the impact of a treatment utilizing a IL-4 targeted cargo protein. These methods can be used to assess the impact in a research setting as well as in a clinical setting. The results of these assays then may be used to alter the targeting moiety, cargo protein or alter the treatment of a subject. Assays for the identification of cancer cells and/or cancer stem cells are provided in US patent application no. 2007/0292389 to Stassi et al. (herein incorporated by reference).

Cancer cells and/or cancer stem cells usually are a subpopulation of tumor cells. Cancer cells and/or cancer stem cells can be found in biological samples derived from cell culture or from subjects (such as a tumor sample). Various compounds such as water, salts, glycerin, glucose, an antimicrobial agent, paraffin, a chemical stabilizing agent, heparin, an anticoagulant, or a buffering agent can be added to the sample. The sample can include blood, serum, urine, bone marrow or interstitial fluid. In another example, the sample is a tissue sample. In a particular example, the tissue sample is breast, brain, skin, colon, lung, liver, ovarian, pancreatic, prostate, renal, bone or skin tissue. In a specific example, the tissue sample is a biopsy of normal or tumor tissue. The amount of biological sample taken from the subject will vary according to the type of biological sample and the method of detection to be employed. In a particular example, the biological sample is blood, serum, urine, or bone marrow and the amount of blood, serum, urine, or bone marrow taken from the subject is 0.1 mL, 0.5 mL, 1 mL, 5 mL, 8 mL, 10 mL or more. In another example, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams.

A test sample can be a sample derived from a subject that has been treated with a IL-4 targeted cargo protein. Test samples can also include control samples. In some examples a control sample is from a subject prior to treatment with a IL-4 targeted cargo protein and in other examples the test sample can be taken from a different location within a subject that has been treated with a IL-4 targeted cargo protein. Control samples can also be derived from cells that have been artificially cultured. The sample can be subjected to one or more pretreatment steps prior to the detection and/or measurement of the cancer stem cell population in the sample. In certain examples, a biological fluid is pretreated by centrifugation, filtration, precipitation, dialysis, or chromatography, or by a combination of such pretreatment steps. In other examples, a tissue sample is pretreated by freezing, chemical fixation, paraffin embedding, dehydration, permeabilization, or homogenization followed by centrifugation, filtration, precipitation, dialysis, or chromatography, or by a combination of such pretreatment steps. In certain examples, the sample is pretreated by removing cells other than stem cells or cancer cells and/or cancer stem cells from the sample, or removing debris from the sample prior to the determination of the amount of cancer cells and/or cancer stem cells in the sample.

In certain examples, the amount of cancer cells and/or cancer stem cells in a subject or a sample from a subject is/are assessed prior to therapy or regimen to establish a baseline. In other examples the sample is derived from a subject that was treated using a IL-4 targeted cargo protein. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the amount of cancer cells and/or cancer stem cells is assessed after a certain number of doses (e.g., after 2, 5, 10, 20, 30 or more doses of a therapy). In other examples, the amount of cancer cells and/or cancer stem cells is assessed after 1 week, 2 weeks, 1 month, 2 months, 1 year, 2 years, 3 years, 4 years or more after receiving one or more therapies.

Targets on cancer cells and/or cancer stem cells are also expressed on normal non-cancerous cells. Therefore, in some examples the identification of cancer cells and/or cancer stem cells can be made by comparing the relative amount of signal generated from target binding in a control sample and comparing it to the test sample for which the presence or absence of cancer cells and/or cancer stem cells is being determined. In such examples, the number, quantity, amount or relative amount of cancer cells and/or cancer stem cells in a sample can be expressed as the percentage of, e.g., overall cells, overall cancerous cells or overall stem cells in the sample.

The results from testing a sample for the presence of cancer cells and/or cancer stem cells and/or the amount of cancer cells and/or cancer stem cells present can be used to alter treatment regimes, including altering the variety of IL-4 targeted cargo protein used. For example, if testing before and after treatment reveals that the population of cancer cells and/or cancer stem cells increased and/or did not decrease treatment can be altered. For example, the dosage of the therapeutic can be altered and/or a IL-4 targeted cargo protein designed to target distinct target can be substituted or added to the treatment regime.

The amount of cancer cells and/or cancer stem cells can be monitored/assessed using standard techniques known to one of ordinary skill in the art. Cancer cells and/or cancer stem cells can be monitored by obtaining a sample, and detecting cancer cells and/or cancer stem cells in the sample. The amount of cancer cells and/or cancer stem cells in a sample (which may be expressed as percentages of, e.g., overall cells or overall cancer cells) can be assessed by detecting the expression of antigens on cancer cells and/or cancer stem cells. Any technique known to those skilled in the art can be used for assessing the population of the cancer cells and/or cancer stem cells. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer cells and/or cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g., prior to, or during therapy). For the purposes of immunoassays one or more of the targets displayed by the cancer stem cell can be used as the target for the immunoassay.

For example, brain cancer cells and/or cancer stem cells can be identified using a CD133+ target, as well as other targets known to be expressed on brain cancer cells and/or cancer stem cells. Additional exemplary markers can be found in Sakariassen et al., Neoplasia 9(11):882-92, 2007 and Vermeulen et al., Cell. Death Differ. 15(6):947-58, 2008 and U.S. patent application 2008/0118518, which is herein incorporated by reference.

In some embodiments, treatment can be monitoring using an IL-4R biomarker expression level, as described in the next section below.

H. Therapeutic Variations

One of ordinary skill in the art will appreciate that targets on cancer cells and/or cancer stem cells can also be expressed on normal healthy cells. For example, CD133 was initially shown to be expressed on primitive hematopoietic stem and progenitor cells and retinoblastoma and then subsequently shown to be expressed on cancer cells and/or cancer stem cells. Therefore, in some examples where a cancer stem cell target is expressed on a class of non-cancerous cells therapy can involve removal of a population of the non-cancerous cells followed by IL-4 targeted cargo protein treatment directed to the cancer stem cell of interest and then reintroducing the non-cancerous cells expressing the target.

In another example, healthy populations of cells that express the same target as that of a cancer stem cell population are protected through the use of two or more IL-4 targeted cargo proteins. A first IL-4 targeted cargo protein is engineered to target a first cancer stem cell target (e.g., CD133). The cargo protein that is included in the first IL-4 targeted cargo protein can be a toxin that is in an inactive form. A second IL-4 targeted cargo protein is engineered to target a second target on the cancer stem cell (e.g., CD24). This second IL-4 targeted cargo protein includes a protein sequence capable of activating the first IL-4 targeted cargo protein. Thus, only a cancer stem cell that expresses the targets for both the first IL-4 targeted cargo protein and the second cargo protein will receive the therapeutic activity of the cargo moiety.

In another therapeutic variation the subject is treated with an agonist to the target displayed on the cancer stem cell. The cancer cells and/or cancer stem cells then display an increased level of the target. The treatment with the agonist can then be administered before, during or after administration of the IL-4 targeted cargo protein. One of ordinary skill in the art will appreciate that the exact timing of administration will depend upon the specific agonist chosen and the specific IL-4 targeted cargo protein.

I. Expression of Mutant IL-4 Gene Products

The nucleic acid molecules described above can be contained within a vector that is capable of directing their expression in, for example, a cell that has been transduced with the vector. Accordingly, in addition to the subject IL-4 muteins, expression vectors containing a nucleic acid molecule encoding a subject IL-4 mutein and cells transfected with these vectors are among the preferred embodiments.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the IL-4 muteins to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction of a Modular Dihydrafolate Reductase cDNA Gene: Analysis of Signals Utilized for Efficient Expression”, Mol. Cell. Biol., 2, pp. 1304-19 (1982)) or glutamine synthetase (“GS”) amplification (see, e.g., U.S. Pat. No. 5,122,464 and European published application 338,841).

In some embodiments, the human IL-4 muteins of the present disclosure will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are included also.

Exemplary recombinant expression vectors can include one or more regulatory sequences, selected on the basis of the host cells to be used for expression, operably linked to the nucleic acid sequence to be expressed.

The expression constructs or vectors can be designed for expression of an IL-4 mutein or variant thereof in prokaryotic or eukaryotic host cells.

Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

Expression of proteins in prokaryotes is most often carried out in Escherichia coli with vectors containing constitutive or inducible promoters. Strategies to maximize recombinant protein expression in E. coli can be found, for example, in Gottesman (1990) in Gene Expression Technology: Methods in Enzymology 185 (Academic Press, San Diego, Calif.), pp. 119-128 and Wada et al. (1992) Nucleic Acids Res. 20:2111-2118. Processes for growing, harvesting, disrupting, or extracting the IL-4 mutein or variant thereof from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

In some embodiments the recombinant IL-4 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2^{nd} ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-4 muteins of the present disclosure can be optimized for expression in the host cell of interest. The G-C content of the sequence can be adjusted to levels average for a given cellular host, as calculated by reference to known genes expressed in the host cell. Methods for codon optimization are well known in the art. Codons within the IL-4 mutein coding sequence can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif.) for use in insect cells.

In some embodiments nucleic acid inserts, which encode the subject IL-4 muteins in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject IL-4 mutein, particularly as regards potential secondary structures. Hosts should be selected by consideration of their compatibility with the chosen vector, the toxicity of the product coded for by the DNA sequences of this invention, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Useful expression vectors for eukaryotic hosts, include, for example, vectors with expression control sequences from SV40, bovine papilloma virus, adenovirus and cytomegalovirus. Useful expression vectors for bacterial hosts include known bacterial plasmids, such as plasmids from E. coli, including col E1, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof. Useful vectors for insect cells include pVL 941 and pFastBac™ 1 (GibcoBRL, Gaithersburg, Md.). Cate et al., “Isolation Of The Bovine And Human Genes For Mullerian Inhibiting Substance And Expression Of The Human Gene In Animal Cells”, Cell, 45, pp. 685-98 (1986).

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject IL-4 mutein disclosed herein are also features of the invention. A cell of the invention is a transfected cell, i.e., a cell into which a nucleic acid molecule, for example a nucleic acid molecule encoding a mutant IL-4 polypeptide, has been introduced by means of recombinant DNA techniques. The progeny of such a cell are also considered within the scope of the invention.

The precise components of the expression system are not critical. For example, an IL-4 mutein can be produced in a prokaryotic host, such as the bacterium E. coli, or in a eukaryotic host, such as an insect cell (e.g., an Sf21 cell), or mammalian cells (e.g., CHO, HEK293, COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, IL-4 muteins obtained will be glycosylated or unglycosylated depending on the host organism used to produce the mutein. If bacteria are chosen as the host then the IL-4 mutein produced will be unglycosylated. Eukaryotic cells, on the other hand, will glycosylate the IL-4 muteins, although perhaps not in the same way as native-IL-4 is glycosylated. The IL-4 mutein produced by the transformed host can be purified according to any suitable method. Various methods are known for purifying IL-4. See, e.g. Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc. IL-4 muteins can be isolated from inclusion bodies generated in E. coli, or from conditioned medium from either mammalian or yeast cultures producing a given mutein using cation exchange, gel filtration, and/or reverse phase liquid chromatography.

Another exemplary method of constructing a DNA sequence encoding the IL-4 muteins is by chemical synthesis. This includes direct synthesis of a peptide by chemical means of the protein sequence encoding for an IL-4 mutein exhibiting the properties described. This method can incorporate both natural and unnatural amino acids at positions that affect the interactions of IL-4 with the IL-4Rα and/or the IL-13Rα1. Alternatively a gene which encodes the desired IL-4 mutein can be synthesized by chemical means using an oligonucleotide synthesizer. Such oligonucleotides are designed based on the amino acid sequence of the desired IL-4 mutein, and preferably selecting those codons that are favored in the host cell in which the recombinant mutein will be produced. In this regard, it is well recognized that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated that for a given DNA sequence encoding a particular IL-4 mutein, there will be many DNA degenerate sequences that will code for that IL-4 mutein. For example, it will be appreciated that in addition to the preferred DNA sequence for mutein H9, there will be many degenerate DNA sequences that code for the IL-4 mutein shown. These degenerate DNA sequences are considered within the scope of this disclosure. Therefore, “degenerate variants thereof” in the context of this invention means all DNA sequences that code for and thereby enable expression of a particular mutein.

The biological activity of the IL-4 muteins can be assayed by any suitable method known in the art. Such assays include PHA-blast proliferation and NK cell proliferation.

J. Anti-PD-1 Antibodies and Combinations

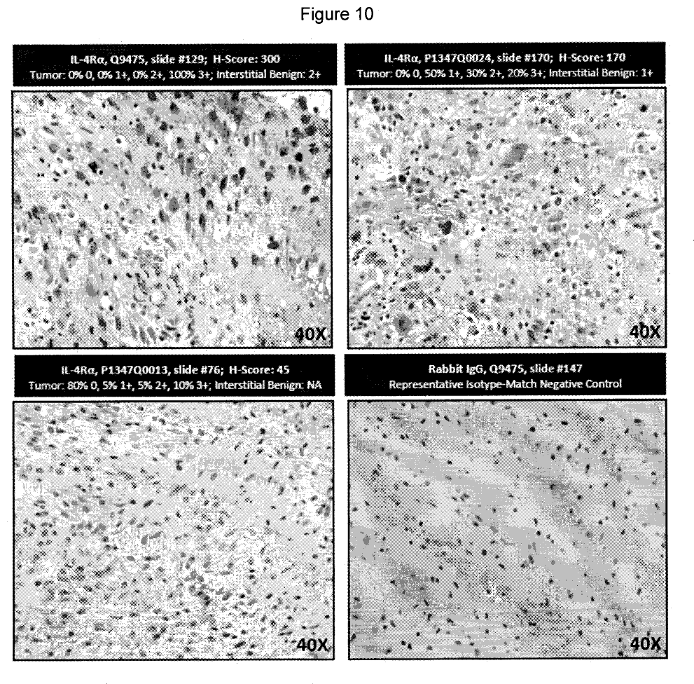

Anti-PD-1 antibodies for use according to the invention and methods described herein include but are not limited to nivolumab, BMS-936558, MDX-1106, ONO-4538, AMP224, CT-011, and MK-3475 (pembrolizumab), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872) and/or a PD-1 antibody as recited in U.S. Patent Publication No. 2017/0081409. There are two approved anti-PD-1 antibodies, pembrolizumab (Keytruda®; MK-3475-033) and nivolumab (Opdivo®; CheckMate078) and many more in development which can be used in combination described herein. Exemplary anti-PD-1 anitbody sequences are shown in FIG. 10 and any of these can be used with the combination methods with the IL-4 muteins as described herein.

K. Anti-PD-L1 Antibodies and Combinations

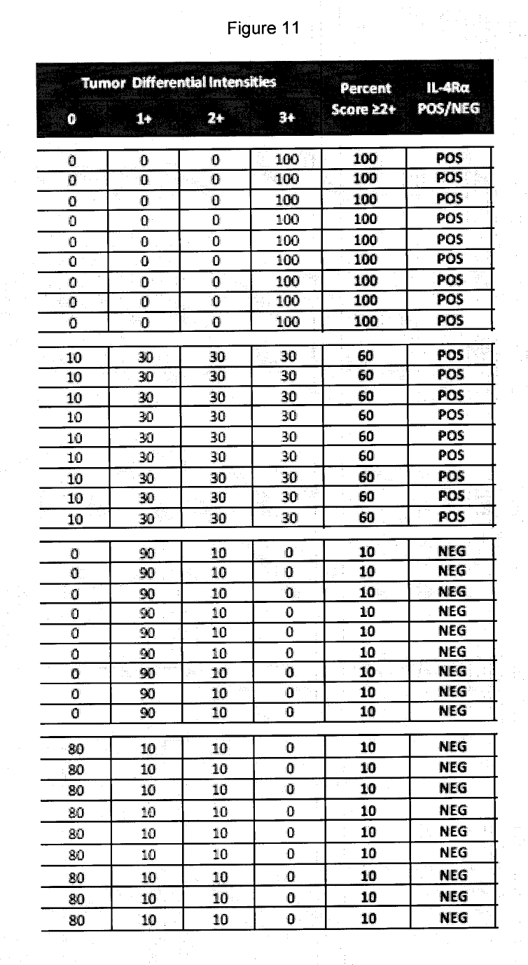

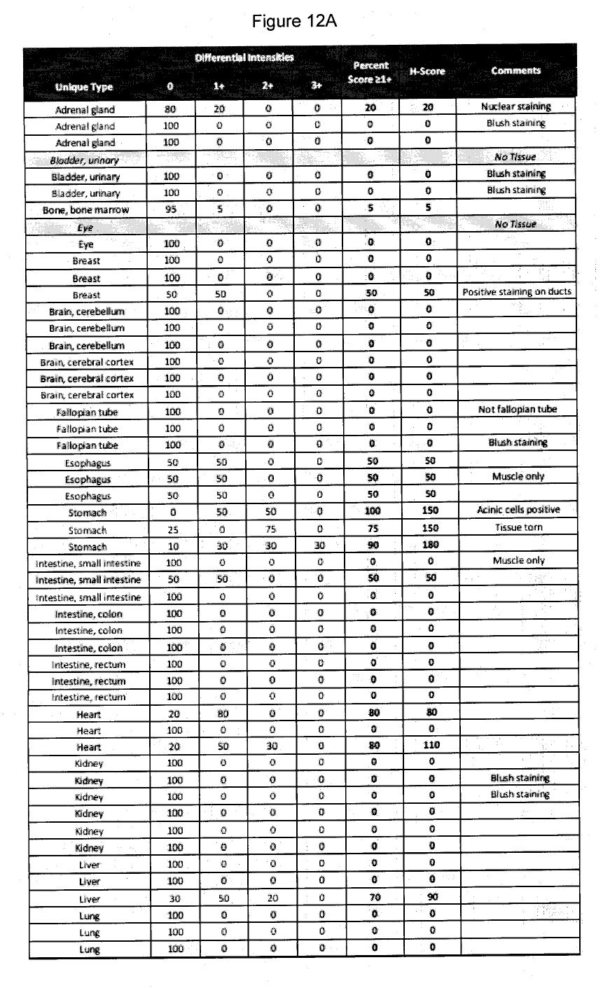

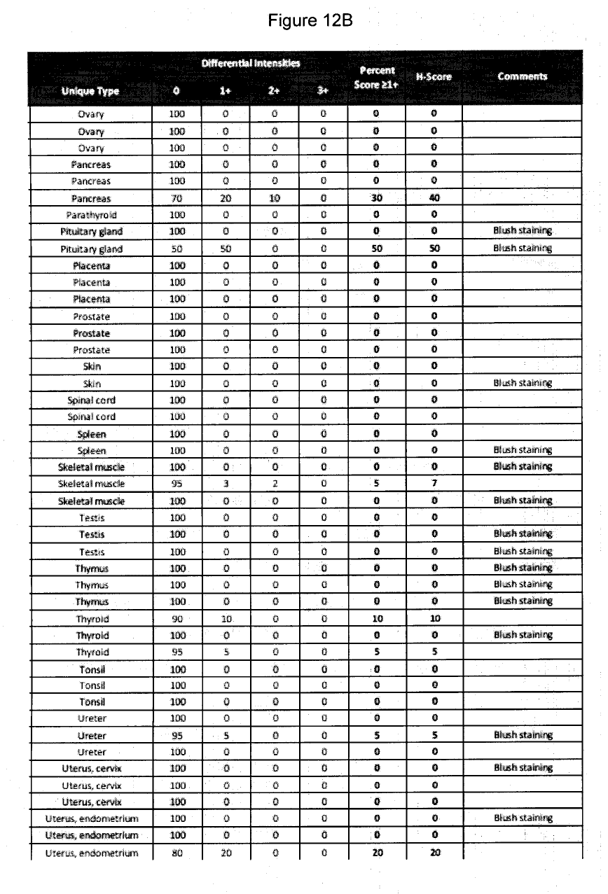

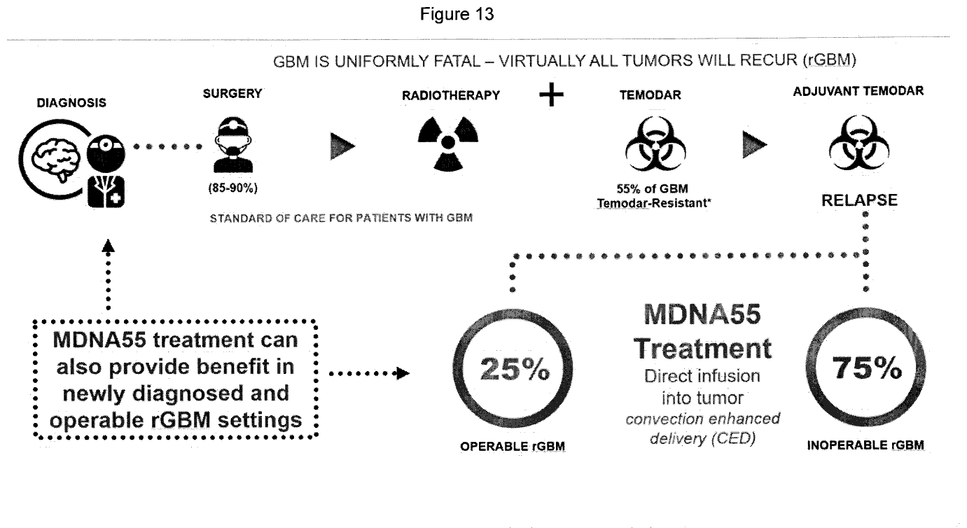

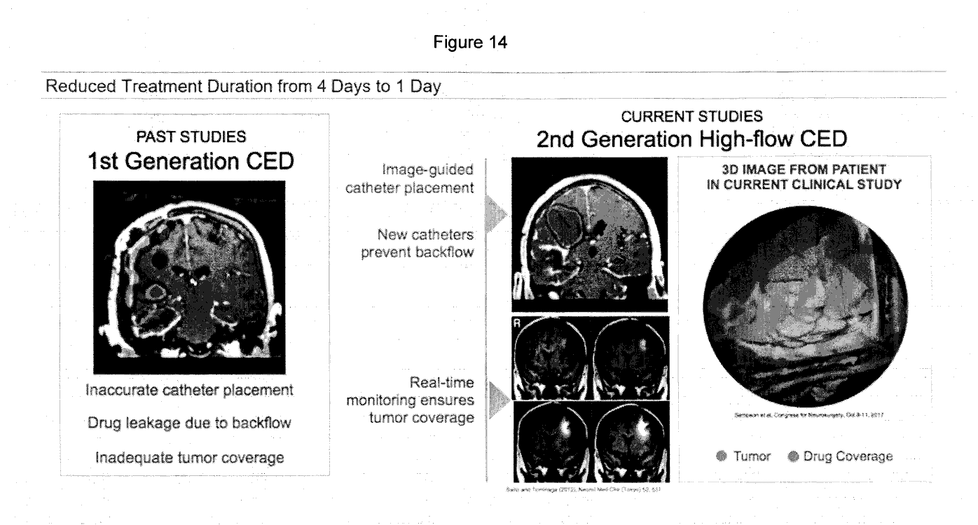

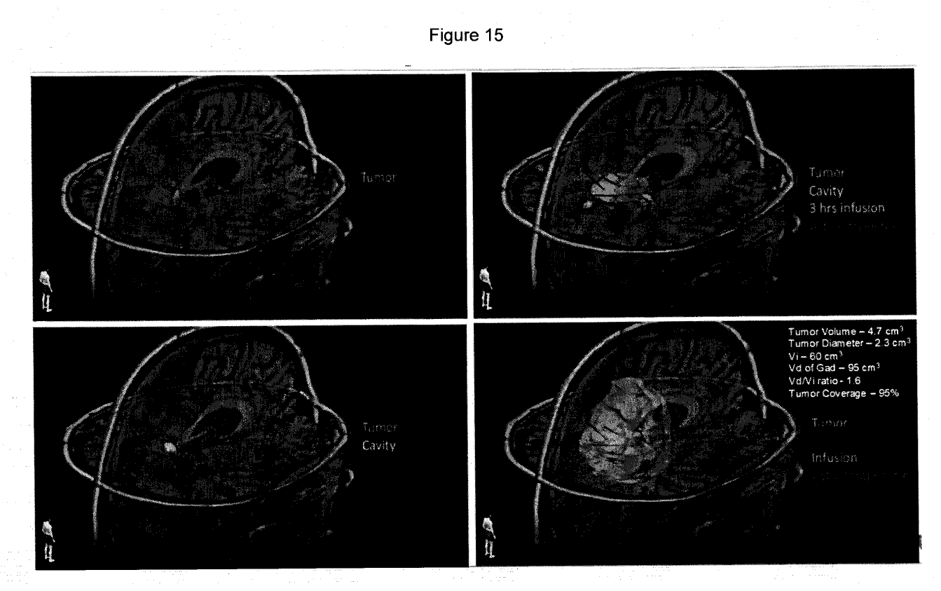

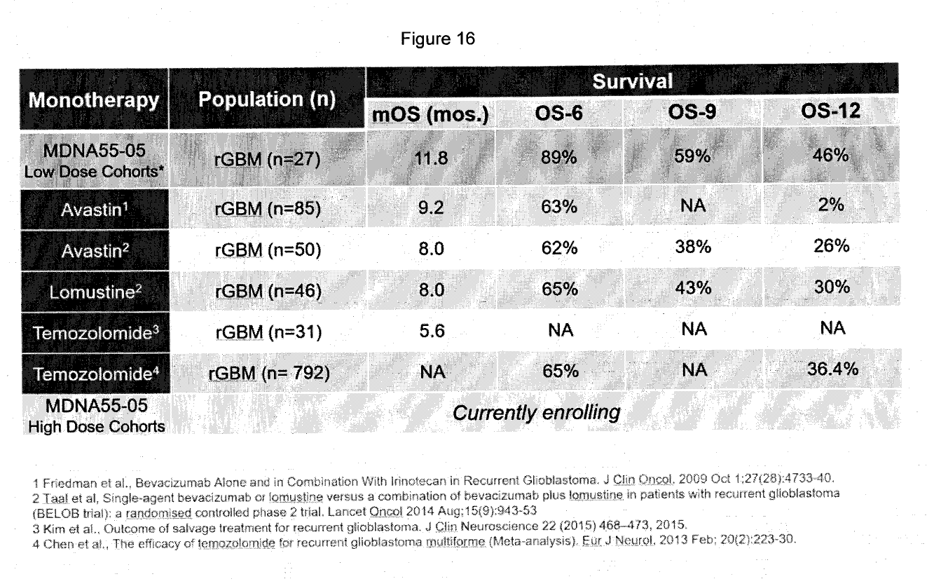

In some embodiments, any of the IL-4 muteins described herein can be used in combination with an anti-PD-1 antibody. There are three approved anti-PD-L1 antibodies, atezolizumab (TECENTRIQ®; MPDL3280A), avelumab (BAVENCIO®; MS0001071 8C), and Durvalumab (MEDI4736), as well as other anti-PD-L1 antibodies in development. Numerous anti-PD-L1 antibodies are available and many more in development which can be used in combination with the IL-4 muteins as described herein. In some embodiments, the PD-L1 antibody is one described in U.S. Patent Publication No. 2017/0281764 as well as International Patent Publication No. WO 2013/079174 (avelumab) and WO 2010/077634 (or U.S. Patent Application No. 20160222117 or U.S. Pat. No. 8,217,149; atezolizumab). In some embodiments, the PD-L1 antibody comprises a heavy chain sequence of SEQ ID NO:34 and a light chain sequence of SEQ ID NO:36 (from US 2017/281764). In some embodiments, the PD-L1 antibody is atezolizumab (TECENTRIQ®; MPDL3280A; IMpower110). In some embodiments, the PD-L1 antibody is avelumab (BAVENCIO®; MSB001071 8C). In some embodiments, the PD-L1 antibody is durvalumab (MEDI4736). In some embodiments, the PD-L1 antibody includes, for example, Atezolizumab (IMpower133), BMS-936559/MDX-1105, and/or RG-7446/MPDL3280A, and/or YW243.55.S70, as well as any of the exemplary anit-PD-L1 antibodies provided herein in FIG. 11.

Anti-PD-1 antibodies for use in combination with the IL-4 muteins disclosed herein for the treatment methods include but are not limited to nivolumab, BMS-936558, MDX-1106, ONO-4538, AMP224, CT-011, and MK-3475.

L. Other Immunotherapy Combinations

Other antibodies and/or immunotherapies for use according to the methods of the present invention include but are not limited to, anti-CTLA4 mAbs, such as ipilimumab, tremelimumab; anti-PD-L1 antagonistic antibodies such as BMS-936559/MDX-1105, MED14736, RG-7446/MPDL3280A; anti-LAG-3 such as IMP-321; agonistic antibodies targeting immunostimulatory proteins, including anti-CD40 mAbs such as CP-870,893, lucatumumab, dacetuzumab; anti-CD137 mAbs (anti-4-1-BB antibodies) such as BMS-663513 urelumab (anti-4-1BB antibody; see, for example, U.S. Pat. Nos. 7,288,638 and 8,962,804, incorporated by reference herein in their entireties); lirilumab (anti-KIR mAB; IPH2102/BMS-986015; blocks NK cell inhibitory receptors) and PF-05082566 (utomilumab; see, for example, U.S. Pat. Nos. 8,821,867; 8,337,850; and 9,468,678, as well as International Patent Application Publication No. WO 2012/032433, incorporated by reference herein in their entireties); anti-OX40 mAbs (see, for example, WO 2006/029879 or WO 2010/096418, incorporated by reference herein in their entireties); anti-GITR mAbs such as TRX518 (see, for example, U.S. Pat. No. 7,812,135, incorporated by reference herein in its entirety); anti-CD27 mAbs, such as varlilumab CDX-1127 (see, for example, WO 2016/145085 and U.S. Patent Publication Nos. US 2011/0274685 and US 2012/0213771, incorporated by reference herein in their entireties) anti-ICOS mAbs (for example, MEDI-570, JTX-2011, and anti-TIM-3 antibodies (see, for example, WO 2013/006490 or U.S. Patent Publication No US 2016/0257758, incorporated by reference herein in their entireties).

Other antibodies can also include monoclonal antibodies to prostate cancer, ovarian cancer, breast cancer, endometrial cancer, multiple myeloma, melanoma, lymphomas, lung cancers including small cell lung cancer, kidney cancer, colorectal cancer, pancreatic cancer, gastric cancer, brain cancer (see, generally www.clinicaltrials.gov).

M. Antibodies can Also Include Antibodies for Antibody-Dependent Cell-Mediated Cytotoxicity (ADCC). Methods of Treatment

In some embodiments, subject IL-4 muteins, and/or nucleic acids expressing them, can be administered to a subject to treat a disorder associated with abnormal apoptosis or a differentiative process (e.g., cellular proliferative disorders or cellular differentiative disorders, such as cancer, by, for example, producing an active or passive immunity). In the treatment of such diseases, the disclosed IL-4 muteins may possess advantageous properties, such as reduced vascular leak syndrome. In some embodiments, the IL-4 mutein is any IL-4 mutein or variant disclosed herein. In some embodiments, the IL-4 mutein sequence is 90% identical to any one of the sequences disclosed herein.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate cancer, ovarian cancer, breast cancer, endometrial cancer, multiple myeloma, melanoma, lymphomas, lung cancers including small cell lung cancer, kidney cancer, liver cancer, colon cancer, colorectal cancer, pancreatic cancer, gastric cancer, and brain cancer.

The mutant IL-4 polypeptides can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues.

Additional examples of proliferative disorders include hematopoietic neoplastic disorders.

### EXAMPLES

### Example 1: IL-4Ra Assay and Data

### Materials and Methods

Immunohistochemical (IHC) staining for IL-4Rα uses a rabbit polyclonal antibody from Abcam (Cat #ab203398) (Table 2) capable of detecting the alpha chain of the interleukin 4 receptor protein (IL-4Rα) in formalin-fixed, paraffin-embedded (FFPE) specimens. There are four steps to the IHC procedure as briefly outlined below.

Step 1: FFPE tissue blocks are cut at 4-5 μm thickness and sections are mounted onto positively-charged, capillary gap glass slides. Slides are baked (60° C., dry heat) prior to use.

Step 2: Tissue sections are de-waxed using organic solvents (100% xylene, four changes) and an alcohol series (100%, 70%, 30% ethanol) descending to distilled water to sufficiently hydrate the tissue and allow proper binding of the primary antibody and other detection reagents. When needed, antigen retrieval is performed after de-waxing using SHIER (steam heat induced epitope recovery) solutions and heat. For IL-4Rα, antigen retrieval with heat and SHIER solutions is not used (referred to as NO SHIER) as it does not contribute to antigen unmasking and is not needed to expose the epitope for binding.

Step 3: Samples are paired with blank microscope slides to form capillary gaps and are tested by IHC according to the general procedure outlined in Table 1 using the TechMate instrumentation platform and the MIP program (which does not include enzymatic digestion with Proteinase K). Sequential detection of antibodies is employed during IHC with a high level of specificity for the antigen or for the primary antibody. The location of the primary antibody is ultimately visualized by the application of a colorimetric chromogen (DAB) that precipitates a discrete insoluble reaction product at the site of antigen in the presence horseradish peroxidase (HRP). Nuclei are counterstained using hematoxylin (blue stain) to assess cell and tissue morphology.

Step 4: Slides are unpaired, rinsed in distilled water, dehydrated in an alcohol series (70%, 95%, 100% ethanol) and in organic solvent (100% xylene, four changes), then permanently coverslipped, using CytoSeal (or equivalent), for interpretation and storage. Slides are examined under a microscope to assess staining.

After de-waxing, the process steps are automated using a TechMate Instrument (Roche Diagnostics) running QML workmate software v3.96. This automated platform uses a capillary gap process for all reagent changes, up to and including counterstaining, and intervening buffer washes. All steps are carried out at room temperature (RT), which is 25° C.

Reagent Manufacturing Buffer (RMB) with Goat Serum is used to prepare working dilutions of IL-4Rα primary antibody, species-match positive controls (Rabbit CD3), and isotype-match negative controls (Rabbit IgG). Target recognition for IL-4Rα at the site of antigen-primary antibody interaction in FFPE sections uses detection reagents from Polink-2 Plus HRP kits from GBI Labs designed for detection of rabbit primary antibodies. Refer to Table 2 for antibody specifications and IHC assay conditions.

### Control Cell Lines/Tissues and Internal Process Controls

Positive controls for IL-4Rα expression included a previously-tested formalin-fixed, paraffin embedded (FFPE) SW480 cell line and a human glioblastoma multiforme (GBM) tissue (Q1360-3) from the QualTek tissue bank. These samples showed high expression of IL-4Rα when tested in QML Project #1397 and as such were included as positive controls in the current study (QML Project #1421).

Negative controls for IL-4Rα expression included a previously-tested FFPE HUVEC cell line (P1397Q0001) and a normal human cortex tissue (Q2908) from the QualTek tissue bank. These samples were nonreactive or showed very low IL-4Rα staining when tested and as such were included as negative controls in the current study.

Species-match positive controls (standard antibodies) with established signal strength in control tissues were used in each run to confirm proper detection reagent performance. The standard positive control used throughout the duration of this project was CD3 (derived in Rabbit) run on FFPE control tonsil tissue from the QualTek tissue bank.

A Rabbit IgG isotype-match negative control was used to determine any non-specific staining inherent in the detection reagents or tissues to help define any possible background reactivity from these sources. For the IL-4Rα assay, no pre-treatment of tissues was needed. In this study, the negative controls were also useful in differentiating pigment from DAB staining. Representative images of Rabbit IgG isotype-match negative control staining can be observed in figures throughout the Results section of this report.

### Human Test Tissues

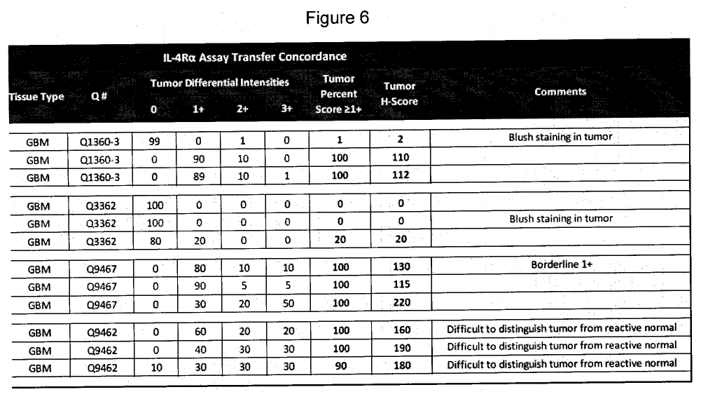

For sensitivity testing, QualTek screened 42 formalin-fixed, paraffin embedded (FFPE) samples for IL-4Rα expression. All tissues were human specimens. One sample (1713-rgbm, 05220643C ##S) could not be confirmed as glioblastoma multiforme (GBM). As such, it was not included in the summary data tables. A total of 41 samples were determined to be evaluable GBM specimens. A general description of these samples is provided in FIG. 6. Detailed information on each GBM sample is included in the sensitivity scoring tables in the Results section. A subset of these samples (Q1360-3, Q3362, Q9467, Q9462, Q9464, Q1351-5, Q1348-1) was used for assay transfer and/or validation testing.

For IL-4Rα specificity testing, FDA multi-normal human tissue microarray (TMA) slides were purchase from Pantomics, Inc (Cat #MN0961). The TMA (designated as P1421Q0001) contains 96 different samples derived from 35 different organs or sites.

### Scoring Scheme

IL-4Rα is reactive in the cytoplasm of tumor cells. However, it can also be observed in serum and occasionally in the cytoplasm of normal cells and normal tissue components. The guidelines used for scoring cytoplasmic IL-4Rα staining observed in tumor cells and observed as interstitial background in benign tissue by IHC in formalin-fixed, paraffin-embedded (FFPE) glioblastoma multiforme (GBM) samples are as follows:
IL-4Rα expression is scored by a board-certified pathologist. The main components to scoring malignant tumor cells include Percent Scores and an H-Scores (derived from percentages that are recorded at differential intensities) as described below.
As best as possible, only obviously malignant tumor cells (large pleomorphic cells) are scored for IL-4Rα positivity using the Percent Score and H-Score methods. That is, any IL-4Rα staining observed in cells that are clearly non-neoplastic is excluded. Morphologically, the distinction between neoplastic and non-neoplastic cells in GBM samples can be difficult to make.
Malignant cells are considered to express IL-4Rα if cytoplasmic tumor cell staining is recognized.
When present, cytoplasmic background staining observed in benign tissue around tumor cells is captured as an average intensity of interstitial signal as described herein.
To gain a full understanding of IL-4Rα expression across GBM samples and to help stratify the patient population, both standard Percent Score and H-Score approaches were used to capture the pattern of staining observed in obviously malignant tumor cells. These methods are described as follows.

### Percent Score Method

Percent Scores are calculated by summing the percentages of intensities in tumor cells at either ≥1+, ≥2+ or ≥3+. Thus, scores range from 0 to 100.
Percent Score ≥1+=(% at 1+)+(% at 2+)+(% at 3+)
Percent Score ≥2+=(% at 2+)+(% at 3+)
Percent Score ≥3+=(% at 3+)

### H-Score Method

The H-Score is calculated by summing the percentage of tumor cells with intensity of expression (brown staining) multiplied by their corresponding intensity a four-point semi-quantitative scale (0, 1+, 2+, 3+). Thus, scores range from 0 to 300.

H-Score=[(% at <1)×0]+[(% at 1+)×1]+[(% at 2+)×2]+[(% at 3+)×3]

For both the Percent Score and H-Score methods, the four-point semi-quantitative intensity scale is described as follows: 0—null, negative or non-specific staining, 1+—low or weak staining, 2+—medium or moderate staining, and 3+—high or strong staining. The percentage at each intensity is estimated directly and typically reported as one of the following, though other increments can also be used: 0, 1, 2, 3, 4, 5, 10, 15, 20, 25, 30, 35, 40, 45, 50, 55, 60, 65, 70, 75, 80, 85, 90, 95, 96, 97, 98, 99, or 100%.

Occasionally, GBM samples included background IL-4Rα staining throughout benign tissue. When present, such interstitial staining was captured with an average intensity score of 1+, 2+, or 3+ to record the level of background cytoplasmic staining present around tumor cells. When absent, this value was recorded as NA (not applicable). High background reactivity could contribute to higher IL-4Rα expression in tumor cells. As such, the interstitial staining score should be taken into consideration when evaluating reactivity scores for malignant tumor cells.

### Results

### Assay Transfer to CLIA Laboratory

Formalin-fixed, paraffin-embedded (FFPE) glioblastoma multiforme (GBM) samples from the QualTek tissue bank were used for CAP/CLIA concordance testing between laboratories. Four samples were chosen (Q1360-3, Q3362, Q9467, Q9462) to represent a range of IL-4Rα staining (low, moderate, and high). This sample set was stained using the IL-4Rα IHC assay as part of the initial precision and reproducibility testing that included replicate testing by multiple operators.

Slides were scored using the H-Score and Percent Score approaches that recorded the percentage of tumor cells staining at differential intensities (0-3+). This scoring was performed before the large sensitivity set of GBM tumors was reviewed and before the final scoring scheme for the GBM indication.

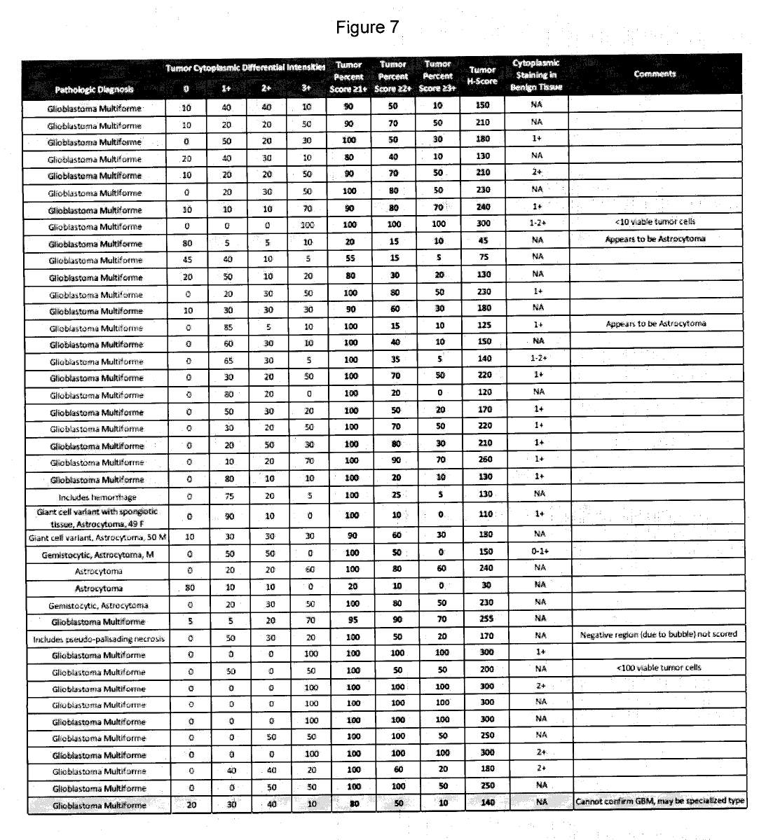

Scoring data comparing the GBM samples stained at QML-Newtown (non-CLIA, GLP) and QML-Santa Barbara (CAP/CLIA) are shown in FIG. 7. This table includes H-Scores and Percent Scores ≥1+ intensity for GBM tumor cells.

### CAP/CLIA Sensitivity Screening in Glioblastoma

A total of 42 formalin-fixed, paraffin-embedded (FFPE) glioblastoma multiforme (GBM) samples were screened for IL-4Rα sensitivity using the IHC assay described above. One sample could not be confirmed as glioblastoma multiforme (GBM). As such, a total of 41 samples were determined to be evaluable GBM specimens.

Sensitivity screening was performed to understand the range of staining intensities and the pattern of IL-4Rα reactivity across a representative sample set from the GBM indication. All samples were stained with H&E for morphological evaluation and were tested by IHC with the Rabbit IgG isotype-match negative control corresponding to the IL-4Rα assay conditions.

This scoring schemes described above were used to evaluate the 42 samples in the current CAP/CLIA IL-4Rα sensitivity screen. The scoring approach also included semi-quantitative Percent Scores [calculated by summing the percentages of intensities ≥1+, ≥2+, and >3+ with values ranging from 0 to 100] and H-Scores [calculated as the sum of each percentage score (0-100%) multiplied by its corresponding intensity score (0, 1+, 2+, 3+) with values ranging from 0 to 300]. Full scoring results for IL-4Rα reactivity in the panel of 42 tissues is presented in FIG. 7.

Representative images of various levels of IL-4Rα reactivity in the GBM indication are shown in FIG. 1. This figure also includes a corresponding representative Rabbit IgG negative control for the biomarker assay. All Rabbit IgG isotype negative controls were nonreactive across the sensitivity panel of GBM tumors tested.

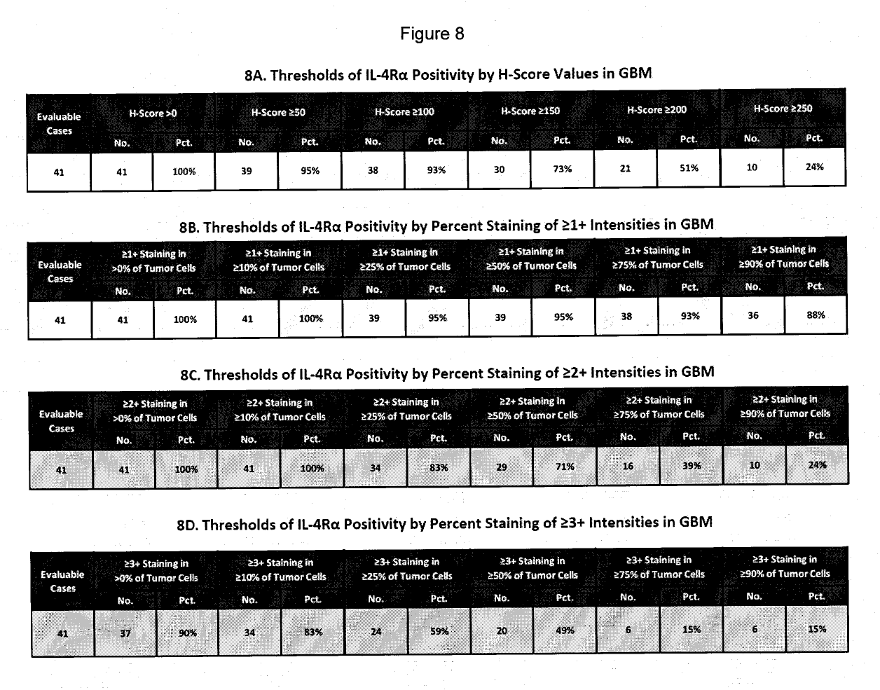

Summarized results of IL-4Rα expression in the 41 evaluable GBM samples grouped by potential reactivity thresholds are presented in FIG. 8A top panel, for H-Score Values, FIG. 8B second panel for Percent Staining of ≥1+ Intensities, FIG. 8C third panel for Percent Staining of ≥2+ Intensities, and FIG. 8D fourth panel for Percent Staining of ≥3+ Intensities. These tables are intended to divide the data based on theoretical thresholds of positivity to help stratify the patient population.

Most of the GBM samples tested showed moderate to high IL-4Rα cytoplasmic reactivity in tumor cells. According to H-Score analysis (Table 6), H-Scores ≥50 were observed in 95% of GBM cases (39/41), H-Scores ≥200 were observed in 51% of GBM cases (21/41), and H-Scores ≥250 were observed in 24% of GBM cases (10/41). Additional breakdowns by H-Score are included Table 6 and other thresholds could also be considered.

According to Percent Score analysis, 95% of GBM cases had ≥1+ intensity in ≥50% of tumor cells (39/41) (Table 7), 71% of GBM cases had ≥2+ intensity in ≥50% of tumor cells (29/41) (Table 8), and 49% of GBM cases had ≥3+ intensity in 50% of tumor cells (20/41) (FIG. 8D). Additional breakdowns by Percent Score are included in FIGS. 8B-8D) and other thresholds could also be considered.

The potential thresholds of IL-4Rα reactivity presented in this example aided in the determination of an appropriate cut-off for IL-4Rα positivity in GBM for clinical testing.

### CAP/CLIA Precision & Reproducibility in Glioblastoma (Part C)

The results from the sensitivity screen helped identify appropriate tissues for testing the precision and reproducibility of the IL-4Rα IHC assay in the GBM. For this validation, 4 tumor samples showing high (Q9464), moderate (Q1351-5), and low (Q1348-1, Q3362) expression of IL-4Rα were selected for use.

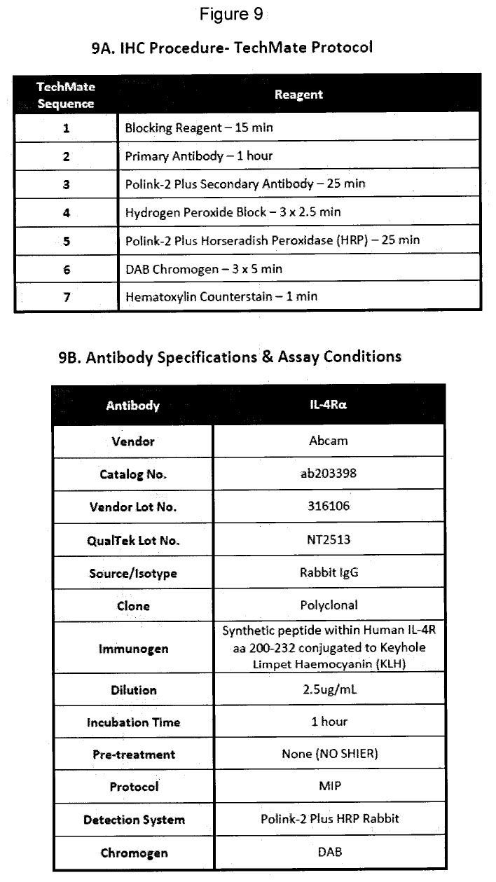

Each GBM sample selected was run in triplicate according to the IHC assay in FIG. 9 in one run for IL-4Rα (precision). In two separate runs, performed on non-consecutive days, the same samples were run in triplicate with IL-4Rα. Appropriate positive and negative controls were included in each staining run. (See, for example, FIG. 10.)

The validation samples were scored using the Percent Score approach for IL-4Rα in which differential intensity staining was determined. For the purposes of this precision and reproducibility testing in GBM, a cut-off for IL-4Rα positivity of ≥2+ staining intensity in ≥25% of tumor cells was used. That is, a sample was called positive (POS) if it showed staining of 2+ or 3+ intensity in 25% or more tumor cells. A sample was called negative (NEG) if staining was absent, present in fewer than 25% of tumor cells, or only present at an intensity of 1+.

Each replicate showed consistent and reproducible cytoplasmic IL-4Rα staining for each GBM tissue (data not shown). Images of corresponding Rabbit IgG negative controls (nonreactive) were included (data not shown).

Acceptance/rejection of validation for the IHC assay for IL-4Rα was determined through evaluation of consistency in staining patterns, statistical analysis of semi-quantitative scores, and the percent of agreement/concordant estimates.

Full validation scoring results (FIG. 10) reflect the equivalent cellular patterns of IL-4Rα reactivity observed in all GBM replicates.

The reference point used for a positive determination for IL-4Rα was the cut-off of ≥2+ staining in ≥25% of tumor used for this Example.

### Specificity Testing in Normal Tissues

The specificity of IL-4Rα to its target was tested in the IHC method described using an FDA-recommended tissue microarray (TMA) of multi-normal human tissues. The TMA used for specificity testing (designated P1421Q0001) was purchased from Pantomics, Inc (Cat #MN0961, multi-normal human tissues, FDA, 96 samples, 35 organs/sites from 3 individuals, 1.5 mm). Sections of the TMA were histology-stained with hematoxylin and eosin (H&E) and IHC-stained with IL-4Rα and Rabbit IgG.

All stained slides of the multi-normal TMA were assessed using the Percent Score and H-Score methods described above to evaluate normal tissue components (as opposed to tumor). Scoring data from this specificity testing includes differential intensities, H-Scores, and total percentage of cells with IL-4Rα staining ≥1+(FIG. 11A-11B). A panel of representative images showing IL-4Rα and Rabbit IgG (corresponding isotype control) staining in normal human tissues was also obtained (data not shown).

Overall, IL-4Rα showed negative (nonreactive) or low staining in normal human tissue components. When present, staining was generally at background levels with 1+ intensity (H-Scores ≤50). Low staining was observed in ducts in normal breast and in normal esophagus (including muscle). Some elevated IL-4Rα staining that included reactivity with 2+ intensity (H-Scores ≥90) was observed in some normal tissue components such as cardiac muscle in heart, hepatocytes in liver, and acinar cells in stomach. Such staining could reflect basal levels of IL-4Rα reactivity. A Rabbit IgG isotype-match negative control was run on the multi-normal TMA and was nonreactive in all normal human tissues tested. (Data not shown.)

This specificity testing indicates that the IL-4Rα antibody and IHC assay is predominantly specific to targets in tumor cells at thresholds above background. IL-4Rα can occasionally be reactive, generally at low levels, in normal and neoplastic tissue components.

### Example 2: The IL-4 Receptor as a Biomarker and Immunotherapeutic Target for Glioblastoma: Preliminary Evidence with MDNA55, a Locally Administered IL-4 Guided Toxin

This example describes the use of the IL-4 receptor (IL-4R) as biomarker for treatment with an IL-4 guided toxin. The IL-4 guided toxin comprises a tumor targeting domain comprising circularly permuted interleukin-4 (cpIL-4) and a tumor killing “cytotoxic” domain comprising the catalytic domain of Pseudomonas Exotoxin A (PE); this construct is also referred to as MDNA55.

This Example further describes a study in 52 Recurrent GBM Patients. The study is laid out in four phases:
Diagnosis: Retrospective IL-4R expression analysis; GBM at 1st or 2nd relapse; KPS≥70.
Planning: MRI—tumor size and location; Optimal catheter trajectory.
Treatment: Image-guided catheter placement; Real-time monitoring of MDNA55 distribution; Low dose and high dose cohorts.
Follow-up: Patient safety; Survival; Tumor response; Quality of life; Correlation of efficacy with IL-4R expression.

No deaths attributed to MDNA55 in current study. No systemic toxicity following doses of 18-180 μg. No clinically significant laboratory abnormalities. Drug-related adverse events were primarily neurological/aggravation of pre-existing neurological deficits characteristic with GBM and had generally been manageable with standard measures. No evidence of a differential rate of neurological toxicities between the two concentrations of MDNA55 used previously in this study (i.e. 1.5 μg/mL×60 mL vs. 3.0 μg/mL×60 mL) and a range of higher concentrations explored in previous studies (up to 15 μg/mL). Archived tissue obtained at first diagnosis of GBM is analyzed for IL-4Rα expression by immunohistochemistry (IHC) as described in Example 1 and in further detail below.

### Introduction

MDNA55 is being developed for the treatment of recurrent/progressive glioblastoma multiforme (GBM). Using current treatment paradigms, most GBM patients experience tumor recurrence/progression after standard first line treatment. Treatment options for patients with recurrent GBM are very limited and the outcome is generally unsatisfactory. Specifically, chemotherapy regimens for recurrent or progressive GBM have been unsuccessful, producing toxicity without benefit (Weller et al., 2013). This is mainly due to the lack of tissue specificity with resultant toxicity to normal tissues and consequently, a narrow therapeutic index. As overall survival remains dismal, novel anti-cancer modalities, with greater tumor specificity, more robust cytotoxic mechanisms and novel delivery techniques are needed for the treatment of recurrent GBM.

MDNA55 is one such novel therapeutic that provides a targeted treatment approach whereby tumor cells are more sensitive to the toxic effects of the drug than normal cells. The target, IL-4R, is an ideal but under-exploited target for the development of cancer therapeutics, as it is frequently and intensely expressed on a wide variety of human carcinomas. Expression levels of IL-4R are low on the surface of healthy and normal cells, but increase several-fold on cancer cells. A majority of cancer biopsy and autopsy samples from adult and pediatric central nervous system (CNS) tumors, including recurrent GBM biopsies, have been shown to over-express the IL-4R. There is little or no IL-4R expression in normal adult and pediatric brain tissue (Joshi, et al., 2001; Table 1).

This differential expression of the IL-4R provides MDNA55 a wide therapeutic window. This feature alone makes MDNA55 an ideal candidate for the treatment of recurrent GB and other CNS tumors that over-express the IL-4R. Cells that do not express the IL-4R target do not bind to MDNA55 and are, therefore, not subject to PE-mediated effects.

TABLE 13
Summary of IL-4R Expression in Adult and Pediatric Brain Tumors and Normal Brain

| | | | | |
|---|---|---|---|---|
| | | Biopsy | Results | |
| Cancer Type | Method | Samples (N) | (% Positive) | Reference |

BRAIN TUMORS

| | | | | |
|---|---|---|---|---|
| Adult Recurrent GB | IHC | 25 | 96% | Merchant et al |
| | | | | (unpublished data) |
| Newly Diagnosed GB/ | IHC | 13/14 | 93%/93% | Merchant et al |
| Recurrent GB | | | | (unpublished data) |
| (Matched Pairs) | | | | |
| Mixed Adult Glioma | IHC | 32 | 83% (GBM) | Joshi et al, 2001 |
| | | | 86% (Astrocytoma) | |
| Adult GBM | RT-PCR | 21 | 76% | Puri et al, 1996 |
| Mixed Pediatric Glioma | IHC | 58 | 76% | Kawakami et al, 2004 |
| Pediatric DIPG | IHC | 17 | 71% | Berlow et al, 2018 |
| Medulloblastoma | RT-PCR | 5 | 100% | Joshi et al, 2002 |
| Adult Pituitary Adenoma | RT-PCR | 30 | 100% | Chen et al, 2007 |
| Meningioma | RT-PCR | 35 | 77% | Puri et al, 2005 |

NORMAL BRAIN

| | | | | |
|---|---|---|---|---|
| Normal Brain Tissue | IHC | 3 | 0% | Joshi et al, 2001 |
| Normal Brain Tissue | RT-PCR | 6 | 0% | Puri et al, 1996a |

### IL-4R Analysis in MDNA55 Clinical Trial

As part of the biomarker evaluation in the MDNA55 Phase 2b clinical study, a retrospective analysis of IL4 receptor (IL-4R) expression from archived tissue obtained at first diagnosis of GBM is being conducted to determine the role of the IL-4R biomarker on treatment response and patient outcomes, and to help guide potential patient selection strategies for future clinical studies. To this end, an immunohistochemistry (IHC)-based assay has been developed to detect IL-4Rα biomarker expression on archived excised tumor tissue/biopsy samples. The assay has been validated for use in a single site CLIA-compliant reference laboratory (QualTek Molecular Laboratories, Santa Barbara, Calif.). See, also Example 1.

The assay uses a rabbit polyclonal antibody to IL-4Rα (Abcam, ab203398). Range and linearity testing of the antibody was evaluated using serial dilutions of anti-IL-4Rα to stain GBM tissue and normal cortex (negative control). Six different antibody concentrations were tested to determine the optimal concentration for use in the assay. Specificity and sensitivity testing were performed using a panel of normal human tissues as well as cases of GBM from tissue banks.

Based on the validation testing, a scoring approach was determined: each GBM sample was scored for IL-4Rα expression using the H-Score method calculated by summing the percentage of tumor cells with intensity of expression (brown staining by 3,3′-Diaminobenzidine, DAB) multiplied by their corresponding intensity on a four-point semi-quantitative scale (0, 1+, 2+, 3+):

GBM samples are scored for cytoplasmic IL-4Rα reactivity using the H-Score method: H-Score=[(% at <1)×0]+[(% at 1+)×1]+[(% at 2+)×2]+[(% at 3+)×3]. Using this approach, H-Scores ranged from 0 to 300 and the level of IL-4R expression were categorized as follows:
H-Scores from 0 to 75=no to low expression
H-Scores from 76 to 150=moderate expression
H-Scores from 151 to 225=high expression
H-Scores from 226 to 300=very high expression

IL-4R Negative=H-Scores ≤75; IL-4R Positive=H-Scores >75

FIG. 10 presents representative images of various levels of IL-4R expression in GBM tissues.

In the current study, comparison of baseline parameters between subjects with moderate to high H-Scores (H-Scores >75) as compared to subjects with low H-Scores (H-Score ≤75) were generally well-matched except that moderate/high H-scores were associated with shorter time to relapse (FIG. 17).

As IL-4R expression has been associated with more aggressive disease and poor clinical outcomes (Kohanbash, et al., 2013; Han and Puri, 2018), the shorter time to relapse from initial diagnosis seen in subjects with moderate/high H-Scores in our study is consistent with published findings.

### Overall Survival by IL-4R Status

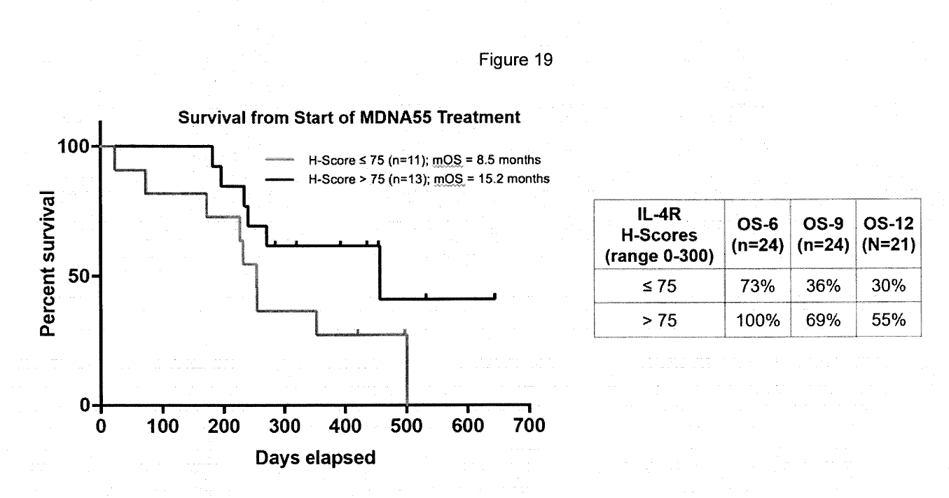

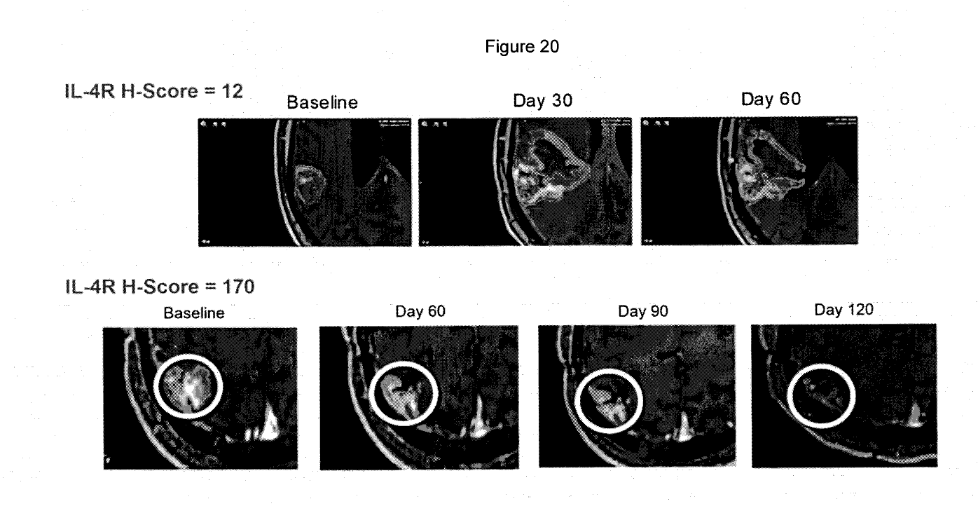

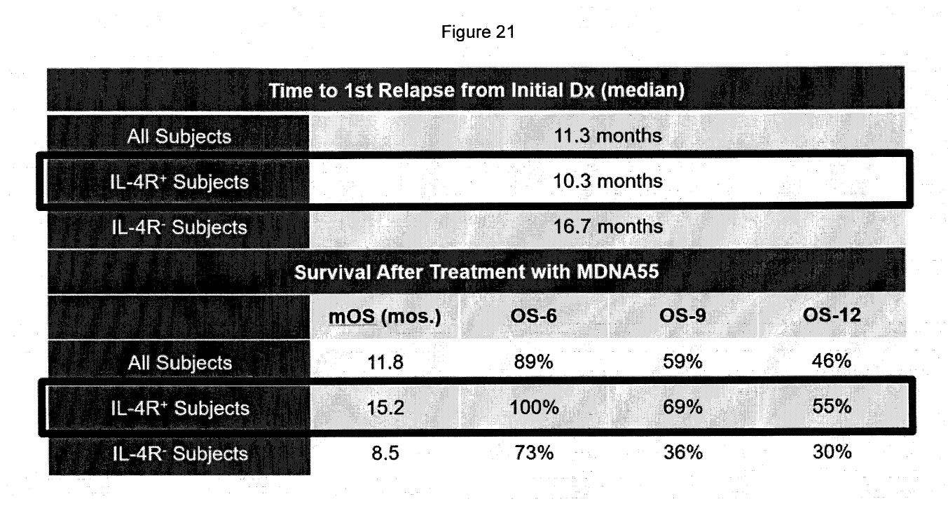

When assessing IL-4R H-Score with overall survival, subjects with moderate/high H-Scores (H-Scores >75) show a trend for longer survival (mOS=15.2 months) than subjects with low H-Scores (H-Score ≤75; mOS=8.5 months) (FIG. 19). Survival rates at 6, 9, and 12 months were also greater for subjects with moderate to high H-Scores as opposed to those with low H-Scores (Table 14).

TABLE 14
Survival Rates of Subjects in Current
Study According to IL-4R H-Score

| IL-4R H-Scores | mOS | OS-6 | OS-9 | OS-12 |
|---|---|---|---|---|
| (range 0-300) | (n = 24) | (n = 24) | (n = 24) | (N = 21) |
| 0-75 | 8.5 mos | 73% | 36% | 30% |
| >75 | 15.2 mos | 100% | 69% | 55% |

### Progression-Free Survival by IL4-R Status

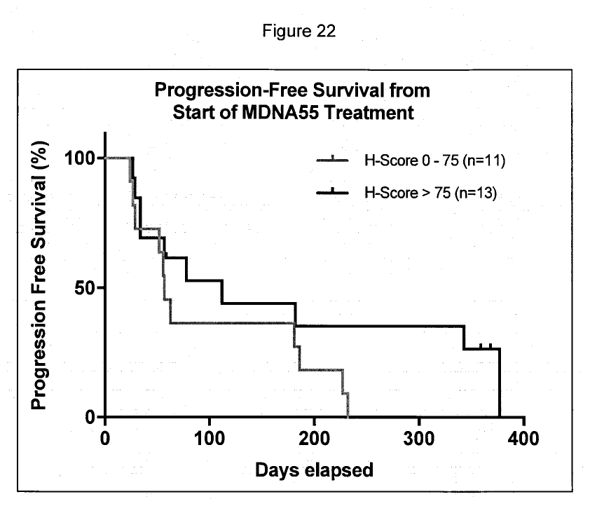

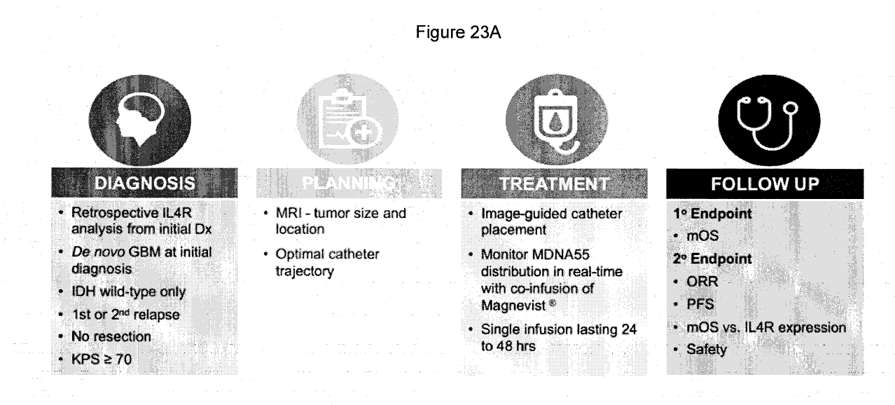

When assessing IL-4R H-Score with PFS, subjects with moderate/high H-Scores show a trend for longer PFS (mPFS=3.7 months) than those with low H-Scores (mPFS=1.9 months) (FIG. 22). PFS rates at 6, 9, and 12 months were also greater for subjects with moderate to high H-Scores (Table 15).

TABLE 15
PFS Rates of Subjects in Current
Study According to IL-4R H-Score

| IL-4R H-Scores | mPFS | PFS-6 | PFS-9 | PFS-12 |
|---|---|---|---|---|
| (range 0-300) | (n = 24) | (n = 24) | (n = 24) | (n = 21) |
| 0-75 | 1.9 mos | 36% | 0% | 0% |
| | | (n = 11) | (n = 11) | (n = 10) |
| >75 | 3.7 mos | 44% | 35% | 27% |
| | | (n = 13) | (n = 13) | (n = 11) |

### Summary & Conclusions

Treatment options for patients with recurrent GBM are very limited and positive outcomes remain very rare. IL-4R is frequently and intensely expressed on a variety of human carcinomas, including GBM, and is associated with aggressive disease and poor survival outcomes. MDNA55 is a novel IL-4R targeted fusion toxin, administered intratumorally via MRI-guided convection enhanced delivery as a single treatment for recurrent GBM. There is early evidence of clinical benefit at low doses of MDNA55, especially in IL-4R positive subjects who show impressive survival outcomes following MDNA55 treatment. As an initial approach, H-score cut-off points were determined pragmatically and may require adjustments to best correlate with therapeutic benefit from MDNA55 treatment as the treatment dataset extends and matures. A such, IL-4R may serve as a rational biomarker and immunotherapeutic target for recurrent GBM.

It is potently toxic to tumor cells, simultaneously purges the tumor microenvironment (TME), and bypasses the BBB via convection enhanced delivery (CED).

>200 Patient Biopsies Analyzed Show IL-4R Over-Expression (Joshi B H, et. al. Cancer Res 2001; 61:8058-8061; Puri R K, et. al., Cancer Res 1996; 56:5631-5637; Kawakami M, et. al., Cancer. 2004 Sep. 1; 101(5):1036-42; Berlow N E, et al. PLoS One. 2018 Apr. 5; 13(4):e0193565; Joshi B H, et. al. British J of Cancer (2002) 86, 285-291; Chen L, et al. Neurosci Lett. 2007 Apr. 24; 417 (1):30-5; and Puri S, et. al., Cancer. 2005 May 15; 103ζ 10):2132-42.) 76% of Glioblastoma; >83% of Mixed Adult Glioma, 76% of Mixed Pediatric Glioma; 71% Pediatric DIPG; 100% of Medulloblastoma; 100% of Adult Pituitary Adenoma; 77% of meningioma; and 0% normal brain tissue.

The present of IL-4R predicts poor survival in GBM (glioblastoma), see, for example, Han J. and Puri R. J of Neuro-Oncology (2018) 136:463-47.

TME-infiltrating MDSCs express IL-4R and also predict poor survival in GBM, see, for example, Kamran N, et. al., (2017). Mol Ther 25:232-248 and Otvos B et. al., (2016). Stem Cells 34:2026-2039.

Current therapies for GBM and other IL-4R expressing tumors do not address key challenges, such as those provided below, and which are address the be IL-4 construct provided in the present example.

TABLE 16
Challenges

| Therapeutic Challenges | Rationale for MDNA55 |
|---|---|
| 55% of GBMs are Temodar- | MDNA55 targets Temodar-resistant |
| resistant 1 | tumors3 |
| Immunosuppressive tumor | IL-4R over-expressed in GBM and |
| microenvironment (TME) | its TME (Myeloid Derived |
| comprises 40% of GBM | Suppressor Cells) but not in |
| tumor mass ² | normal brain4 |
| Blood Brain Barrier (BBB) blocks | Delivery by CED infusion of |
| transport of therapeutic to tumor | MDNA55 by-passes the BBB |
| High doses are required due to | Precision delivery achieves high |
| BBB causing systemic toxicities | doses without systemic exposure |

1. Hegi M E (2005). N Engl J Med; 352(10): 997-1003.
² Kennedy B, et al (2013). J Oncol. Vo; 2013: 486912.
3Shimamura, et al. (2007. Cancer Res; 67: 9903-9912.
4Kohanbash et al (2013). Cancer Res.; 73(21): 6413-23

Treatment options for patients with recurrent GBM are very limited and positive outcomes remain very rare.

IL-4R is frequently and intensely expressed on a variety of human carcinomas, including GBM, and is associated with aggressive disease and poor survival outcomes.

MDNA55 is a novel IL-4R targeted fusion toxin, administered intratumorally via MRI-guided convection enhanced delivery as a single treatment for recurrent GBM.

There is early evidence of clinical benefit and improved survival at low doses of MDNA55.

IL-4R+ subjects show impressive survival outcomes following MDNA55 treatment.

IL-4R may serve as a biomarker and immunotherapeutic target for recurrent GBM.

### REFERENCES

Berlow N E, Svalina M N, Quist M J, Settelmeyer T P, Zherebitskiy V, Kogiso M, Qi L, Du Y, Hawkins C E, Hulleman E, Li X N, Gultekin S H, Keller C. IL-13 receptors as possible therapeutic targets in diffuse intrinsic pontine glioma. PLoS One. 2018 Apr. 5; 13ζ 4):e0193565.
Chen L, Liu Y, Hou Y, Kato Y, Sano H, Kanno T. Expression and structure of interleukin 4 receptor (IL-4R) complex in human invasive pituitary adenomas. Neurosci Lett. 2007 Apr. 24; 417(1):30-5. Epub 2007 Mar. 2.
Han J and Puri R K. Analysis of the cancer genome atlas (TCGA) database identifies an inverse relationship between interleukin-13 receptor α1 and α2 gene expression and poor prognosis and drug resistance in subjects with glioblastoma multiforme. J Neurooncol. 2018 February; 136(3):463-474.
Joshi B H, Leland P, Asher A, Prayson R A, Varricchio F, Puri R K. In situ expression of interleukin-4 (IL-4) receptors in human brain tumors and cytotoxicity of a recombinant IL-4 cytotoxin in primary glioblastoma cell cultures. Cancer Res 2001; 61:8058-8061.
Joshi B H, Leland P, Silber J, Kreitman R J, Pastan I, Berger M, Puri R K. IL-4 receptors on human medulloblastoma tumours serve as a sensitive target for a circular permuted IL-4-Pseudomonas exotoxin fusion protein. British Journal of Cancer (2002) 86, 285-291.
Kawakami M, Kawakami K, Puri R K. Interleukin-4-Pseudomonas exotoxin chimeric fusion protein for malignant glioma therapy. J Neurooncol. 2003 October; 65(1):15-25.
Kohanbash G, McKaveney K, Sakaki M, Ueda R, Mintz A H, Amankulor N, Fujita M, Ohlfest J R, Okada H. G M-CSF promotes the immunosuppressive activity of glioma-infiltrating myeloid cells through interleukin-4 receptor-α. Cancer Res. 2013 Nov. 1; 73ζ 21):6413-23.
Lewis O, Woolley M, Johnson D, et al. Chronic, intermittent convection-enhanced delivery devices. J Neurosci Methods. 2016 Feb. 1; 259:47-56.
Puri R K, Hoon D S, Leland P, Snoy P, Rand R W, Pastan I, and Kreitman R J. Preclinical development of a recombinant toxin containing circularly permuted interleukin 4 and truncated Pseudomonas exotoxin for therapy of malignant astrocytoma. Cancer Res 1996; 56:5631-5637.
Puri S, Joshi B H, Sarkar C, Mahapatra A K, Hussain E, Sinha S. Expression and structure of interleukin 4 receptors in primary meningeal tumors. Cancer. 2005 May 15; 103(10):2132-42.
Vogelbaum M A, Brewer C, Barnett G H, et al. First-in-human evaluation of the Cleveland Multiport Catheter for convection-enhanced delivery of topotecan in recurrent high-grade glioma: results of pilot trial 1. J Neurosurg. 2018 Apr. 1:1-10.
Weller M, Cloughesy T, Perry J, Wick W. Standards of care for treatment of recurrent glioblastoma—are we there yet? Neuro-Oncology 15(1):4-27, 2013.

### Example 3: Clinical Data from Phase 2B Recurrent Glioblastoma Trial of MDNA55—Strong Interim Survival Trend (15.2 Vs 8.5 Months) Emerging in IL-4R Positive Patients

This example describes interim data from its on-going Phase 2b trial of MDNA55 for the treatment of recurrent glioblastoma (“rGBM”). MDNA55 is a fusion protein designed to target the Type II interleukin-4 receptor (consisting of the IL-4Rα and IL-13Rα1), a biomarker that is over-expressed in a majority of GBM patients but not in healthy brain. GBM patients with a positive Type 2 IL-4R profile typically have a worse prognosis than the overall glioblastoma population, including poor long term survival. Results demonstrate promising signs of clinical benefit, particularly in recurrent patients with an aggressive form of GBM.

MDNA55 provides impressive prolonged survival, especially in IL-4R positive tumors, a marker highly expressed on brain cancers and the tumor microenvironment and known to be associated with aggressive disease. Seeing evidence of clinical benefit in trial participants treated thus far with low doses of MDNA55 offers promise for patients with rGBM given the overall bleak prognosis and response to current therapy for this population.

### Summary of Results

Following treatment with MDNA55 at the low dose, the IL-4R positive group showed a remarkable increase in median overall survival (“mOS”) of 15.2 months when compared to 8.5 months in the IL-4R negative group. Survival rates at 6, 9, and 12 months were 100%, 67% and 55% versus 73%, 40%, and 30%, in the IL-4R positive and negative groups, respectively.

Irrespective of IL-4R expression, mOS was 11.8 months in all patients following a single treatment with MDNA55 at the low dose with an overall survival rate of 89% at 6 months, 59% at 9 months and 46% at 12 months, substantially exceeding landmark mOS and survival rates reported for approved drugs for rGBM (mOS is 8 months for Avastin and Lomustine and survival rates at 6, 9 and 12 months are 62%, 38%, 26% and 65%, 43%, 30%, respectively). (Taal et al., Lancet Oncol 2014 August; 15(9):943-53.)

In the above participants, patients with IL-4R positive tumors showed a faster time to relapse (10.3 months) following initial diagnosis of GBM when compared to patients with low to no expression of IL-4R (16.7 months) supporting published research showing that the Type 2 IL-4R is a key biomarker for more aggressive forms of GBM. (Kohanbash G, McKaveney K, Sakaki M, Ueda R, Mintz A H, Amankulor N, Fujita M, Ohlfest J R, Okada H. GM-CSF promotes the immunosuppressive activity of glioma-infiltrating myeloid cells through interleukin-4 receptor-α. Cancer Res. 2013 Nov. 1; 73ζ 21):6413-23; and Han J and Puri R K. Analysis of the cancer genome atlas (TCGA) database identifies an inverse relationship between interleukin-13 receptor α1 and α2 gene expression and poor prognosis and drug resistance in subjects with glioblastoma multiforme. J Neurooncol. 2018 February; 136(3):463-474.)

These preliminary data showing longer median survival in MDNA55-treated subjects with positive IL-4R expression are highly encouraging and could help determine which subjects will receive optimal therapeutic benefit from MDNA55 treatment. As the second half of our trial continues to enroll at higher doses of MDNA55, it is expected that more data supporting IL-4R as an important biomarker and immunotherapeutic target for rGBM and to improve the benefit-risk profile for subjects treated with MDNA55 will be obtained. The safety and tolerability of MDNA55 has generally remained within the profile established in previous studies.

### MDNA55-05 Clinical Trial

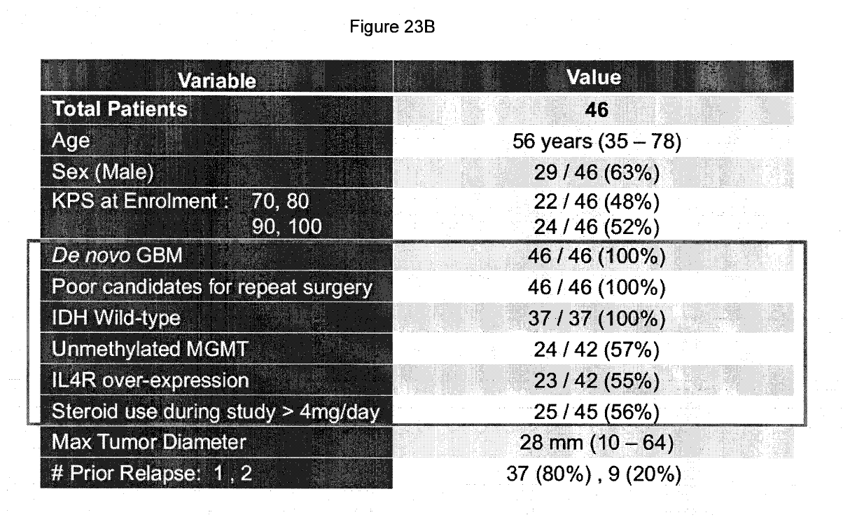

MDNA55-05 is a 46 subject open-label study of MDNA55, an IL-4R directed toxin, in patients with primary (de novo) GBM at first or second relapse/recurrence (including this recurrence) after treatment including surgery and radiotherapy with or without chemotherapy and following discontinuation of any previous standard or investigational lines of therapy. In the study, investigators administer MDNA55 only once directly into the brain tumor using a technique known as Convection Enhanced Delivery (CED). CED allows precision delivery of MDNA55 directly into the tumor tissue and the surrounding healthy brain containing infiltrative tumor cells, while avoiding exposure to the rest of the body. Retrospective analysis of GBM tissue obtained at first diagnosis is performed by immunohistochemistry for IL-4Ra expression. Biopsy samples are categorized based on IL-4Rα expression levels (high or low) and compared against survival outcomes. The study design summary is illustrated in FIG. 23A. Patients demographics enrolled in the clinical study is shown in FIG. 23B.

MDNA55 doses administered range from 18-240 μg. The safety profile of MDNA55 was assessed in patients and the maximum tolerated dose was established to be 240 μg. No death was attributed to MDNA55; no systemic toxicity was found; no clinically significant laboratory abnormalities were found; and MDNA55-related adverse events were primarily neurological/aggravation of pre-existing neurological deficits characteristic with GBM, and had generally manageable with standard measures.

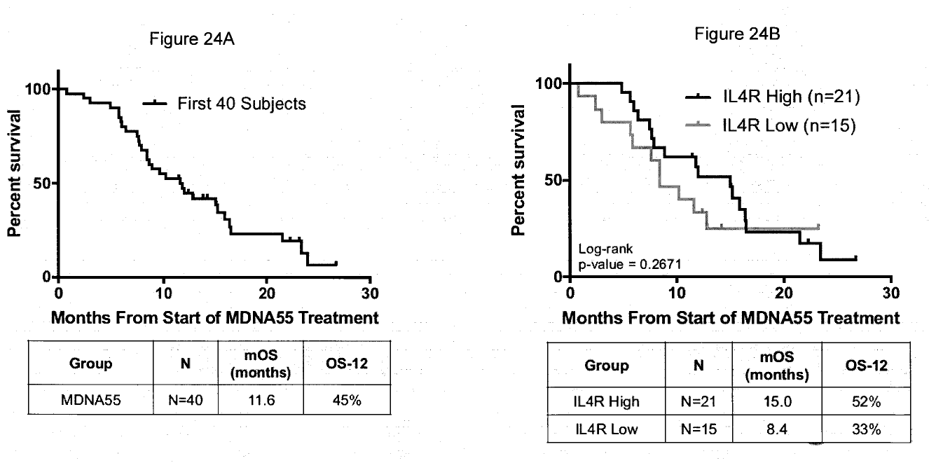

Patient survival rate was measured after the administration of MDNA55. As shown in FIG. 24A, the medium overall survival (mOS) of the first 40 enrolled patients was 11.6 months. The 12-month survival rate (OS-12) was 45%. 36 of the 40 patients were also evaluated for IL-4R expression. As shown in FIG. 24B, the survival rate improved in the patients with high IL-4R expression compared to the patients with low IL-4R expression. The mOS of patients with high IL-4R was 15 months and the OS-12 was 52%. The mOS of patients with low IL-4R was 8.4 months and the OS-12 was 33%. The data cut point was Oct. 31, 2019.

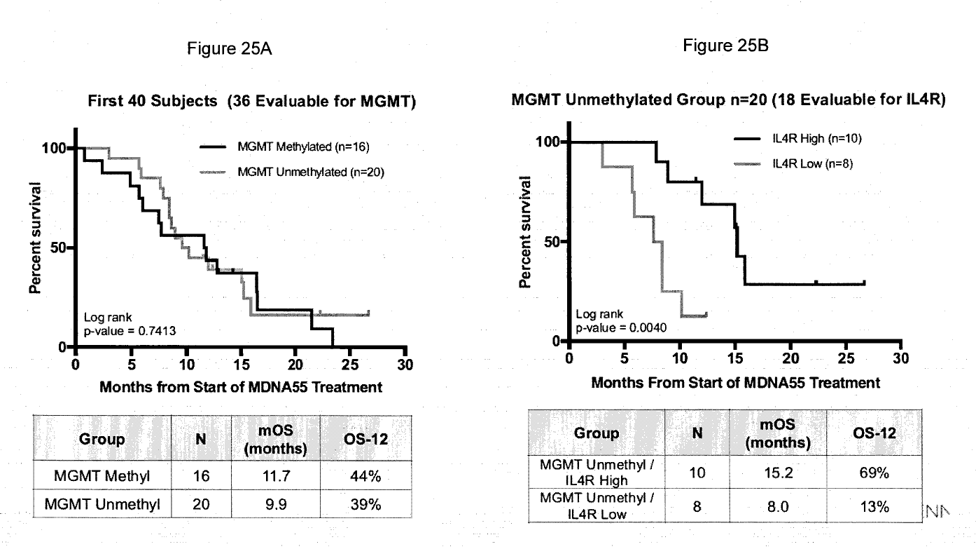

O6-methylguanine-DNA methyltransferase (MGMT) gene promoter methylation status was also evaluated in 36 of the first 40 patients enrolled. 18 patients were found to have methylated MGMT gene promoter, and 20 patients were found to have unmethylated MGMT gene promoter. MGMT gene promoter methylation status had no significant impact on the survival rate after MDNA55 treatment (FIG. 25A). Furthermore, the IL-4R expression level was evaluated in 18 out of the 20 patients who had unmethylated MGMT gene promoter. As shown in FIG. 25B, the patient with unmethylated MGMT but high IL-4R expression level showed significantly improved survival compare to the patients with unmethylated MGMT and low IL-4R expression level.

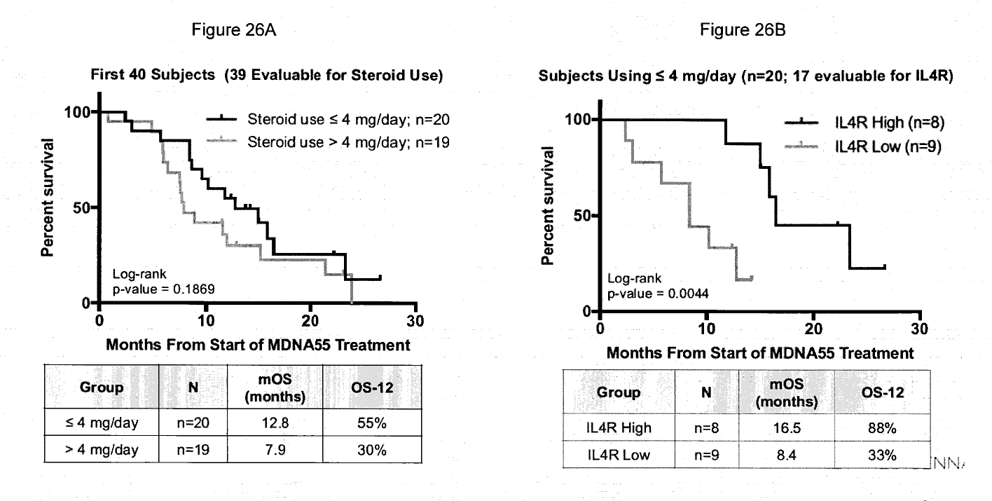

Some of the patients enrolled in the clinical trial used steroid during the treatment with MDNA55. Out of the 39 patients, 19 used higher than 4 mg/day of steroid, and 20 used lower or equal to 4 mg/day of steroid. As shown in FIG. 26A, lower steroid use (e.g., < or =4 mg/day) is associated with longer survival after MDNA55 treatment. Out of the 20 patients who used < or =4 mg/day of steroid, patients with high IL-4R expression level showed improved survival compare to the patients with low IL-4R expression level (FIG. 26B).

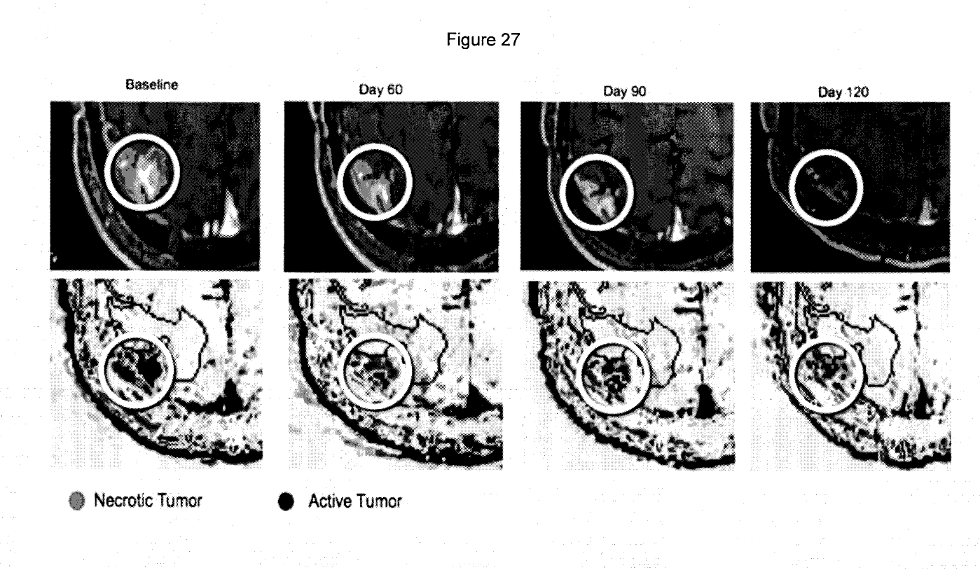

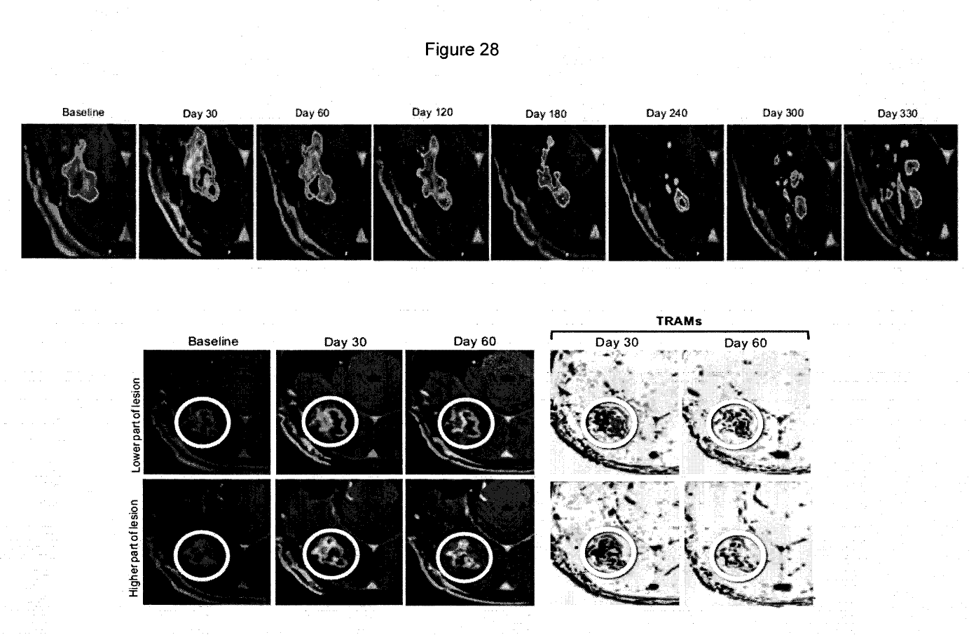

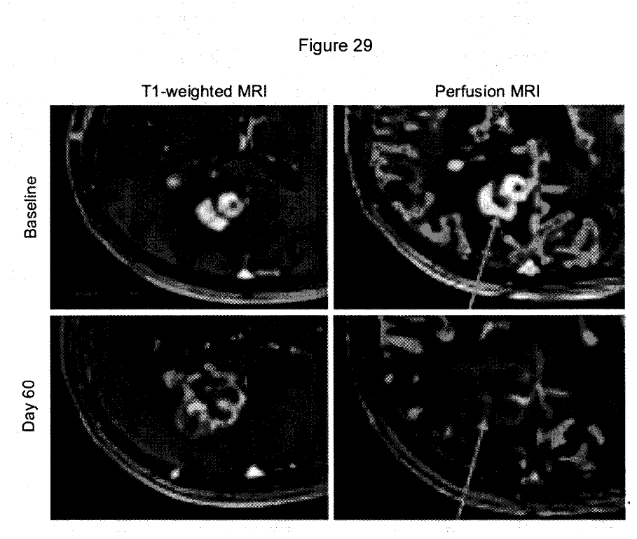

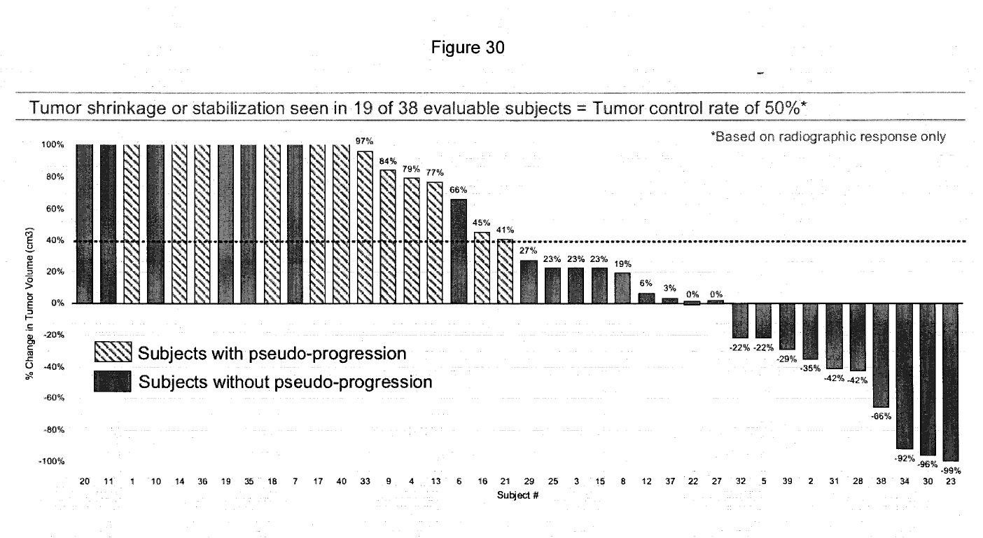

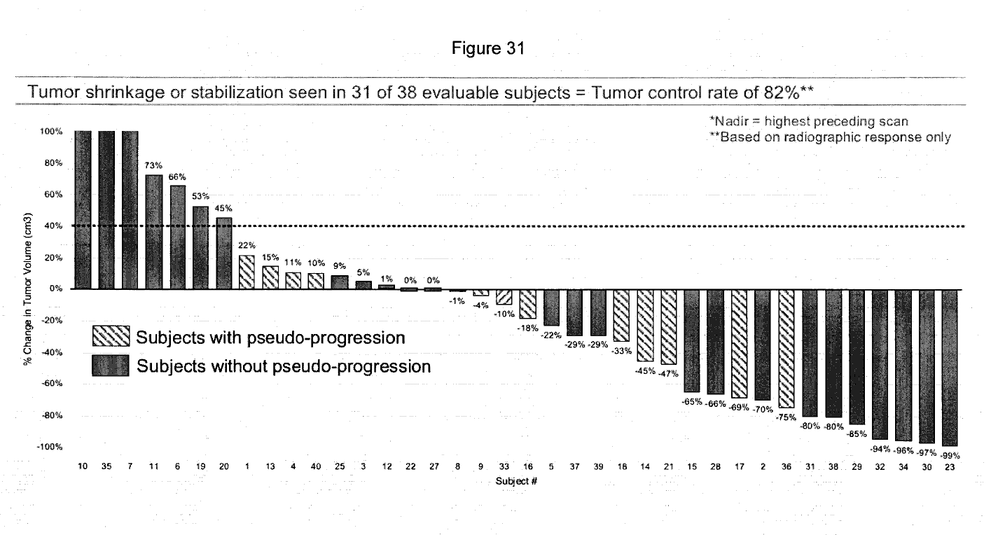

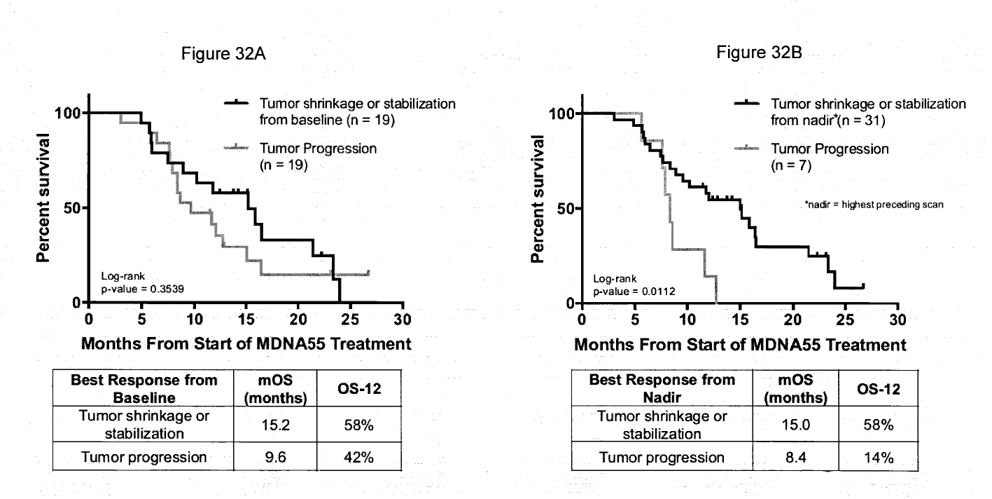

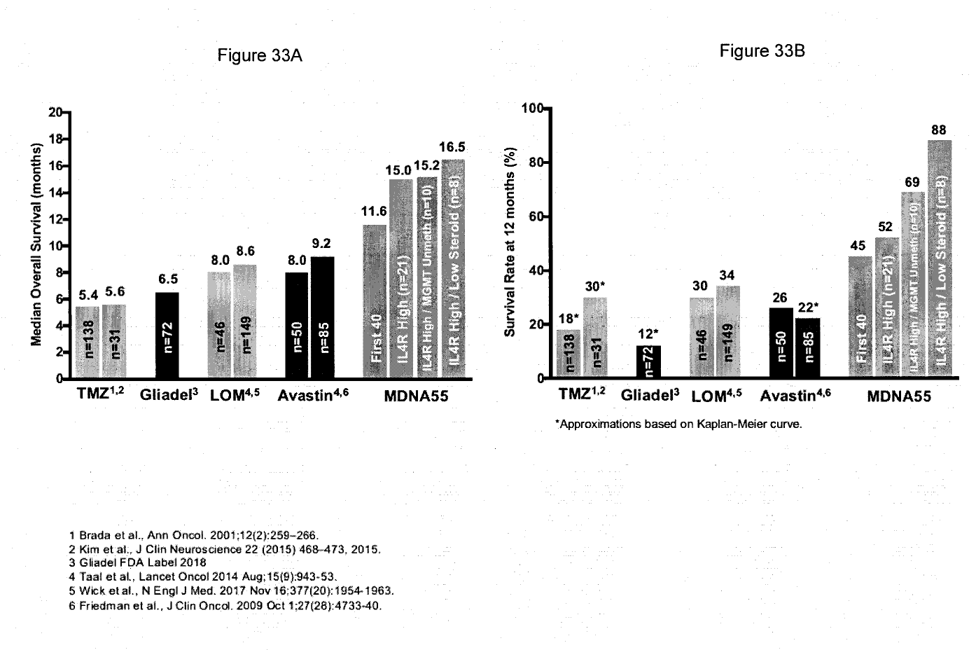

The timeline of response after MDNA55 treatment varies among patients. FIG. 27, FIG. 28 and FIG. 29 show exemplary early or late onset of response after the treatment with MDNA55. 50%-82% of the patients treated with MDNA55 showed shrinked or stabilized tumor volume (FIGS. 30-31). Furthermore, tumor volume shrinkage and stabilization is associated with higher survival rate (FIG. 32A-32B).

Overall, MDNA55 has better efficacy in treating GBM (including recurrent GBM) patients compared to existing therapies including Temozolomide (TMZ), Carmustine (brand name Gliadel®), lomustine (LOM), bevacizumab (brand name Avastin®), especially for GBM patients expressing high level of IL-4R (FIG. 33A-33B). Furthermore, MDNA55 is effective in combination with low doses of steroid to treat GBM (including recurrent GBM) patients.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for determining a cancer patient population for treatment with an IL-4 targeted cargo protein, the method comprising:
a) measuring the level of IL-4 receptor (IL-4R) expression in a biological sample obtained from a cancer or tumor in the cancer patient,
b) quantitating the measurement of the level of IL-4R expression in the biological sample, and
c) treating the cancer patient with an IL-4 targeted cargo protein when the level of IL-4R expression is moderate or high.

2. (canceled)

3. A method for altering the regimen of treatment for a patient with cancer, the method comprising:
a) measuring the level of IL-4 receptor (IL-4R) expression in a biological obtained from a cancer or tumor in the cancer patient,
b) quantitating the measurement of the level of IL-4R expression in the biological sample, wherein a moderate or high level of IL-4R expression is indicative of treatment efficacy,
c) correlating the level of IL-4R expression with the efficacy of treatment, wherein a high level of IL-4R expression is indicative of altering the treatment regimen for treatment with an IL-4 targeted cargo protein, and
d) altering the treatment regimen to include an IL-4 targeted cargo protein when a moderate or high level of IL-4R expression is measured.

4. A method for predicting or determining cancer disease prognosis and/or progression, the method comprising:
a) measuring the level of IL-4 receptor (IL-4R) expression in a biological sample from a tumor in the cancer patient,
b) quantitating the measurement of the level of IL-4R expression in the biological sample, wherein a moderate or high level of IL-4R expression is indicative of the disease prognosis and/or progression, and
c) correlating the level of IL-4R expression with the disease prognosis and/or progression, wherein a moderate or high level of IL-4R expression is indicative of severe disease prognosis and/or progression;
wherein when a high level of IL-4R expression is measured, the method further comprises treating the cancer patient with an IL-4 targeted cargo protein.

5.-9. (canceled)

10. The method according to claim 4, wherein a moderate level of IL-4R expression is indicated by H-Scores from 76 to 150.

11. The method according to claim 4, wherein a high level of IL-4R expression is indicated by H-Scores from 151 to 225.

12. The method according to claim 4, wherein a high level of IL-4R expression is indicated by H-Scores from 226 to 300.

13. (canceled)

14. The method according to claim 4, wherein the level of IL-4R expression is the level of Type 2 IL-4R (Type II IL-R4, comprising IL-4Rα and IL13Rα1) expression.

15. (canceled)

16. The method according to claim 4, wherein the cancer or tumor is selected from the group consisting of prostate cancer, ovarian cancer, breast cancer, endometrial cancer, multiple myeloma, melanoma, lymphomas, lung cancers including small cell lung cancer, kidney cancer, liver cancer, colon cancer, colorectal cancer, pancreatic cancer, gastric cancer, and brain cancer and CNS tumors.

17. The method according to claim 4, wherein the CNS tumor is selected from the group consisting of glioma, glioblastoma, glioblastoma multiforme (GBM), refractory glioblastoma multiforme (rGBM), astrocytoma, medulloblastoma, craniopharyogioma, ependymoma, pinealoma, hemangioblastoma, acoustic neuroma, oligodendroglia, menangioma, meningioma, neuroblastoma, retinoblastoma, medulloblastoma, adult pituitary adenoma, an O6-methylguanine-methyltransferase (MGMT) positive or negative CNS tumor, and furin positive CNS tumor.

18.-22. (canceled)

23. The method according to claim 4, wherein the IL-4 targeted cargo protein comprises a toxin, wherein the toxin comprises a bacterial toxin, animal toxin, or plant toxin.

24. (canceled)

25. The method of claim 23, wherein the toxin comprises a pore-forming toxin.

26.-27. (canceled)

28. The method of claim 23, wherein the bacterial toxin comprises a toxin selected from the group consisting of Pseudomonas exotoxin, cholera toxin, or diphtheria toxin.

29. The method according to claim 4, wherein the IL-4 targeted cargo protein comprises pro-apoptosis member of the BCL-2 family selected from the group consisting of BAX, BAD, BAT, BAK, BIK, BOK, BID BIM, BMF, and BOK.

30. The method according to claim 4, wherein the IL-4 targeted cargo protein comprises MDNA55 (SEQ ID NO:65) or a derivative or variant thereof.

31. (canceled)

32. The method according to claim 4, wherein the IL-4 targeted cargo protein comprises in IL-4R antibody as the targeting moiety.

33. (canceled)

34. The method according to claim 4, wherein the IL-4 targeted cargo protein comprises a fusion protein.

35.-36. (canceled)

37. The method according to claim 4, wherein the IL-4 targeted cargo protein is formulation in an artificial cerebral spinal fluid (CSF) solution and albumin, wherein the formulation is co-administered with a surrogate tracer to a subject in need thereof.

38.-39. (canceled)

40. The method of claim 4, wherein the surrogate tracer is selected from the group consisting of gadolinium-diethylenetriamine pentaacetic acid (Gd-DTPA) and gadolinium-bound albumin (Gd-albumin).

41.-50. (canceled)

51. The method according to claim 4, wherein the IL-4 targeted cargo protein is administered as a single dose of about 1.5 μg/mL to about 3 g/mL.

52.-59. (canceled)

60. A kit comprising an IL-4 targeted cargo protein as described in claim 4, wherein the kit comprises an IL-4R antibody, instructions for using the IL-4R antibody in an immunohistochemistry (IHC)-based assay, and instructions for determining the percent score or the H-Score.


SEQUENCE LISTING (133 sequences)

SEQ ID NO: 1 - protein, 113 aa, Artificial Sequence
  IL-13 wildtype
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 113 aa, Artificial Sequence
  IL-13 variant
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 3 - protein, 113 aa, Artificial Sequence
  IL-13 variant
PGPVPPSTAIRELIEELINITQNQKAPLCNGSMVWSINLTAGMYCAALESLINVSGCSAIEKTQRMLSGFCPHKVSAGQFSSLHVRGSKIEVAQFVKDLLHHLRALFREGQFN

SEQ ID NO: 4 - protein, 113 aa, Artificial Sequence
  IL-13 variant
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 5 - protein, 113 aa, Artificial Sequence
  IL-13 variant
PGPVPPSTALIELIEELINITQNQKAPLCNGSMVWSINLTAGIYCAALESLINVSGCSAIEKTQRMLSGFCPHKVSAGQFSSLHVKGSKIEVAQFVKDLLHHLRALMREGQFN

SEQ ID NO: 6 - protein, 113 aa, Artificial Sequence
  IL-13 variant
PGPVPPSTAIRELIEELLNITQNQKAPLCNGSMVWSINLTAGMYCAALESLINVSGCSAIEKTQRMLSGFCPHKVSAGQFSSLHVMKSKIEVAQFVKDLLHHLRALFREGQFN

SEQ ID NO: 7 - protein, 113 aa, Artificial Sequence
  IL-13 variant
PGPVPPSTAIRELIEELINITQNQKAPLCNGSMVWSINLTAGMYCAALESLINVSGCSAIEKTQRMLSGFCPHKVSAGQFSSLHVRSSRIEVAQFVKDLLHHLRTLFREGQFN

SEQ ID NO: 8 - protein, 113 aa, Artificial Sequence
  IL-13 variant
PGPVPPSTALRELIEELINITQNEKAPLCNGSMVWSINLTAGIYCAALESLINVSGCSAIEKTQRMLSGFCPHKVSAGQFSSLHVTGSKIEVAQFVKDLLYHLRALFREGQFN

SEQ ID NO: 9 - protein, 113 aa, Artificial Sequence
  IL-13 variant
PGPVPPSTALSELIEELINITQNQKAPLCNGSMVWSINPTAGMYCAALESLINVSGCSAIEKTQRMLSGFCPHKVAAGQFSSLHDKGSMIEVAQFVKDLLYHLRTLFREGQFN

SEQ ID NO: 10 - protein, 113 aa, Artificial Sequence
  IL-13 variant
PGPVPPSTATRELIEELINITQNQKAPLCNGSMVWSINLTADMYCAALESLINVSGCSAIEKTQRMLSGFCPHKVSVGQFSSLHVRGSKIEVAQFVKDLLYHLRTLFREGQFN

SEQ ID NO: 11 - protein, 113 aa, Artificial Sequence
  IL-13 variant
PGPVPPSTADIELIAELINITQNQKAPLCNGSMVWSINLTADMYCAALESLINVSGCSAIEKTQRMLSGFCPHKVSAGQFSSLHVKKTRIEVAQFVKDLLLHLKKLFKEGQFN

SEQ ID NO: 12 - protein, 113 aa, Artificial Sequence
  IL-13 variant
PGPVPPSTAARELIEELVNITQNQKAPLCNGSMVWSINLTAGMYCAALESLINVSGCSAIEKTQRMLSGFCPHKVSAGQLSSLHVTGKRIEVAQFVKDLLNHLRALFKEGQFN

SEQ ID NO: 13 - protein, 113 aa, Artificial Sequence
  IL-13 variant
PGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 14 - protein, 113 aa, Artificial Sequence
  IL-13 variant
PGPVPPSTALSELMEELVNITQNQKAPLCNGSMVWSINLTAGMYCAALESLINVSGCSAIEKTQRMLSGFCPHKVSAGQFSSLHVRDSKIEVAQFVKDLLNHLKALFKEGQFN

SEQ ID NO: 15 - protein, 112 aa, Artificial Sequence
  IL-13 variant
GPVPPSTAFRELIEELVNITQNQKAPLCNGSMVWSINLTAGMYCAALESLINVSGCSAIEKTQRMLSGFCPHKVSPGQFSSLHVTNSRIEVAQFVKDLLNHLKALFKEGQYN

SEQ ID NO: 16 - protein, 112 aa, Artificial Sequence
  IL-13 variant
GPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 17 - protein, 113 aa, Artificial Sequence
  IL-13 variant
PGPVPPSTAHLELIEELINITQNQKAPLCNGSMVWSINPTAGMYCAALESLINVSGCSAIEKTQRMLSGFCPHKVSAGQFSSLHVMDTRIEVAQFVKDLLLHLKKLFKEGQFN

SEQ ID NO: 18 - protein, 113 aa, Artificial Sequence
  IL-13 variant
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 19 - protein, 113 aa, Artificial Sequence
  IL-13 variant
PGPVPPSTAHRELIEELVNITQNQKAPLCNGSMVWRINRTAGMYCAALESLINVSGCSAIEKTQRMLSGFCPHKVSAGQFSSLHVMDSRIEVAQFVKDLLNHLRALFKEGQFN

SEQ ID NO: 20 - protein, 113 aa, Artificial Sequence
  IL-13 variant
PGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 21 - protein, 113 aa, Artificial Sequence
  Exemplary sequence comprising R11I, V18I, R86K, D87G, T88S, L101H, K104R, K105A, F107M, referred to herein as A5
PGPVPPSTALIELIEELINITQNQKAPLCNGSMVWSINLTAGMYCAALESLINVSGCSAIEKTQRMLSGFCPHKVSAGQFSSLHVKGSKIEVAQFVKDLLHHLRALMREGQFN

SEQ ID NO: 22 - protein, 113 aa, Artificial Sequence; identical to SEQ ID NO: 6
  Exemplary sequence comprising L10I, V18L, R86M, D87K, T88S, L101H, K104R, K105A, referred to herein as A6

SEQ ID NO: 23 - protein, 113 aa, Artificial Sequence; identical to SEQ ID NO: 3
  Exemplary sequence comprising L10I, V18I, D87G, T88S, L101H, K104R, K105A, referred to herein as A7

SEQ ID NO: 24 - protein, 113 aa, Artificial Sequence; identical to SEQ ID NO: 7
  Exemplary sequence comprising L10I, V18I, D87S, T88S, K89R, L101H, K104R, K105T; referred to herein as A8

SEQ ID NO: 25 - protein, 113 aa, Artificial Sequence
  Exemplary sequence comprising L10V, V18I, D87S, T88S, L101F, K104R, K105T, referred to herein as A11 variant 1
PGPVPPSTAVRELIEELINITQNQKAPLCNGSMVWSINLTAGMYCAALESLINVSGCSAIEKTQRMLSGFCPHKVSAGQFSSLHVRSSKIEVAQFVKDLLFHLRTLFREGQFN

SEQ ID NO: 26 - protein, 113 aa, Artificial Sequence
  Exemplary sequence comprising V18I, R86T, D87G, T88S, L101Y, K104R, K105A, referred to herein as B2
PGPVPPSTALRELIEELINITQNQKAPLCNGSMVWSINLTAGMYCAALESLINVSGCSAIEKTQRMLSGFCPHKVSAGQFSSLHVTGSKIEVAQFVKDLLYHLRALFREGQFN

SEQ ID NO: 27 - protein, 113 aa, Artificial Sequence
  Exemplary sequence comprising R11S, V18I, R86K, D87G, T88S, K89M, L101Y, K104R, K105T, referred to herein as B4
PGPVPPSTALSELIEELINITQNQKAPLCNGSMVWSINLTAGMYCAALESLINVSGCSAIEKTQRMLSGFCPHKVSAGQFSSLHVKGSMIEVAQFVKDLLYHLRTLFREGQFN

SEQ ID NO: 28 - protein, 113 aa, Artificial Sequence
  Exemplary sequence comprising L10T, V18I, D87G, T88S, K89K, L10Y1, K104R, K105T, referred to herein as B6
PGPVPPSTATRELIEELINITQNQKAPLCNGSMVWSINLTAGMYCAALESLINVSGCSAIEKTQRMLSGFCPHKVSAGQFSSLHVRGSKIEVAQFVKDLLYHLRTLFREGQFN

SEQ ID NO: 29 - protein, 113 aa, Artificial Sequence
  Exemplary sequence comprising L10D, R11I, V18I, R86K, D87K, K89R, R108K, referred to herein as C2
PGPVPPSTADIELIEELINITQNQKAPLCNGSMVWSINLTAGMYCAALESLINVSGCSAIEKTQRMLSGFCPHKVSAGQFSSLHVKKTRIEVAQFVKDLLLHLKKLFKEGQFN

SEQ ID NO: 30 - protein, 113 aa, Artificial Sequence
  Exemplary sequence comprising L10A, R86T, D87G, T88K, K89R, L101N, K104R, K105A, R108K, referred to herein as C3
PGPVPPSTAARELIEELVNITQNQKAPLCNGSMVWSINLTAGMYCAALESLINVSGCSAIEKTQRMLSGFCPHKVSAGQFSSLHVTGKRIEVAQFVKDLLNHLRALFKEGQFN

SEQ ID NO: 31 - protein, 113 aa, Artificial Sequence; identical to SEQ ID NO: 13
  Exemplary sequence comprising L10V, K89R, L101N, K105E, R108T, referred to herein as C4

SEQ ID NO: 32 - protein, 113 aa, Artificial Sequence; identical to SEQ ID NO: 14
  Exemplary sequence comprising R11S, I14M, T88S, L101N, K105A, R108K, referred to herein as C7

SEQ ID NO: 33 - protein, 113 aa, Artificial Sequence
  Exemplary sequence comprising L10H, R11L, V18I, R86K, D87E, K89R, L101N, K105T, R108K, refered to herein as C9
PGPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 34 - protein, 113 aa, Artificial Sequence
  Exemplary sequence comprising L10H, R11L, V18I, R86M, K89R, R108K, referred to herein as C10
PGPVPPSTAHLELIEELINITQNQKAPLCNGSMVWSINLTAGMYCAALESLINVSGCSAIEKTQRMLSGFCPHKVSAGQFSSLHVMDTRIEVAQFVKDLLLHLKKLFKEGQFN

SEQ ID NO: 35 - protein, 113 aa, Artificial Sequence; identical to SEQ ID NO: 18
  Exemplary sequence comprising L10H, R86T, D87G, T88R, R108K, referred to herein as C11

SEQ ID NO: 36 - protein, 113 aa, Artificial Sequence
  Exemplary sequence comprising L10H, R86M, T88S, K89R, L101N, K104R, K105A, R108K, referred to herein as C12
PGPVPPSTAHRELIEELVNITQNQKAPLCNGSMVWSINLTAGMYCAALESLINVSGCSAIEKTQRMLSGFCPHKVSAGQFSSLHVMDSRIEVAQFVKDLLNHLRALFKEGQFN

SEQ ID NO: 37 - protein, 113 aa, Artificial Sequence
  Exemplary sequence comprising L10A, V18F, R86F, D87F, K89R, L101I, K104R, R108K, referred to herein as D7
PGPVPPSTAARELIEELFNITQNQKAPLCNGSMVWSINLTAGMYCAALESLINVSGCSAIEKTQRMLSGFCPHKVSAGQFSSLHVKKTRIEVAQFVKDLLIHLRKLFKEGQFN

SEQ ID NO: 38 - protein, 113 aa, Artificial Sequence
  Exemplary sequence comprising L10V, E12A, V18I, R65D, D87S, T88S, L101F, K104R, K105T, referred to herein as IL-13dn
PGPVPPSTAVRALIEELINITQNQKAPLCNGSMVWSINLTAGMYCAALESLINVSGCSAIEKTQDMLSGFCPHKVSAGQFSSLHVRSSKIEVAQFVKDLLFHLRTLFREGQFN

SEQ ID NO: 39 - protein, 146 aa, Artificial Sequence
  Signal Peptide
MHPLLNPLLLALGLMALLLTTVIALTCLGGFASPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 40 - protein, 113 aa, Artificial Sequence; identical to SEQ ID NO: 2
  Exemplary sequence comprising L10V, E12A, V18I, R65D, D87S, T88S, L101F, K104R, K105T, referred to herein as IL-13DN variant 1

SEQ ID NO: 41 - protein, 113 aa, Artificial Sequence; identical to SEQ ID NO: 38
  Exemplary sequence comprising L10V, E12A, V18I, R65D, D87S, T88S, L101F, K104R, K105T, referred to herein as IL-13DN variant 2

SEQ ID NO: 42 - protein, 114 aa, Artificial Sequence
  wild-type IL-13 including an additional methionine at the N-terminus
MPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 43 - protein, 117 aa, Artificial Sequence
  circularly permuted IL-13
MYCAALESLINVSGCSAIEKTQRMLSGFCPHKVSAGQFSSLHVRDTKIEVAQFVKDLLLHLKKLFREGQFNGGSGPGPVPPSTALRELIEELVNITQNQKAPLCNGSMVWSINLTAG

SEQ ID NO: 44 - protein, 118 aa, Artificial Sequence
  Circularly permuted IL-13
MYCAALESLINVSGCSAIEKTQRMLSGFCPHKVSAGQFSSLHVRDTKIEVAQFVKDLLLHLKKLFREGQFNGGSGMPGPVPPSTALRELIEELVNITQNQKAPLCNGSMVWSINLTAG

SEQ ID NO: 45 - protein, 117 aa, Artificial Sequence
  circularly permuted IL-13 “A11” variant
MYCAALESLINVSGCSAIEKTQRMLSGFCPHKVSAGQFSSLHVRSSKIEVAQFVKDLLFHLRTLFREGQFNGGSGPGPVPPSTAVRELIEELINITQNQKAPLCNGSMVWSINRTAG

SEQ ID NO: 46 - protein, 118 aa, Artificial Sequence
  circularly permuted IL-13
MYCAALESLINVSGCSAIEKTQRMLSGFCPHKVSAGQFSSLHVRSSKIEVAQFVKDLLFHLRTLFREGQFNGGSGMPGPVPPSTAVRELIEELINITQNQKAPLCNGSMVWSINRTAG

SEQ ID NO: 47 - protein, 117 aa, Artificial Sequence
  circularly permuted IL-13 “DN” variant
MYCAALESLINVSGCSAIEKTQDMLSGFCPHKVSAGQFSSLHVRSSKIEVAQFVKDLLFHLRTLFREGQFNGGSGPGPVPPSTAVRALIEELINITQNQKAPLCNGSMVWSINLTAG

SEQ ID NO: 48 - protein, 118 aa, Artificial Sequence
  circular permuted IL-13
MYCAALESLINVSGCSAIEKTQDMLSGFCPHKVSAGQFSSLHVRSSKIEVAQFVKDLLFHLRTLFREGQFNGGSGMPGPVPPSTAVRALIEELINITQNQKAPLCNGSMVWSINLTAG

SEQ ID NO: 49 - protein, 153 aa, Artificial Sequence [sequence omitted]

SEQ ID NO: 50 - protein, 130 aa, Artificial Sequence
  IL-4 including an additional methionine at the N-terminus“ starting
MHKCDITLQEIIKTLNSLTEQKTLCTELTVTDIFAASKDTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 51 - protein, 128 aa, Artificial Sequence
  KFR
KCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSS

SEQ ID NO: 52 - protein, 134 aa, Artificial Sequence
  RGA
MDTTEKETFCRAATVLRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAAS

SEQ ID NO: 53 - protein, 134 aa, Artificial Sequence
  cirularly permuted wild-type IL-4
MDTTEKETFCRAATVLRQFYSHHEKDTRCLGATAQQFHRHKQLIRFLKRLDRNLWGLAGLNSCPVKEANQSTLENFLERLKTIMREKYSKCSSGGNGGHKCDITLQEIIKTLNSLTEQKTLCTELTVTDIFAAS

SEQ ID NO: 54 - protein, 252 aa, Artificial Sequence
  circularly permuted ”KFR“ IL-4 variant
MDTTEKETFCRAATVLRQFYSHHEKDTRCLGATAQQFHRHKQLIRFLKRLDRNLWGLAGLNSCPVKEANQSTLENFLERLKTIMKEKFRKCSSGGNGGHKCDITLQEIIKTLNSLTEQKTLCTELTVTDIFAASRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAAS

SEQ ID NO: 55 - protein, 133 aa, Artificial Sequence
  circularly permuted ”KF“ IL-4 variant
MDTTEKETFCRAATVLRQFYSHHEKDTRCLGATAQQFHRHKQLIRFLKRLDRNLWGLAGLNSCPVKEANQSTLENFLERLKTIMKEKFKCSSGGNGGHKCDITLQEIIKTLNSLTEQKTLCTELTVTDIFAAS

SEQ ID NO: 56 - protein, 286 aa, Artificial Sequence
  IL13-BAD (targeting IL13Ra2; referred to as C11; GGGGS linker)
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFNGGGGSMFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 57 - protein, 286 aa, Artificial Sequence
  A11-BAD (A11 is an IL13Ra1 agonist; GGGGS linker)
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFNGGGGSMFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 58 - protein, 301 aa, Artificial Sequence
  KFR-BAD (KFR targets Type 2 IL-4R; GGGGS linker)
KCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSSGGGGSMFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 59 - protein, 309 aa, Artificial Sequence
  pKFR4-Bad-H6
MDTTEKETFCRAATVLRQFYSHHEKDTRCLGATAQQFHRHKQLIRFLKRLDRNLWGLAGLNSCPVKEANQSTLENFLERLKTIMKEKFRKCSSGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQHHHHHH

SEQ ID NO: 60 - protein, 303 aa, Artificial Sequence
  cpKFR4-Bad fusion; GS linker
MDTTEKETFCRAATVLRQFYSHHEKDTRCLGATAQQFHRHKQLIRFLKRLDRNLWGLAGLNSCPVKEANQSTLENFLERLKTIMKEKFRKCSSGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 61 - protein, 303 aa, Artificial Sequence
  cpIL4-BAD; GS linker
MDTTEKETFCRAATVLRQFYSHHEKDTRCLGATAQQFHRHKQLIRFLKRLDRNLWGLAGLNSCPVKEANQSTLENFLERLKTIMREKYSKCSSGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 62 - protein, 309 aa, Artificial Sequence
  cpIL-4-BAD H6; GS linker
MDTTEKETFCRAATVLRQFYSHHEKDTRCLGATAQQFHRHKQLIRFLKRLDRNLWGLAGLNSCPVKEANQSTLENFLERLKTIMREKYSKCSSGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQHHHHHH

SEQ ID NO: 63 - protein, 295 aa, Artificial Sequence
  IL13-BAD (targets IL13Ra1 and is referred to as IL13DN)
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFNGGGGSGGGGSGGGGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 64 - protein, 366 aa, Artificial Sequence
  IL-4-BclxL; GGGGS linker
KCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 65 - protein, 485 aa, Artificial Sequence
  MDNA55
MDTTEKETFCRAATVLRQFYSHHEKDTRCLGATAQQFHRHKQLIRFLKLRDRNLWGLAGLNSCPVKEANQSTLENFLERLKTIMREKYSKCSSGGNGGHKCDITLQEIIKTLNSLTEQKTLCTELTVTDIFAASKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 66 - protein, 309 aa, Artificial Sequence
  cpS4-Bad-H6
MDTTEKETFCRAATVLRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQHHHHHH

SEQ ID NO: 67 - protein, 303 aa, Artificial Sequence
  cpS4-Bad
MDTTEKETFCRAATVLRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAASGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 68 - protein, 305 aa, Artificial Sequence
  IL-4-Bad-H6
MHKCDITLQEIIKTLNSLTEQKTLCTELTVTDIFAASKDTTEKETFCRAATVLRQFYSHHEKDTRCLGATAQQFHRHKQLIRFLKRLDRNLWGLAGLNSCPVKEANQSTLENFLERLKTIMREKYSKCSSGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQHHHHHH

SEQ ID NO: 69 - protein, 299 aa, Artificial Sequence
  IL-4-Bad-H6
MHKCDITLQEIIKTLNSLTEQKTLCTELTVTDIFAASKDTTEKETFCRAATVLRQFYSHHEKDTRCLGATAQQFHRHKQLIRFLKRLDRNLWGLAGLNSCPVKEANQSTLENFLERLKTIMREKYSKCSSGSFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 70 - protein, 330 aa, Artificial Sequence [sequence omitted]

SEQ ID NO: 71 - protein, 326 aa, Artificial Sequence
  IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 72 - protein, 377 aa, Artificial Sequence
  IgG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 73 - protein, 327 aa, Artificial Sequence
  IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 168 aa, Artificial Sequence
  BAD amino acid sequence
MFQIPEFEPSEQEDSSSAERGLGPSPAGDGPSGSGKHHRQAPGLLWDASHQQEQPTSSSHHGGAGAVEIRSRHSAYPAGTEDDEGMGEEPSPFRGRSRAAPPNLWAAQRYGRELRRMSDEFVDSFKKGLPRPKSAGTATQMRQSSSWTRVFQSWWDRNLGRGSSAPSQ

SEQ ID NO: 75 - protein, 239 aa, Artificial Sequence
  Bcl-2 amino acid sequence
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 76 - protein, 168 aa, Artificial Sequence
  >HsBAD_Q92934-1(UniProtKB)
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 77 - protein, 192 aa, Artificial Sequence
  >HsBAX_Q07812-1(UniProtKB)
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 78 - protein, 211 aa, Artificial Sequence
  >HsBAK1_Q16611-1(UniProtKB)
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFKS

SEQ ID NO: 79 - protein, 160 aa, Artificial Sequence
  >HsBIK_Q13323-1(UniProtKB)
MSEVRPLSRDILMETLLYEQLLEPPTMEVLGMTDSEEDLDPMEDFDSLECMEGSDALALRLACIGDEMDVSLRAPRLAQLSEVAMHSLGLAFIYDQTEDIRDVLRSFMDGFTTLKENIMRFWRSPNPGSWVSCEQVLLALLLLLALLLPLLSGGLHLLLK

SEQ ID NO: 80 - protein, 195 aa, Artificial Sequence
  >HsBID_P55957-1(UniProtKB)
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 81 - protein, 120 aa, Artificial Sequence
  Variable heavy (vh) domain
EVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVSS

SEQ ID NO: 82 - protein, 447 aa, Artificial Sequence
  Full length HC
EVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 83 - protein, 112 aa, Artificial Sequence
  Variable light (vl) domain
DIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKR

SEQ ID NO: 84 - protein, 218 aa, Artificial Sequence
  Full length light chain
DIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 447 aa, Artificial Sequence
  Full length HC
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 218 aa, Artificial Sequence
  Full length light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 113 aa, Artificial Sequence [sequence omitted]

SEQ ID NO: 88 - protein, 440 aa, Artificial Sequence
  Full length HC
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 108 aa, Artificial Sequence
  Variable light (vl) domain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 90 - protein, 214 aa, Artificial Sequence
  Full length light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 440 aa, Artificial Sequence; identical to SEQ ID NO: 88
  Full length heavy chain

SEQ ID NO: 92 - protein, 214 aa, Artificial Sequence; identical to SEQ ID NO: 90
  Full length light chain

SEQ ID NO: 93 - protein, 117 aa, Artificial Sequence [sequence omitted]

SEQ ID NO: 94 - protein, 107 aa, Artificial Sequence [sequence omitted]

SEQ ID NO: 95 - protein, 444 aa, Artificial Sequence
  Full length heavy chain
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 214 aa, Artificial Sequence
  Full length light chain
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 121 aa, Artificial Sequence
  Heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 98 - protein, 108 aa, Artificial Sequence
  Light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 99 - protein, 447 aa, Artificial Sequence
  Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 214 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLFTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 118 aa, Artificial Sequence
  Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 102 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 103 - protein, 122 aa, Artificial Sequence
  Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 104 - protein, 108 aa, Artificial Sequence; identical to SEQ ID NO: 102
  Light chain

SEQ ID NO: 105 - protein, 118 aa, Artificial Sequence [sequence omitted]

SEQ ID NO: 106 - protein, 108 aa, Artificial Sequence; identical to SEQ ID NO: 102
  Light chain

SEQ ID NO: 107 - protein, 118 aa, Artificial Sequence
  Heavy chain
EVQLVESCGGLVQPGGSLRLSCAASGFTFSDSWIHWVRQAPGKGLEWVAWISPYGGSTYYADSVKGRFTISRDTSKNTAYLQMNSLRAEDTAVYYCARRHWPGGFDYWGQGTLVTVSA

SEQ ID NO: 108 - protein, 118 aa, Artificial Sequence
  Heavy chain
EVQLVESGGGLVQPGGSLRLSCAASGFTFSDSWIHWVRQAPGKGLEWVAWISPYGGSTYYADSVKGRFTISRDTSKNTAYLQMNSLRAEDTAVYYCARRHWPGGFDYWGQGTLVTVSA

SEQ ID NO: 109 - protein, 118 aa, Artificial Sequence
  Heavy chain
EVQLVESGGGLVQPGGSLRLSCAASGFTFSGSWIHWVRQAPGKGLEWVAWILPYGGSSYYADSVKGRFTISRDTSKNTAYLQMNSLRAEDTAVYYCARRHWPGGFDYWGQGTLVTVSA

SEQ ID NO: 110 - protein, 108 aa, Artificial Sequence; identical to SEQ ID NO: 102
  Light chain

SEQ ID NO: 111 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYYNVPWTFGQGTKVEIKR

SEQ ID NO: 112 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYYAPPWTFGQGTKVEIKR

SEQ ID NO: 113 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYYTVPWTFGQGTKVEIKR

SEQ ID NO: 114 - protein, 108 aa, Artificial Sequence
  Light chain
DIQMTQSPSSLSASVGDRVTITCRASQVINTFLAWYQQKPGKAPKLLIYSASTLASGVPSRFSGSGSGTDFTLTISSLQPEDFATYYCQQYYTVPRTFGQGTKVEIKR

SEQ ID NO: 115 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQGYGVPRTFGQGTKVEIKR

SEQ ID NO: 116 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLFTPPTFGQGTKVEIKR

SEQ ID NO: 117 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYFITPTTFGQGTKVEIKR

SEQ ID NO: 118 - protein, 107 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYYTPPTFGQGTKVEIKR

SEQ ID NO: 119 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQFFYTPPTFGQGTKVEIKR

SEQ ID NO: 120 - protein, 450 aa, Artificial Sequence
  Heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 216 aa, Artificial Sequence
  Light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 122 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQSLFTPPTFGQGTKVEIKR

SEQ ID NO: 123 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQSLYTPPTFGQGTKVEIKR

SEQ ID NO: 124 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQSWYHPPTFGQGTKVEIKR

SEQ ID NO: 125 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYFYIPPTFGQGTKVEIKR

SEQ ID NO: 126 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYWYTPYTFGQGTKVEIKR

SEQ ID NO: 127 - protein, 108 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQSYFIPPTFGQGTKVEIKR

SEQ ID NO: 128 - protein, 118 aa, Artificial Sequence; identical to SEQ ID NO: 101
  Heavy chain (VH)

SEQ ID NO: 129 - protein, 106 aa, Artificial Sequence
  Light chain (VL)
DIQMTQSPSSLSASVGDRVTITCRASQDVSTAVAWYQQKPGKAPKWYSASFLYSGVPSRFSGSGSGTDFTLTISSLQPEDFATYYCQQYLYHPATFGQGTKVEIKR

SEQ ID NO: 130 - protein, 448 aa, Artificial Sequence
  Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 214 aa, Artificial Sequence
  Light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 113 aa, Artificial Sequence; identical to SEQ ID NO: 4
  Exemplary sequence comprising L10V, V18I, D87S, T88S, L101F, K104R, K105T, referred to herein as A11 variant 2

SEQ ID NO: 133 - protein, 5 aa, Artificial Sequence
  linker
GGGGS